SEQUENCE LISTING (94 sequences)

SEQ ID NO: 1 - protein, 19 aa, Artificial Sequence
  Signal peptide 1
MGWSCIILFLVATATGVHS

SEQ ID NO: 2 - protein, 22 aa, Artificial Sequence
  Signal peptide 2
MDMRVPAQLLGLLLLWLRGARC

SEQ ID NO: 3 - protein, 214 aa, Artificial Sequence
  antiCD40 antibody light chain
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 233 aa, Artificial Sequence
  antiCD40 antibody light chain with signal peptide 1
MGWSCIILFLVATATGVHSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 236 aa, Artificial Sequence
  antiCD40 antibody light chain with signal peptide 2
MDMRVPAQLLGLLLLWLRGARCDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 451 aa, Artificial Sequence
  antiCD40 antibody heavy chain hIgG2 dK
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 7 - protein, 470 aa, Artificial Sequence
  antiCD40 antibody heavy chain hIgG2 dK with signal peptide 1
MGWSCIILFLVATATGVHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 8 - protein, 473 aa, Artificial Sequence
  antiCD40 antibody heavy chain hIgG2 dK with signal peptide 2
MDMRVPAQLLGLLLLWLRGARCQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 9 - protein, 452 aa, Artificial Sequence
  antiCD40 antibody heavy chain hIgG2
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 471 aa, Artificial Sequence
  antiCD40 antibody heavy chain hIgG2 with signal peptide 1
MGWSCIILFLVATATGVHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - protein, 474 aa, Artificial Sequence
  antiCD40 antibody heavy chain hIgG2 with signal peptide 2
MDMRVPAQLLGLLLLWLRGARCQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 231 aa, Artificial Sequence
  antiCD40 antibody hIgG2 Fab region heavy chain
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVE

SEQ ID NO: 13 - protein, 250 aa, Artificial Sequence
  antiCD40 antibody hIgG2 Fab region heavy chain with signal peptide 1
MGWSCIILFLVATATGVHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVE

SEQ ID NO: 14 - protein, 166 aa, Homo sapiens
  IFN beta
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 15 - protein, 166 aa, Artificial Sequence
  IFN beta C17S
MSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 16 - protein, 166 aa, Artificial Sequence
  IFN beta C17S,N80Q
MSYNLLGFLQRSSNFQSQKLLWQLNGRLEYCLKDRMNFDIPEEIKQLQQFQKEDAALTIYEMLQNIFAIFRQDSSSTGWQETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 17 - protein, 165 aa, Homo sapiens
  IFN alpha 2a
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 18 - protein, 175 aa, Homo sapiens
  IFN lambda 2
VPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 19 - protein, 143 aa, Homo sapiens
  IFN gamma
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 20 - protein, 4 aa, Artificial Sequence
  RL linker
PAPA

SEQ ID NO: 21 - protein, 13 aa, Artificial Sequence
  GST linker
SGGTSGSTSGTGS

SEQ ID NO: 22 - protein, 12 aa, Artificial Sequence
  HL linker
AEAAAKEAAAKA

SEQ ID NO: 23 - protein, 24 aa, Artificial Sequence
  HL2 linker
AEAAAKEAAAKAAEAAAKEAAAKA

SEQ ID NO: 24 - protein, 10 aa, Artificial Sequence
  (G4S)2 linker
GGGGSGGGGS

SEQ ID NO: 25 - protein, 15 aa, Artificial Sequence
  (G4S)3 linker
GGGGSGGGGSGGGGS

SEQ ID NO: 26 - protein, 20 aa, Artificial Sequence
  (G4S)4 linker
GGGGSGGGGSGGGGSGGGGS

SEQ ID NO: 27 - protein, 13 aa, Artificial Sequence
  TEV-6His tag
ENLYFQSHHHHHH

SEQ ID NO: 28 - protein, 392 aa, Artificial Sequence
  antiCD40_LC--HL--IFN beta
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAEAAAKEAAAKAMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 29 - protein, 392 aa, Artificial Sequence
  antiCD40_LC--HL--IFN beta_C17S
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAEAAAKEAAAKAMSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 30 - protein, 621 aa, Artificial Sequence
  antiCD40_hIgG2_dK_HC--RL--IFN beta
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGPAPAMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 31 - protein, 621 aa, Artificial Sequence
  antiCD40_hIgG2_dK_HC--RL--IFN beta_C17S
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGPAPAMSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 32 - protein, 629 aa, Artificial Sequence
  antiCD40_hIgG2_dK_HC--HL--IFN beta
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGAEAAAKEAAAKAMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 33 - protein, 629 aa, Artificial Sequence
  antiCD40_hIgG2_dK_HC--HL--IFN beta_C17S
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGAEAAAKEAAAKAMSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 34 - protein, 384 aa, Artificial Sequence
  antiCD40_LC-- RL--IFN beta
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPAPAMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 35 - protein, 384 aa, Artificial Sequence
  antiCD40_LC--RL--IFN beta_C17S
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPAPAMSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 36 - protein, 393 aa, Artificial Sequence
  antiCD40_LC--GST--IFN beta_C17S
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGTSGSTSGTGSMSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 37 - protein, 404 aa, Artificial Sequence
  antiCD40_LC--HL2--IFN beta_C17S
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAEAAAKEAAAKAAEAAAKEAAAKAMSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 38 - protein, 626 aa, Artificial Sequence
  antiCD40_hIgG2_dK_HC--(G4S)2--IFN alpha 2a
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 39 - protein, 631 aa, Artificial Sequence
  antiCD40_hIgG2_dK_HC--(G4S)3--IFN alpha 2a
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 40 - protein, 636 aa, Artificial Sequence
  antiCD40_hIgG2_dK_HC--(G4S)4--IFN alpha 2a
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 41 - protein, 389 aa, Artificial Sequence
  antiCD40_LC--(G4S)2--IFN alpha 2a
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 42 - protein, 394 aa, Artificial Sequence
  antiCD40_LC--(G4S)3--IFN alpha 2a
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 43 - protein, 399 aa, Artificial Sequence
  antiCD40_LC--(G4S)4--IFN alpha 2a
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 44 - protein, 395 aa, Artificial Sequence
  IFN beta--(G4S)3--antiCD40_LC
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 400 aa, Artificial Sequence
  antiCD40_LC--(G4S)4--IFN beta
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 46 - protein, 636 aa, Artificial Sequence
  IFN beta--(G4S)3--antiCD40_HC_IgG1_NNAS_dK
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNGGGGSGGGGSGGGGSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNNASRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 641 aa, Artificial Sequence
  antiCD40_HC_IgG1_NNAS_dK--(G4S)4--IFN beta
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNNASRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 48 - protein, 456 aa, Artificial Sequence
  antiCD40 antibody hIgG1 heavy chain NNAS
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNNASRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 455 aa, Artificial Sequence
  antiCD40 antibody hIgG1 heavy chain -NNAS-dK
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNNASRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 244 aa, Artificial Sequence
  antiCD40 antibody hIgG2 Fab region heavy chain --TEV--6His tag
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVEENLYFQSHHHHHH

SEQ ID NO: 51 - protein, 107 aa, Artificial Sequence
  antiCD40 antibody VL domain
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 52 - protein, 11 aa, Artificial Sequence
  antiCD40 antibody CDRL1
RASQGIYSWLA

SEQ ID NO: 53 - protein, 7 aa, Artificial Sequence
  antiCD40 antibody CDRL2
TASTLQS

SEQ ID NO: 54 - protein, 9 aa, Artificial Sequence
  antiCD40 antibody CDRL3
QQANIFPLT

SEQ ID NO: 55 - protein, 126 aa, Artificial Sequence
  antiCD40 antibody VH domain
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 56 - protein, 6 aa, Artificial Sequence
  antiCD40 antibody CDRH1
TGYYMH

SEQ ID NO: 57 - protein, 17 aa, Artificial Sequence
  antiCD40 antibody CDRH2
WINPDSGGTNYAQKFQG

SEQ ID NO: 58 - protein, 17 aa, Artificial Sequence
  antiCD40 antibody CDRH3
DQPLGYCTNGVCSYFDY

SEQ ID NO: 59 - protein, 213 aa, Artificial Sequence
  antiCD40 antibody_light chain
EIVMTQSPATLSVSPGERATLSCRASQSVRSNLAWYQQKPGQAPRLLIYGASTRATGIPARFSGSGSGTEFTLTINSLQSEDFAVYYCQQHNKWITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 232 aa, Artificial Sequence
  antiCD40 antibody_light chain with signal peptide 1
MGWSCIILFLVATATGVHSEIVMTQSPATLSVSPGERATLSCRASQSVRSNLAWYQQKPGQAPRLLIYGASTRATGIPARFSGSGSGTEFTLTINSLQSEDFAVYYCQQHNKWITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 447 aa, Artificial Sequence
  antiCD40 antibody_heavy chain hIgG2 dK
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 62 - protein, 466 aa, Artificial Sequence
  antiCD40 antibody_heavy chain hIgG2 dK with Signal peptide 1
MGWSCIILFLVATATGVHSQVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 63 - protein, 227 aa, Artificial Sequence
  antiCD40 antibody Fab region heavy chain hIgG2
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVE

SEQ ID NO: 64 - protein, 246 aa, Artificial Sequence
  antiCD40 antibody Fab region heavy chain hIgG2 with signal peptide 1
MGWSCIILFLVATATGVHSQVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVE

SEQ ID NO: 65 - protein, 240 aa, Artificial Sequence
  antiCD40 antibody Fab region region heavy chain hIgG2--TEV--6His tag
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVEENLYFQSHHHHHH

SEQ ID NO: 66 - protein, 616 aa, Artificial Sequence
  antiCD40_hIgG2_dK_HC--RL--IFN beta dM
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGPAPASYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 67 - protein, 616 aa, Artificial Sequence
  antiCD40_hIgG2_dK_HC--RL--IFN beta dM_C17S
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGPAPASYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 68 - protein, 624 aa, Artificial Sequence
  antiCD40_hIgG2_dK_HC--HL--IFN beta dM
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGAEAAAKEAAAKASYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 69 - protein, 624 aa, Artificial Sequence
  antiCD40_hIgG2_dK_HC--HL--IFN beta dM_C17S
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGAEAAAKEAAAKASYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 70 - protein, 403 aa, Artificial Sequence
  antiCD40_LC--HL2--IFN beta_C17S
EIVMTQSPATLSVSPGERATLSCRASQSVRSNLAWYQQKPGQAPRLLIYGASTRATGIPARFSGSGSGTEFTLTINSLQSEDFAVYYCQQHNKWITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECAEAAAKEAAAKAAEAAAKEAAAKAMSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 71 - protein, 394 aa, Artificial Sequence
  antiCD40_LC--(G4S)3--IFN beta_C17S
EIVMTQSPATLSVSPGERATLSCRASQSVRSNLAWYQQKPGQAPRLLIYGASTRATGIPARFSGSGSGTEFTLTINSLQSEDFAVYYCQQHNKWITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSMSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 72 - protein, 622 aa, Artificial Sequence
  antiCD40_hIgG2_dK_HC--(G4S)2--IFN alpha 2a
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 73 - protein, 627 aa, Artificial Sequence
  antiCD40_hIgG2_dK_HC--(G4S)3--IFN alpha 2a
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 74 - protein, 632 aa, Artificial Sequence
  antiCD40_hIgG2_dK_HC--(G4S)4--IFN alpha 2a
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 75 - protein, 624 aa, Artificial Sequence
  antiCD40_hIgG2_dK_HC--HL--IFN alpha 2a
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGAEAAAKEAAAKACDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 76 - protein, 165 aa, Artificial Sequence
  IFN beta dM
SYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 77 - protein, 165 aa, Artificial Sequence
  IFN beta dM C17S
SYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 78 - DNA, 1947 bp, Artificial Sequence
  Nucleic acid encoding SEQ ID NO 32
atgggctggtcctgcatcattctgtttctggtggccacagccacaggcgtgcactctcaggttcaactggttcagtctggcgccgaagtgaagaaaccaggcgccagcgtgaaggtgtcctgtaaagccagcggctacacctttaccggctactacatgcactgggtccgacaggctccaggacagggacttgagtggatgggctggatcaatcctgacagcggcggcaccaactacgcccagaaattccagggcagagtgaccatgaccagagacaccagcatcagcaccgcctacatggaactgaaccggctgagatccgacgacaccgccgtgtactattgcgccagagatcagcctctgggctactgcacaaatggcgtgtgcagctacttcgactactggggccagggcacactggttacagtgtctagcgcctctacaaagggcccctccgtttttcctctggctccttgttctagaagcaccagcgagtctacagccgctctgggctgtctggtcaaggactactttcctgagcctgtgaccgtgtcctggaatagcggagcactgacatccggcgtgcacacatttccagctgtgctgcagagcagcggcctgtactctctgtctagcgtggtcaccgtgcctagcagcaatttcggcacccagacctacacctgtaacgtggaccacaagcctagcaacaccaaggtggacaagaccgtggaacggaagtgctgcgtggaatgccctccttgtcctgctcctccagtggccggaccttccgtgtttctgttccctccaaagcctaaggacaccctgatgatcagcagaacccctgaagtgacctgcgtggtggtggatgtgtctcacgaggatcccgaggtgcagttcaattggtacgtggacggcgtggaagtgcacaacgccaagaccaagcctagagaggaacagttcaacagcaccttcagagtggtgtccgtgctgaccgtggtgcatcaggactggctgaacggcaaagagtacaagtgcaaggtgtccaacaagggcctgcctgctcctatcgagaaaaccatcagcaagaccaaaggccagcctcgcgagcctcaggtttacacactgcctccaagccgggaagagatgaccaagaatcaggtgtccctgacctgcctcgtgaagggcttctacccttccgatatcgccgtggaatgggagagcaatggccagcctgagaacaactacaagaccacacctcctatgctggacagcgacggctcattcttcctgtacagcaagctgacagtggacaagtccagatggcagcagggcaacgtgttcagctgttctgtgatgcacgaggccctgcacaaccactacacccagaagtctctgtctctgagccctggcgctgaagccgctgctaaagaagctgccgccaaggccatgagctacaacctgctgggctttctgcagcggagcagcaacttccagtgccagaaactgctgtggcagctgaatggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggatagcagcagcaccggatggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctcaaggccaaagagtatagccactgcgcctggaccatcgtgcgcgtggaaatcctgcggaacttctacttcatcaacagactgaccggctacctgcgcaactga

SEQ ID NO: 79 - protein, 174 aa, Artificial Sequence
  IFN omega
LGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 80 - protein, 187 aa, Artificial Sequence
  IFN epsilon
LDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 81 - protein, 628 aa, Artificial Sequence
  antiCD40_hIgG2 dK_HC--HL--IFN alpha 2A
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGAEAAAKEAAAKACDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 82 - protein, 398 aa, Artificial Sequence
  antiCD40_LC-derivative--HL--IFN alpha 2A
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEKSLSLSPGAEAAAKEAAAKACDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 83 - protein, 377 aa, Artificial Sequence
  antiCD40_LC--(G4S)4--IFN gamma
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 84 - protein, 614 aa, Artificial Sequence
  antiCD40_hIgG2 dK_HC--(G4S)4--IFN gamma
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 85 - protein, 409 aa, Artificial Sequence
  antiCD40_LC--(G4S)4--IFN lambda 2
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 86 - protein, 646 aa, Artificial Sequence
  antiCD40_hIgG2 dK_HC--(G4S)4--IFN lambda 2
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSGGGGSVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 87 - protein, 408 aa, Artificial Sequence
  antiCD40_LC--(G4S)4--IFN omega
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 88 - protein, 658 aa, Artificial Sequence
  antiCD40_hIgG2 dK_HC--(G4S)4--IFN epsilon
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSGGGGSLDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 89 - protein, 460 aa, Artificial Sequence
  eVi-5 D1.3-h1v2_hIgG2 dK_HC
MGWSCIILFLVATATGVHSEVKLQESGPGLVAPSQSLSITCTVSGFSLTGYGVNWVRQPPGKGLEWLGMIWGDGNTDYNSALKSRLSISKDNSKSQVFLKMNSLHTDDTARYYCARERDYRLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 90 - protein, 232 aa, Artificial Sequence
  evi-5 D1.3-hk-LC
MGWSCIILFLVATATGVHSIELTQSPASLSASVGETVTITCRASGNIHNYLAWYQQKQGKSPQLLVYYTTTLADGVPSRFSGSGSGTQYSLKINSLQPEDFGSYYCQHFWSTPRTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 635 aa, Artificial Sequence
  eVi-5 D1.3-h1v2_hIgG2 dK_HC--(G4S)2--IFN alpha 2a
MGWSCIILFLVATATGVHSEVKLQESGPGLVAPSQSLSITCTVSGFSLTGYGVNWVRQPPGKGLEWLGMIWGDGNTDYNSALKSRLSISKDNSKSQVFLKMNSLHTDDTARYYCARERDYRLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 92 - protein, 645 aa, Artificial Sequence
  eVi-5 D1.3-h1v2_hIgG2 dK_HC--(G4S)4--IFN alpha 2a
MGWSCIILFLVATATGVHSEVKLQESGPGLVAPSQSLSITCTVSGFSLTGYGVNWVRQPPGKGLEWLGMIWGDGNTDYNSALKSRLSISKDNSKSQVFLKMNSLHTDDTARYYCARERDYRLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSGGGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 93 - protein, 610 aa, Artificial Sequence
  antiCD40 _hIgG2 dK_HC--(G4S)4--IFN gamma
QVQLVESGGGVVQPGKSLRLSCAASGFTFSSNGIHWVRQAPGKGLEWVAVIWSDGSNKFYADSVKGRFTISRDNSKNTLYLQMNSLRAEDTAVYYCARASGSGSYYNFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSGGGGSGGGGSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 94 - protein, 395 aa, Artificial Sequence
  antiCD40_LC--(G4S)3--IFN beta_C17S
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSMSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

(19) United States
(12) Patent Application Publication
(10) Pub. No.: US 2025/0320302 A1
(43) Pub. Date: Oct. 16, 2025

(54) INTERFERON-ASSOCIATED ANTIGEN BINDING PROTEINS FOR USE FOR THE TREATMENT OR PREVENTION OF CORONAVIRUS INFECTION

(71) Applicant: EVOTEC INTERNATIONAL GMBH, Hamburg (DE)

(72) Inventors: Kara CARTER, Saco, ME (US); Grégory Kévin Jean-René NEVEU, Brindas (FR); Marion Rachel France DAJON, Lyon (FR); Xavier MARNIQUET, Lyon (FR); Antoine ALAM, Lyon (FR)

(21) Appl. No.: 18/568,017

(22) PCT Filed: Jun. 8, 2022
(86) PCT No.: PCT/EP2022/065610

§ 371 Date: Dec. 7, 2023

(30) Foreign Application Priority Data
Jun. 9, 2021 (EP) 21305786.2

(51) Int. Cl. C07K 16/28; A61K 39/00; A61P 31/14; C07K 14/56; C07K 14/565

(52) U.S. Cl. CPC C07K 16/2878; A61P 31/14; C07K 14/56; C07K 14/565; A61K 2039/505; C07K 2317/52; C07K 2317/75; C07K 2317/92; C07K 2319/00

(57) ABSTRACT

The present invention relates to methods for treating or preventing Coronavirus infection in a subject by administering a combination of a tumor necrosis factor receptor superfamily (TNFRSF) agonist and an interferon (IFN), for example an IFN-associated antigen binding protein, such as an IFN-fused antibody, or nucleic acids and expression vectors coding therefor. The present invention also relates to the use of corresponding pharmaceutical compositions for use in treating Coronavirus infection.

### CROSS-REFERENCE TO RELATED APPLICATIONS

This application is a national stage application of PCT/EP2022/065610 filed 8 Jun. 2022, which claims priority to European Patent Application No. 21305786.2 filed 9 Jun. 2021, the entire disclosures of each application are herein incorporated by reference.

### SEQUENCE LISTING

The instant application contains a Sequence Listing which has been submitted electronically in ASCII format and is hereby incorporated by reference in its entirety. Said ASCII copy, created on 4 Dec. 2023, is named DFMP-140-PCT-US_Sequence_Listing.txt and is 280,467 bytes in size.

### FIELD OF THE INVENTION

The present invention relates to methods for treating or preventing Coronavirus infection in a subject. The present invention also relates to novel interferon-associated antigen binding proteins as well as nucleic acids and expression vectors encoding such interferon-associated antigen binding proteins for use in therapy, more particularly for use in treating or preventing Coronavirus infection. This includes interferon-fused antibodies or interferon-fused antigen binding fragments thereof, which are also referred to herein as “IFAs”. The present invention also relates to pharmaceutical compositions comprising such interferon-associated antigen binding proteins or nucleic acids or expression vectors for use in therapy, more particularly for use in treating Coronavirus infection. The present invention further provides methods of treatment using such interferon-associated antigen binding proteins or nucleic acids or expression vectors or pharmaceutical compositions. Said novel interferon-associated antigen binding proteins afford beneficial improvements over the current state of the art, for example in that they may effectively rescue cells from Coronavirus-induced cell death and/or from Coronavirus-induced cytopathic effect.

### BACKGROUND

The ongoing coronavirus disease 2019 (COVID-19) global pandemic is caused by the severe acute respiratory syndrome coronavirus 2 (SARS-CoV-2; also known as 2019-nCoV or HCoV-19). Together with SARS-CoV-1, identified in 2003, and Middle East respiratory syndrome coronavirus (MERS-CoV), identified in 2012, SARS-CoV-2 belongs to the β-coronavirus genus within the Coronaviridae family (Zhu et al., N Engl J Med 382(8):727-733 (2020); Jiang et al., Emerg. Microbes Infect. 9, 275-277 (2020); Jiang et al., Lancet 395, 949 (2020); Zhou et al., Nature 579, 270-273 (2020); Zhu et al., N. Engl. J. Med. 382, 727-733 (2020)). SARS-CoV-2 is an enveloped virus, containing a positive sense single-stranded ˜30 kb RNA genome, which encodes 16 nonstructural proteins (nsp1-16), 4 structural proteins [spike (S), envelop (E), membrane (M), and nucleocapsid (N)], and 8 accessory proteins (ORF3a, ORF3b, ORF6, ORF7a, ORF7b, ORF8, ORF9b, and ORF10). The nsps are responsible for viral replication, the structural proteins for virion formation, and the accessory proteins facilitate viral infection, but are not essential for viral replication (Yoshimoto, The Protein Journal 39, 198-216 (2020)).

The rapid international spread of SARS-CoV-2 is associated with numerous mutations that alter viral fitness. Mutations have been documented in all 4 structural proteins encoded by the viral genome. The most prominent mutations are in the spike protein, which mediates entry of the virus into cells by engaging with the angiotensin-converting enzyme 2 (ACE2) receptor (Cai et al., Science 369:1586-92 (2020); Walls et al., Cell 181:281-92. (2020); Lan et al., Nature 581:215-20 (2020); Benton et al., Nature 588:327-30 (2020)). Mutations that emerge in the receptor binding domain (RBD) of the spike protein are especially of interest given their high potential to alter the kinetics and strength of the interaction of the virus with target cells. These mutations could also affect the binding of antibodies capable of binding and blocking engagement of the virus with ACE2. In December 2020, new variants of SARS-CoV-2 carrying several mutations in the spike protein were documented in the UK (SARS-CoV-2 VOC202012/01) and South Africa (501Y.V2) (World Health Organization. Geneva: World Health Organization; (2020); Lauring et al., JAMA 325(6):529-31. (2021)). Early epidemiological and clinical findings have indicated that these variants show increased transmissibility in the population (Davies et al., Estimated transmissibility and severity of novel SARS-CoV-2 Variant of Concern 202012/01 in England. medRxiv. 2021 Feb. 7).

Interferons are among the first cytokines to be upregulated in virus-infected cells and represent key components of the host innate immune system responsible for eliminating the virus at the early stage of infection. SARS-CoV-2 has evolved multiple strategies to prevent interferon release and thus to evade the innate immune response and facilitate viral replication, transmission, and pathogenesis (Xia and Shi, Journal of Interferon & Cytokine Research Volume 40, Number 12 (2020)). Several SARS-CoV-2 nonstructural proteins have been shown to block the interferon pathway including nsp1, nsp3, nsp6, nsp13, nsp 14 and nsp15. Different groups have reported SARS-Cov-2 nsp1 is a potent IFN-I antagonist that significantly decreases >95% expression of IFN-I and ISGs (Lei et al., Nat Commun 11(1) (2020):3810; Xia et al. Cell Rep 33(1):108234. (2020); Yuen et al., Emerg Microbes Infect 9(1):1418-1428. (2020)).

SARS-CoV-2 Nsp3, known as papain-like protease (PLpro), limited IFN-I production by directly cleaving IRF3 or by cleaving the ubiquitin-like protein ISG15 and decreasing the phosphorylated IRF3 resulting in decreased IRF3 activation and IFN-I production (Shin et al., Nature 587(7835):657-662 (2020); Moustaqil et al., Emerg Microbes Infect 10(1):178-195(2021)).

Nsp6 reduces the phosphorylation of STAT1 and STAT2 during IFN-I signaling. Notably, SARS-CoV-2 nsp6 exhibits more efficient suppression of RIG-I-induced IFN-I production and IFN-I-stimulated ISGs production than those nsp6 from SARS-CoV and MERS-CoV do, which confers higher viral replication in an IFN-I-stimulated transient replicon system (Xia et al., Cell Rep 33(1):108234. (2020)).

The helicase nsp13 has a strong inhibitory effect on IFN-I production and signaling. Nsp13 binds to TBK1, leading to decreased phosphorylation of TBK1 and inactivation of IRF3. In addition, nsp13 is identified as a potent antagonist of IFN-I signaling through inhibiting STAT1 and STAT2 activation, resulting in the retention of STAT1 in the cytoplasm and compromised stimulation of ISRE promoter (Lei et al., Nat Commun 11(1):3810. (2020); Xia et al. Cell Rep 33(1):108234 (2020); Yuen et al., Emerg Microbes Infect 9(1):1418-1428 (2020)).

The most common symptoms of infection with SARS-CoV-2 initially are fever, dry cough and tiredness. More severe infection of the lower respiratory tract can lead to more serious symptoms, such as difficulty in breathing or shortness of breath and chest pain or pressure. At this point patients may need to be hospitalized and if the oxygen saturation level of the blood is reduced they will require supplemental oxygen or ventilator support in order to relieve symptoms. Systemic inflammation and serious morbidity or death can follow.

Novel methods for treating and preventing Coronavirus infection, in particular SARS-CoV-2 infection, are needed. In particular, methods for rescuing cells from Coronavirus-induced cell death and from Coronavirus-induced cytopathic effect, in particular from SARS-CoV-2-induced cell death and from SARS-CoV-2-induced cytopathic effect, are needed.

### SUMMARY OF THE INVENTION

In one aspect the invention relates to a tumor necrosis factor receptor superfamily (TNFRSF) agonist or a functional fragment thereof for use in the treatment or prevention of a Coronavirus infection, wherein the TNFRSF agonist or a functional fragment thereof is administered in combination with an interferon (IFN) or a functional fragment thereof.

In another aspect, the invention further relates to an interferon (IFN) or a functional fragment thereof for use in the treatment or prevention of a Coronavirus infection, wherein the IFN or a functional fragment thereof is administered in combination with a tumor necrosis factor receptor superfamily (TNFRSF) agonist or a functional fragment thereof.

In another aspect, the invention also relates to a combination of a tumor necrosis factor receptor superfamily (TNFRSF) agonist or a functional fragment thereof and an interferon (IFN) or a functional fragment thereof, for use in the treatment or prevention of a Coronavirus infection.

In another aspect the invention relates to an interferon-associated antigen binding protein comprising (I) an agonistic anti-CD40 antibody or an agonistic antigen binding fragment thereof, and (II) an Interferon (IFN) or a functional fragment thereof for use in the treatment or prevention of a Coronavirus infection.

According to any of the aspects of the invention, the agonistic anti-CD40 antibody or the agonistic antigen binding fragment thereof may comprise (a) a heavy chain or a fragment thereof comprising a complementarity determining region (CDR) CDRH1 that is at least 90% identical to SEQ ID NO 56, a CDRH2 that is at least 90% identical to SEQ ID NO 57, and a CDRH3 that is at least 90% identical to SEQ ID NO 58; and (b) a light chain or a fragment thereof comprising a CDRL1 that is at least 90% identical to SEQ ID NO 52, a CDRL2 that is at least 90% identical to SEQ ID NO 53, and a CDRL3 that is at least 90% identical to SEQ ID NO 54. Alternatively, the agonistic anti-CD40 antibody or the agonistic antigen binding fragment thereof may comprise (a) a heavy chain or a fragment thereof comprising a complementarity determining region (CDR) CDRH1 that is identical to SEQ ID NO 56, a CDRH2 that is identical to SEQ ID NO 57, and a CDRH3 that is identical to SEQ ID NO 58; and (b) a light chain or a fragment thereof comprising a CDRL1 that is identical to SEQ ID NO 52, a CDRL2 that is identical to SEQ ID NO 53, and a CDRL3 that is identical to SEQ ID NO 54.

According to one embodiment, the agonistic anti-CD40 antibody, or the agonistic antigen binding fragment thereof, comprises a light chain variable region V_{L} comprising the sequence as set forth in SEQ ID NO 51, or a sequence at least 90% identical thereto; and/or a heavy chain variable region V_{H} comprising the sequence as set forth in SEQ ID NO 55, or a sequence at least 90% identical thereto.

According to another embodiment, the agonistic anti-CD40 antibody or the agonistic antigen binding fragment thereof comprises a light chain (LC) that comprises a sequence as set forth in SEQ ID NO 3, or a sequence at least 90% identical thereto; and/or a heavy chain (HC) that comprises a sequence selected from the group consisting of SEQ ID NO 6, SEQ ID NO 9, SEQ ID NO 49 and SEQ ID NO 48, or a sequence at least 90% identical thereto.

According to a further embodiment, the IFN or the functional fragment thereof may be selected from the group consisting of a Type I IFN, a Type II IFN and a Type III IFN, or a functional fragment thereof. Preferably, the type I IFN or the functional fragment thereof is IFNα or IFNβ, or a functional fragment thereof.

According to another embodiment, the IFN or the functional fragment thereof is IFNα2a, or a functional fragment thereof. According to a preferred embodiment, the IFNα2a comprises the sequence as set forth in SEQ ID NO 17, or a sequence at least 90% identical thereto.

According to another embodiment, the IFN or the functional fragment thereof is IFNβ, or a functional fragment thereof. In a preferred embodiment, the IFNβ comprises the sequence as set forth in SEQ ID NO 14, or a sequence at least 90% identical thereto.

According to another embodiment, the IFN or the functional fragment thereof is fused to a light chain of the agonistic anti-CD40 antibody or the agonistic antigen binding fragment thereof, preferably to the C-terminus.

According to a further embodiment, the IFN or the functional fragment thereof is fused to a heavy chain of the agonistic anti-CD40 antibody or the agonistic antigen binding fragment thereof, preferably to the C-terminus.

According to another embodiment, the agonistic anti-CD40 antibody or an agonistic antigen binding fragment thereof, and the IFN or the functional fragment thereof, are fused to each other via a linker. In a preferred embodiment, the linker comprises a sequence as set forth in SEQ ID NO 20, SEQ ID NO 21, SEQ ID NO 24, SEQ ID NO 25 or SEQ ID NO 26.

According to another embodiment, the interferon-associated antigen binding protein is an interferon-fused agonistic anti-CD40 antibody or an interferon-fused agonistic antigen binding fragment thereof comprising one of the sequence combinations disclosed in Table 9, in particular Table 9A or Table 9B, more particularly Table 9A.

According to another embodiment, the use comprises administering the interferon-associated antigen binding protein to a subject in need thereof by means of genetic delivery with RNA or DNA sequences encoding the interferon-associated antigen binding protein, or a vector or vector system encoding the interferon-associated antigen binding protein.

According to yet another embodiment, the interferon-associated antigen binding protein is comprised in a pharmaceutical composition.

### BRIEF DESCRIPTION OF THE DRAWINGS

FIG. 1: This schematic drawing depicts exemplary interferon-associated antigen binding protein formats. The interferon-associated antigen binding protein is an interferon-fused agonistic anti-CD40 antibody or an interferon-fused agonistic antigen binding fragment thereof. IFNs are associated via linkers to different positions on the antibody or the antigen binding fragment thereof: N-terminal or C-terminal part of the light chain (LC) or the heavy chain (HC). In particular, IFNs are chosen from Type I, Type II and Type III interferon families.

FIG. 2A depicts an exemplary map of a pcDNA3.1 plasmid encoding SEQ ID NO 32 under the control of the pCMV promoter. The nucleic acid sequence encoding for SEQ ID NO 32 (=SEQ ID NO 78) is also shown at the bottom. Italic: signal peptide sequence; black color: CP870,893 heavy chain coding sequence; underlined: HL linker coding sequence; bold: IFNβ coding sequence.

FIG. 2B shows examples of SDS PAGE in reduced conditions of some IFAs, with IFNα or IFNβ fused either at the heavy chain or the light chain. Migration of the parental CP870,893 is also shown on the left.

FIG. 3A-3B graphically depict a dose dependent effect of a number of IFA molecules with IFNβ fusions on activating the CD40-mediated NFκB pathway reporter assay in HEK-Blue™ CD40L cells. FIG. 3A shows examples of anti-CD40 activities for IFAs with IFNβ fused to the C-terminal part of the heavy chain (HC). FIG. 3B shows examples of anti-CD40 activities for IFAs with IFNβ fused to the N-terminal part of the LC (IFA34) or the HC (IFA36) and the corresponding fusions on the C-terminal part (IFA35 and IFA37). Purification yield of the latter group of IFAs was very low, thus to test their activity, the supernatants from HEK transfected cells were used and serially diluted to evaluate the anti-CD40 activity on HEK-Blue™ CD40L cells.

FIGS. 3C-3D graphically depict a dose dependent effect of a number of IFA molecules with IFNβ fusions on activating the Type I IFN-pathway in reporter HEK-Blue-IFN-α/β cells. FIG. 3C shows examples of IFN activity for IFAs with IFNβ fused to the C-terminal part of the HC. FIG. 3D shows examples of IFN activity for IFAs with IFNβ fused to the N-terminal part of the LC (IFA34) or the HC (IFA36) and the corresponding fusions on the C-terminal part (IFA35 and IFA37). The same supernatants from HEK transfected cells as in FIG. 3B were used and serially diluted to evaluate the IFN activity. Parental antibody CP870,893 was used as negative control and recombinant human IFNβ was used as positive control. NS: Non Stimulated.

FIG. 4A graphically depicts a dose effect of a number of IFA molecules with IFNα fusions on activating the CD40-mediated NFκB pathway reporter assay in HEK-Blue™ CD40L cells.

FIG. 4B graphically depicts a dose effect of a number of IFA molecules with IFNα fusions on activating the Type I IFN-mediated pathway in reporter HEK-Blue-IFN-α/β cells. The activity of Pegasys is indicated in the insert in the lower right corner.

FIG. 4C graphically depicts the effect of IFA molecules with IFNα fusions and HL linker on HC (IFA38) or LC (IFA39) on activating the CD40-mediated NFκB pathway reporter assay in HEK-Blue™ CD40L cells.

FIG. 4D graphically depicts the effect of IFA38 and IFA39 on activation of the Type I IFN-pathway in reporter HEK-Blue-IFNα/β cells.

FIG. 5A graphically depicts the effect of S309 antibody on Vero E6 cells viability after infection with SARS-CoV-2 (isolate USA-WA1/2020). For neutralization experiment, the virus was pre-incubated with a dose range of S309 antibody for 1 h at 37° C. and the virus/antibody was subsequently added onto the cells and incubated for 3 days at 37° C. Cell viability was assessed at 72 h post-infection by CellTiter Glo (CTG) assay.

FIG. 5B graphically depicts the effect of Remdesivir on Vero E6 cells viability after infection with SARS-CoV-2 (isolate USA-WA1/2020). For Remdesivir evaluation, cells were infected with SARS-CoV-2 (MOI (multiplicity of infection): 0.05). One hour later, the virus was removed and Remdesivir added in a dose range. Cell viability was assessed at 72 h post-infection by CellTiter Glo.

FIG. 5C graphically depicts the expression of CD40 on Vero E6 cells evaluated by flow cytometry.

FIGS. 5D-G graphically depict the effect of IFA27 in comparison to Remdesivir (RDV) on Vero E6 cells viability after infection with SARS-CoV-2 (isolate USA-WA1/2020). FIG. 5D graphically depicts the effect of IFA27 treatment after infection (post-treatment). FIG. 5E graphically depicts the effect of Remdesivir (RDV) treatment after infection (post-treatment). FIG. 5F graphically depicts the effect of IFA27 treatment before infection (pre-treatment). FIG. 5G graphically depicts the effect of Remdesivir (RDV) treatment before infection (pre-treatment). Cells were infected with SARS-CoV-2 (MOI 0.05). In the post-treatment setting, after 1 h of infection, the virus was removed, the cells were washed and IFA27 or Remdesivir were added at the indicated concentrations for a duration of 72 h. In the pre-treatment setting, Vero E6 cells were treated with IFA27 or Remdesivir for 1 h. Cells were then washed and infected with SARS-CoV-2 (isolate USA-WA1/2020) for a duration of 72 h. In both experiments, cell viability was assessed 3 days after infection by CellTiter-Glo.

FIGS. 5D.bis-G.bis graphically depict the effect of IFA25 in comparison to Remdesivir (RDV) on Vero E6 cells viability after infection with SARS-CoV-2 (isolate USA-WA1/2020). FIG. 5D.bis graphically depicts the effect of IFA25 treatment after infection (post-treatment). FIG. 5E.bis graphically depicts the effect of Remdesivir (RDV) treatment after infection (post-treatment). FIG. 5F.bis graphically depicts the effect of IFA25 treatment before infection (pre-treatment).

FIG. 5G.bis graphically depicts the effect of Remdesivir (RDV) treatment before infection (pre-treatment). Cells were infected with SARS-CoV-2 (MOI 0.05). In the post-treatment setting, after 1 h of infection, the virus was removed, the cells were washed and IFA25 or Remdesivir were added at the indicated concentrations for a duration of 72 h. In the pre-treatment setting, Vero E6 cells were treated with IFA25 or Remdesivir for 1 h. Cells were then washed and infected with SARS-CoV-2 (isolate USA-WA1/2020, MOI 0.05) and kept for a duration of 72 h. In both experiments, cell viability was assessed 3 days after infection by CellTiter-Glo.

FIG. 5H graphically depicts the effect of IFA27 in comparison to parental antibody (CP870,893) or control IFA201 (without the anti-CD40 agonistic activity) in a post-treatment setting. Vero E6 cells were infected with SARS-CoV-2 (isolate USA-WA1/2020; MOI 0.05). After one hour infection, the virus was removed, the cells were washed and fresh medium was added in the presence of a dose range of the indicated treatment for a duration of 72 h. Cell viability was assessed by CTG assay.

FIG. 5I graphically depicts the effect of IFA25 in comparison to Pegasys® or control IFA202 (without the anti-CD40 agonistic activity) in a post-treatment setting. Vero E6 cells were infected with SARS-CoV-2 (isolate USA-WA1/2020; MOI 0.05). After one hour infection, the virus was removed, the cells were washed and fresh medium was added in the presence of a dose range of the indicated treatment for a duration of 72 h. Cell viability was assessed by CTG assay.

FIG. 5I.bis graphically depicts the effect of IFA25 alone or in combination with the isotype EVI5 (to have the same antibody load as in the CP870,893 and IFA202 combination) in comparison to CP870,893 or control IFA202 (without the anti-CD40 agonistic activity), alone or in combination, in a post-treatment setting. Vero E6 cells were infected with SARS-CoV-2 (isolate USA-WA1/2020; MOI 0.05). After one hour infection, the virus was removed, the cells were washed and fresh medium was added in the presence of a dose range of the indicated treatment for a duration of 72 h. Cell viability was assessed by CTG assay.

FIGS. 5J-R graphically depict the effect of IFA25 on Vero E6 cells viability after infection with various SARS-CoV-2 isolates. FIG. 5J shows the effect after infection with isolate USA-WA1/2020. FIG. 5K shows the effect after infection with isolate Germany/BavPat1/2020. FIG. 5L shows the effect after infection with isolate USA-CA_CDC_5574-2020. FIG. 5M shows the effect after infection with isolate hCoV-19_England_204820464_2020. FIG. 5N shows the effect after infection with isolate South Africa/KRISP-EC-K005321/2020. FIG. 5O shows the effect after infection with isolate South Africa/KRISP-EC-K005325/2020. FIG. 5P shows the effect after infection with isolate hCoV-19/Japan/TY7-503/2021 (Gamma variant). FIG. 5Q shows the effect after infection with isolate hCoV-19/USA/PHC658/2021 (Delta variant). FIG. 5R shows the effect after infection with isolate hCoV-19/USA/MD-HP20874/2021 (Omicron variant). Cells were infected with SARS-CoV-2 at MOI 0.05 (except for Gamma and Omicron variants, at MOI 0.5 and 0.1, respectively). One hour later, the virus was removed, the cells were washed and IFA25 was added in a dose range. Cell viability was assessed 3 days after infection by CTG assay.

FIGS. 5S-U graphically depict the effect of IFA126 on Vero E6 cells viability after infection with various SARS-CoV-2 isolates. FIG. 5S shows the effect after infection with isolate USA-WA1/2020 (MOI 0.05). FIG. 5T shows the effect after infection with isolate hCoV-19/USA/PHC658/2021 (Delta variant, MOI 0.05). FIG. 5U shows the effect after infection with isolate hCoV-19/USA/MD-HP20874/2021 (Omicron variant, MOI 0.1).

FIGS. 6A-C graphically depict the results concerning the setup of the Real Time Cell Analysis using the xCELLigence system upon infection of Vero E6 cells with SARS-CoV-2. Vero E6 cells were infected with SARS-CoV-2 (isolate USA-WA1/2020). After one hour infection, the virus was removed, the cells were washed and fresh medium was added in the presence of the indicated treatment. FIG. 6A shows the normalized Cell Index (CI) for E-Plate wells that were inoculated with a negative control (Non infected) or different numbers of plaque forming units (PFU) of SARS-CoV-2 for 75 h. The horizontal line denotes the point at which CI has dropped to 50% of its initial value. The time required to reach this point is referred to as CIT₅₀. FIG. 6B displays the effect of S309 antibody on SARS-CoV-2-induced cytopathic effect in Vero E6 cells. The cells were inoculated with a negative control (Non infected), an infected control at a MOI of 0.025, or a mixture of virus (MOI 0.025) with 1 μg/mL of S309 antibody pre-incubated for 1 h at 37° C. FIG. 6C depicts the effect of Remdesivir (RDV) on SARS-CoV-2-induced cytopathic effect in Vero E6 cells. The cells were inoculated with a negative control (NI), an infected control at a MOI of 0.01, or escalating concentrations of Remdesivir (RDV).

FIGS. 6D-6I graphically depict the effect of IFA25, Pegasys® and control IFA202 on Vero E6 cells viability after infection with SARS-CoV-2 (USA-WA1/2020). Cells were infected with SARS-CoV-2 (MOI 0.05). One hour later, the virus was removed and IFA25 was added in a dose range. Cell viability was measured using xCELLigence Real Time Cell Analysis (RTCA) technology.

FIG. 7 depicts results from an in vitro Cytokines Release Assay of Human Whole Blood Cells (WBCs): Example of data obtained after stimulation of WBCs from 4 healthy volunteer donors. WBC were left Non-Stimulated (NS), treated with LPS (10 ng/mL) or with IFA1 (1 μg/mL) for 24 h. Supernatants were collected and submitted to cytokines release quantification using the MSD u-Plex kit for human cytokines. Results represent the mean of two independent stimulations from each donor. The profile of CXCL10 (IP10), IL6, IL1β and TNFα are shown.

Tables 11a-b: These tables summarize data obtained after in vitro stimulation of whole blood cells (WBCs) obtained from healthy volunteers. Each IFA was tested on WBCs from four different donors. WBCs were left Non-Treated (NT), treated with LPS (10 ng/mL) or with IFAs (1 μg/mL) for 24 h. Supernatants were collected and submitted to cytokines release quantification using the MSD u-Plex kit for human cytokines. Results represent the mean of two independent stimulations from each donor and are expressed in pg/mL (nd: not detected).

FIG. 8: Pharmacokinetic profile of IFA25, IFA26, IFA27, IFA28, IFA29, and IFA30 after 0.5 mg/kg (IFAs) or 0.3 mg/kg (Pegasys) intravenous bolus injection to mice. Data expressed as mean+/−SD on semi-logarithmic scale. Samples were collected up to 10 days after administration. ELISA assay using anti-IFNα as secondary antibody for quantification method was used for IFA27, IFA29 and IFA30 (FIG. 8A) and for IFA25, IFA26 and IFA28 (FIG. 8B). ELISA assay using anti-IgG2 as secondary antibody for quantification method was used for IFA25 and IFA27 (FIG. 8C). FIG. 8D: Pegasys quantification was done using human IFNα matched antibody pairs. The marked line (LLOQ) denotes the limit of detection for the Pegasys assay.

Table 12A: PK Report Summary: PK parameters for CP870,893, IFA27, IFA29 and IFA30 following single intravenous administration of 0.5 mg/kg to male CD1 Swiss mice. PK parameters for CP870,893 were explored in a 7-day experiment and those for IFA27, IFA29 and IFA30 in 10-day experiments (quantification for IFA27 was performed using 2 different ELISA approaches).

Table 12B: PK Report Summary: PK parameters for CP870,893, Pegasys and for three different IFAs (IFA25, IFA26 and IFA28) following single intravenous bolus administration of 0.5 mg/kg to male CD1 Swiss mice. PK parameters for CP870,893 and IFA25, IFA26, IFA28 and Pegasys were explored in 21-day experiments (quantification for IFA25 was performed using 2 different ELISA approaches).

FIG. 9A depicts CD40 agonistic activity in a dose dependent manner of IFA50 and IFA51 with no Fc region in comparison to the parental anti-CD40 antibody in reporter HEK-Blue™ CD40L cells. FIG. 9B depicts the IFNα activity in a dose dependent manner of IFA50 and IFA51 in reporter HEK-Blue™ hIFN-α/β cells.

FIG. 10A depicts CD40 agonistic activity in a dose dependent manner of IFNε based IFA49, in comparison to parental anti-CD40 antibody, in HEK-Blue™ CD40L reporter cells. IFA49 corresponds to fusion of IFNβ to the HC via a peptide linker. FIG. 10B depicts the IFN activity in a dose dependent manner of IFA49 on reporter HEK-Blue™ hIFN-α/β reporter cells which are activated by Type I interferons.

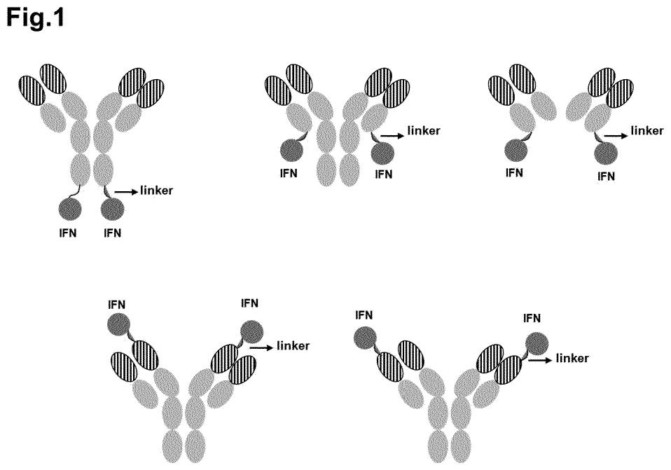

FIG. 1C-10E graphically depict the effect of IFA49 on Vero E6 cells viability after infection with various SARS-CoV-2 isolates. FIG. 10C shows the effect after infection with isolate USA-WA1/2020 (MOI 0.05). FIG. 10D shows the effect after infection with isolate hCoV-19/USA/PHC658/2021(Delta variant, MOI 0.05). FIG. 10E shows the effect after infection with isolate hCoV-19/USA/MD-HP20874/2021 (Omicron variant, MOI 0.1).

FIG. 11A depicts CD40 agonistic activity in a dose dependent manner of IFNω based IFA46, in comparison to parental anti-CD40 antibody, in HEK-Blue™ CD40L reporter cells. IFA46 correspond to fusion of IFNω to the LC via a peptide linker. FIG. 11B depicts the IFN activity in a dose dependent manner of IFA46 on reporter HEK-Blue™ hIFN-α/β reporter cells which are activated by Type I interferons.

FIG. 11C-11E graphically depict the effect of IFA46 on Vero E6 cells viability after infection with various SARS-CoV-2 isolates. FIG. 11C shows the effect after infection with isolate USA-WA1/2020 (MOI 0.05). FIG. 11D shows the effect after infection with isolate hCoV-19/USA/PHC658/2021 (Delta variant, MOI 0.05). FIG. 11E shows the effect after infection with isolate hCoV-19/USA/MD-HP20874/2021 (Omicron variant, MOI 0.1).

FIG. 12A depicts CD40 agonistic activity in a dose dependent manner of IFNγ based IFAs (IFA42 and IFA43), in comparison to parental anti-CD40 antibody, in HEK-Blue™ CD40L reporter cells. IFA42 corresponds to fusion of IFNγ to the LC via a peptide linker and IFA43 corresponds to fusion of IFNγ to the HC via a peptide linker. FIG. 12B depicts the IFN activity in a dose dependent manner of IFA42 and IFA43 in reporter HEK-Blue-hIFNγ cells.

FIG. 12C-12E graphically depict the effect of IFA42 on Vero E6 cells viability after infection with various SARS-CoV-2 isolates. FIG. 12C shows the effect after infection with isolate USA-WA1/2020 (MOI 0.05). FIG. 12D shows the effect after infection with isolate hCoV-19/USA/PHC658/2021(Delta variant, MOI 0.05). FIG. 12E shows the effect after infection with isolate hCoV-19/USA/MD-HP20874/2021 (Omicron variant, MOI 0.1).

FIG. 12F-12H graphically depict the effect of IFA43 on Vero E6 cells viability after infection with various SARS-CoV-2 isolates. FIG. 12F shows the effect after infection with isolate USA-WA1/2020 (MOI 0.05). FIG. 12G shows the effect after infection with isolate hCoV-19/USA/PHC658/2021(Delta variant, MOI 0.05). FIG. 12H shows the effect after infection with isolate hCoV-19/USA/MD-HP20874/2021(Omicron variant, MOI 0.1).

FIG. 13A depicts CD40 agonistic activity in a dose dependent manner of IFNλ based IFAs (IFA44 and IFA45), in comparison to parental anti-CD40 antibody, in HEK-Blue™ CD40L reporter cells. IFA44 corresponds to fusion of IFNλ to the LC via a peptide linker and IFA45 correspond to fusion of IFNλ to the HC via a peptide linker. FIG. 13B depicts the IFN activity in a dose dependent manner of IFA44 and IFA45 in reporter HEK-Blue-hIFNλ cells.

FIG. 13C-13E graphically depict the effect of IFA44 on Vero E6 cells viability after infection with various SARS-CoV-2 isolates. FIG. 13C shows the effect after infection with isolate USA-WA1/2020 (MOI 0.05). FIG. 13D shows the effect after infection with isolate hCoV-19/USA/PHC658/2021) (Delta variant, MOI 0.05). FIG. 13E shows the effect after infection with isolate hCoV-19/USA/MD-HP20874/2021 (Omicron variant, MOI 0.1).

FIG. 13F-13H graphically depict the effect of IFA45 on Vero E6 cells viability after infection with various SARS-CoV-2 isolates. FIG. 13F shows the effect after infection with isolate USA-WA1/2020 (MOI 0.05). FIG. 13G shows the effect after infection with isolate hCoV-19/USA/PHC658/2021(Delta variant, MOI 0.05). FIG. 13H shows the effect after infection with isolate hCoV-19/USA/MD-HP20874/2021(Omicron strain, MOI 0.1).

FIG. 14 shows examples of SDS PAGE in reduced conditions of some IFAs, with IFNα or IFNβ fused on the heavy chain of 3G5-antiCD40 antibody. Migration of the parental 3G5 antiCD40 antibody is also shown on the left.

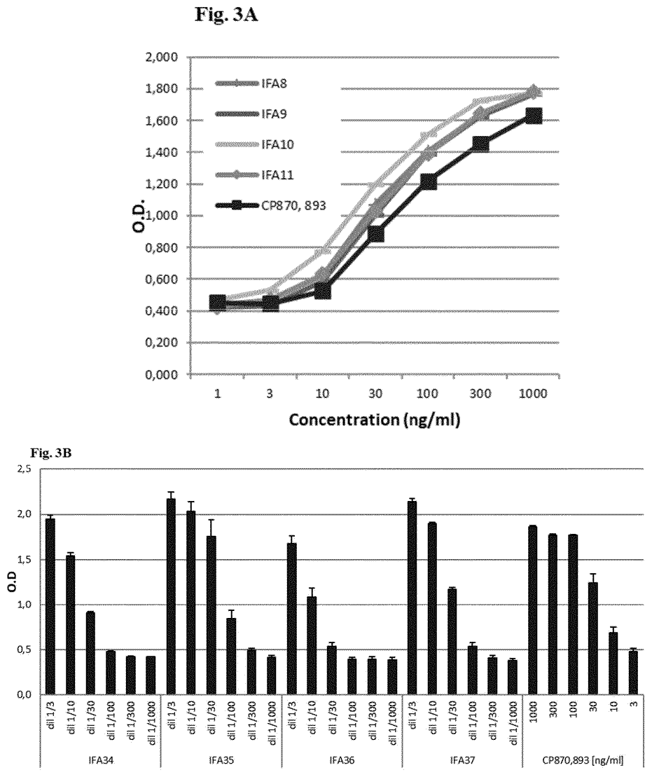

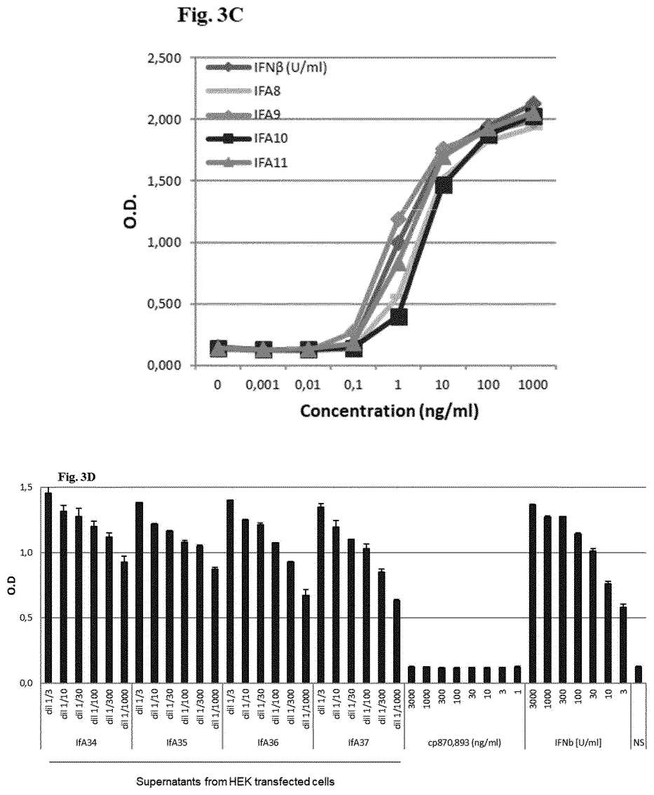

FIGS. 15A-B graphically show a dose dependent effect of a number of 3G5-based IFA molecules with IFNβ fusions on activating the CD40-mediated NFκB pathway reporter assay in HEK-Blue™ CD40L cells. Comparison to the parental antibody 3G5 (designated in this figure as CDX-3G5) is likewise shown. FIG. 15A shows examples of anti-CD40 activities for IFAs with fusion of IFNβ to the C-terminal part of the heavy chain (HC). Purification yield of IFAs with fusions of IFNβ on the light chain was very low, thus to test their activity, supernatants from HEK transfected cells were used and serially diluted to evaluate the anti-CD40 activity on HEK-Blue™ CD40L cells; an example of activity is shown in FIG. 15B and 3G5 containing supernatant was used as control.

FIGS. 15C-D graphically show a dose dependent effect of a number of IFA molecules with IFNβ fusions on activating the Type I IFN-pathway in reporter HEK-Blue-IFN-α/p cells. FIG. 15C shows examples of IFN activity for IFAs with fusion of IFNβ to the C-terminal part of the HC. FIG. 15D shows IFN activity of IFAs with IFNβ fused on the light chain; the production level of these proteins was very low and thus an example of activity for two IFAs is shown in FIG. 15D using the same supernatant as in FIG. 15B.

FIG. 16A graphically shows a dose effect of four IFAs molecules with IFNα fusions on activating the CD40-mediated NFκB pathway reporter assay in HEK-Blue™ CD40L cells. Comparison to the parental antibody 3G5 (designated in this figure as CDX-3G5) is likewise shown.

FIG. 16B graphically shows a dose effect of a number of IFAs molecules with IFNα fusions on activating the Type I IFN-mediated pathway in reporter HEK-Blue-IFN-α/β cells.

FIGS. 16C-E graphically depict the effect of IFA125 (IFNγ fusion) on Vero E6 cells viability after infection with various SARS-CoV-2 isolates. FIG. 16C shows the effect after infection with isolate USA-WA1/2020 (MOI 0.05). FIG. 16D shows the effect after infection with isolate hCoV-19/USA/PHC658/2021(Delta variant, MOI 0.05). FIG. 16E shows the effect after infection with isolate hCoV-19/USA/MD-HP20874/2021(Omicron variant, MOI 0.1).

FIG. 17: In vitro Cytokines Release Assay of Human Whole Blood Cells (WBCs): Example of data obtained after stimulation of WBCs from 4 healthy volunteer donors. WBCs were left non-treated (NT), treated with LPS (10 ng/mL) or with IFA109 (1 μg/mL) for 24 h. Supernatants were collected and submitted to cytokines release quantification using the MSD u-Plex kit for human cytokines. Results represent the mean of two independent stimulations from each donor. The profile of CXCL10 (IP10), IL6, IL1β and TNFα are shown.

Table 13: This table summarizes data obtained after in vitro stimulation of whole blood cells obtained from healthy volunteers. IFA109 was tested on WBCs from four different donors. WBCs were left Non-Treated (NT), treated with LPS (10 ng/mL) or with IFA109 (1 μg/mL) for 24 h. Supernatants were collected and submitted to cytokines release quantification using the MSD u-Plex kit for human cytokines. Results represent the mean of two independent stimulations from each donor and are expressed in pg/mL (nd: not detected).

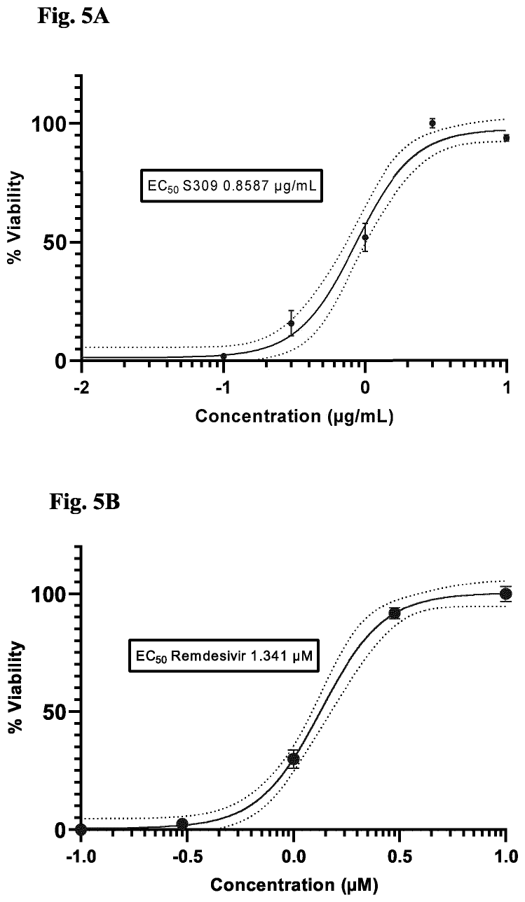

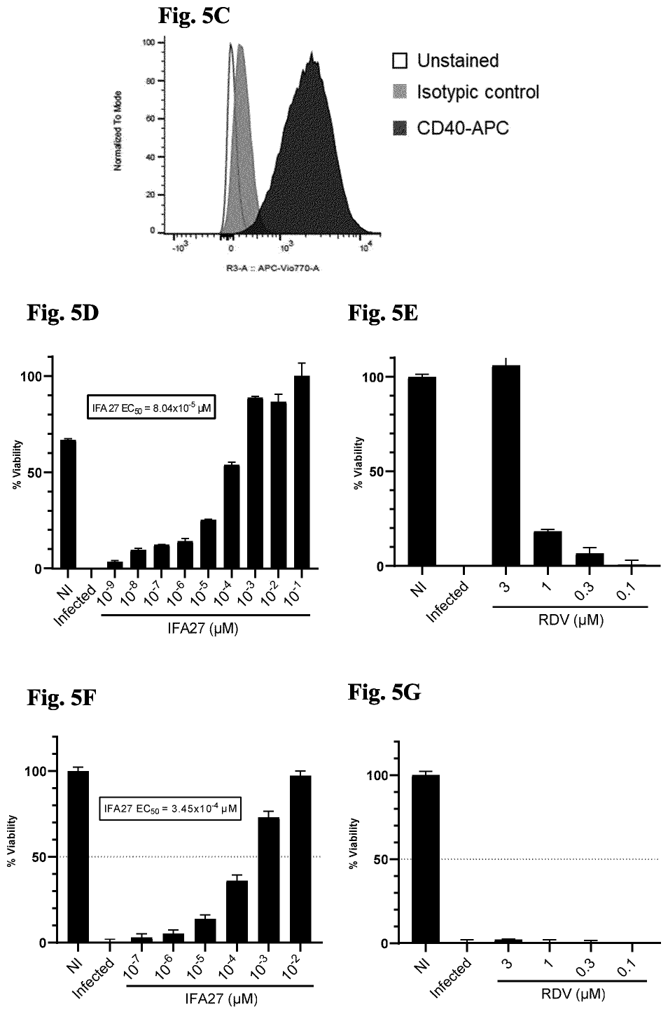

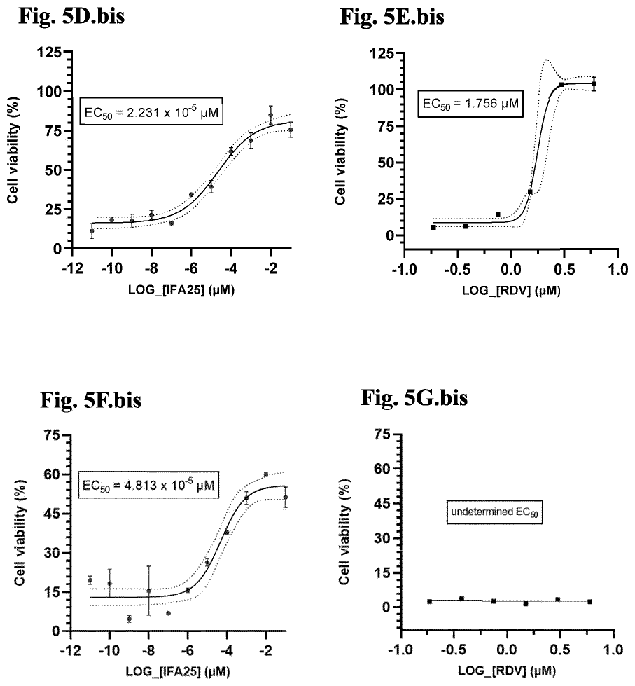

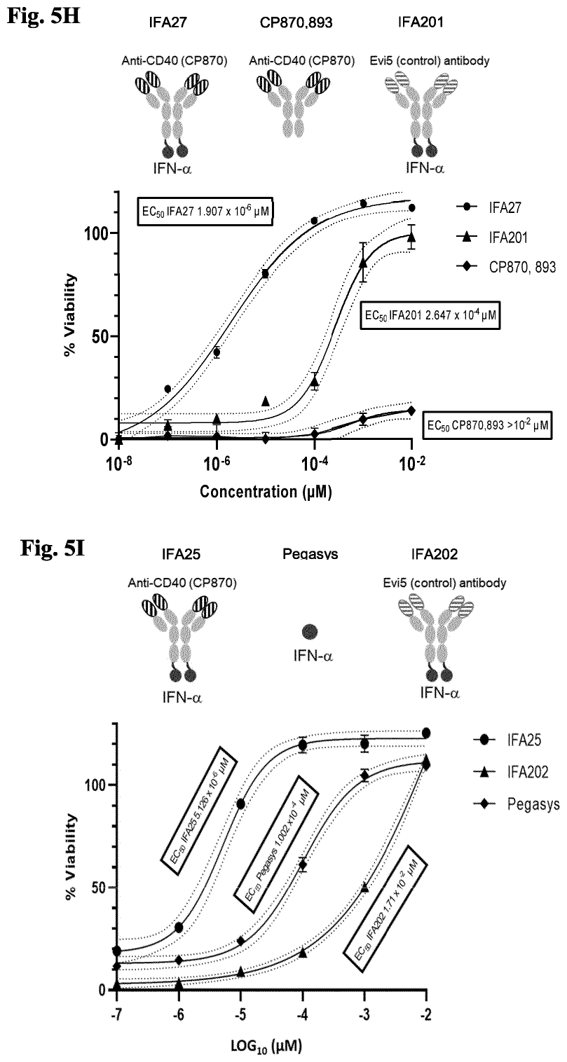

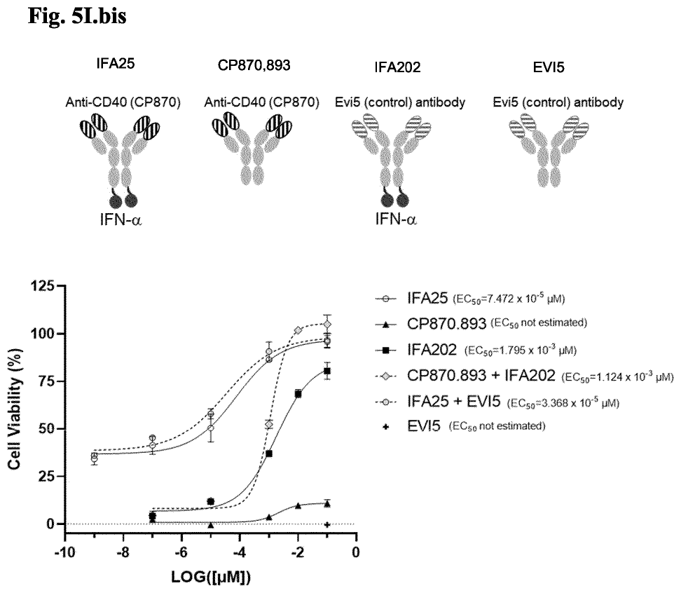

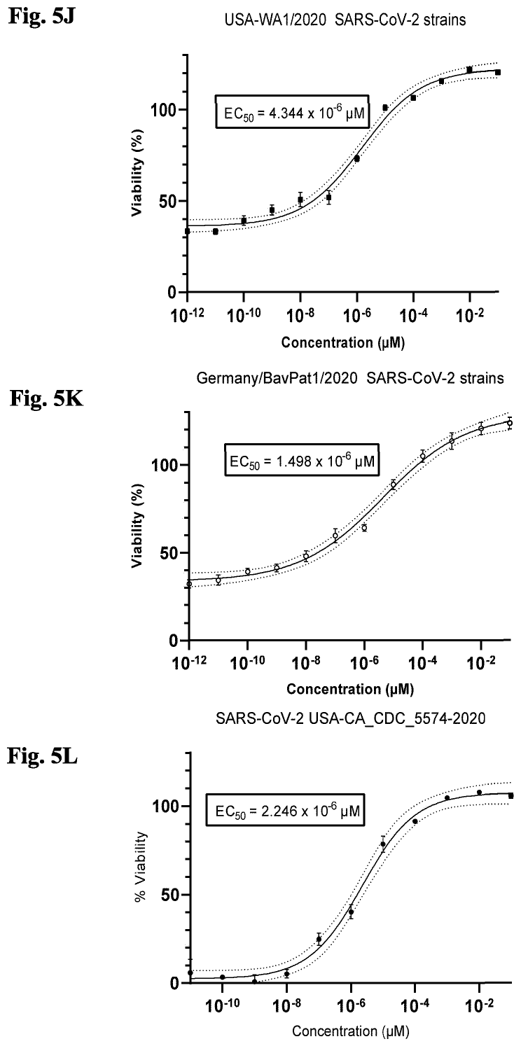

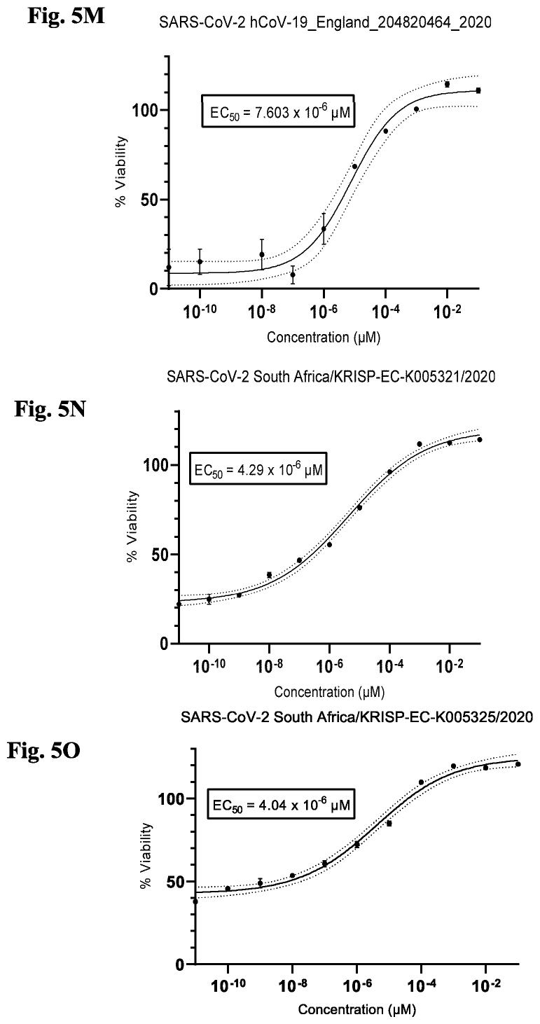

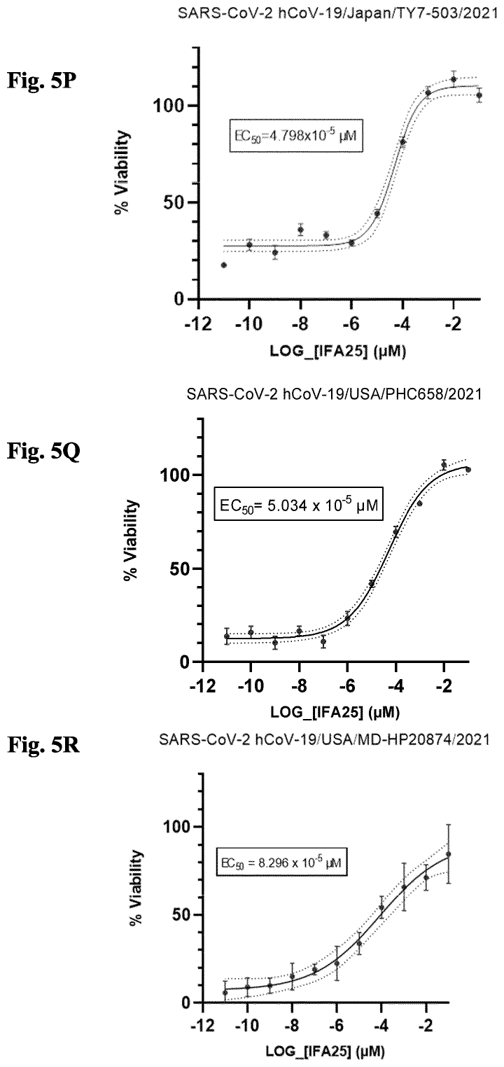

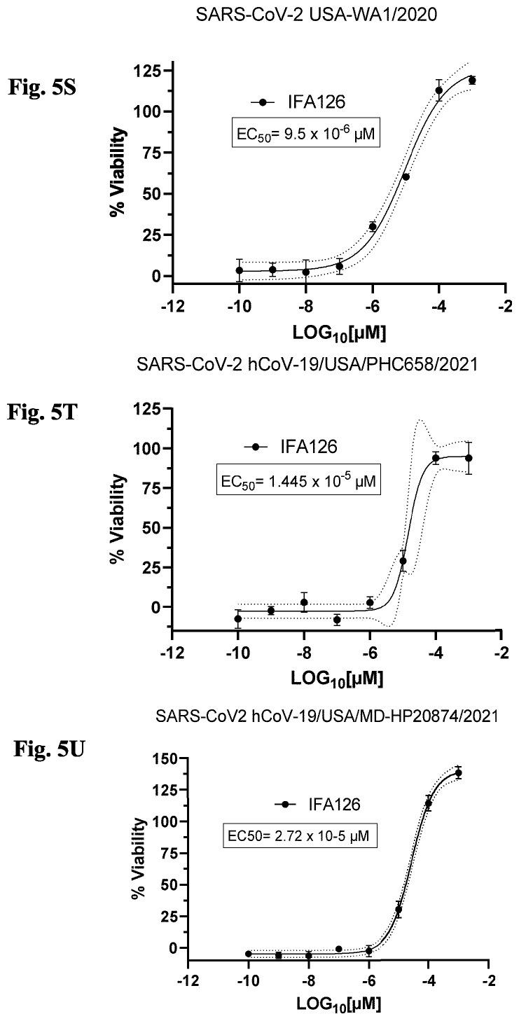

Table 14: This table reports the LogAbsoluteEC50 values of IFA25 and Remdesivir estimated against each SARS-CoV-2 isolate. In the experiments presented in FIGS. 5J to 5R evaluating the effect of IFA25 on Vero E6 cells viability, Remdesivir was tested in the same conditions. The absolute EC50 was defined according to the Guidelines for accurate EC50/IC50 estimation (Sebaugh J. L.; Guidelines for accurate EC50/IC50 estimation. Pharmaceut. Statist. 2010; DOI: 10.1002/pst.426.), as the concentration that gives 50% response, defined as the mean of the 0% (non-infected CTR condition) and the 100% (Infected-CTR condition). Thus, the LogAbsoluteEC50 was estimated with PRISM, using a 4-PL model, by inverse prediction setting Y equal to a 50% response (the 50% control mean).

FIG. 18 graphically depicts CD40, IFNAR1 and IFNAR2 expression in primary human nasal and bronchial cells, in non-infected and in SARS-CoV-2 infected condition. The RNA expression of CD40, IFNAR1 and IFNAR2 was assessed by RT-qPCR analysis. For this assessment, 500 ng of total RNA extracted from primary human nasal and bronchial cells (non-infected or infected) were first used as template for cDNA synthesis following manufacturer instructions (cat #11754050) as detailed in example VIII. Then, TLDA receptor arrays card (Thermofisher Scientific-detailed in the method section) were performed to simultaneously quantify the mRNA level of specific receptors. House-keeping genes mRNA expression (GAPDH, GUSB, TBP and RPLPO) was also included in TLDA assay to assess potential mRNA variation in all 4 conditions tested. Each of the 4 house-keeping genes had similar expression level in nasal and bronchial cells and with or without infection thus enabling direct Ct comparison. (FIG. 18A). The CD40 and IFNAR target expression was confirmed at the protein level in non-infected cells by cytometry analysis. Primary human nasal and bronchial cells were incubated with an anti-hCD40-APC, anti-hIFNAR1-APC and anti-hIFNAR2-APC or matching isotype controls. Cells were acquired on MACSQuant16 flow cytometer. Isotype controls are represented by dashed line histograms and interest markers by full grey histograms (FIG. 18B).

FIG. 19 graphically depicts effect of IFA27 treatments (post-treatment applied at 1 h, 48 h and 96 h in a 168 h kinetic) on SARS-CoV-2 virus load in primary human nasal cells cultured in air-liquid interface (ALI) system (batch, MD0713) and infected with the isolate USA-WA1/2020. Cells were cultured in MucilAir™ culture medium from receipt and were infected at day 0 of the experiment with isolate USA-WA1/2020 at 0.1 MOI. After 1 hour, the infection was washed and fresh medium containing IFA27 or EVI5 isotype control or Remdesivir treatments were applied and renewed at 2 and 4 days after the infection. Additional non-infected and infected non-treated conditions were performed. The final washes and tissue collection was performed 7 days after the infection. The viral load was assessed by RT-qPCR at the terminal end-point 7 days after the infection by measuring SARS-CoV-2 RNA copies in the nasal tissue (FIG. 19A) and nasal apical washes (FIG. 19B) after RNA extraction as described in Example VIII. Infectious virus was evaluated in apical washes at the terminal end-point 7 days after the infection (FIG. 19C) using a 50% tissue culture infectious dose (TCID50) assay as described in Example VIII.

FIG. 20 graphically depicts the effect of IFA27 treatments (post-treatment applied at 1 h, 48 h and 96 h in a 168 h kinetic) on SARS-CoV-2 virus load in primary human bronchial cells cultured in air-liquid interface (ALI) system (Batch, MD0713) and infected with the isolate USA-WA1/2020. Cells were cultured in MucilAir™ culture medium from receipt and were infected at day 0 of the experiment with isolate USA-WA1/2020 at 0.1 MOI. After 1 hour, the infection was washed and fresh medium containing IFA27 or EVI5 isotype control or Remdesivir treatments were applied and renewed at 2 and 4 days after the infection. Additional non-infected and infected non-treated conditions were performed. The final washes and tissue collection was performed 7 days after the infection. The viral load was assessed by RT-qPCR at the terminal end-point 7 days after the infection by measuring SARS-CoV-2 RNA copies in the bronchial tissue (FIG. 20A) and bronchial apical washes (FIG. 20B) after RNA extraction as described in Example VIII.

FIG. 21 graphically depicts the effect of IFA25 treatments (post-treatment applied at 1 h and 48 h in a 96 h kinetic) on SARS-CoV-2 virus load in primary human nasal cells cultured in air-liquid interface (ALI) system (batch, MD0853) and infected with the isolate USA-WA1/2020. Cells were cultured in MucilAir™ culture medium from receipt and were infected at day 0 of the experiment with isolate USA-WA1/2020 at 0.1 MOI. After 1 hour, the infection was washed and fresh medium containing IFA25 or EVI5 isotype control or Remdesivir treatments were applied and renewed at 2 days after the infection. Additional non-infected and infected non-treated conditions were performed. The viral load was assessed by RT-qPCR at the terminal end-point 4 days after the infection by measuring SARS-CoV-2 RNA copies in the nasal tissue (FIG. 21A) and nasal apical washes (FIG. 21B) after RNA extraction as described in Example VIII. Infectious virus was evaluated in apical washes at the terminal end-point 4 days after the infection (FIG. 21C) using a 50% tissue culture infectious dose (TCID50) assay as described in Example VIII.

FIG. 22 graphically depicts effect of pre-treatment with IFA25 on SARS-CoV-2 virus load in primary human nasal cells cultured in air-liquid interface (ALI) system (batch, MD0853) and infected with the isolate USA-WA1/2020 strain. Cells were culture in MucilAir™ culture medium from receipt and were treated 3 hours before the infection with IFA25 or EVI5 isotype control or Remdesivir. Additional non-infected and infected non-treated conditions were performed. Treatments were removed and the infection was performed for 1 hour with isolate USA-WA1/2020 strain at 0.1 MOI. After 1 hour infection, apical compartment was washed and fresh medium was added. The final washes and tissue collection was performed 2 days after the infection. The viral load was assessed by RT-qPCR at the terminal end-point, 2 days after the infection by measuring SARS-CoV-2 RNA copies in the nasal tissue (FIG. 22A) and nasal apical washes (FIG. 22B) after RNA extraction as described in Example VIII. Infectious virus was evaluated in apical washes at the terminal end-point 2 days after the infection (FIG. 22C) using a 50% tissue culture infectious dose (TCID50) assay as described in example VIII.

FIG. 23 graphically depicts the effect of IFA25 treatments (post-treatment applied at 1 h and 48 h in a 96 h kinetic) on SARS-CoV-2 virus load in the primary human bronchial cells cultured in air-liquid interface (ALI) system (batch, MD0868) and infected with the isolate USA-WA1/2020. Cells were cultured in MucilAir™ culture medium from receipt and were infected at day 0 of the experiment with isolate USA-WA1/2020 at 0.1 MOI. After 1 hour, the infection was washed and fresh medium containing IFA25 or EVI5 isotype control or Remdesivir treatments were applied and renewed at 2 days after the infection. Additional non-infected and infected non-treated conditions were performed. The viral load was assessed by RT-qPCR at the terminal end-point 4 days after the infection by measuring SARS-CoV-2 RNA copies in the bronchial tissue (FIG. 23A) and bronchial apical washes (FIG. 23B) after RNA extraction as described in Example VIII. Infectious virus was evaluated in apical washes at the terminal end-point 4 days after the infection (FIG. 23C) using a 50% tissue culture infectious dose (TCID50) assay as described in example VIII.

FIG. 24 graphically depicts effect of IFA25 pre-treatment on SARS-CoV-2 virus load in primary human bronchial cells cultured in air-liquid interface (ALI) system (batch, MD0868) and infected with the isolate USA-WA1/2020. Cells were cultured in MucilAir™ culture medium from receipt and were treated 3 hours before the infection with IFA25 or EVI5 isotype control or Remdesivir. Additional non-infected and infected non-treated conditions were performed. Treatments were removed and the infection was performed for 1 hour with isolate USA-WA1/2020 at 0.1 MOI. After 1 hour infection, apical compartment was washed and fresh medium was added. The final washes and tissue collection was performed 2 days after the infection. The viral load was assessed by RT-qPCR at the terminal end-point, 2 days after the infection by measuring SARS-CoV-2 RNA copies in the bronchial tissue (FIG. 24A) and bronchial apical washes (FIG. 24B) after RNA extraction as described in Example VIII.

“Cell viability (%)” and “Viability (%)” are used interchangeably throughout the document.

The foregoing and other features and advantages of the present invention will be more fully understood from the following detailed description of illustrative embodiments taken in conjunction with the accompanying drawings.

### DETAILED DESCRIPTION

It will be understood that any of the definitions and embodiments described and/or claimed herein are intended to be definitions and embodiments applicable to all aspects, embodiments, items and matters of the invention. For example, it will be understood that the teaching and explanations provided herein in respect of suitable ways or embodiments of preparing, formulating and administering the interferon-associated antigen binding proteins of the invention, or nucleic acids encoding or expressing same, and routes of their administration, suitable dosages and administration regimens therefor, apply mutatis mutandis to the tumor necrosis factor receptor superfamily (TNFRSF) agonists or functional fragments thereof, the interferons (IFNs) or functional fragments thereof, or nucleic acids encoding or expressing same, or the combinations thereof as described or claimed herein.

The present invention is based in part on the discovery of a therapy that is based on the use of “interferon-associated antigen-binding proteins”, variants or derivatives thereof comprising (I) an agonistic anti-CD40 antibody or an agonistic antigen binding fragment thereof, and (II) an interferon (IFN) or a functional fragment thereof in Coronavirus therapy. Said interferon-associated antigen-binding proteins rescue cells from Coronavirus-induced cell death and from Coronavirus-induced cytopathic effect and enhance the IFN pathway in uninfected and infected cells, and may even act in a synergistic fashion. Coronavirus therapy comprising administering an interferon-associated antigen-binding protein to a Coronavirus-infected cell, or a subject infected with Coronavirus, is provided.

The invention may be more readily understood in the light of the selected terms defined below.

As used herein, a tumor necrosis factor (ligand) superfamily member (or TNFSF) refers to a protein belonging to a superfamily of protein ligands that share a hallmark extracellular TNF homology domain (THD) (Bremer ISRN Oncology (2013), Article ID 371854, 25 pages, online access: dx.doi.org/10.1155/2013/371854). The THD triggers formation of non-covalent homotrimers. TNF ligands are typically expressed as type II transmembrane proteins, but most can be subject to proteolytic processing into a soluble ligand. TNF ligands exert their biological function by binding to and activating members of the TNFRSF. TNFRSFs are typically expressed as trimeric type I transmembrane proteins and contain one to six cysteine-rich domains (CRDs) in their extracellular domain. An important function of the TNF superfamily is the provision of co-stimulatory signals at distinct stages of an immune response. Some ligands have the capacity to bind and activate different receptors (e.g., LTa3 which binds and activates TNFRSFlA, TNFRSFlB and TNFRSF14 and LIGHT (TNFSF14) which binds and activates TNFRSF3 and TNFRSF14). Exemplary TNFSF gene family members are recited below in Table A, derived from the HUGO Gene Nomenclature Committee (HGNC) (see, Gray et al. Nucleic Acids Res. 43: D1079-1085 (2015); HGNC Database, HUGO Gene Nomenclature Committee (HGNC), EMBL Outstation—Hinxton, European Bioinformatics Institute, Wellcome Trust Genome Campus, Hinxton, Cambridgeshire, CBl0 lSD, UK www.genenames.org). The Approved Symbol denotes the HGNC symbol applied to a particular gene and the Approved Name corresponds to the full spelling of the gene. Previous Symbols denotes any previous symbol used by HGNC or refer to a particular gene. Synonyms refer to alternative, synonymous names for a particular gene.

TABLE A
Exemplary TNFSF gene family members

| Approved | | Previous | |
|---|---|---|---|
| Symbol | Approved Name | Symbols | Synonyms |
| TNF | tumor necrosis | TNFA | TNFSF2, DIF, |
| | factor | | TNFα |
| LTA | lymphotoxin alpha | TNFB | LT, TNFSF1 |
| LTB | lymphotoxin beta | TNFC | TNFSF3, p33 |
| TNFSF4 | TNF superfamily | TXGP1 | OX-40L, |
| | member 4 | | gp34, CD252 |
| CD40LG | CD40 ligand | TNFSF5, | CD40L, TRAP, |
| | | HIGM1, | gp39, hCD40L, |
| | | IMD3 | CD154 |
| FASLG | Fas ligand | TNFSF6, | FasL, CD178 |
| | | APT1LG1 | |
| CD70 | CD70 molecule | TNFSF7, | CD27L |
| | | CD27L | |
| TNFSF8 | TNF superfamily | | CD153 |
| | member 8 | | |
| TNFSF9 | TNF superfamily | | 4-1BB-L |
| | member 9 | | |
| TNFSF10 | TNF superfamily | | TRAIL, Apo-2L, |
| | member 10 | | TL2, CD253 |
| TNFSF11 | TNF superfamily | | TRANCE, RANKL, |
| | member 11 | | OPGL, ODF, |
| | | | CD254 |
| TNFSF12 | TNF superfamily | | TWEAK, DR3LG, |
| | member 12 | | APO3L |
| TNFSF13 | TNF superfamily | | APRIL, CD256 |
| | member 13 | | |
| TNFSF13B | TNF superfamily | TNFSF20 | BAFF, THANK, |
| | member 13B | | BLYS, TALL-1, |
| | | | TALL1, CD257 |
| TNFSF14 | TNF superfamily | | LIGHT, LTγ, |
| | member 14 | | HVEM-L, CD258 |
| TNFSF15 | TNF superfamily | | TL1, VEGI, TL1A, |
| | member 15 | | VEGI192A, |
| | | | MGC129934, |
| | | | MGC129935 |
| TNFSF18 | TNF superfamily | | AITRL, TL6, |
| | member 18 | | hGITRL |
| EDA | ectodysplasin A | ED1, | EDA1, XLHED, |
| | | EDA2, | HED, XHED, ED1- |
| | | ODT1 | A1, ED1-A2, EDA- |
| | | | A1, EDA-A2 |

TABLE B
Exemplary TNFRSF gene family members

| Approved | | Previous | |
|---|---|---|---|
| Symbol | Approved Name | Symbols | Synonyms |
| EDAR | ectodysplasin | ED3, DL | ED5, EDA3, Edar, |
| | A receptor | | ED1R, EDA1R |
| TNFRSF1A | TNF receptor | TNFR1 | TNF-R, TNFAR, |
| | superfamily | | TNFR60, TNF-R-I, |
| | member 1A | | CD120a, TNF-R55 |
| TNFRSF1B | TNF receptor | TNFR2 | TNFBR, TNFR80, |
| | superfamily | | TNF-R75, TNF-R- |
| | member 1B | | II, p75, CD120b |
| LBTR | Lymphotoxin | D12S370 | TNF-R-III, TNFCR, |
| | beta receptor | | TNFRSF3, TNFR2- |
| | | | RP, TNFR-RP |
| TNFRSF4 | TNF receptor | TXGP1L | ACT35, OX40, |
| | superfamily | | CD134 |
| | member 4 | | |
| CD40 | CD40 molecule | TNFRSF5 | p50, Bp50 |
| FAS | Fas cell surface | APT1, FAS1, | CD95, APO-1 |
| | death receptor | TNFRSF6 | |
| TNFRSF6b | TNF receptor | | DcR3, DCR3, TR6, |
| | superfamily | | M68 |
| | member 6b | | |
| CD27 | CD27 molecule | TNFRSF7 | S152, Tp55 |
| TNFRSF8 | TNF receptor | CD30, | KI-1 |
| | superfamily | D1S166E | |
| | member 8 | | |
| TNFRSF9 | TNF receptor | ILA | CD137, 4-1BB |
| | superfamily | | |
| | member 9 | | |
| TNFRSF10a | TNF receptor | | DR4, Apo2, |
| | superfamily | | TRAILR-1, CD261, |
| | member 10a | | TRAILR1 |
| TNFRSF10b | TNF receptor | | DR5, KILLER, |
| | superfamily | | TRICK2A, TRAIL- |
| | member 10b | | R2, TRICKB, |
| | | | CD262, TRAILR2 |
| TNFRSF10c | TNF receptor | | DcR1, TRAILR3, |
| | superfamily | | LIT, TRID, CD263 |
| | member 10c | | |
| TNFRSF10d | TNF receptor | | DcR2, TRUNDD, |
| | superfamily | | TRAILR4, CD264 |
| | member 10d | | |
| TNFRSF11a | TNF receptor | PDB2, | RANK, CD265, |
| | superfamily | LOH18CR1 | FEO |
| | member 11a | | |
| TNFRSF11b | TNF receptor | OPG | OCIF, TR1 |
| | superfamily | | |
| | member 11b | | |
| TNFRSF12A | TNF receptor | | FN14, TweakR, |
| | superfamily | | CD266 |
| | member 12A | | |
| TNFRSF13B | TNF receptor | | TACI, CD267, |
| | superfamily | | IGAD2 |
| | member 13B | | |
| TNFRSF13C | TNF receptor | | BAFFR, CD268 |
| | superfamily | | |
| | member 13C | | |
| TNFRSF14 | TNF receptor | | HVEM, ATAR, |
| | superfamily | | TR2, LIGHTR, |
| | member 14 | | HVEA, CD270 |
| NGFR | Nerve grwoth | | TNFRSF16, |
| | factor receptor | | p75NTR, CD271 |
| TNFRSF17 | TNF receptor | BCMA | BCM, CD269, |
| | superfamily | | TNFRSF13A |
| | member 17 | | |
| TNFRSF18 | TNF receptor | | AITR, GITR, |
| | superfamily | | CD357 |
| | member 18 | | |
| TNFRSF19 | TNF receptor | | TAJ-alpha, TROY, |
| | superfamily | | TAJ, TRADE |
| | member 19 | | |
| RELT | RELT, TNF | TNFRSF19L | FLJ14993 |
| | receptor | | |
| TNFRSF21 | TNF receptor | | DR6, CD358 |
| | superfamily | | |
| | member 21 | | |
| TNFRSF25 | TNF receptor | TNFRSF12 | DR3, TRAMP, |
| | superfamily | | WSL-1, LARD, |
| | member 25 | | WSL-LR, DDR3, |
| | | | TR3, APO-3 |
| EDA2R | Ectodysplasin | | XEDAR, EDAA2R, |
| | A2 receptor | | EDA-A2R, |
| | | | TNFRSF27 |

As used herein, a “TNFRSF agonist” refers to a compound (e.g., protein, a fusion protein, a polypeptide, an antibody, an antigen-binding fragment of an antibody or the like) that activates a TNFRSF, e.g., a TNFRSF listed in Table B. Table B is derived from the HGNC, as for Table A above. For example, a TNFRSF agonist may be an agonistic antibody directed against a member of the TNFRSF, a soluble TNFRSF agonist including but not limited to its natural ligand or a functional fragment thereof.

In certain exemplary embodiments, a TNFRSF agonist includes, but is not limited to, a LTa3 receptors (TNFRSFlA, TNFRSFlB, or TNFRSF14) agonist, a LTP receptor (TNFRSF3) agonist (e.g., LIGHT or LTP), a herpesvirus entry mediator (HVEM or TNFRSF14) agonist (LIGHT), a tumor necrosis factor-like receptor weak inducer of apoptosis (TNFRSF12A) agonist (e.g., TWEAK also known as TNFSF12), a cluster of differentiation factor 40 (CD40, TNFRSF5) agonist (CD40L), a CD27 (TNFRSF7) agonist (CD70), a CD30 (TNFRSF8) agonist, a 4-1BB (CD137, TNFRSF9) agonist, a receptor activator of nuclear factor KB (RANK, TNFRSF 1 1A) agonist, a Troy (TNFRSF 19) agonist, and an OX40 receptor (TNFRSF4) agonist.

As used herein, the term “functional fragment” refers to a fragment of a substance that retains one or more functional activities of the original substance, preferably all of the functional activities. For example, a functional fragment of a TNFRSF agonist refers to a fragment of a TNFRSF agonist that retains a function of the TNFRSF agonist as described and/or claimed herein, e.g., it activates a target TNFRSF.

As used herein, the term “ligand” refers to any substance capable of binding, or of being bound, to another substance. A ligand may be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and any combination thereof. Preferably, the ligand is a polypeptide.

As used herein, the term “CD40” refers to “Cluster of differentiation 40”, a member of the tumor necrosis factor receptor (TNFR) superfamily. CD40 is a costimulatory protein found on antigen presenting cells (e.g., B cells, dendritic cells, monocytes), hematopoietic precursors, endothelial cells, smooth muscle cells, epithelial cells, as well as the majority of human tumors (Grewal & Flavell, Ann. Rev. Immunol., 1996, 16: 111-35; Toes & Schoenberger, Seminars in Immunology, 1998, 10(6): 443-8). The binding of the natural ligand CD154 (CD40L) on TH cells to CD40 activates antigen presenting cells and induces a variety of downstream effects. The TNF-receptor associated factor adaptor proteins TRAF1, TRAF2, TRAF6 and TRAF5 interact with CD40 and serve as mediators of the signal transduction. Ultimately, CD40 signaling activates both the canonical and the noncanonical NF-κB pathways.

### Agonistic Anti-CD40 Antibodies and Antigen Binding Fragments Thereof

As used herein, the term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated VH or V_{H}) and a heavy chain constant region (CH or CH). The heavy chain constant region comprises three domains, CH1, CH2 and CH3. Each light chain comprises a light chain variable region (abbreviated VL or V_{L}) and a light chain constant region (CL or C_{L}). The light chain constant region comprises one domain (CL1). The VH and VL regions can be further subdivided into regions of hypervariability, termed “complementarity determining regions (CDRs)”, interspersed with regions that are more conserved, termed “framework regions” (FR). Each VH and VL is composed of three CDRs and four FRs, arranged from amino-terminus to carboxy-terminus in the following order: FR1, CDR1, FR2, CDR2, FR3, CDR3, FR4. Framework regions can aid in maintaining the proper conformation of the CDRs to promote binding between the antigen binding region and an antigen.

The most commonly used immunoglobulin for therapeutic applications is immunoglobulin G (or IgG), a tetrameric glycoprotein. In a naturally-occurring immunoglobulin, each tetramer is composed of two identical pairs of polypeptide chains, each pair having one light (about 25 kDa) and one heavy chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Immunoglobulins can be assigned to different classes depending on the amino acid sequence of the constant domain of their heavy chains.

Heavy chains are classified as mu (μ), delta (δ), gamma (γ), alpha (α), and epsilon (ε), and define the antibody's isotype as IgM, IgD, IgG, IgA, and IgE, respectively. Several of these may be further divided into subclasses or isotypes, e.g. IgG1, IgG2, IgG3, IgG4, IgA1 and IgA2. Different isotypes have different effector functions; for example, IgG1 and IgG3 isotypes have antibody-dependent cellular cytotoxicity (ADCC) activity. In preferred embodiments, the agonistic antiCD40 antibodies or agonistic antigen binding fragments thereof comprised in the interferon-associated antigen binding proteins according to the invention are of the IgG class. In more preferred embodiments, the agonistic antiCD40 antibodies or agonistic antigen binding fragments thereof comprised in the interferon-associated antigen binding proteins according to the invention are of the IgG1 or IgG3 subclasses. In specifically preferred embodiments, the agonistic antiCD40 antibodies or agonistic antigen binding fragments thereof comprised in the interferon-associated antigen binding proteins according to the invention are of the IgG1 subclass. In other more preferred embodiments, the agonistic antiCD40 antibodies or agonistic antigen binding fragments thereof comprised in the interferon-associated antigen binding proteins according to the invention are of the IgG2 or IgG4 subclasses. In specifically preferred embodiments, the agonistic antiCD40 antibodies or agonistic antigen binding fragments thereof comprised in the interferon-associated antigen binding proteins according to the invention are of the IgG2 subclass.

Human light chains are classified as kappa (κ) and lambda (λ) light chains. Accordingly, in some embodiments, the agonistic antiCD40 antibodies or agonistic antigen binding fragments thereof comprised in the interferon-associated antigen binding proteins according to the invention comprise a light chain of the x class. In other embodiments, the agonistic antiCD40 antibodies or agonistic antigen binding fragments thereof comprised in the interferon-associated antigen binding proteins according to the invention comprise a light chain of the λ class. Within light and heavy chains, the variable and constant regions are joined by a “J” region of about 12 or more amino acids, wherein the heavy chain additionally includes a “D” region of about 10 more amino acids. See generally, Fundamental Immunology, Ch. 7 (Paul, W., ed., 2nd ed. Raven Press, N.Y. (1989)).

The term “antibody” further includes, but is not limited to, monoclonal antibodies, bispecific antibodies, minibodies, domain antibodies, synthetic antibodies (sometimes referred to as “antibody mimetics”), chimeric antibodies, humanized antibodies, human antibodies, and fragments thereof, respectively. Unless otherwise indicated, the term “antibody” includes, in addition to antibodies comprising two full-length heavy chains and two full-length light chains, derivatives, variants, antigen binding fragments, and muteins thereof, examples of which are described below.

As used herein, the term “agonistic CD40 antibody” or “agonistic anti-CD40 antibody” refers to an antibody that binds to CD40 and mediates CD40 signaling. In a preferred embodiment, it binds to human CD40. As described below, binding to CD40 may be determined using surface plasmon resonance, preferably using the BIAcore® system. The agonistic anti-CD40 antibody may increase one or more CD40 activities by at least about 20% when added to a cell, tissue or organism expressing CD40. In some embodiments, the antibody activates CD40 activity by at least 40%, at least 50%, at least 60%, at least 70%, at least 80%, or at least 85%. CD40 activity of the agonistic anti-CD40 antibody may be measured using a whole blood surface molecule upregulation assay or using an in vitro reporter cell assay, e.g., using HEK-Blue™ CD40L cells (InvivoGen Cat. #: hkb-cd40), as described in greater detail in Example I. These reporter cells were generated by stable transfection of HEK293 cells with the human CD40 gene and an NFκB-inducible secreted embryonic alkaline phosphatase (SEAP) construct to measure the activity of CD40 agonists. Stimulation of CD40 leads to NFκB activation and thus to production of SEAP, which can be detected in the supernatant using chromogenic substrates such as QUANTI-Blue™.

In the context of the present invention, the interferon-associated antigen binding proteins activate both the CD40 and an IFN pathway. In certain embodiments, the interferon-associated antigen binding protein activates the CD40 pathway with an EC₅₀ of less than 400, 300, 200, 150, 100, 70, 60, 50, 40, 30, 25, 20, or 15 ng/mL, wherein CD40 activity is preferably determined using an in vitro reporter cell assay, optionally using HEK-Blue™ CD40L cells, as described for instance in Example I.

In more specific embodiments, the interferon-associated antigen binding protein activates the CD40 pathway with an EC₅₀ ranging from 10 to 200 ng/mL. In even more specific embodiments, the interferon-associated antigen binding protein activates the CD40 pathway with an EC₅₀ ranging from 10 to 50 ng/mL, preferably 10 to 30 ng/mL.

Examples of suitable agonistic anti-CD40 antibodies include, but are not limited to, CP870,893 (Pfizer/Roche), SGN-40 (Seattle Genetics), ADC-1013 (Janssen/Alligator BioSciences), Chi Lob 7/4 (University of Southampton), dacetumumab (Seattle Genetics), APX005M (Apexigen, Inc.), 3G5 (Celldex) and CDX-1140 (Celldex). Exemplary light and heavy chain sequences of the agonistic anti-CD40 antibody CP870,893 are shown in Table 7. Exemplary light and heavy chain sequences of the agonistic anti-CD40 antibody 3G5 are shown in Table 8.

As used herein, the term “agonistic antigen binding fragment” of an agonistic anti-CD40 antibody refers to a fragment of an agonistic anti-CD40 antibody that retains one or more functional activities of the original antibody, such as the ability to bind to and act as an agonist of CD40 signaling in a cell, e.g., it mediates CD40 pathway signaling. Such fragment may compete with the intact antibody for binding to CD40.

Agonistic antigen binding fragments of an agonistic anti-CD40 antibody can be produced by recombinant DNA techniques, or can be produced by enzymatic or chemical cleavage of an anti-CD40 antibody. Agonistic antigen binding fragments include, but are not limited to, a Fab fragment, a diabody (heavy chain variable domain on the same polypeptide as a light chain variable domain, connected via a short peptide linker that is too short to permit pairing between the two domains on the same chain), a Fab′ fragment, a F(ab′)₂ fragment, a Fv fragment, domain antibodies and single-chain antibodies, and can be derived from any mammalian source, including but not limited to human, mouse, rat, camelid or rabbit.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. Variable regions of different antibodies differ extensively in amino acid sequence even among antibodies derived from the same species or of the same class. Exemplary V_{L} and V_{H} domain sequences of the agonistic anti-CD40 antibody CP870,893 are shown in Table 1. The variable region of an antibody typically determines specificity of a particular antibody for its target as it contains the CDRs. Table 1 also shows exemplary CDR sequences of the agonistic anti-CD40 antibody CP870,893.

TABLE 1
Anti-CD40 antibody heavy/light chain variable regions and CDRs of the
agonistic anti-CD40 antibody CP870, 893. Bold italicized sequences
correspond to CDR regions according to the Kabat definition.

| Anti-CD40 antibody regions | Sequence |
|---|---|
| antiCD40 Antibody V_{L} domain | DIQMTQSPSSVSASVGDRVTITCRASQGIYSWLA |
| (SEQ ID NO 51) | WYQQKPGKAPNLLIYTASTLQSGVPSRFSGSGS |
| | GTDFTLTISSLQPEDFATYYCQQANIFPLTFGGG |
| | TKVEIK |
| antiCD40 Antibody CDRL1 | RASQGIYSWLA |
| (SEQ ID NO 52) | |
| antiCD40 Antibody CDRL2 | TASTLQS |
| (SEQ ID NO 53) | |
| antiCD40 Antibody CDRL3 | QQANIFPLT |
| (SEQ ID NO 54) | |
| antiCD40 Antibody V_{H} domain | QVQLVQSGAEVKKPGASVKVSCKASGYTFTGY |
| (SEQ ID NO 55) | YMHWVRQAPGQGLEWMGWINPDSGGTNYAQ |
| | KFQGRVTMTRDTSISTAYMELNRLRSDDTAVY |
| | YCARDQPLGYCTNGVCSYFDYWGQGTLVTVSS |
| antiCD40 Antibody CDRH1 | TGYYMH |
| (SEQ ID NO 56) | |
| antiCD40 Antibody CDRH2 | WINPDSGGTNYAQKFQG |
| (SEQ ID NO 57) | |
| antiCD40 Antibody CDRH3 | DQPLGYCTNGVCSYFDY |
| (SEQ ID NO 58) | |

Delineation of a CDR and identification of residues comprising the binding site of an antibody may be accomplished by solving the structure of the antibody and/or solving the structure of the antibody-ligand complex. This can be accomplished by any of a variety of techniques known to those skilled in the art, such as X-ray crystallography. Various methods of analysis can be employed to identify or approximate the CDR regions. Examples of such methods include, but are not limited to, the Kabat definition, the Chothia definition, the AbM definition and the contact definition.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

In certain embodiments, the complementarity determining regions (CDRs) of the light and heavy chain variable regions of an agonistic anti-CD40 antibody, or an agonistic antigen binding fragment thereof, can be grafted to framework regions (FRs) from the same, or another, species. In certain embodiments, the CDRs of the light and heavy chain variable regions of an agonistic anti-CD40 antibody, or an agonistic antigen binding fragment thereof, can be grafted to consensus human FRs. To create consensus human FRs, in certain embodiments, FRs from several human heavy chain or light chain amino acid sequences are aligned to identify a consensus amino acid sequence. In certain embodiments, the FRs of the heavy chain or light chain of an agonistic anti-CD40 antibody, or an agonistic antigen binding fragment thereof, are replaced with the FRs from a different heavy chain or light chain. In certain embodiments, rare amino acids in the FRs of the heavy and light chains of an agonistic anti-CD40 antibody, or an agonistic antigen binding fragment thereof, are not replaced, while the rest of the FR amino acids are replaced. Rare amino acids are specific amino acids that are in positions in which they are not usually found in FRs. In certain embodiments, the grafted variable regions from an agonistic anti-CD40 antibody, or an agonistic antigen binding fragment thereof, can be used with a constant region that is different from the constant region of an agonistic anti-CD40 antibody, or an agonistic antigen binding fragment thereof. In certain embodiments, the grafted variable regions are part of a single chain Fv antibody. CDR grafting is described, e.g., in U.S. Pat. Nos. 6,180,370, 6,054,297, 5,693,762, 5,859,205, 5,693,761, 5,565,332, 5,585,089, and 5,530,101, and in Jones et al., Nature, 321: 522-525 (1986); Riechmann et al., Nature, 332: 323-327 (1988); Verhoeyen et al., Science, 239:1534-1536 (1988), Winter, FEBS Letts., 430:92-94 (1998), which are hereby incorporated by reference for any purpose.

An “Fc” region typically comprises two heavy chain fragments comprising the C_{H}2 and C_{H}3 domains of an antibody. The two heavy chain fragments are held together by two or more disulfide bonds and by hydrophobic interactions of the C_{H}3 domains.

A “Fab fragment” comprises one full-length light chain as well as the C_{H}1 and variable regions of one heavy chain (the combination of the V_{H} and C_{H}1 regions is referred to herein as “fab region heavy chain”).

A “Fab′ fragment” comprises one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649 and U.S. Pat. Nos. 4,946,778 and 5,260,203, the disclosures of which are incorporated by reference.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody can target the same or different antigens.

An antibody or antigen binding protein, such as an interferon-associated antigen binding protein according to the invention, preferably binds to its target antigen with a dissociation constant (K_{d}) of ≤10⁻⁷ M. The antibody or antigen binding protein binds its antigen with “high affinity” when the K_{d} is ≤5×10⁻⁹ M, and with “very high affinity” when the K_{d} is <5×10⁻¹ M. More preferably, the antibody or antigen binding protein has a K_{d} of <10⁻⁹ M. In some embodiment, the off-rate is <1×10⁻⁵. In other embodiments, the antibody or antigen binding protein will bind to human CD40 with a K_{d} of between about 10⁻⁹ M and 10⁻¹³ M, and in yet another embodiment the antibody or antigen binding protein will bind with a K_{d}<5×10⁻¹⁰. As will be appreciated by one of skill in the art, in some embodiments, any or all of the antigen binding fragments can bind to CD40. Preferably, said constants are determined using surface plasmon resonance, more preferably using the BIAcore® system.

The term “surface plasmon resonance” means an optical phenomenon that allows for the analysis of real-time biospecific interactions by detection of alterations in protein concentrations within a biosensor matrix, for example using the BIAcore® system (BIAcore International AB, a GE Healthcare company, Uppsala, Sweden and Piscataway, N.J.). For further descriptions, see Jönsson et al. (1993) Ann. Biol. Clin. 51:19-26. The term “Kₒₙ” means the on rate constant for association of a binding protein (e.g., an antibody or antigen binding protein) to the antigen to form the, e.g., antigen binding protein/antigen complex. The term “Kₒₙ”, or “on-rate” also means “association rate constant”, or “ka”, as is used interchangeably herein. This value indicating the binding rate of a binding protein to its target antigen or the rate of complex formation between a binding protein, e.g., an antibody or an antigen binding protein, and antigen also is shown by the equation below:

Antibody(“Ab”)+Antigen(“Ag”)→Ab−Ag

The term “K_{off}”, or “off-rate”, means the off rate constant for dissociation, or “dissociation rate constant”, of a binding protein (e.g., an antibody or antigen binding protein) from the, e.g., antigen binding protein/antigen complex as is known in the art. This value indicates the dissociation rate of a binding protein, e.g., an antibody or an antigen binding protein, from its target antigen or separation of Ab-Ag complex over time into free antibody and antigen as shown by the equation below:

Ab+Ag←Ab−Ag

The terms “K_{d}” and “equilibrium dissociation constant” means the value obtained in a titration measurement at equilibrium, or by dividing the dissociation rate constant (K_{off}) by the association rate constant (Kₒₙ). The association rate constant, the dissociation rate constant and the equilibrium dissociation constant, are used to represent the binding affinity of a binding protein (e.g., an antibody or an antigen binding protein) to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay, can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.), can also be used.

An antigen binding protein according to the invention may bind to one target with an affinity at least one order of magnitude, preferably at least two orders of magnitude higher than for a second target.

The term “target” refers to a molecule or a portion of a molecule capable of being bound by an antigen binding protein. In certain embodiments, a target can have one or more epitopes. It will therefore be understood that the target may serve as “antigen” for the “antigen binding protein” of the present invention.

The term “epitope” includes any determinant capable of being bound by an antigen binding protein, such as an antibody. An epitope is a region of an antigen that is bound by an antigen binding protein that targets that antigen, and when the antigen is a protein, includes specific amino acids that directly contact the antigen binding protein. Most often, epitopes reside on proteins, but in some instances can reside on other kinds of molecules, such as nucleic acids. Epitope determinants can include chemically active surface groupings of molecules such as amino acids, sugar side chains, phosphoryl or sulfonyl groups, and can have specific three-dimensional structural characteristics, and/or specific charge characteristics. Generally, antibodies specific for a particular target antigen will preferentially/specifically recognize an epitope on the target antigen in a complex mixture of proteins and/or macromolecules.

In exemplary embodiments, the agonistic anti-CD40 antibody, or the agonistic antigen binding fragment thereof forming part (I) of the interferon-associated antigen binding proteins of the invention comprises three light chain complementarity determining regions (CDRs) that are at least 90% identical to the CDRL1, CDRL2 and CDRL3 sequences within SEQ ID NO 3; and three heavy chain CDRs that are at least 90% identical to the CDRH1, CDRH2 and CDRH3 sequences within SEQ ID NO 6. The agonistic anti-CD40 antibody, or the agonistic antigen binding fragment thereof, may also comprise three light chain complementarity determining regions (CDRs) that are identical to the CDRL1, CDRL2 and CDRL3 sequences within SEQ ID NO 3; and three heavy chain CDRs that are identical to the CDRH1, CDRH2 and CDRH3 sequences within SEQ ID NO 6. In such embodiments, each CDR is defined in accordance with the Kabat definition, the Chothia definition, the AbM definition, or the contact definition of CDR; preferably wherein each CDR is defined in accordance with the CDR definition of Kabat or the CDR definition of Chothia. In particular embodiments, each CDR is defined in accordance with the Kabat definition. In other particular embodiments, each CDR is defined in accordance with the Chothia definition.

Alternatively, the agonistic anti-CD40 antibody, or the agonistic antigen binding fragment thereof forming part (I) of the interferon-associated antigen binding proteins of the invention may comprise (a) a heavy chain or a fragment thereof comprising a complementarity determining region (CDR) CDRH1 that is at least 90%, at least 95%, at least 98% or at least 99% identical to SEQ ID NO 56, a CDRH2 that is at least 90%, at least 95%, at least 98% or at least 99% identical to SEQ ID NO 57, and a CDRH3 that is at least 90%, at least 95%, at least 98% or at least 99% identical to SEQ ID NO 58; and (b) a light chain or a fragment thereof comprising a CDRL1 that is at least 90%, at least 95%, at least 98% or at least 99% identical to SEQ ID NO 52, a CDRL2 that is at least 90%, at least 95%, at least 98% or at least 99% identical to SEQ ID NO 53, and a CDRL3 that is at least 90%, at least 95%, at least 98% or at least 99% identical to SEQ ID NO 54.

In some embodiments, the agonistic anti-CD40 antibody, or the agonistic antigen binding fragment thereof, comprises (a) a heavy chain or a fragment thereof comprising a complementarity determining region (CDR) CDRH1 that is identical to SEQ ID NO 56, a CDRH2 that is identical to SEQ ID NO 57, and a CDRH3 that is identical to SEQ ID NO 58; and (b) a light chain or a fragment thereof comprising a CDRL1 that is identical to SEQ ID NO 52, a CDRL2 that is identical to SEQ ID NO 53, and a CDRL3 that is identical to SEQ ID NO 54.

More specifically the agonistic anti-CD40 antibody, or the agonistic antigen binding fragment thereof, comprises a light chain variable region V_{L} comprising the sequence as set forth in SEQ ID NO 51, or a sequence at least 90%, at least 95%, at least 98% or at least 99% identical thereto; and/or a heavy chain variable region V_{H} comprising the sequence as set forth in SEQ ID NO 55, or a sequence at least 90%, at least 95%, at least 98% or at least 99% identical thereto.

The interferon-associated antigen binding proteins of the invention may also comprise an agonistic anti-CD40 antibody or an agonistic antigen binding fragment thereof, comprising a Fab region heavy chain comprising an amino acid sequence as set forth in SEQ ID NO 12, or a sequence at least 90%, at least 95%, at least 98% or at least 99% identical thereto.

In some embodiments, the agonistic anti-CD40 antibody or the agonistic antigen binding fragment thereof comprises a light chain (LC) that comprises a sequence as set forth in SEQ ID NO 3, or a sequence at least 90%, at least 95%, at least 98% or at least 99% identical thereto; and/or a heavy chain (HC) that comprises a sequence selected from the group consisting of SEQ ID NO 6, SEQ ID NO 9, SEQ ID NO 49, SEQ ID NO 12 and SEQ ID NO 50, or a sequence at least 90%, at least 95%, at least 98% or at least 99% identical thereto.

In more specific embodiments, the agonistic anti-CD40 antibody or the agonistic antigen binding fragment thereof comprises a light chain (LC) that comprises a sequence as set forth in SEQ ID NO 3, or a sequence at least 90%, at least 95%, at least 98% or at least 99% identical thereto; and/or a heavy chain (HC) that comprises a sequence as set forth in SEQ ID NO 6, or a sequence at least 90%, at least 95%, at least 98% or at least 99% identical thereto.

In more specific embodiments, the agonistic anti-CD40 antibody or the agonistic antigen binding fragment thereof comprises a light chain (LC) that comprises a sequence as set forth in SEQ ID NO 3, or a sequence at least 90%, at least 95%, at least 98% or at least 99% identical thereto; and/or a heavy chain (HC) that comprises a sequence as set forth in SEQ ID NO 9, or a sequence at least 90%, at least 95%, at least 98% or at least 99% identical thereto.

In other more specific embodiments, the agonistic anti-CD40 antibody or the agonistic antigen binding fragment thereof comprises a light chain (LC) that comprises a sequence as set forth in SEQ ID NO 3, or a sequence at least 90%, at least 95%, at least 98% or at least 99% identical thereto; and/or a heavy chain (HC) that comprises a sequence as set forth in SEQ ID NO 49, or a sequence at least 90%, at least 95%, at least 98% or at least 99% identical thereto.

In other more specific embodiments, the agonistic anti-CD40 antibody or the agonistic antigen binding fragment thereof comprises a light chain (LC) that comprises a sequence as set forth in SEQ ID NO 3, or a sequence at least 90%, at least 95%, at least 98% or at least 99% identical thereto; and/or a heavy chain (HC) that comprises a sequence as set forth in SEQ ID NO 12, or a sequence at least 90%, at least 95%, at least 98% or at least 99% identical thereto.

In other more specific embodiments, the agonistic anti-CD40 antibody or the agonistic antigen binding fragment thereof comprises a light chain (LC) that comprises a sequence as set forth in SEQ ID NO 3, or a sequence at least 90%, at least 95%, at least 98% or at least 99% identical thereto; and/or a heavy chain (HC) that comprises a sequence as set forth in SEQ ID NO 50, or a sequence at least 90%, at least 95%, at least 98% or at least 99% identical thereto.

In some embodiments, the agonistic anti-CD40 antibody or the agonistic antigen binding fragment thereof comprises a light chain (LC) that comprises a sequence as set forth in SEQ ID NO 59, or a sequence at least 90%, at least 95%, at least 98% or at least 99% identical thereto; and/or a heavy chain (HC) that comprises a sequence selected from the group consisting of SEQ ID NO 61, SEQ ID NO 63 and SEQ ID NO 65, or a sequence at least 90%, at least 95%, at least 98% or at least 99% identical thereto.

In more specific embodiments, the agonistic anti-CD40 antibody or the agonistic antigen binding fragment thereof comprises a light chain (LC) that comprises a sequence as set forth in SEQ ID NO 59, or a sequence at least 90%, at least 95%, at least 98% or at least 99% identical thereto; and/or a heavy chain (HC) that comprises a sequence as set forth in SEQ ID NO 61, or a sequence at least 90%, at least 95%, at least 98% or at least 99% identical thereto.

In other more specific embodiments, the agonistic anti-CD40 antibody or the agonistic antigen binding fragment thereof comprises a light chain (LC) that comprises a sequence as set forth in SEQ ID NO 59, or a sequence at least 90%, at least 95%, at least 98% or at least 99% identical thereto; and/or a heavy chain (HC) that comprises a sequence as set forth in SEQ ID NO 63, or a sequence at least 90%, at least 95%, at least 98% or at least 99% identical thereto.

In other more specific embodiments, the agonistic anti-CD40 antibody or the agonistic antigen binding fragment thereof comprises a light chain (LC) that comprises a sequence as set forth in SEQ ID NO 59, or a sequence at least 90%, at least 95%, at least 98% or at least 99% identical thereto; and/or a heavy chain (HC) that comprises a sequence as set forth in SEQ ID NO 65, or a sequence at least 90%, at least 95%, at least 98% or at least 99% identical thereto.

### Variants and Derivatives of Interferon-Associated Antigen Binding Protein or Components Thereof

A “variant” of a polypeptide (e.g., an interferon-associated antigen binding protein, an interferon-fused agonistic anti-CD40 antibody or an interferon-fused agonistic antigen binding fragment thereof, an antibody, an antigen binding protein, or an IFN, or components thereof) comprises an amino acid sequence wherein one, two, three, four, five or more amino acid residues are inserted into, deleted from and/or substituted into the amino acid sequence relative to another polypeptide sequence. Preferably, the variant comprises up to ten insertions, deletions and/or substitutions, more preferably up to eight insertions, deletions and/or substitutions. More specifically, the variant may comprise up to ten, more preferably up to eight insertions. The variant may also comprise up to ten, more preferably up to eight deletions. In even more preferred embodiments, the variant comprises up to ten substitutions, most preferably up to eight substitutions. In some embodiments, these substitutions are conservative amino acid substitution as described below.

A “variant” of a polynucleotide sequence (e.g., RNA or DNA) comprises one or more mutations within the polynucleotide sequence relative to another polynucleotide sequence, wherein one, two, three, four, five or more nucleic acid residues are inserted into, deleted from and/or substituted into the nucleic acid sequence. Preferably, the variant comprises up to ten insertions, deletions and/or substitutions, more preferably up to eight insertions, deletions and/or substitutions. More specifically, the variant may comprise up to ten, more preferably up to eight insertions. The variant may also comprise up to ten, more preferably up to eight deletions. In even more preferred embodiments, the variant comprises up to ten substitutions, most preferably up to eight substitutions. Said one, two, three, four, five or more mutations can cause one, two, three, four, five or more amino acid exchanges within the amino acid sequence the variant encodes for as compared to another amino acid sequence (i.e. a “non-silent mutation”). Variants also include nucleic acid sequences wherein one, two, three, four, five or more codons have been replaced by their synonyms which does not cause an amino acid exchange and is thus called a “silent mutation”.

The term “identity” or “homology”, in the context of variants of polypeptide or nucleotide sequences, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. Preferably, identity is determined over the full length of a sequence. It is understood that the expression “at least 80% identical”, includes embodiments wherein the described or claimed sequence is at least 85%, preferably at least 90%, more preferably at least 95%, more preferably at least 98% or still more preferably at least 99% identical to the reference sequence. The expression “at least 90% identical” includes embodiments wherein the described or claimed sequence is at least 90%, preferably at least 91%, more preferably at least 92%, more preferably at least 93%, more preferably at least 94%, more preferably at least 95%, more preferably at least 96%, more preferably at least 97%, more preferably at least 98% or still more preferably at least 99% identical to the reference sequence.

For the calculation of percent identity, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, WI). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3×the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSum 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSum 62 comparison matrix) is also used by the algorithm.

Examples of parameters that can be employed in determining percent identity for polypeptides or nucleotide sequences using the GAP program are the following:
Algorithm: Needleman et al., 1970, J. Mol. Biol. 48:443-453
Comparison matrix: BLOSum 62 from Henikoff et al., 1992, supra
Gap Penalty: 12 (but with no penalty for end gaps)
Gap Length Penalty: 4
Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 or at least 100, preferably the entire length, of contiguous amino acids of the target polypeptide.

Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties.

Naturally occurring residues can be divided into classes based on common side chain properties:
1) hydrophobic: Norleucine, Met, Ala, Val, Leu, Ile;
2) neutral hydrophilic: Cys, Ser, Thr, Asn, Gln;
3) acidic: Asp, Glu;
4) basic: His, Lys, Arg;
5) residues that influence chain orientation: Gly, Pro; and
6) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the interferon-associated antigen binding protein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within +2 is included, in certain embodiments, those which are within +1 are included, and in certain embodiments, those within +0.5 are included.

Exemplary amino acid substitutions are set forth in Table 2.

TABLE 2
Amino Acid Substitutions.

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric Acid, | Arg |
| | Gln, Asn | |
| Met | Leu, Phe, Ile | Leu |
| Phe | Leu, Val, Ile, Ala, | Leu |
| | Tyr | |
| Pro | Ala, Gly | Ala |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

In light of the present invention, a skilled artisan will be able to determine suitable variants of the interferon-associated antigen binding proteins as set forth herein using well-known techniques. In certain embodiments, one skilled in the art can identify suitable areas of the molecule that may be changed without destroying activity by targeting regions not believed to be important for activity. In certain embodiments, one can identify residues and portions of the molecules that are conserved among similar polypeptides. In certain embodiments, even areas that can be important for biological activity or for structure can be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues which are important for activity or structure in similar proteins. One skilled in the art can opt for chemically similar amino acid substitutions for such predicted important amino acid residues.

One skilled in the art can also analyze the three-dimensional structure and amino acid sequence in relation to that structure in similar proteins or protein domains. In view of such information, one skilled in the art can predict the alignment of amino acid residues of interferon-associated antigen binding protein, an antibody or an antigen binding fragment thereof or an interferon or a functional fragment thereof as described herein with respect to its three dimensional structure. In certain embodiments, one skilled in the art can choose not to make radical changes to amino acid residues predicted to be on the surface of the protein, since such residues can be involved in important interactions with other molecules. Moreover, one skilled in the art can generate test variants containing a single amino acid substitution at each desired amino acid residue. The variants can then be screened using activity assays known to those skilled in the art. Such variants can be used to gather information about suitable variants. For example, if one discovered that a change to a particular amino acid residue resulted in destroyed, undesirably reduced, or unsuitable activity, variants with such a change can be avoided. In other words, based on information gathered from such experiments, one skilled in the art can readily determine the amino acids where further substitutions should be avoided either alone or in combination with other mutations.

According to certain embodiments, amino acid substitutions are those which: (1) reduce susceptibility to proteolysis, (2) reduce susceptibility to oxidation, (3) alter binding affinity for forming protein complexes, (4) alter binding affinities, and/or (5) confer or modify other physicochemical or functional properties on such polypeptides. According to certain embodiments, single or multiple amino acid substitutions (in certain embodiments, conservative amino acid substitutions) can be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden & J. Tooze, eds., Garland Publishing, New York, N.Y. (1991)); and Thornton et al., Nature, 354:105 (1991), which are each incorporated herein by reference.

The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified interferon-associated antigen binding protein can have a greater circulating half-life than an interferon-associated antigen binding protein that is not chemically modified. In certain embodiments, a chemically modified interferon-associated antigen binding protein can have improved targeting capacity for desired cells, tissues, and/or organs. In some embodiments, a derivative interferon-associated antigen binding protein is covalently modified to include one or more water-soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative interferon-associated antigen binding protein comprises one or more polymer, including, but not limited to, monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain embodiments, a derivative of an interferon-associated antigen binding protein as described herein is covalently modified with polyethylene glycol (PEG) subunits. In certain embodiments, one or more water-soluble polymer is bonded at one or more specific position, for example at the amino terminus, of a derivative. In certain embodiments, one or more water-soluble polymer is randomly attached to one or more side chains of a derivative. In certain embodiments, PEG is used to improve the therapeutic capacity of the interferon-associated antigen binding protein. Certain such methods are discussed, for example, in U.S. Pat. No. 6,133,426, which is hereby incorporated by reference for any purpose.

In certain embodiments, interferon-associated antigen binding protein variants include glycosylation variants wherein the number and/or type of glycosylation site has been altered compared to the amino acid sequences of a parent polypeptide. In certain embodiments, protein variants comprise a greater number of N-linked glycosylation sites than the native protein. In other embodiments, protein variants comprise a lesser number of N-linked glycosylation sites than the native protein. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one, two, three, four, five or more N-linked glycosylation sites (typically those that are naturally occurring) are eliminated and one or more new N-linked sites are created. Additional preferred variants include cysteine variants wherein one or more cysteine residues are deleted from or substituted for another amino acid (e.g., serine) as compared to the parent amino acid sequence. Cysteine variants can be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

### Coronavirus Infection

The interferon-associated antigen binding proteins, the nucleic acids, vectors, vector systems, methods and compositions described herein can be used to treat Coronavirus infection, in particular SARS-CoV-2 infection. As used herein, “treat Coronavirus infection” and “treatment of Coronavirus infection” refers to one or more of: (i) rescuing cells from Coronavirus-induced cell death; (ii) rescuing cells from Coronavirus-induced cytopathic effect (iii) decreasing one or more Coronavirus-related disorders; and (iv) decreasing one or more Coronavirus-related symptoms in a subject.

The terms “viral load” and “viral titer” refer to the number of viral particles in a cell, an organ or a bodily fluid such as blood or serum. Viral load or viral titer is often expressed as viral particles, or infectious particles per mL depending on the type of assay. Today, viral load is usually measured using international units per milliliter (IU/mL). Viral load or viral titer may alternatively be determined as so-called viral genome equivalent. A higher viral burden, titer, or viral load often correlates with the severity of an active viral infection. Accordingly, reducing the viral load or viral titer correlates with a reduced number of infectious viral particles, e.g., in the serum. Viral load is usually determined using nucleic acid amplification based tests (NATs or NAATss). NAT/NAAT tests utilize, for example, PCR, (quantitative) reverse transcription polymerase chain reaction (RT-PCR or qRT-PCR), nucleic acid sequence based amplification (NASBA) or probe-based assays. Due to the ease of detection of viral nucleic acids using nucleic acid amplification based tests, the viral load is useful in clinical settings to monitor success during treatment.

The terms “patient” and “subject” are used interchangeably and include human and non-human animal subjects, preferably human subjects, as well as those with formally diagnosed disorders, those without formally recognized disorders, those receiving medical attention, those at risk of developing the disorders, etc.

As used herein, a “Coronavirus-related disorder”, in particular such related to SARS-CoV-2 infection, refers to a disorder that results from infection of a subject by Coronavirus. Coronavirus-related disorders include, but are not limited to Covid-19, respiratory illness, pneumonia, and symptoms and/or complications arising from any of these disorders.

As used herein, a “Coronavirus-related symptom,” a “symptom of Coronavirus infection” or a “Coronavirus-related complication” includes one or more physical dysfunctions related to Coronavirus infection, in particular related to SARS-CoV-2 infection. Coronavirus symptoms and complications include, but are not limited to, fever, dry cough, tiredness, difficulty in breathing or shortness of breath, chest pain or pressure, ageusia, parageusia, hypogeusia, anosmia, parosmia, hyposmia, and the like.

### Interferons

As used herein, an “interferon” or “IFN” refers to a cytokine, or derivative thereof, that is typically produced and released by cells in response to the presence of a pathogen or a tumor cell. IFNs include type I IFNs (e.g., IFNα, IFNβ, IFNε, IFNκ, IFNτ, IFNζ and IFNω), type II IFNs (e.g., IFNγ) and type III IFNs (e.g., IFNλ1, IFNλ2 and IFNλ3). The term “interferon” or “IFN” includes without limitation full-length IFN, a variant or a derivative thereof (e.g., a chemically (e.g., PEGylated) modified derivative or mutein), or a functionally active fragment thereof, that retains one or more signaling activities of a full-length IFN.

As used herein, the term “functional fragment” refers to a fragment of a substance that retains one or more functional activities of the original substance. For example, a functional fragment of an interferon refers to a fragment of an interferon that retains an IFN function as described herein, e.g., it mediates IFN pathway signaling.

The IFN may increase one or more IFN receptor activities by at least about 20% when added to a cell, tissue or organism expressing a cognate IFN receptor (IFNAR for IFNα, IFNBR for IFNβ, etc). In some embodiments, the interferon activates IFN receptor activity by at least 40%, at least 50%, at least 60%, at least 70%, at least 80%, or at least 85%. The activity of the IFN (i.e., the “IFN activity”) may be measured, e.g., using an in vitro reporter cell assay, e.g., using HEK-Blue™ IFN-α/β cells (InvivoGen, Cat. #: hkb-ifnαβ), HEK-Blue™ IFN-λ (InvivoGen, Cat. #: hkb-ifnl) or HEK-Blue™ Dual IFN-γ cells (InvivoGen, Cat. #: hkb-ifng), as described in greater detail in Example I. These reporter cells were generated by stable transfection of HEK293 cells with human IFN receptor genes and an IFN-stimulated response element-controlled secreted embryonic alkaline phosphatase (SEAP) construct to measure the activity of IFNs. HEK-Blue™ IFN-cells are designed to monitor the activation of the JAK/STAT/ISGF3 pathways induced by type I, type II or type III interferons. Activation of these pathways induces the production and release of SEAP.

In the context of the present invention, the interferon-associated antigen binding proteins activate both the CD40 and an IFN pathway. In certain embodiments, the interferon-associated antigen binding protein activates the IFN pathway with an EC₅₀ of less than 100, 60, 50, 40, 30, 20, 10, or 1 ng/mL, preferably with an EC₅₀ of less than 11 ng/mL, more preferably with an EC₅₀ of less than 6 ng/mL, wherein IFN activity is preferably determined using an in vitro reporter cell assay, optionally using HEK-Blue™ IFN-cells, as described for instance in Example I.

In some of these embodiments, the IFN pathway is the IFNα (interferon alpha), IFNβ (interferon beta), IFNε (interferon epsilon), IFNω (interferon omega), IFNγ (interferon gamma), or IFNλ (interferon lambda) pathway.

According to certain exemplary embodiments, an interferon-associated antigen binding protein as described herein comprises full-length IFN, a variant or a derivative thereof (e.g., a chemically (e.g., PEGylated) modified derivative or mutein), or a functionally active fragment thereof, that retains one or more signaling activities of a full-length IFN. In certain embodiments, the IFN is a human IFN.

In certain embodiments, an interferon-associated antigen binding protein as described herein comprises an IFN or a functional fragment thereof selected from the group consisting of a Type I IFN, a Type II IFN and a Type III IFN, or a functional fragment thereof.

In particular embodiments, the IFN or the functional fragment thereof is a Type I IFN, or a functional fragment thereof. In specific embodiments, the type I IFN or the functional fragment thereof is IFNα, IFNβ, IFNω or IFNε, or a functional fragment thereof. In more specific embodiments, the type I IFN or the functional fragment thereof is IFNα or IFNβ, or a functional fragment thereof. In other more specific embodiments, the type I IFN or the functional fragment thereof is IFN a, or a functional fragment thereof. In other more specific embodiments, the type I IFN or the functional fragment thereof is IFN 3, or a functional fragment thereof. In other more specific embodiments, the type I IFN or the functional fragment thereof is IFNω, or a functional fragment thereof. In other more specific embodiments, the type I IFN or the functional fragment thereof is IFNε, or a functional fragment thereof.

In particular embodiments, the IFN or the functional fragment thereof is IFNα, IFNβ, IFNγ, IFNλ, IFNε or IFNω, or a functional fragment thereof. In specific embodiments, the IFN or a functional fragment thereof is IFNα or IFNβ, or a functional fragment thereof.

In some embodiments, the IFN or the functional fragment thereof is IFNα, or a functional fragment thereof. In more specific embodiments, the IFN or functional fragment thereof is IFNα2a, or a functional fragment thereof. The IFNα2a may comprise the sequence as set forth in SEQ ID NO 17, or a sequence at least 90% identical thereto.

In some embodiments, the IFN or the functional fragment thereof is IFNβ, or a functional fragment thereof. The IFNβ may comprise the sequence as set forth in SEQ ID NO 14, or a sequence at least 90% identical thereto. The IFNβ or the functional fragment thereof may comprise one or two amino acid substitution(s) relative to SEQ ID NO 14, selected from C17S and N80Q. In some embodiments, the IFNβ or the functional fragment thereof comprises the amino acid substitution C17S relative to SEQ ID NO 14. In some embodiments, the IFNβ comprises the amino acid sequence as set forth in SEQ ID NO 15. In other embodiments, the IFNβ comprises the amino acid substitutions C17S and N80Q relative to SEQ ID NO 14. In yet other embodiments, the IFNβ comprises the amino acid sequence as set forth in SEQ ID NO 16.

In some embodiments, the IFN or the functional fragment thereof is IFNγ or IFNλ, or a functional fragment thereof. In specific embodiments, the IFN or functional fragment thereof is IFNγ, or a functional fragment thereof. In more specific embodiments, the IFNγ comprises the sequence as set forth in SEQ ID NO 19, or a sequence at least 90% identical thereto. In other specific embodiments, the IFN or functional fragment thereof is IFNλ, or a functional fragment thereof. In more specific embodiments, the IFNλ or the functional fragment thereof is IFNλ2, or a functional fragment thereof. The IFNλ2 may comprise the sequence as set forth in SEQ ID NO 18, or a sequence at least 90% identical thereto.

In some embodiments, the IFN or the functional fragment thereof is IFNε, or a functional fragment thereof. The IFNε may comprise the sequence as set forth in SEQ ID NO 80, or a sequence at least 90% identical thereto.

In some embodiments, the IFN or the functional fragment thereof is IFNω, or a functional fragment thereof. The IFNω may comprise the sequence as set forth in SEQ ID NO 79, or a sequence at least 90% identical thereto.

In certain embodiments, the expression level of one or more IFN signaling pathway biomarkers is altered, i.e., upregulated or downregulated, in a Coronavirus-infected cell treated with an interferon-associated antigen binding protein described herein. According to certain exemplary embodiments, the expression level of one or more IFN pathway biomarkers is upregulated in a Coronavirus-infected cell treated with an interferon-associated antigen binding protein described herein. In this context, a “biomarker” is to be understood as a characteristic that is objectively measured and evaluated as an indicator of normal biological processes, pathogenic processes, or pharmacologic responses to a therapeutic intervention.

According to certain embodiments, a suitable IFN pathway biomarker featured herein is a chemokine, e.g., a C-X-C chemokine, selected from the group consisting of CXCL9, CXCL10 and CXCL11. In certain exemplary embodiments, a suitable biomarker induced by the IFN pathway is CXCL9, CXCL10 and/or CXCL11, and also the interferon stimulated gene ISG20. Cytokine induction or release may be quantified using techniques known in the art, such as ELISA. Alternatively, induction may also be determined using RNA-based assays such as RNAseq or qRT-PCR. In certain embodiments, upregulation may refer to an at least at 1.5-fold, at least 2-fold, at least 2.5-fold, at least 3-fold, at least 4-fold, at least 5-fold or at least 10-fold increased expression or secretion of these cytokines.

In these or in other exemplary embodiments, the expression level of pro-inflammatory cytokines, e.g., IL10, IL1β and/or IL2 is not significantly upregulated in human Whole Blood cells upon treatment with an interferon-associated antigen binding protein of the invention. In some embodiments, the expression level of IL10 is not significantly upregulated in human Whole Blood cells upon treatment with an interferon-associated antigen binding protein of the invention. In some embodiments, the expression level of IL1β is not significantly upregulated in human Whole Blood cells upon treatment with an interferon-associated antigen binding protein of the invention. In some embodiments, the expression level of IL2 is not significantly upregulated in a Coronavirus-infected cell upon treatment with an interferon-associated antigen binding protein of the invention. In some embodiments, the expression levels of IL10 and IL1β are not significantly upregulated in a Coronavirus-infected cell upon treatment with an interferon-associated antigen binding protein of the invention. In some embodiments, the expression levels of IL10 and IL2 are not significantly upregulated in a Coronavirus-infected cell upon treatment with an interferon-associated antigen binding protein of the invention. In some embodiments, the expression levels of IL1β and IL2 are not significantly upregulated in a Coronavirus-infected cell upon treatment with an interferon-associated antigen binding protein of the invention. In some embodiments, the expression levels of IL10, IL1β and IL2 are not significantly upregulated in a Coronavirus-infected cell upon treatment with an interferon-associated antigen binding protein of the invention.

### Interferon-Associated Antigen Binding Proteins

The term “associated”, as used herein, generally refers to a covalent or non-covalent linkage of two (or more) molecules. Associated proteins are created by joining two or more distinct peptides or proteins, resulting in a protein with one or more functional properties derived from each of the original proteins. In the context of the present invention, the interferon-associated antigen binding proteins activate both the CD40 and an IFN pathway. An associated protein encompasses monomeric and multimeric, e.g., dimeric, trimeric, tetrameric or the like, complexes of distinct associated or fused proteins. In this context, non-covalent linkage results from strong interactions between two protein surface regions, usually via ionic, Van-der-Waals, and/or hydrogen bond interactions. Covalent linkage, on the other hand, requires the presence of actual chemical bonds, such as peptide bonds, disulphide bridges, etc. The term “fused” as used herein, generally refers to the joining of two or more distinct peptides or proteins in a covalent fashion via a peptide bond. Thus, a “fused protein” refers to single protein created by joining two or more distinct peptides or proteins via a peptide bond with one or more functional properties derived from each of the original proteins. In certain embodiments, two or more distinct peptides or proteins may be fused to one another via one or more peptide linkers (“L”).

In a certain aspect of the invention, an interferon-associated antigen binding protein is a protein comprising an agonistic anti-CD40 antibody or an agonistic antigen binding fragment thereof and an IFN or a functional fragment thereof.

In some embodiments, the IFN or the functional fragment thereof is non-covalently associated with the agonistic anti-CD40 antibody or the agonistic antigen binding fragment thereof. In more specific embodiments, the IFN or the functional fragment thereof is non-covalently associated with the agonistic anti-CD40 antibody or the agonistic antigen binding fragment thereof via ionic, Van-der-Waals, and/or hydrogen bond interactions.

In other embodiments, the IFN or the functional fragment thereof is covalently associated with the agonistic anti-CD40 antibody or the agonistic antigen binding fragment thereof. In preferred embodiments, the IFN or the functional fragment thereof is fused to the agonistic anti-CD40 antibody or the agonistic antigen binding fragment thereof. The IFN or the functional fragment thereof may be fused to a light chain of the agonistic anti-CD40 antibody or the agonistic antigen binding fragment thereof. In some embodiments, the IFN or the functional fragment thereof is fused to the N-terminus of a light chain of the agonistic anti-CD40 antibody or the agonistic antigen binding fragment thereof. In other embodiments, the IFN or the functional fragment thereof is fused to the C-terminus of a light chain of the agonistic anti-CD40 antibody or the agonistic antigen binding fragment thereof. The IFN or the functional fragment thereof may be also be fused to a heavy chain of the agonistic anti-CD40 antibody or the agonistic antigen binding fragment thereof. In some embodiments, the IFN or the functional fragment thereof is fused to the N-terminus of a heavy chain of the agonistic anti-CD40 antibody or the agonistic antigen binding fragment thereof. In other embodiments, the IFN or the functional fragment thereof is fused to the C-terminus of a heavy chain of the agonistic anti-CD40 antibody or the agonistic antigen binding fragment thereof. In any of these embodiments, the agonistic anti-CD40 antibody or an agonistic antigen binding fragment thereof, and the IFN or the functional fragment thereof may be fused to each other via a linker.

The term “linker” or “L,” as used herein, refers to any moiety that covalently joins one or more agonistic anti-CD40 antibody or an agonistic antigen binding fragment thereof to one or more interferon, or a functional fragment thereof. In exemplary embodiments, a linker is a peptide linker. The term “peptide linker”, as used herein, refers to a peptide adapted to link two or more moieties. A peptide linker referred to herein may have one or more of the properties outlined in the following. The sequences of peptide linker according to certain exemplary embodiments are set forth in Table 7.

A peptide linker may have any length, i.e., comprise any number of amino acid residues. In exemplary embodiments, the linker comprises at least 1, at least 2, at least 3, at least 4, at least 5 amino acids. The linker may comprise at least 4 amino acids. The linker may comprise at least 11 amino acids. The linker may comprise at least 12 amino acids. The linker may comprise at least 13 amino acids. The linker may comprise at least 15 amino acids. The linker may comprise at least 20 amino acids. The linker may comprise at least 21 amino acids. The linker may comprise at least 24 amino acids.

A linker is typically long enough to provide an adequate degree of flexibility to prevent the linked moieties from interfering with each other's activity, e.g., the ability of a moiety to bind to a receptor. In exemplary embodiments, the linker comprises up to 10, up to 20, up to 30, up to 40, up to 50, up to 60, up to 70, up to 80, up to 90, or up to 100 amino acids. The linker may comprise up to 80 amino acids. The linker may comprise up to 40 amino acids. The linker may comprise up to 24 amino acids. The linker may comprise up to 21 amino acids. The linker may comprise up to 20 amino acids. The linker may comprise up to 15 amino acids. The linker may comprise up to 13 amino acids. The linker may comprise up to 12 amino acids. The linker may comprise up to 11 amino acids. The linker may comprise up to 4 amino acids.

In some embodiments, the linker is selected from the group comprising rigid, flexible and/or helix-forming linkers. It is understood that helix-forming linkers can also be rigid linkers, since an α-helix has less degrees of freedom than a peptide assuming a more random-coil conformation. In some embodiments, the linker is a rigid linker. An exemplary rigid linker comprises a sequence as set forth in SEQ ID NO 20. Further exemplary rigid linkers comprise a sequence as set forth in SEQ ID NO 22 or SEQ ID NO 23. In related embodiments, the linker is a helix-forming linker. Exemplary helix-forming linkers comprise a sequence as set forth in SEQ ID NO 22 or SEQ ID NO 23. In other embodiments, the linker is a flexible linker. Exemplary flexible linkers comprise a sequence as set forth in SEQ ID NO 21, SEQ ID NO 24, SEQ ID NO 25 or SEQ ID NO 26.

The linker can also have different chemical properties. A linker can be selected from acidic, basic or neutral linkers. Typically, acidic linkers contain one or more acidic amino acid, such as Asp or Glu. Basic linkers typically contain one or more basic amino acids, such as Arg, His and Lys. Both types of amino acids are very hydrophilic. In some embodiments, the linker is an acidic linker. Exemplary acidic linkers comprise a sequence as set forth in SEQ ID NO 22 or SEQ ID NO 23. In other embodiments, the linker is a basic linker. In yet other embodiments, the linker is a neutral linker. Exemplary neutral linkers comprise a sequence as set forth in SEQ ID NO 20, SEQ ID NO 21, SEQ ID NO 24, SEQ ID NO 25 or SEQ ID NO 26.

In preferred embodiments, the linker is Gly-Ser or a Gly-Ser-Thr linker composed of multiple glycine, serine and, where applicable, threonine residues. In some of these embodiments, the linker comprises the amino acids glycine and serine. In more specific embodiments, the linker comprises the sequence as set forth in SEQ ID NO 21, SEQ ID NO 24, SEQ ID NO 25, SEQ ID NO 26. In some embodiments, the linker further comprises the amino acid threonine. In a more specific embodiment, the linker comprises the sequence as set forth in SEQ ID NO 21.

In exemplary embodiments of the present invention, the interferon-associated antigen binding protein comprises a linker comprising a sequence selected from the sequences as set forth in SEQ ID NOs 20 to 26, preferably from the sequences as set forth in SEQ ID NO 24, SEQ ID NO 25 or SEQ ID NO 26. In a preferred embodiment, the linker comprises a sequence as set forth in SEQ ID NO 24. In another preferred embodiment, the linker comprises a sequence as set forth in SEQ ID NO 25. In another preferred embodiment, the linker comprises a sequence as set forth in SEQ ID NO 26.

In various embodiments of any one of the aspects of the invention, the interferon-associated antigen binding protein comprises no amino acids other than those forming (I) said agonistic anti-CD40 antibody, or agonistic antigen binding fragment thereof and (II) said IFN or functional fragment thereof. In related embodiments, the interferon-associated antigen binding protein comprises no amino acids other than those forming (I) said agonistic anti-CD40 antibody, or agonistic antigen binding fragment thereof, (II) said IFN or functional fragment thereof and (III) said linker.

Exemplary embodiments representing the various different configurations of (I) the agonistic anti-CD40 antibody or the agonistic antigen binding fragment thereof, (II) the interferon (IFN) or the functional fragment thereof and (III) the linker are outlined in the following.

In certain preferred embodiments, the IFN or a functional fragment thereof is fused to the C-terminus of a heavy chain of the agonistic anti-CD40 antibody, or the agonistic antigen binding fragment thereof, via the linker, as set forth in Table 3A or Table 3B. In these embodiments, the heavy chain of the agonistic anti-CD40 antibody, or the agonistic antigen binding fragment thereof, may comprise a sequence as set forth in SEQ ID NO 6, SEQ ID NO 9, SEQ ID NO 12, SEQ ID NO 48 or SEQ ID NO 49, SEQ ID NO 61, or SEQ ID NO 63. The IFNα2a may comprise the sequence as set forth in SEQ ID NO 17. The IFNβ may comprise the sequence as set forth in SEQ ID NO 14, SEQ ID NO 15 or SEQ ID NO 16. The IFNβ may comprise the sequence as set forth in SEQ ID NO 14. The IFNβ_C17S may comprise the sequence as set forth in SEQ ID NO 15. The IFNβ_C17S,N80Q may comprise the sequence as set forth in SEQ ID NO 16. The IFNγ may comprise the sequence as set forth in SEQ ID NO 19. The IFNλ2 may comprise the sequence as set forth in SEQ ID NO 18. The IFNε may comprise the sequence as set forth in SEQ ID NO 80. The IFNω may comprise the sequence as set forth in SEQ ID NO 79. The linkers referred to are those listed in Table 7.

In the embodiments where the IFN is fused to the C-terminus of the heavy chain of the agonistic anti-CD40 antibody, or the agonistic antigen binding fragment thereof, the interferon-associated antigen binding protein further comprises a light chain of an agonistic anti-CD40 antibody, or an agonistic antigen binding fragment thereof. In more specific embodiments, a heavy chain comprises a sequence as set forth in SEQ ID NO 6, SEQ ID NO 9, SEQ ID NO 12, SEQ ID NO 48, or SEQ ID NO 49 and a light chain comprises a sequence as set forth in SEQ ID NO 3. In other more specific embodiments, a heavy chain comprises a sequence as set forth in SEQ ID NO 61 or SEQ ID NO 63 and a light chain comprises a sequence as set forth in SEQ ID NO 59.

TABLE 3
Interferon or a functional fragment thereof fused to the C-terminus of a heavy chain
of the anti-CD40 antibody or an agonistic antigen binding fragment thereof.

| A | IFNα2a | IFNβ | IFNβ_C17S | IFNβ_C17S, N80Q | IFNγ | IFNλ2 |
|---|---|---|---|---|---|---|
| RL | antiCD40_HC-- | antiCD40_HC-- | antiCD40_HC-- | antiCD40_HC--RL-- | antiCD40_HC-- | antiCD40_HC-- |
| linker | RL--IFNα2a | RL--IFNβ | RL--IFNβ_C17S | IFNβ_C17S, N80Q | RL--IFNγ | RL--IFNλ2 |
| GST | antiCD40_HC-- | antiCD40_HC-- | antiCD40_HC-- | antiCD40_HC-- | antiCD40_HC-- | antiCD40_HC-- |
| linker | GST--IFNα2a | GST--IFNβ | GST-- | GST-- | GST--IFNγ | GST--IFNλ2 |
| | | | IFNβ_C17S | IFNβ_C17S, N80Q | | |
| HL | antiCD40_HC-- | antiCD40_HC-- | antiCD40_HC-- | antiCD40_HC--HL-- | antiCD40_HC-- | antiCD40_HC-- |
| linker | HL--IFNα2a | HL--IFNβ | HL--IFNβ_C17S | IFNβ_C17S, N80Q | HL--IFNγ | HL--IFNλ2 |
| HL2 | antiCD40_HC-- | antiCD40_HC-- | antiCD40_HC-- | antiCD40_HC--HL2-- | antiCD40_HC-- | antiCD40_HC-- |
| linker | HL2--IFNα2a | HL2--IFNβ | HL2-- | IFNβ_C17S, N80Q | HL2--IFNγ | HL2--IFNλ2 |
| | | | IFNβ_C17S | | | |
| (G4S)2 | antiCD40_HC-- | antiCD40_HC-- | antiCD40_HC-- | antiCD40_HC-- | antiCD40_HC-- | antiCD40_HC-- |
| linker | (G4S)2--IFNα2a | (G4S)2--IFNβ | (G4S)2-- | (G4S)2-- | (G4S)2--IFNγ | (G4S)2--IFNλ2 |
| | | | IFNβ_C17S | IFNβ_C17S, N80Q | | |
| (G4S)3 | antiCD40_HC-- | antiCD40_HC-- | antiCD40_HC-- | antiCD40_HC-- | antiCD40_HC-- | antiCD40_HC-- |
| linker | (G4S)3--IFNα2a | (G4S)3--IFNβ | (G4S)3-- | (G4S)3-- | (G4S)3--IFNγ | (G4S)3--IFNλ2 |
| | | | IFNβ_C17S | IFNβ_C17S, N80Q | | |
| (G4S)4 | antiCD40_HC-- | antiCD40_HC-- | antiCD40_HC-- | antiCD40_HC-- | antiCD40_HC-- | antiCD40_HC-- |
| linker | (G4S)4--IFNα2a | (G4S)4--IFNβ | (G4S)4-- | (G4S)4-- | (G4S)4--IFNγ | (G4S)4--IFNλ2 |
| | | | IFNβ_C17S | IFNβ_C17S, N80Q | | |

| B | IFNε | IFNω |
|---|---|---|
| RL | antiCD40_HC-- | antiCD40_HC-- |
| linker | RL--IFNε | RL--IFNω |
| GST | antiCD40_HC-- | antiCD40_HC-- |
| linker | GST--IFNε | GST--IFNω |
| HL | antiCD40_HC-- | antiCD40_HC-- |
| linker | HL--IFNε | HL--IFNω |
| HL2 | antiCD40_HC-- | antiCD40_HC-- |
| linker | HL2--IFNε | HL2--IFNω |
| (G4S)2 | antiCD40_HC-- | antiCD40_HC-- |
| linker | (G4S)2--IFNε | (G4S)2--IFNω |
| (G4S)3 | antiCD40_HC-- | antiCD40_HC-- |
| linker | (G4S)3--IFNε | (G4S)3--IFNω |
| (G4S)4 | antiCD40_HC-- | antiCD40_HC-- |
| linker | (G4S)4--IFNε | (G4S)4--IFNω |

In certain preferred embodiments, the IFN or a functional fragment thereof is fused to the N-terminus of a heavy chain of the agonistic anti-CD40 antibody, or the agonistic antigen binding fragment thereof, via the linker, as set forth in Table 4A or Table 4B. In these embodiments, the heavy chain of the agonistic anti-CD40 antibody, or the agonistic antigen binding fragment thereof, may comprise a sequence as set forth in SEQ ID NO 6, SEQ ID NO 9, SEQ ID NO 12, SEQ ID NO 48, SEQ ID NO 49, SEQ ID NO 50, SEQ ID NO 61, SEQ ID NO 63 or SEQ ID NO 65. The IFNα2a may comprise the sequence as set forth in SEQ ID NO 17. The IFNβ may comprise the sequence as set forth in SEQ ID NO 14, SEQ ID NO 15 or SEQ ID NO 16. The IFNβ may comprise the sequence as set forth in SEQ ID NO 14. The IFNβ_C17S may comprise the sequence as set forth in SEQ ID NO 15. The IFNβ_C17S,N80Q may comprise the sequence as set forth in SEQ ID NO 16. The IFNγ may comprise the sequence as set forth in SEQ ID NO 19. The IFNλ2 may comprise the sequence as set forth in SEQ ID NO 18. The IFNε may comprise the sequence as set forth in SEQ ID NO 80. The IFNω may comprise the sequence as set forth in SEQ ID NO 79. The linkers referred to are those listed in Table 7.

In the embodiments where the IFN is fused to the N-terminus of a heavy chain of the agonistic anti-CD40 antibody, or the agonistic antigen binding fragment thereof, the interferon-associated antigen binding protein further comprises a light chain of an agonistic anti-CD40 antibody, or an agonistic antigen binding fragment thereof. In more specific embodiments, a heavy chain comprises a sequence as set forth in SEQ ID NO 6, SEQ ID NO 9, SEQ ID NO 12, SEQ ID NO 48, SEQ ID NO 49 or SEQ ID NO 50 and a light chain comprises a sequence as set forth in SEQ ID NO 3. In other more specific embodiments, a heavy chain comprises a sequence as set forth in SEQ ID 61, SEQ ID 63 or SEQ ID 65 and a light chain comprises a sequence as set forth in SEQ ID NO 59.

TABLE 4
Interferon or a functional fragment thereof fused to the N-terminus of a heavy chain
of the anti-CD40 antibody or an agonistic antigen binding fragment thereof.

| A | IFNα2a | IFNβ | IFNβ_C17S | IFNβ_C17S, N80Q | IFNγ | IFNλ2 |
|---|---|---|---|---|---|---|
| RL | IFNα2a--RL-- | IFNβ--RL-- | IFNβ_C17S-- | IFNβ_C17S, N80Q-- | IFNγ--RL-- | IFNλ2--RL-- |
| linker | antiCD40_HC | antiCD40_HC | RL-- | RL--antiCD40_HC | antiCD40_HC | antiCD40_HC |
| | | | antiCD40_HC | | | |
| GST | IFNα2a--GST-- | IFNβ--GST-- | IFNβ_C17S-- | IFNβ_C17S, N80Q-- | IFNγ--GST-- | IFNλ2--GST-- |
| linker | antiCD40_HC | antiCD40_HC | GST-- | GST--antiCD40_HC | antiCD40_HC | antiCD40_HC |
| | | | antiCD40_HC | | | |
| HL | IFNα2a--HL-- | IFNβ--HL-- | IFNβ_C17S-- | IFNβ_C17S, N80Q-- | IFNγ--HL-- | IFNλ2--HL-- |
| linker | antiCD40_HC | antiCD40_HC | HL-- | HL--antiCD40_HC | antiCD40_HC | antiCD40_HC |
| | | | antiCD40_HC | | | |
| HL2 | IFNα2a--HL2-- | IFNβ--HL2-- | IFNβ_C17S-- | IFNβ_C17S, N80Q-- | IFNγ--HL2-- | IFNλ2--HL2-- |
| linker | antiCD40_HC | antiCD40_HC | HL2-- | HL2--antiCD40_HC | antiCD40_HC | antiCD40_HC |
| | | | antiCD40_HC | | | |
| (G4S)2 | IFNα2a-- | IFNβ--(G4S)2-- | IFNβ_C17S-- | IFNβ_C17S, N80Q-- | IFNγ--(G4S)2-- | IFNλ2--(G4S)2-- |
| linker | (G4S)2-- | antiCD40_HC | (G4S)2-- | (G4S)2-- | antiCD40_HC | antiCD40_HC |
| | antiCD40_HC | | antiCD40_HC | antiCD40_HC | | |
| (G4S)3 | IFNα2a-- | IFNβ--(G4S)3-- | IFNβ_C17S-- | IFNβ_C17S, N80Q-- | IFNγ--(G4S)3-- | IFNλ2--(G4S)3-- |
| linker | (G4S)3-- | antiCD40_HC | (G4S)3-- | (G4S)3-- | antiCD40_HC | antiCD40_HC |
| | antiCD40_HC | | antiCD40_HC | antiCD40_HC | | |
| (G4S)4 | IFNα2a-- | IFNβ--(G4S)4-- | IFNβ_C17S-- | IFNβ_C17S, N80Q-- | IFNγ--(G4S)4-- | IFNλ2--(G4S)4-- |
| linker | (G4S)4-- | antiCD40_HC | (G4S)4-- | (G4S)4-- | antiCD40_HC | antiCD40_HC |
| | antiCD40_HC | | antiCD40_HC | antiCD40_HC | | |

| B | IFNε | IFNω |
|---|---|---|
| RL | IFNε--RL-- | IFNω--RL-- |
| linker | antiCD40_HC | antiCD40_HC |
| GST | IFNε--GST-- | IFNω--GST-- |
| linker | antiCD40_HC | antiCD40_HC |
| HL | IFNε--HL-- | IFNω--HL-- |
| linker | antiCD40_HC | antiCD40_HC |
| HL2 | IFNε--HL2-- | IFNω--HL2-- |
| linker | antiCD40_HC | antiCD40_HC |
| (G4S)2 | IFNε--(G4S)2-- | IFNω--(G4S)2-- |
| linker | antiCD40_HC | antiCD40_HC |
| (G4S)3 | IFNε--(G4S)3-- | IFNω--(G4S)3-- |
| linker | antiCD40_HC | antiCD40_HC |
| (G4S)4 | IFNε--(G4S)4-- | IFNω--(G4S)4-- |
| linker | antiCD40_HC | antiCD40_HC |

In certain preferred embodiments, the IFN is fused to the C-terminus of a light chain of the agonistic anti-CD40 antibody, or the agonistic antigen binding fragment thereof, via the linker, as set forth in Table 5A or Table 5B. In these embodiments, the light chain of the agonistic anti-CD40 antibody, or the agonistic antigen binding fragment thereof, may comprise a sequence as set forth in SEQ ID NO 3. In other embodiments, the light chain may comprise a sequence as set forth in SEQ ID NO 59. The IFNα2a may comprise the sequence as set forth in SEQ ID NO 17. The IFNβ may comprise the sequence as set forth in SEQ ID NO 14, SEQ ID NO 15 or SEQ ID NO 16. The IFNβ may comprise the sequence as set forth in SEQ ID NO 14. The IFNβ_C17S may comprise the sequence as set forth in SEQ ID NO 15. The IFNβ_C17S,N80Q may comprise the sequence as set forth in SEQ ID NO 16. The IFNγ may comprise the sequence as set forth in SEQ ID NO 19. The IFNλ2 may comprise the sequence as set forth in SEQ ID NO 18. The IFNε may comprise the sequence as set forth in SEQ ID NO 80. The IFNω may comprise the sequence as set forth in SEQ ID NO 79. The linkers referred to are those listed in Table 7.

In the embodiments where the IFN is fused to the C-terminus of a light chain of the agonistic anti-CD40 antibody, or the agonistic antigen binding fragment thereof, the interferon-associated antigen binding protein further comprises a heavy chain of an agonistic anti-CD40 antibody, or an agonistic antigen binding fragment thereof. In more specific embodiments, a light chain comprises a sequence as set forth in SEQ ID NO 3 and a heavy chain comprises a sequence as set forth in SEQ ID NO 6, SEQ ID NO 9, SEQ ID NO 49, SEQ ID NO 48, SEQ ID NO 50 or SEQ ID NO 12. In other more specific embodiments, a light chain comprises a sequence as set forth in SEQ ID NO 59 and a heavy chain comprises a sequence as set forth in SEQ ID NO 61, SEQ ID NO 63 or SEQ ID NO 65.

TABLE 5
Interferon or a functional fragment thereof fused to the C-terminus of a light chain
of the anti-CD40 antibody or an agonistic antigen binding fragment thereof.

| A | IFNα2a | IFNβ | IFNβ_C17S | IFNβ_C17S, N80Q | IFNγ | IFNλ2 |
|---|---|---|---|---|---|---|
| RL | antiCD40_LC- | antiCD40_LC-- | antiCD40_LC-- | antiCD40_LC--RL-- | antiCD40_LC-- | antiCD40_LC-- |
| linker | RL--IFNα2a | RL--IFNβ | RL-- | IFNβ_C17S, N80Q | RL--IFNγ | RL--IFNλ2 |
| | | | IFNβ_C17S | | | |
| GST | antiCD40_LC-- | antiCD40_LC-- | antiCD40_LC-- | antiCD40_LC--GST-- | antiCD40_LC-- | antiCD40_LC-- |
| linker | GST--IFNα2a | GST--IFNβ | GST-- | IFNβ_C17S, N80Q | GST--IFNγ | GST--IFNλ2 |
| | | | IFNβ_C17S | | | |
| HL | antiCD40_LC-- | antiCD40_LC-- | antiCD40_LC-- | antiCD40_LC--HL-- | antiCD40_LC-- | antiCD40_LC-- |
| linker | HL--IFNα2a | HL--IFNβ | HL-- | IFNβ_C17S, N80Q | HL--IFNγ | HL--IFNλ2 |
| | | | IFNβ_C17S | | | |
| HL2 | antiCD40_LC-- | antiCD40_LC-- | antiCD40_LC-- | antiCD40_LC--HL2-- | antiCD40_LC-- | antiCD40_LC-- |
| linker | HL2--IFNα2a | HL2--IFNβ | HL2-- | IFNβ_C17S, N80Q | HL2--IFNγ | HL2--IFNλ2 |
| | | | IFNβ_C17S | | | |
| (G4S)2 | antiCD40_LC-- | antiCD40_LC-- | antiCD40_LC-- | antiCD40_LC-- | antiCD40_LC-- | antiCD40_LC-- |
| linker | (G4S)2--IFNα2a | (G4S)2--IFNβ | (G4S)2-- | (G4S)2-- | (G4S)2--IFNγ | (G4S)2--IFNλ2 |
| | | | IFNβ_C17S | IFNβ_C17S, N80Q | | |
| (G4S)3 | antiCD40_LC-- | antiCD40_LC-- | antiCD40_LC-- | antiCD40_LC-- | antiCD40_LC-- | antiCD40_LC-- |
| linker | (G4S)3--IFNα2a | (G4S)3--IFNβ | (G4S)3-- | (G4S)3-- | (G4S)3--IFNγ | (G4S)3--IFNλ2 |
| | | | IFNβ_C17S | IFNβ_C17S, N80Q | | |
| (G4S)4 | antiCD40_LC-- | antiCD40_LC-- | antiCD40_LC-- | antiCD40_LC-- | antiCD40_LC-- | antiCD40_LC-- |
| linker | (G4S)4--IFNα2a | (G4S)4--IFNβ | (G4S)4-- | (G4S)4-- | (G4S)4--IFNγ | (G4S)4--IFNλ2 |
| | | | IFNβ_C17S | IFNβ_C17S, N80Q | | |

| B | IFNε | IFNω |
|---|---|---|
| RL | antiCD40_LC-- | antiCD40_LC-- |
| linker | RL--IFNε | RL--IFNω |
| GST | antiCD40_LC-- | antiCD40_LC-- |
| linker | GST--IFNε | GST--IFNω |
| HL | antiCD40_LC-- | antiCD40_LC-- |
| linker | HL--IFNε | HL--IFNω |
| HL2 | antiCD40_LC-- | antiCD40_LC-- |
| linker | HL2--IFNε | HL2--IFNω |
| (G4S)2 | antiCD40_LC-- | antiCD40_LC-- |
| linker | (G4S)2--IFNε | (G4S)2--IFNω |
| (G4S)3 | antiCD40_LC-- | antiCD40_LC-- |
| linker | (G4S)3--IFNε | (G4S)3--IFNω |
| (G4S)4 | antiCD40_LC-- | antiCD40_LC-- |
| linker | (G4S)4--IFNε | (G4S)4--IFNω |

In certain preferred embodiments, the IFN is fused to the N-terminus of a light chain of the agonistic anti-CD40 antibody, or the agonistic antigen binding fragment thereof, via the linker, as set forth in Table 6A or Table 6B. In these embodiments, the light chain of the agonistic anti-CD40 antibody, or the agonistic antigen binding fragment thereof, may comprise a sequence as set forth in SEQ ID NO 3 or SEQ ID NO 59. The IFNα2a may comprise the sequence as set forth in SEQ ID NO 17. The IFNβ may comprise the sequence as set forth in SEQ ID NO 14, SEQ ID NO 15 or SEQ ID NO 16. The IFNβ may comprise the sequence as set forth in SEQ ID NO 14. The IFNβ_C17S may comprise the sequence as set forth in SEQ ID NO 15. The IFNβ_C17S,N80Q may comprise the sequence as set forth in SEQ ID NO 16. The IFNγ may comprise the sequence as set forth in SEQ ID NO 19. The IFNλ2 may comprise the sequence as set forth in SEQ ID NO 18. The IFNε may comprise the sequence as set forth in SEQ ID NO 80. The IFNω may comprise the sequence as set forth in SEQ ID NO 79. The linkers referred to are those listed in Table 7.

In the embodiments where the IFN is fused to the N-terminus of a light chain of the agonistic anti-CD40 antibody, or the agonistic antigen binding fragment thereof, the interferon-associated antigen binding protein further comprises a heavy chain of an agonistic anti-CD40 antibody, or an agonistic antigen binding fragment thereof. In more specific embodiments, a light chain comprises a sequence as set forth in SEQ ID NO 3 and a heavy chain comprises a sequence as set forth in SEQ ID NO 6, SEQ ID NO 9, SEQ ID NO 49, SEQ ID NO 48, SEQ ID NO 12 or SEQ ID NO 50. In other more specific embodiments, a light chain comprises a sequence as set forth in SEQ ID NO 59 and a heavy chain comprises a sequence as set forth in SEQ ID NO 61, SEQ ID NO 63 or SEQ ID NO 65.

TABLE 6
Interferon or a functional fragment thereof fused to the N-terminus of a light chain
of the anti-CD40 antibody or an agonistic antigen binding fragment thereof.

| A | IFNα2a | IFNβ | IFNβ_C17S | IFNβ_C17S, N80Q | IFNγ | IFNλ2 |
|---|---|---|---|---|---|---|
| RL | IFNα2a--RL-- | IFNβ--RL-- | IFNβ_C17S-- | IFNβ_C17S, N80Q-- | IFNγ--RL-- | IFNλ2--RL-- |
| linker | antiCD40_LC | antiCD40_LC | RL-- | RL--antiCD40_LC | antiCD40_LC | antiCD40_LC |
| | | | antiCD40_LC | | | |
| GST | IFNαa--GST-- | IFNβ--GST-- | IFNβ_C17S-- | IFNβ_C17S, N80Q-- | IFNγ--GST-- | IFNλ2--GST-- |
| linker | antiCD40_LC | antiCD40_LC | GST-- | GST--antiCD40_LC | antiCD40_LC | antiCD40_LC |
| | | | antiCD40_LC | | | |
| HL | IFNα2a--HL-- | IFNβ--HL-- | IFNβ_C17S- | IFNβ_C17S, N80Q-- | IFNγ--HL-- | IFNλ2--HL-- |
| linker | antiCD40_LC | antiCD40_LC | HL-- | HL--antiCD40_LC | antiCD40_LC | antiCD40_LC |
| | | | antiCD40_LC | | | |
| HL2 | IFNα2a--HL2-- | IFNβ--HL2-- | IFNβ_C17S-- | IFNβ_C17S, N80Q-- | IFNγ--HL2-- | IFNλ2--HL2-- |
| linker | antiCD40_LC | antiCD40_LC | HL2-- | HL2--antiCD40_LC | antiCD40_LC | antiCD40_LC |
| | | | antiCD40_LC | | | |
| (G4S)2 | IFNα2a-- | IFNβ--(G4S)2-- | IFNβ_C17S- | IFNβ_C17S, N80Q-- | IFNγ--(G4S)2-- | IFNλ2--(G4S)2-- |
| linker | (G4S)2-- | antiCD40_LC | (G4S)2-- | (G4S)2-- | antiCD40_LC | antiCD40_LC |
| | antiCD40_LC | | antiCD40_LC | antiCD40_LC | | |
| (G4S)3 | IFNα2a-- | IFNβ--(G4S)3-- | IFNβ_C17S-- | IFNβ_C17S, N80Q-- | IFNγ--(G4S)3-- | IFNλ2--(G4S)3-- |
| linker | (G4S)3-- | antiCD40_LC | (G4S)3-- | (G4S)3-- | antiCD40_LC | antiCD40_LC |
| | antiCD40_LC | | antiCD40_LC | antiCD40_LC | | |
| (G4S)4 | IFNα2a-- | IFNβ--(G4S)4-- | IFNβ_C17S-- | IFNβ_C17S, N80Q-- | IFNγ--(G4S)4-- | IFNλ2--(G4S)4-- |
| linker | (G4S)4-- | antiCD40_LC | (G4S)4-- | (G4S)4-- | antiCD40_LC | antiCD40_LC |
| | antiCD40_LC | | antiCD40_LC | antiCD40_LC | | |

| B | IFNε | IFNω |
|---|---|---|
| RL | IFNε--RL-- | IFNω--RL-- |
| linker | antiCD40_LC | antiCD40_LC |
| GST | IFNε--GST-- | IFNω--GST-- |
| linker | antiCD40_LC | antiCD40_LC |
| HL | IFNε--HL-- | IFNω--HL-- |
| linker | antiCD40_LC | antiCD40_LC |
| HL2 | IFNε--HL2-- | IFNω--HL2-- |
| linker | antiCD40_LC | antiCD40_LC |
| (G4S)2 | IFNε--(G4S)2-- | IFNω--(G4S)2-- |
| linker | antiCD40_LC | antiCD40_LC |
| (G4S)3 | IFNε--(G4S)3-- | IFNω--(G4S)3-- |
| linker | antiCD40_LC | antiCD40_LC |
| (G4S)4 | IFNε--(G4S)4-- | IFNω--(G4S)4-- |
| linker | antiCD40_LC | antiCD40_LC |

Exemplary sequences comprised in interferon-associated antigen binding proteins of the invention or precursors thereof are listed in Table 7.

In exemplary preferred embodiments, the interferon-associated antigen binding protein comprises an interferon-fused agonistic anti-CD40 antibody or an interferon-fused agonistic antigen binding fragment thereof comprising a sequence selected from SEQ ID NOs 28-47 or SEQ ID NOs 66-75. In other exemplary embodiments, the interferon-associated antigen binding protein comprises an interferon-fused agonistic anti-CD40 antibody or an interferon-fused agonistic antigen binding fragment thereof comprising a sequence selected from SEQ ID NOs 81-88. In exemplary preferred embodiments, the interferon-associated antigen binding protein is an interferon-fused agonistic anti-CD40 antibody or an interferon-fused agonistic antigen binding fragment thereof comprising a sequence selected from SEQ ID NOs 28-47 or SEQ ID NOs 66-75. In other exemplary embodiments, the interferon-associated antigen binding protein is an interferon-fused agonistic anti-CD40 antibody or an interferon-fused agonistic antigen binding fragment thereof comprising a sequence selected from SEQ ID NOs 81-88. In other exemplary embodiments, the interferon-associated antigen binding protein is an interferon-fused agonistic anti-CD40 antibody or an interferon-fused agonistic antigen binding fragment thereof comprising a sequence selected from SEQ ID NOs 93-94.

In certain exemplary embodiments, the interferon-associated antigen binding protein comprises an interferon-fused agonistic anti-CD40 antibody or an interferon-fused agonistic binding fragment thereof comprising a sequence as set forth in SEQ ID NO 93. In another exemplary embodiment, the interferon-associated antigen binding protein is an interferon-fused agonistic anti-CD40 antibody or an interferon-fused agonistic binding fragment thereof comprising a sequence as set forth in SEQ ID NO 93.

In certain exemplary embodiments, the interferon-associated antigen binding protein comprises an interferon-fused agonistic anti-CD40 antibody or an interferon-fused agonistic binding fragment thereof comprising a sequence as set forth in SEQ ID NO 94. In another exemplary embodiment, the interferon-associated antigen binding protein is an interferon-fused agonistic anti-CD40 antibody or an interferon-fused agonistic binding fragment thereof comprising a sequence as set forth in SEQ ID NO 94.

In certain exemplary embodiments, the interferon-associated antigen binding protein comprises an interferon-fused agonistic anti-CD40 antibody or an interferon-fused agonistic binding fragment thereof comprising a sequence as set forth in SEQ ID NO 81. In another exemplary embodiment, the interferon-associated antigen binding protein is an interferon-fused agonistic anti-CD40 antibody or an interferon-fused agonistic binding fragment thereof comprising a sequence as set forth in SEQ ID NO 81.

In certain exemplary embodiments, the interferon-associated antigen binding protein comprises an interferon-fused agonistic anti-CD40 antibody or an interferon-fused agonistic binding fragment thereof comprising a sequence as set forth in SEQ ID NO 82. In another exemplary embodiment, the interferon-associated antigen binding protein is an interferon-fused agonistic anti-CD40 antibody or an interferon-fused agonistic binding fragment thereof comprising a sequence as set forth in SEQ ID NO 82.

In certain exemplary embodiments, the interferon-associated antigen binding protein comprises an interferon-fused agonistic anti-CD40 antibody or an interferon-fused agonistic binding fragment thereof comprising a sequence as set forth in SEQ ID NO 83. In another exemplary embodiment, the interferon-associated antigen binding protein is an interferon-fused agonistic anti-CD40 antibody or an interferon-fused agonistic binding fragment thereof comprising a sequence as set forth in SEQ ID NO 83.

In certain exemplary embodiments, the interferon-associated antigen binding protein comprises an interferon-fused agonistic anti-CD40 antibody or an interferon-fused agonistic binding fragment thereof comprising a sequence as set forth in SEQ ID NO 84. In another exemplary embodiment, the interferon-associated antigen binding protein is an interferon-fused agonistic anti-CD40 antibody or an interferon-fused agonistic binding fragment thereof comprising a sequence as set forth in SEQ ID NO 84.

In certain exemplary embodiments, the interferon-associated antigen binding protein comprises an interferon-fused agonistic anti-CD40 antibody or an interferon-fused agonistic binding fragment thereof comprising a sequence as set forth in SEQ ID NO 85. In another exemplary embodiment, the interferon-associated antigen binding protein is an interferon-fused agonistic anti-CD40 antibody or an interferon-fused agonistic binding fragment thereof comprising a sequence as set forth in SEQ ID NO 85.

In certain exemplary embodiments, the interferon-associated antigen binding protein comprises an interferon-fused agonistic anti-CD40 antibody or an interferon-fused agonistic binding fragment thereof comprising a sequence as set forth in SEQ ID NO 86. In another exemplary embodiment, the interferon-associated antigen binding protein is an interferon-fused agonistic anti-CD40 antibody or an interferon-fused agonistic binding fragment thereof comprising a sequence as set forth in SEQ ID NO 86.

In certain exemplary embodiments, the interferon-associated antigen binding protein comprises an interferon-fused agonistic anti-CD40 antibody or an interferon-fused agonistic binding fragment thereof comprising a sequence as set forth in SEQ ID NO 87. In another exemplary embodiment, the interferon-associated antigen binding protein is an interferon-fused agonistic anti-CD40 antibody or an interferon-fused agonistic binding fragment thereof comprising a sequence as set forth in SEQ ID NO 87.

In certain exemplary embodiments, the interferon-associated antigen binding protein comprises an interferon-fused agonistic anti-CD40 antibody or an interferon-fused agonistic binding fragment thereof comprising a sequence as set forth in SEQ ID NO 88. In another exemplary embodiment, the interferon-associated antigen binding protein is an interferon-fused agonistic anti-CD40 antibody or an interferon-fused agonistic binding fragment thereof comprising a sequence as set forth in SEQ ID NO 88.

In more preferred embodiments, the interferon-associated antigen binding protein comprises an interferon-fused agonistic anti-CD40 antibody or an interferon-fused agonistic antigen binding fragment thereof comprising a sequence selected from SEQ ID NO 38, SEQ ID NO 39, SEQ ID NO 40, SEQ ID NO 41, SEQ ID NO 42 or SEQ ID NO 43. In more preferred embodiments, the interferon-associated antigen binding protein is an interferon-fused agonistic anti-CD40 antibody or an interferon-fused agonistic antigen binding fragment thereof comprising a sequence selected from SEQ ID NO 38, SEQ ID NO 39, SEQ ID NO 40, SEQ ID NO 41, SEQ ID NO 42 or SEQ ID NO 43. In other more preferred embodiments, the interferon-associated antigen binding protein comprises an interferon-fused agonistic anti-CD40 antibody or an interferon-fused agonistic antigen binding fragment thereof comprising a sequence selected from SEQ ID NO 72, SEQ ID NO 73, SEQ ID NO 74 and SEQ ID NO 75. In still other more preferred embodiments, the interferon-associated antigen binding protein is an interferon-fused agonistic anti-CD40 antibody or an interferon-fused agonistic antigen binding fragment thereof comprising a sequence selected from SEQ ID NO 72, SEQ ID NO 73, SEQ ID NO 74 and SEQ ID NO 75.

In an even more preferred embodiment, the interferon-associated antigen binding protein comprises an interferon-fused agonistic anti-CD40 antibody or an interferon-fused agonistic binding fragment thereof comprising a sequence as set forth in SEQ ID NO 38. In still another even more preferred embodiment, the interferon-associated antigen binding protein is an interferon-fused agonistic anti-CD40 antibody or an interferon-fused agonistic binding fragment thereof comprising a sequence as set forth in SEQ ID NO 38.

In another even more preferred embodiment, the interferon-associated antigen binding protein comprises an interferon-fused agonistic anti-CD40 antibody or an interferon-fused agonistic binding fragment thereof comprising a sequence as set forth in SEQ ID NO 39. In another even more preferred embodiment, the interferon-associated antigen binding protein is an interferon-fused agonistic anti-CD40 antibody or an interferon-fused agonistic binding fragment thereof comprising a sequence as set forth in SEQ ID NO 39.

In another even more preferred embodiment, the interferon-associated antigen binding protein comprises an interferon-fused agonistic anti-CD40 antibody or an interferon-fused agonistic binding fragment thereof comprising a sequence as set forth in SEQ ID NO 40. In another even more preferred embodiment, the interferon-associated antigen binding protein is an interferon-fused agonistic anti-CD40 antibody or an interferon-fused agonistic binding fragment thereof comprising a sequence as set forth in SEQ ID NO 40.

In another even more preferred embodiment, the interferon-associated antigen binding protein comprises an interferon-fused agonistic anti-CD40 antibody or an interferon-fused agonistic binding fragment thereof comprising a sequence as set forth in SEQ ID NO 41. In another even more preferred embodiment, the interferon-associated antigen binding protein is an interferon-fused agonistic anti-CD40 antibody or an interferon-fused agonistic binding fragment thereof comprising a sequence as set forth in SEQ ID NO 41.

In another even more preferred embodiment, the interferon-associated antigen binding protein comprises an interferon-fused agonistic anti-CD40 antibody or an interferon-fused agonistic binding fragment thereof comprising a sequence as set forth in SEQ ID NO 42. In another even more preferred embodiment, the interferon-associated antigen binding protein is an interferon-fused agonistic anti-CD40 antibody or an interferon-fused agonistic binding fragment thereof comprising a sequence as set forth in SEQ ID NO 42.

In another even more preferred embodiment, the interferon-associated antigen binding protein comprises an interferon-fused agonistic anti-CD40 antibody or an interferon-fused agonistic binding fragment thereof comprising a sequence as set forth in SEQ ID NO 43. In another even more preferred embodiment, the interferon-associated antigen binding protein is an interferon-fused agonistic anti-CD40 antibody or an interferon-fused agonistic binding fragment thereof comprising a sequence as set forth in SEQ ID NO 43.

In another even more preferred embodiment, the interferon-associated antigen binding protein comprises an interferon-fused agonistic anti-CD40 antibody or an interferon-fused agonistic binding fragment thereof comprising a sequence as set forth in SEQ ID NO 72. In another even more preferred embodiment, the interferon-associated antigen binding protein is an interferon-fused agonistic anti-CD40 antibody or an interferon-fused agonistic binding fragment thereof comprising a sequence as set forth in SEQ ID NO 72.

In another even more preferred embodiment, the interferon-associated antigen binding protein comprises an interferon-fused agonistic anti-CD40 antibody or an interferon-fused agonistic binding fragment thereof comprising a sequence as set forth in SEQ ID NO 73. In another even more preferred embodiment, the interferon-associated antigen binding protein is an interferon-fused agonistic anti-CD40 antibody or an interferon-fused agonistic binding fragment thereof comprising a sequence as set forth in SEQ ID NO 73.

In another even more preferred embodiment, the interferon-associated antigen binding protein comprises an interferon-fused agonistic anti-CD40 antibody or an interferon-fused agonistic binding fragment thereof comprising a sequence as set forth in SEQ ID NO 74. In another even more preferred embodiment, the interferon-associated antigen binding protein is an interferon-fused agonistic anti-CD40 antibody or an interferon-fused agonistic binding fragment thereof comprising a sequence as set forth in SEQ ID NO 74.

In another even more preferred embodiment, the interferon-associated antigen binding protein comprises an interferon-fused agonistic anti-CD40 antibody or an interferon-fused agonistic binding fragment thereof comprising a sequence as set forth in SEQ ID NO 75. In another even more preferred embodiment, the interferon-associated antigen binding protein is an interferon-fused agonistic anti-CD40 antibody or an interferon-fused agonistic binding fragment thereof comprising a sequence as set forth in SEQ ID NO 75.

TABLE 7
Sequences of exemplary interferon-associated antigen binding protein and
components thereof based on the antiCD40 antibody CP870, 893. Italic sequences
correspond to signal peptides. Bold italic sequences in SEQ ID NOs 3 and 6
correspond to CDR regions. Bold non-italic sequences correspond to linkers.
Mutated amino acids are underlined.

| Name/ | |
|---|---|
| SEQ ID Number | Sequence |
| Signal peptide 1 | MGWSCIILFLVATATGVHS |
| (SEQ ID NO 1) | |
| Signal peptide 2 | MDMRVPAQLLGLLLLWLRGARC |
| (SEQ ID NO 2) | |
| antiCD40 | DIQMTQSPSSVSASVGDRVTITCRASQGIYSWLAWYQQKPG |
| antibody light | KAPNLLIYTASTLQSGVPSRFSGSGSGTDFTLTISSLQPEDFAT |
| chain | YYCQQANIFPLTFGGGTKVEIKRTVAAPSVFIFPPSDEQLKS |
| (SEQ ID NO 3) | GTASVVCLLNNFYPREAKVQWKVDNALQSGNSQESVTEQD |
| | SKDSTYSLSSTLTLSKADYEKHKVYACEVTHQGLSSPVTKSF |
| | NRGEC |
| antiCD40 | MGWSCIILFLVATATGVHSDIQMTQSPSSVSASVGDRVTITCR |
| antibody light | ASQGIYSWLAWYQQKPGKAPNLLIYTASTLQSGVPSRFSGS |
| chain | GSGTDFTLTISSLQPEDFATYYCQQANIFPLTFGGGTKVEIKR |
| with signal peptide | TVAAPSVFIFPPSDEQLKSGTASVVCLLNNFYPREAKVQWK |
| 1 | VDNALQSGNSQESVTEQDSKDSTYSLSSTLTLSKADYEKHK |
| (SEQ ID NO 4) | VYACEVTHQGLSSPVTKSFNRGEC |
| antiCD40 | MDMRVPAQLLGLLLLWLRGARCDIQMTQSPSSVSASVGDRVT |
| antibody light | ITCRASQGIYSWLAWYQQKPGKAPNLLIYTASTLQSGVPSRF |
| chain | SGSGSGTDFTLTISSLQPEDFATYYCQQANIFPLTFGGGTKVE |
| with signal peptide | IKRTVAAPSVFIFPPSDEQLKSGTASVVCLLNNFYPREAKVQ |
| 2 | WKVDNALQSGNSQESVTEQDSKDSTYSLSSTLTLSKADYEK |
| (SEQ ID NO 5) | HKVYACEVTHQGLSSPVTKSFNRGEC |
| antiCD40 | QVQLVQSGAEVKKPGASVKVSCKASGYTFTGYYMHWVRQ |
| antibody heavy | APGQGLEWMGWINPDSGGTNYAQKFQGRVTMTRDTSISTA |
| chain hIgG2 dK | YMELNRLRSDDTAVYYCARDQPLGYCTNGVCSYFDYWGQ |
| (SEQ ID NO 6) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPG |
| antiCD40 | MGWSCIILFLVATATGVHSQVQLVQSGAEVKKPGASVKVSCK |
| antibody heavy | ASGYTFTGYYMHWVRQAPGQGLEWMGWINPDSGGTNYA |
| chain hIgG2 dK | QKFQGRVTMTRDTSISTAYMELNRLRSDDTAVYYCARDQP |
| with signal peptide | LGYCTNGVCSYFDYWGQGTLVTVSSASTKGPSVFPLAPCSR |
| 1 | STSESTAALGCLVKDYFPEPVTVSWNSGALTSGVHTFPAVL |
| (SEQ ID NO 7) | QSSGLYSLSSVVTVPSSNFGTQTYTCNVDHKPSNTKVDKTV |
| | ERKCCVECPPCPAPPVAGPSVFLFPPKPKDTLMISRTPEVTCV |
| | VVDVSHEDPEVQFNWYVDGVEVHNAKTKPREEQFNSTFRV |
| | VSVLTVVHQDWLNGKEYKCKVSNKGLPAPIEKTISKTKGQP |
| | REPQVYTLPPSREEMTKNQVSLTCLVKGFYPSDIAVEWESN |
| | GQPENNYKTTPPMLDSDGSFFLYSKLTVDKSRWQQGNVFSC |
| | SVMHEALHNHYTQKSLSLSPG |
| antiCD40 | MDMRVPAQLLGLLLLWLRGARCQVQLVQSGAEVKKPGASVK |
| antibody heavy | VSCKASGYTFTGYYMHWVRQAPGQGLEWMGWINPDSGGT |
| chain hIgG2 dK | NYAQKFQGRVTMTRDTSISTAYMELNRLRSDDTAVYYCAR |
| with signal peptide | DQPLGYCTNGVCSYFDYWGQGTLVTVSSASTKGPSVFPLAP |
| 2 | CSRSTSESTAALGCLVKDYFPEPVTVSWNSGALTSGVHTFPA |
| (SEQ ID NO 8) | VLQSSGLYSLSSVVTVPSSNFGTQTYTCNVDHKPSNTKVDK |
| | TVERKCCVECPPCPAPPVAGPSVFLFPPKPKDTLMISRTPEVT |
| | CVVVDVSHEDPEVQFNWYVDGVEVHNAKTKPREEQFNSTF |
| | RVVSVLTVVHQDWLNGKEYKCKVSNKGLPAPIEKTISKTKG |
| | QPREPQVYTLPPSREEMTKNQVSLTCLVKGFYPSDIAVEWES |
| | NGQPENNYKTTPPMLDSDGSFFLYSKLTVDKSRWQQGNVF |
| | SCSVMHEALHNHYTQKSLSLSPG |
| antiCD40 | QVQLVQSGAEVKKPGASVKVSCKASGYTFTGYYMHWVRQ |
| antibody heavy | APGQGLEWMGWINPDSGGTNYAQKFQGRVTMTRDTSISTA |
| chain hIgG2 | YMELNRLRSDDTAVYYCARDQPLGYCTNGVCSYFDYWGQ |
| (SEQ ID NO 9) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPGK |
| antiCD40 | MGWSCIILFLVATATGVHSQVQLVQSGAEVKKPGASVKVSCK |
| antibody heavy | ASGYTFTGYYMHWVRQAPGQGLEWMGWINPDSGGTNYA |
| chain hIgG2 with | QKFQGRVTMTRDTSISTAYMELNRLRSDDTAVYYCARDQP |
| signal peptide 1 | LGYCTNGVCSYFDYWGQGTLVTVSSASTKGPSVFPLAPCSR |
| (SEQ ID NO 10) | STSESTAALGCLVKDYFPEPVTVSWNSGALTSGVHTFPAVL |
| | QSSGLYSLSSVVTVPSSNFGTQTYTCNVDHKPSNTKVDKTV |
| | ERKCCVECPPCPAPPVAGPSVFLFPPKPKDTLMISRTPEVTCV |
| | VVDVSHEDPEVQFNWYVDGVEVHNAKTKPREEQFNSTFRV |
| | VSVLTVVHQDWLNGKEYKCKVSNKGLPAPIEKTISKTKGQP |
| | REPQVYTLPPSREEMTKNQVSLTCLVKGFYPSDIAVEWESN |
| | GQPENNYKTTPPMLDSDGSFFLYSKLTVDKSRWQQGNVFSC |
| | SVMHEALHNHYTQKSLSLSPGK |
| antiCD40 | MDMRVPAQLLGLLLLWLRGARCQVQLVQSGAEVKKPGASVK |
| antibody heavy | VSCKASGYTFTGYYMHWVRQAPGQGLEWMGWINPDSGGT |
| chain hIgG2 with | NYAQKFQGRVTMTRDTSISTAYMELNRLRSDDTAVYYCAR |
| signal peptide 2 | DQPLGYCTNGVCSYFDYWGQGTLVTVSSASTKGPSVFPLAP |
| (SEQ ID NO 11) | CSRSTSESTAALGCLVKDYFPEPVTVSWNSGALTSGVHTFPA |
| | VLQSSGLYSLSSVVTVPSSNFGTQTYTCNVDHKPSNTKVDK |
| | TVERKCCVECPPCPAPPVAGPSVFLFPPKPKDTLMISRTPEVT |
| | CVVVDVSHEDPEVQFNWYVDGVEVHNAKTKPREEQFNSTF |
| | RVVSVLTVVHQDWLNGKEYKCKVSNKGLPAPIEKTISKTKG |
| | QPREPQVYTLPPSREEMTKNQVSLTCLVKGFYPSDIAVEWES |
| | NGQPENNYKTTPPMLDSDGSFFLYSKLTVDKSRWQQGNVF |
| | SCSVMHEALHNHYTQKSLSLSPGK |
| antiCD40 | QVQLVQSGAEVKKPGASVKVSCKASGYTFTGYYMHWVRQ |
| antibody hIgG1 | APGQGLEWMGWINPDSGGTNYAQKFQGRVTMTRDTSISTA |
| heavy chain- | YMELNRLRSDDTAVYYCARDQPLGYCTNGVCSYFDYWGQ |
| NNAS | GTLVTVSSASTKGPSVFPLAPSSKSTSGGTAALGCLVKDYFP |
| (SEQ ID NO 48) | EPVTVSWNSGALTSGVHTFPAVLQSSGLYSLSSVVTVPSSSL |
| | GTQTYICNVNHKPSNTKVDKKVEPKSCDKTHTCPPCPAPEL |
| | LGGPSVFLFPPKPKDTLMISRTPEVTCVVVDVSHEDPEVKFN |
| | WYVDGVEVHNAKTKPREEQYNNASRVVSVLTVLHQDWLN |
| | GKEYKCKVSNKALPAPIEKTISKAKGQPREPQVYTLPPSRDE |
| | LTKNQVSLTCLVKGFYPSDIAVEWESNGQPENNYKTTPPVL |
| | DSDGSFFLYSKLTVDKSRWQQGNVFSCSVMHEALHNHYTQ |
| | KSLSLSPGK |
| antiCD40 | QVQLVQSGAEVKKPGASVKVSCKASGYTFTGYYMHWVRQ |
| antibody hIgG1 | APGQGLEWMGWINPDSGGTNYAQKFQGRVTMTRDTSISTA |
| heavy chain- | YMELNRLRSDDTAVYYCARDQPLGYCTNGVCSYFDYWGQ |
| NNAS-dK | GTLVTVSSASTKGPSVFPLAPSSKSTSGGTAALGCLVKDYFP |
| (SEQ ID NO 49) | EPVTVSWNSGALTSGVHTFPAVLQSSGLYSLSSVVTVPSSSL |
| | GTQTYICNVNHKPSNTKVDKKVEPKSCDKTHTCPPCPAPEL |
| | LGGPSVFLFPPKPKDTLMISRTPEVTCVVVDVSHEDPEVKEN |
| | WYVDGVEVHNAKTKPREEQYNNASRVVSVLTVLHQDWLN |
| | GKEYKCKVSNKALPAPIEKTISKAKGQPREPQVYTLPPSRDE |
| | LTKNQVSLTCLVKGFYPSDIAVEWESNGQPENNYKTTPPVL |
| | DSDGSFFLYSKLTVDKSRWQQGNVFSCSVMHEALHNHYTQ |
| | KSLSLSPG |
| antiCD40 | QVQLVQSGAEVKKPGASVKVSCKASGYTFTGYYMHWVRQ |
| antibody hIgG2 | APGQGLEWMGWINPDSGGTNYAQKFQGRVTMTRDTSISTA |
| Fab region heavy | YMELNRLRSDDTAVYYCARDQPLGYCTNGVCSYFDYWGQ |
| chain | GTLVTVSSASTKGPSVFPLAPCSRSTSESTAALGCLVKDYFP |
| (SEQ ID NO 12) | EPVTVSWNSGALTSGVHTFPAVLQSSGLYSLSSVVTVPSSNF |
| | GTQTYTCNVDHKPSNTKVDKTVERKCCVE |
| antiCD40 | MGWSCIILFLVATATGVHSQVQLVQSGAEVKKPGASVKVSCK |
| antibody hIgG2 | ASGYTFTGYYMHWVRQAPGQGLEWMGWINPDSGGTNYA |
| Fab region heavy | QKFQGRVTMTRDTSISTAYMELNRLRSDDTAVYYCARDQP |
| chain with signal | LGYCTNGVCSYFDYWGQGTLVTVSSASTKGPSVFPLAPCSR |
| peptide 1 | STSESTAALGCLVKDYFPEPVTVSWNSGALTSGVHTFPAVL |
| (SEQ ID NO 13) | QSSGLYSLSSVVTVPSSNFGTQTYTCNVDHKPSNTKVDKTV |
| | ERKCCVE |
| antiCD40 | QVQLVQSGAEVKKPGASVKVSCKASGYTFTGYYMHWVRQ |
| antibody hIgG2 | APGQGLEWMGWINPDSGGTNYAQKFQGRVTMTRDTSISTA |
| Fab region heavy | YMELNRLRSDDTAVYYCARDQPLGYCTNGVCSYFDYWGQ |
| chain | GTLVTVSSASTKGPSVFPLAPCSRSTSESTAALGCLVKDYFP |
| --TEV--6His tag | EPVTVSWNSGALTSGVHTFPAVLQSSGLYSLSSVVTVPSSNF |
| (SEQ ID NO 50) | GTQTYTCNVDHKPSNTKVDKTVERKCCVEENLYFQSHHHH |
| | HH |
| IFNβ dM | SYNLLGFLQRSSNFQCQKLLWQLNGRLEYCLKDRMNFDIPE |
| (SEQ ID NO 76) | EIKQLQQFQKEDAALTIYEMLQNIFAIFRQDSSSTGWNETIVE |
| | NLLANVYHQINHLKTVLEEKLEKEDFTRGKLMSSLHLKRYY |
| | GRILHYLKAKEYSHCAWTIVRVEILRNFYFINRLTGYLRN |
| IFNβ dM C17S | SYNLLGFLQRSSNFQSQKLLWQLNGRLEYCLKDRMNFDIPE |
| (SEQ ID NO 77) | EIKQLQQFQKEDAALTIYEMLQNIF AIFRQDSSSTGWNETIVE |
| | NLLANVYHQINHLKTVLEEKLEKEDFTRGKLMSSLHLKRYY |
| | GRILHYLKAKEYSHCAWTIVRVEILRNFYFINRLTGYLRN |
| IFNβ | MSYNLLGFLQRSSNFQCQKLLWQLNGRLEYCLKDRMNFDI |
| (SEQ ID NO 14) | PEEIKQLQQFQKEDAALTIYEMLQNIFAIFRQDSSSTGWNETI |
| | VENLLANVYHQINHLKTVLEEKLEKEDFTRGKLMSSLHLKR |
| | YYGRILHYLKAKEYSHCAWTIVREILRNFYFINRLTGYLRN |
| IFNβ C17S | MSYNLLGFLQRSSNFQSQKLLWQLNGRLEYCLKDRMNFDIP |
| (SEQ ID NO 15) | EEIKQLQQFQKEDAALTIYEMLQNIFAIFRQDSSSTGWNETIV |
| | ENLLANVYHQINHLKTVLEEKLEKEDFTRGKLMSSLHLKRY |
| | YGRILHYLKAKEYSHCAWTIVRVEILRNFYFINRLTGYLRN |
| IFNβ C17S, N80Q | MSYNLLGFLQRSSNFQSQKLLWQLNGRLEYCLKDRMNFDIP |
| (SEQ ID NO 16) | EEIKQLQQFQKEDAALTIYEMLQNIFAIFRQDSSSTGWQETIV |
| | ENLLANVYHQINHLKTVLEEKLEKEDFTRGKLMSSLHLKRY |
| | YGRILHYLKAKEYSHCAWTIVRVEILRNFYFINRLTGYLRN |
| IFNα2a | CDLPQTHSLGSRRTLMLLAQMRKISLFSCLKDRHDFGFPQEE |
| (SEQ ID NO 17) | FGNQFQKAETIPVLHEMIQQIFNLFSTKDSSAAWDETLLDKF |
| | YTELYQQLNDLEACVIQGVGVTETPLMKEDSILAVRKYFQR |
| | ITLYLKEKKYSPCAWEVVRAEIMRSFSLSTNLQESLRSKE |
| IFNλ2 | VPVARLHGALPDARGCHIAQFKSLSPQELQAFKRAKDALEE |
| (SEQ ID NO 18) | SLLLKDCRCHSRLFPRTWDLRQLQVRERPMALEAELALTLK |
| | VLEATADTDPALVDVLDQPLHTLHHILSQFRACIQPQPTAGP |
| | RTRGRLHHWLYRLQEAPKKESPGCLEASVTFNLFRLLTRDL |
| | NCVASGDLCV |
| IFNγ | QDPYVKEAENLKKYFNAGHSDVADNGTLFLGILKNWKEES |
| (SEQ ID NO 19) | DRKIMQSQIVSFYFKLFKNFKDDQSIQKSVETIKEDMNVKFF |
| | NSNKKKRDDFEKLTNYSVTDLNVQRKAIHELIQVMAELSPA |
| | AKTGKRKRSQMLFRGRRASQ |
| IFNω | LGCDLPQNHGLLSRNTLVLLHQMRRISPFLCLKDRRDFRFPQ |
| (SEQ ID NO 79) | EMVKGSQLQKAHVMSVLHEMLQQIFSLFHTERSSAAWNMT |
| | LLDQLHTGLHQQLQHLETCLLQVVGEGESAGAISSPALTLR |
| | RYFQGIRVYLKEKKYSDCAWEVVRMEIMKSLFLSTNMQER |
| | LRSKDRDLGSS |
| IFNϵ | LDLKLIIFQQRQVNQESLKLLNKLQTLSIQQCLPHRKNFLLPQ |
| (SEQ ID NO 80) | KSLSPQQYQKGHTLAILHEMLQQIFSLFRANISLDGWEENHT |
| | EKFLIQLHQQLEYLEALMGLEAEKLSGTLGSDNLRLQVKMY |
| | FRRIHDYLENQDYSTCAWAIVQVEISRCLFFVFSLTEKLSKQ |
| | GRPLNDMKQELTTEFRSPR |
| RL linker | PAPA |
| (SEQ ID NO 20) | |
| GST linker | SGGTSGSTSGTGS |
| (SEQ ID NO 21) | |
| HL linker | AEAAAKEAAAKA |
| (SEQ ID NO 22) | |
| HL2 linker | AEAAAKEAAAKAAEAAAKEAAAKA |
| (SEQ ID NO 23) | |
| (G4S)2 linker | GGGGSGGGGS |
| (SEQ ID NO 24) | |
| (G4S)3 linker | GGGGSGGGGSGGGGS |
| (SEQ ID NO 25) | |
| (G4S)4 linker | GGGGSGGGGGGGGSGGGGS |
| (SEQ ID NO 26) | |
| TEV-6His tag | ENLYFQSHHHHHH |
| (SEQ ID NO 27) | |
| antiCD40 LC-- | DIQMTQSPSSVSASVGDRVTITCRASQGIYSWLAWYQQKPG |
| HL--IFNβ | KAPNLLIYTASTLQSGVPSRFSGSGSGTDFTLTISSLQPEDFAT |
| (SEQ ID NO 28) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECAEAAAKEAAAKAMSYNLLGFLQRSSNFQCQKLLW |
| | QLNGRLEYCLKDRMNFDIPEEIKQLQQFQKEDAALTIYEML |
| | QNIFAIFRQDSSSTGWNETIVENLLANVYHQINHLKTVLEEK |
| | LEKEDFTRGKLMSSLHLKRYYGRILHYLKAKEYSHCAWTIV |
| | RVEILRNFYFINRLTGYLRN |
| antiCD40_LC-- | DIQMTQSPSSVSASVGDRVTITCRASQGIYSWLAWYQQKPG |
| HL--IFNβ_C17S | KAPNLLIYTASTLQSGVPSRFSGSGSGTDFTLTISSLQPEDFAT |
| (SEQ ID NO 29) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECAEAAAKEAAAKAMSYNLLGFLQRSSNFQSQKLLW |
| | QLNGRLEYCLKDRMNFDIPEEIKQLQQFQKEDAALTIYEML |
| | QNIF AIFRQDSSSTGWNETIVENLLANVYHQINHLKTVLEEK |
| | LEKEDFTRGKLMSSLHLKRYYGRILHYLKAKEYSHCAWTIV |
| | RVEILRNFYFINRLTGYLRN |
| antiCD40_hIgG2_ | QVQLVQSGAEVKKPGASVKVSCKASGYTFTGYYMHWVRQ |
| dK_HC--RL-- | APGQGLEWMGWINPDSGGTNYAQKFQGRVTMTRDTSISTA |
| IFNβ | YMELNRLRSDDTAVYYCARDQPLGYCTNGVCSYFDYWGQ |
| (SEQ ID NO 30) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPGPAPAMSYNLLGFLQRSSNFQCQKLLWQLNGRLEYCLK |
| | DRMNFDIPEEIKQLQQFQKEDAALTIYEMLQNIFAIFRQDSSS |
| | TGWNETIVENLLANVYHQINHLKTVLEEKLEKEDFTRGKLM |
| | SSLHLKRYYGRILHYLKAKEYSHCAWTIVRVEILRNFYFINR |
| | LTGYLRN |
| antiCD40_hIgG2_ | QVQLVQSGAEVKKPGASVKVSCKASGYTFTGYYMHWVRQ |
| dK_HC--RL-- | APGQGLEWMGWINPDSGGTNYAQKFQGRVTMTRDTSISTA |
| IFNβ_C17S | YMELNRLRSDDTAVYYCARDQPLGYCTNGVCSYFDYWGQ |
| (SEQ ID NO 31) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPGPAPAMSYNLLGFLQRSSNFQSQKLLWQLNGRLEYCLK |
| | DRMNFDIPEEIKQLQQFQKEDAALTIYEMLQNIFAIFRQDSSS |
| | TGWNETIVENLLANVYHQINHLKTVLEEKLEKEDFTRGKLM |
| | SSLHLKRYYGRILHYLKAKEYSHCAWTIVRVEILRNFYFINR |
| | LTGYLRN |
| antiCD40_hIgG2_ | QVQLVQSGAEVKKPGASVKVSCKASGYTFTGYYMHWVRQ |
| dK_HC--HL-- | APGQGLEWMGWINPDSGGTNYAQKFQGRVTMTRDTSISTA |
| IFNβ | YMELNRLRSDDTAVYYCARDQPLGYCTNGVCSYFDYWGQ |
| (SEQ ID NO 32) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPGAEAAAKEAAAKAMSYNLLGFLQRSSNFQCQKLLWQL |
| | NGRLEYCLKDRMNFDIPEEIKQLQQFQKEDAALTIYEMLQNI |
| | FAIFRQDSSSTGWNETIVENLLANVYHQINHLKTVLEEKLEK |
| | EDFTRGKLMSSLHLKRYYGRILHYLKAKEYSHCAWTIVRVE |
| | ILRNFYFINRLTGYLRN |
| antiCD40_hIgG2_ | QVQLVQSGAEVKKPGASVKVSCKASGYTFTGYYMHWVRQ |
| dK_HC--HL-- | APGQGLEWMGWINPDSGGTNYAQKFQGRVTMTRDTSISTA |
| IFNβ_C17S | YMELNRLRSDDTAVYYCARDQPLGYCTNGVCSYFDYWGQ |
| (SEQ ID NO 33) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPGAEAAAKEAAAKAMSYNLLGFLQRSSNFQSQKLLWQL |
| | NGRLEYCLKDRMNFDIPEEIKQLQQFQKEDAALTIYEMLQNI |
| | FAIFRQDSSSTGWNETIVENLLANVYHQINHLKTVLEEKLEK |
| | EDFTRGKLMSSLHLKRYYGRILHYLKAKEYSHCAWTIVRVE |
| | ILRNFYFINRLTGYLRN |
| antiCD40_LC-- | DIQMTQSPSSVSASVGDRVTITCRASQGIYSWLAWYQQKPG |
| RL--IFNβ | KAPNLLIYTASTLQSGVPSRFSGSGSGTDFTLTISSLQPEDFAT |
| (SEQ ID NO 34) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECPAPAMSYNLLGFLQRSSNFQCQKLLWQLNGRLEYC |
| | LKDRMNFDIPEEIKQLQQFQKEDAALTIYEMLQNIFAIFRQD |
| | SSSTGWNETIVENLLANVYHQINHLKTVLEEKLEKEDFTRG |
| | KLMSSLHLKRYYGRILHYLKAKEYSHCAWTIVRVEILRNFY |
| | FINRLTGYLRN |
| antiCD40_LC-- | DIQMTQSPSSVSASVGDRVTITCRASQGIYSWLAWYQQKPG |
| RL--IFNβ_C17S | KAPNLLIYTASTLQSGVPSRFSGSGSGTDFTLTISSLQPEDFAT |
| (SEQ ID NO 35) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECPAPAMSYNLLGFLQRSSNFQSQKLLWQLNGRLEYC |
| | LKDRMNFDIPEEIKQLQQFQKEDAALTIYEMLQNIFAIFRQD |
| | SSSTGWNETIVENLLANVYHQINHLKTVLEEKLEKEDFTRG |
| | KLMSSLHLKRYYGRILHYLKAKEYSHCAWTIVRVEILRNFY |
| | FINRLTGYLRN |
| antiCD40_LC-- | DIQMTQSPSSVSASVGDRVTITCRASQGIYSWLAWYQQKPG |
| GST--IFNβ_C17S | KAPNLLIYTASTLQSGVPSRFSGSGSGTDFTLTISSLQPEDFAT |
| (SEQ ID NO 36) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECSGGTSGSTSGTGSMSYNLLGFLQRSSNFQSQKLLW |
| | QLNGRLEYCLKDRMNFDIPEEIKQLQQFQKEDAALTIYEML |
| | QNIFAIFRQDSSSTGWNETIVENLLANVYHQINHLKTVLEEK |
| | LEKEDFTRGKLMSSLHLKRYYGRILHYLKAKEYSHCAWTIV |
| | RVEILRNFYFINRLTGYLRN |
| antiCD40_LC-- | DIQMTQSPSSVSASVGDRVTITCRASQGIYSWLAWYQQKPG |
| HL2--IFNβ_C17S | KAPNLLIYTASTLQSGVPSRFSGSGSGTDFTLTISSLQPEDFAT |
| (SEQ ID NO 37) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECAEAAAKEAAAKAAEAAAKEAAAKAMSYNLLGFL |
| | QRSSNFQSQKLLWQLNGRLEYCLKDRMNFDIPEEIKQLQQF |
| | QKEDAALTIYEMLQNIFAIFRQDSSSTGWNETIVENLLANVY |
| | HQINHLKTVLEEKLEKEDFTRGKLMSSLHLKRYYGRILHYL |
| | KAKEYSHCAWTIVRVEILRNFYFINRLTGYLRN |
| antiCD40_hIgG2_ | QVQLVQSGAEVKKPGASVKVSCKASGYTFTGYYMHWVRQ |
| dK_HC--(G4S)2-- | APGQGLEWMGWINPDSGGTNYAQKFQGRVTMTRDTSISTA |
| IFNα2a | YMELNRLRSDDTAVYYCARDQPLGYCTNGVCSYFDYWGQ |
| (SEQ ID NO 38) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPGGGGGSGGGGSCDLPQTHSLGSRRTLMLLAQMRKISLF |
| | SCLKDRHDFGFPQEEFGNQFQKAETIPVLHEMIQQIFNLFSTK |
| | DSSAAWDETLLDKFYTELYQQLNDLEACVIQGVGVTETPL |
| | MKEDSILAVRKYFQRITLYLKEKKYSPCAWEVVRAEIMRSF |
| | SLSTNLQESLRSKE |
| antiCD40_hIgG2_ | QVQLVQSGAEVKKPGASVKVSCKASGYTFTGYYMHWVRQ |
| dK_HC--(G4S)3-- | APGQGLEWMGWINPDSGGTNYAQKFQGRVTMTRDTSISTA |
| IFNα2a | YMELNRLRSDDTAVYYCARDQPLGYCTNGVCSYFDYWGQ |
| (SEQ ID NO 39) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPGGGGGSGGGGSGGGGSCDLPQTHSLGSRRTLMLLAQ |
| | MRKISLFSCLKDRHDFGFPQEEFGNQFQKAETIPVLHEMIQQI |
| | FNLFSTKDSSAAWDETLLDKFYTELYQQLNDLEACVIQGVG |
| | VTETPLMKEDSILAVRKYFQRITLYLKEKKYSPCAWEVVRA |
| | EIMRSFSLSTNLQESLRSKE |
| antiCD40_hIgG2_ | QVQLVQSGAEVKKPGASVKVSCKASGYTFTGYYMHWVRQ |
| dK_HC--(G4S)4-- | APGQGLEWMGWINPDSGGTNYAQKFQGRVTMTRDTSISTA |
| IFNα2a | YMELNRLRSDDTAVYYCARDQPLGYCTNGVCSYFDYWGQ |
| (SEQ ID NO 40) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPGGGGGSGGGGSGGGGSGGGGSCDLPQTHSLGSRRTL |
| | MLLAQMRKISLFSCLKDRHDFGFPQEEFGNQFQKAETIPVLH |
| | EMIQQIFNLFSTKDSSAAWDETLLDKFYTELYQQLNDLEAC |
| | VIQGVGVTETPLMKEDSILAVRKYFQRITLYLKEKKYSPCA |
| | WEVVRAEIMRSFSLSTNLQESLRSKE |
| antiCD40_LC-- | DIQMTQSPSSVSASVGDRVTITCRASQGIYSWLAWYQQKPG |
| (G4S)2--IFNα2a | KAPNLLIYTASTLQSGVPSRFSGSGSGTDFTLTISSLQPEDFAT |
| (SEQ ID NO 41) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECGGGGSGGGGSCDLPQTHSLGSRRTLMLLAQMRKIS |
| | LFSCLKDRHDFGFPQEEFGNQFQKAETIPVLHEMIQQIFNLFS |
| | TKDSSAAWDETLLDKFYTELYQQLNDLEACVIQGVGVTETP |
| | LMKEDSILAVRKYFQRITLYLKEKKYSPCAWEVVRAEIMRS |
| | FSLSTNLQESLRSKE |
| antiCD40_LC-- | DIQMTQSPSSVSASVGDRVTITCRASQGIYSWLAWYQQKPG |
| (G4S)3--IFNα2a | KAPNLLIYTASTLQSGVPSRFSGSGSGTDFTLTISSLQPEDFAT |
| (SEQ ID NO 42) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECGGGGSGGGGSGGGGSCDLPQTHSLGSRRTLMLLA |
| | QMRKISLFSCLKDRHDFGFPQEEFGNQFQKAETIPVLHEMIQ |
| | QIFNLFSTKDSSAAWDETLLDKFYTELYQQLNDLEACVIQG |
| | VGVTETPLMKEDSILAVRKYFQRITLYLKEKKYSPCAWEVV |
| | RAEIMRSFSLSTNLQESLRSKE |
| antiCD40_LC-- | DIQMTQSPSSVSASVGDRVTITCRASQGIYSWLAWYQQKPG |
| (G4S)4--IFNα2a | KAPNLLIYTASTLQSGVPSRFSGSGSGTDFTLTISSLQPEDFAT |
| (SEQ ID NO 43) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECGGGGSGGGGSGGGGSGGGGSCDLPQTHSLGSRRT |
| | LMLLAQMRKISLFSCLKDRHDFGFPQEEFGNQFQKAETIPVL |
| | HEMIQQIFNLFSTKDSSAAWDETLLDKFYTELYQQLNDLEA |
| | CVIQGVGVTETPLMKEDSILAVRKYFQRITLYLKEKKYSPCA |
| | WEVVRAEIMRSFSLSTNLQESLRSKE |
| IFNβ--(G4S)3-- | MSYNLLGFLQRSSNFQCQKLLWQLNGRLEYCLKDRMNFDI |
| antiCD40_LC) | PEEIKQLQQFQKEDAALTIYEMLQNIFAIFRQDSSSTGWNETI |
| (SEQ ID NO 44) | VENLLANVYHQINHLKTVLEEKLEKEDFTRGKLMSSLHLKR |
| | YYGRILHYLKAKEYSHCAWTIVRVEILRNFYFINRLTGYLRN |
| | GGGGSGGGGSGGGGSDIQMTQSPSSVSASVGDRVTITCRA |
| | SQGIYSWLAWYQQKPGKAPNLLIYTASTLQSGVPSRFSGSGS |
| | GTDFTLTISSLQPEDFATYYCQQANIFPLTFGGGTKVEIKRTV |
| | AAPSVFIFPPSDEQLKSGTASVVCLLNNFYPREAKVQWKVD |
| | NALQSGNSQESVTEQDSKDSTYSLSSTLTLSKADYEKHKVY |
| | ACEVTHQGLSSPVTKSFNRGEC |
| antiCD40_LC-- | DIQMTQSPSSVSASVGDRVTITCRASQGIYSWLAWYQQKPG |
| (G4S)4--IFNβ | KAPNLLIYTASTLQSGVPSRFSGSGSGTDFTLTISSLQPEDFAT |
| (SEQ ID NO 45) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECGGGGSGGGGSGGGGSGGGGSMSYNLLGFLQRSSN |
| | FQCQKLLWQLNGRLEYCLKDRMNFDIPEEIKQLQQFQKEDA |
| | ALTIYEMLQNIFAIFRQDSSSTGWNETIVENLLANVYHQINH |
| | LKTVLEEKLEKEDFTRGKLMSSLHLKRYYGRILHYLKAKEY |
| | SHCAWTIVRVEILRNFYFINRLTGYLRN |
| IFNβ--(G4S)3-- | MSYNLLGFLQRSSNFQCQKLLWQLNGRLEYCLKDRMNFDI |
| antiCD40_HC_IgG1_ | PEEIKQLQQFQKEDAALTIYEMLQNIFAIFRQDSSSTGWNETI |
| NNAS_dK | VENLLANVYHQINHLKTVLEEKLEKEDFTRGKLMSSLHLKR |
| (SEQ ID NO 46) | YYGRILHYLKAKEYSHCAWTIVRVEILRNFYFINRLTGYLRN |
| | GGGGSGGGGSGGGGSQVQLVQSGAEVKKPGASVKVSCK |
| | ASGYTFTGYYMHWVRQAPGQGLEWMGWINPDSGGTNYA |
| | QKFQGRVTMTRDTSISTAYMELNRLRSDDTAVYYCARDQP |
| | LGYCTNGVCSYFDYWGQGTLVTVSSASTKGPSVFPLAPSSK |
| | STSGGTAALGCLVKDYFPEPVTVSWNSGALTSGVHTFPAVL |
| | QSSGLYSLSSVVTVPSSSLGTQTYICNVNHKPSNTKVDKKVE |
| | PKSCDKTHTCPPCPAPELLGGPSVFLFPPKPKDTLMISRTPEV |
| | TCVVVDVSHEDPEVKFNWYVDGVEVHNAKTKPREEQYNN |
| | ASRVVSVLTVLHQDWLNGKEYKCKVSNKALPAPIEKTISKA |
| | KGQPREPQVYTLPPSRDELTKNQVSLTCLVKGFYPSDIAVE |
| | WESNGQPENNYKTTPPVLDSDGSFFLYSKLTVDKSRWQQG |
| | NVFSCSVMHEALHNHYTQKSLSLSPG |
| antiCD40_HC_IgG1_ | QVQLVQSGAEVKKPGASVKVSCKASGYTFTGYYMHWVRQ |
| NNAS_dK-- | APGQGLEWMGWINPDSGGTNYAQKFQGRVTMTRDTSISTA |
| (G4S)4--IFNβ | YMELNRLRSDDTAVYYCARDQPLGYCTNGVCSYFDYWGQ |
| (SEQ ID NO 47) | GTLVTVSSASTKGPSVFPLAPSSKSTSGGTAALGCLVKDYFP |
| | EPVTVSWNSGALTSGVHTFPAVLQSSGLYSLSSVVTVPSSSL |
| | GTQTYICNVNHKPSNTKVDKKVEPKSCDKTHTCPPCPAPEL |
| | LGGPSVFLFPPKPKDTLMISRTPEVTCVVVDVSHEDPEVKEN |
| | WYVDGVEVHNAKTKPREEQYNNASRVVSVLTVLHQDWLN |
| | GKEYKCKVSNKALPAPIEKTISKAKGQPREPQVYTLPPSRDE |
| | LTKNQVSLTCLVKGFYPSDIAVEWESNGQPENNYKTTPPVL |
| | DSDGSFFLYSKLTVDKSRWQQGNVFSCSVMHEALHNHYTQ |
| | KSLSLSPGGGGGSGGGGSGGGGSGGGGSMSYNLLGFLQR |
| | SSNFQCQKLLWQLNGRLEYCLKDRMNFDIPEEIKQLQQFQK |
| | EDAALTIYEMLQNIFAIFRQDSSSTGWNETIVENLLANVYHQ |
| | INHLKTVLEEKLEKEDFTRGKLMSSLHLKRYYGRILHYLKA |
| | KEYSHCAWTIVRVEILRNFYFINRLTGYLRN |
| antiCD40_hIgG2_ | QVQLVQSGAEVKKPGASVKVSCKASGYTFTGYYMHWVRQ |
| dK_HC--HL-- | APGQGLEWMGWINPDSGGTNYAQKFQGRVTMTRDTSISTA |
| IFNα2A | YMELNRLRSDDTAVYYCARDQPLGYCTNGVCSYFDYWGQ |
| (SEQ ID NO 81) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPGAEAAAKEAAAKACDLPQTHSLGSRRTLMLLAQMRKI |
| | SLFSCLKDRHDFGFPQEEFGNQFQKAETIPVLHEMIQQIFNLF |
| | STKDSSAAWDETLLDKFYTELYQQLNDLEACVIQGVGVTET |
| | PLMKEDSILAVRKYFQRITLYLKEKKYSPCAWEVVRAEIMR |
| | SFSLSTNLQESLRSKE |
| antiCD40_LC- | DIQMTQSPSSVSASVGDRVTITCRASQGIYSWLAWYQQKPG |
| derivative--HL-- | KAPNLLIYTASTLQSGVPSRFSGSGSGTDFTLTISSLQPEDFAT |
| IFNα2A | YYCQQANIFPLTFGGGTKVEIKRTVAAPSVFIFPPSDEQLKSG |
| (SEQ ID NO 82) | TASVVCLLNNFYPREAKVQWKVDNALQSGNSQESVTEQDS |
| | KDSTYSLSSTLTLSKADYEKHKVYACEVTHQGLSSPVTKSF |
| | NRGEKSLSLSPGAEAAAKEAAAKACDLPQTHSLGSRRTLM |
| | LLAQMRKISLFSCLKDRHDFGFPQEEFGNQFQKAETIPVLHE |
| | MIQQIFNLFSTKDSSAAWDETLLDKFYTELYQQLNDLEACVI |
| | QGVGVTETPLMKEDSILAVRKYFQRITLYLKEKKYSPCAWE |
| | VVRAEIMRSFSLSTNLQESLRSKE |
| antiCD40_LC-- | DIQMTQSPSSVSASVGDRVTITCRASQGIYSWLAWYQQKPG |
| (G4S)4--IFNγ | KAPNLLIYTASTLQSGVPSRFSGSGSGTDFTLTISSLQPEDFAT |
| (SEQ ID NO 83) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECGGGGSGGGGSGGGGSGGGGSQDPYVKEAENLKK |
| | YFNAGHSDVADNGTLFLGILKNWKEESDRKIMQSQIVSFYF |
| | KLFKNFKDDQSIQKSVETIKEDMNVKFFNSNKKKRDDFEKL |
| | TNYSVTDLNVQRKAIHELIQVMAELSPAAKTGKRKRSQMLF |
| | RGRRASQ |
| antiCD40_hIgG2 | QVQLVQSGAEVKKPGASVKVSCKASGYTFTGYYMHWVRQ |
| dK_HC--(G4S)4-- | APGQGLEWMGWINPDSGGTNYAQKFQGRVTMTRDTSISTA |
| IFNγ | YMELNRLRSDDTAVYYCARDQPLGYCTNGVCSYFDYWGQ |
| (SEQ ID NO 84) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPGGGGGSGGGGSGGGGSGGGGSQDPYVKEAENLKKYF |
| | NAGHSDVADNGTLFLGILKNWKEESDRKIMQSQIVSFYFKL |
| | FKNFKDDQSIQKSVETIKEDMNVKFFNSNKKKRDDFEKLTN |
| | YSVTDLNVQRKAIHELIQVMAELSPAAKTGKRKRSQMLFRG |
| | RRASQ |
| antiCD40_LC-- | DIQMTQSPSSVSASVGDRVTITCRASQGIYSWLAWYQQKPG |
| (G4S)4--IFNλ2 | KAPNLLIYTASTLQSGVPSRFSGSGSGTDFTLTISSLQPEDFAT |
| (SEQ ID NO 85) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECGGGGSGGGGSGGGGSGGGGSVPVARLHGALPDA |
| | RGCHIAQFKSLSPQELQAFKRAKDALEESLLLKDCRCHSRLF |
| | PRTWDLRQLQVRERPMALEAELALTLKVLEATADTDPALV |
| | DVLDQPLHTLHHILSQFRACIQPQPTAGPRTRGRLHHWLYR |
| | LQEAPKKESPGCLEASVTFNLFRLLTRDLNCVASGDLCV |
| antiCD40_hIgG2 | QVQLVQSGAEVKKPGASVKVSCKASGYTFTGYYMHWVRQ |
| dK_HC--(G4S)4-- | APGQGLEWMGWINPDSGGTNYAQKFQGRVTMTRDTSISTA |
| IFNλ2 | YMELNRLRSDDTAVYYCARDQPLGYCTNGVCSYFDYWGQ |
| (SEQ ID NO 86) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPGGGGGSGGGGSGGGGSGGGGSVPVARLHGALPDARG |
| | CHIAQFKSLSPQELQAFKRAKDALEESLLLKDCRCHSRLFPR |
| | TWDLRQLQVRERPMALEAELALTLKVLEATADTDPALVDV |
| | LDQPLHTLHHILSQFRACIQPQPTAGPRTRGRLHHWLYRLQE |
| | APKKESPGCLEASVTFNLFRLLTRDLNCVASGDLCV |
| antiCD40_LC-- | DIQMTQSPSSVSASVGDRVTITCRASQGIYSWLAWYQQKPG |
| (G4S)4--IFNω | KAPNLLIYTASTLQSGVPSRFSGSGSGTDFTLTISSLQPEDFAT |
| (SEQ ID NO 87) | YYCQQANIFPLTFGGGTKVEIKRTVAAPSVFIFPPSDEQLKSG |
| | TASVVCLLNNFYPREAKVQWKVDNALQSGNSQESVTEQDS |
| | KDSTYSLSSTLTLSKADYEKHKVYACEVTHQGLSSPVTKSF |
| | NRGECGGGGSGGGGSGGGGSGGGGSLGCDLPQNHGLLSR |
| | NTLVLLHQMRRISPFLCLKDRRDFRFPQEMVKGSQLQKAHV |
| | MSVLHEMLQQIFSLFHTERSSAAWNMTLLDQLHTGLHQQL |
| | QHLETCLLQVVGEGESAGAISSPALTLRRYFQGIRVYLKEKK |
| | YSDCAWEVVRMEIMKSLFLSTNMQERLRSKDRDLGSS |
| antiCD40_hIgG2 | QVQLVQSGAEVKKPGASVKVSCKASGYTFTGYYMHWVRQ |
| dK_HC--(G4S)4-- | APGQGLEWMGWINPDSGGTNYAQKFQGRVTMTRDTSISTA |
| IFNϵ | YMELNRLRSDDTAVYYCARDQPLGYCTNGVCSYFDYWGQ |
| (SEQ ID NO 88) | GTLVTVSSASTKGPSVFPLAPCSRSTSESTAALGCLVKDYFP |
| | EPVTVSWNSGALTSGVHTFPAVLQSSGLYSLSSVVTVPSSNF |
| | GTQTYTCNVDHKPSNTKVDKTVERKCCVECPPCPAPPVAGP |
| | SVFLFPPKPKDTLMISRTPEVTCVVVDVSHEDPEVQFNWYV |
| | DGVEVHNAKTKPREEQFNSTFRVVSVLTVVHQDWLNGKEY |
| | KCKVSNKGLPAPIEKTISKTKGQPREPQVYTLPPSREEMTKN |
| | QVSLTCLVKGFYPSDIAVEWESNGQPENNYKTTPPMLDSDG |
| | SFFLYSKLTVDKSRWQQGNVFSCSVMHEALHNHYTQKSLS |
| | LSPGGGGGSGGGGSGGGGSGGGGSLDLKLIIFQQRQVNQE |
| | SLKLLNKLQTLSIQQCLPHRKNFLLPQKSLSPQQYQKGHTLA |
| | ILHEMLQQIFSLFRANISLDGWEENHTEKFLIQLHQQLEYLE |
| | ALMGLEAEKLSGTLGSDNLRLQVKMYFRRIHDYLENQDYS |
| | TCAWAIVQVEISRCLFFVFSLTEKLSKQGRPLNDMKQELTTE |
| | FRSPR |
| antiCD40 LC-- | DIQMTQSPSSVSASVGDRVTITCRASQGIYSWLAWYQQKPG |
| (G4S)3-- | KAPNLLIYTASTLQSGVPSRFSGSGSGTDFTLTISSLQPEDFAT |
| IFNβ_C17S | YYCQQANIFPLTFGGGTKVEIKRTVAAPSVFIFPPSDEQLKSG |
| (SEQ ID NO 94) | TASVVCLLNNFYPREAKVQWKVDNALQSGNSQESVTEQDS |
| | KDSTYSLSSTLTLSKADYEKHKVYACEVTHQGLSSPVTKSF |
| | NRGECGGGGSGGGGSGGGGSMSYNLLGFLQRSSNFQSQK |
| | LLWQLNGRLEYCLKDRMNFDIPEEIKQLQQFQKEDAALTIY |
| | EMLQNIFAIFRQDSSSTGWNETIVENLLANVYHQINHLKTVL |
| | EEKLEKEDFTRGKLMSSLHLKRYYGRILHYLKAKEYSHCA |
| | WTIVRVEILRNFYFINRLTGYLRN |

TABLE 8
Sequences of exemplary interferon-associated antigen binding protein and
components thereof based on the antiCD40 antibody 3G5. Italic sequences
correspond to signal peptides. Bold non-italic sequences correspond to linkers.
Mutated amino acids are underlined.

| Name/ | |
|---|---|
| SEQ ID Number | Sequence |
| antiCD40_light | EIVMTQSPATLSVSPGERATLSCRASQSVRSNLAWYQQKPG |
| chain | QAPRLLIYGASTRATGIPARFSGSGSGTEFTLTINSLQSEDFA |
| (SEQ ID NO 59) | VYYCQQHNKWITFGQGTRLEIKRTVAAPSVFIFPPSDEQLKS |
| | GTASVVCLLNNFYPREAKVQWKVDNALQSGNSQESVTEQD |
| | SKDSTYSLSSTLTLSKADYEKHKVYACEVTHQGLSSPVTKSF |
| | NRGEC |
| antiCD40_light | MGWSCIILFLVATATGVHSEIVMTQSPATLSVSPGERATLSCR |
| chain with signal | ASQSVRSNLAWYQQKPGQAPRLLIYGASTRATGIPARFSGS |
| peptide 1 | GSGTEFTLTINSLQSEDFAVYYCQQHNKWITFGQGTRLEIKR |
| (SEQ ID NO 60) | TVAAPSVFIFPPSDEQLKSGTASVVCLLNNFYPREAKVQWK |
| | VDNALQSGNSQESVTEQDSKDSTYSLSSTLTLSKADYEKHK |
| | VYACEVTHQGLSSPVTKSFNRGEC |
| antiCD40_heavy | QVQLVESGGGVVQPGKSLRLSCAASGFTFSSNGIHWVRQAP |
| chain hIgG2 dK | GKGLEWVAVIWSDGSNKFYADSVKGRFTISRDNSKNTLYL |
| (SEQ ID NO 61) | QMNSLRAEDTAVYYCARASGSGSYYNFFDYWGQGTLVTVS |
| | SASTKGPSVFPLAPCSRSTSESTAALGCLVKDYFPEPVTVSW |
| | NSGALTSGVHTFPAVLQSSGLYSLSSVVTVPSSNFGTQTYTC |
| | NVDHKPSNTKVDKTVERKCCVECPPCPAPPVAGPSVFLFPP |
| | KPKDTLMISRTPEVTCVVVDVSHEDPEVQFNWYVDGVEVH |
| | NAKTKPREEQFNSTFRVVSVLTVVHQDWLNGKEYKCKVSN |
| | KGLPAPIEKTISKTKGQPREPQVYTLPPSREEMTKNQVSLTC |
| | LVKGFYPSDIAVEWESNGQPENNYKTTPPMLDSDGSFFLYS |
| | KLTVDKSRWQQGNVFSCSVMHEALHNHYTQKSLSLSPG |
| antiCD40_heavy | MGWSCIILFLVATATGVHSQVQLVESGGGVVQPGKSLRLSCA |
| chain hIgG2 dK | ASGFTFSSNGIHWVRQAPGKGLEWVAVIWSDGSNKFYADS |
| with Signal | VKGRFTISRDNSKNTLYLQMNSLRAEDTAVYYCARASGSGS |
| peptide 1 | YYNFFDYWGQGTLVTVSSASTKGPSVFPLAPCSRSTSESTAA |
| (SEQ ID NO 62) | LGCLVKDYFPEPVTVSWNSGALTSGVHTFPAVLQSSGLYSL |
| | SSVVTVPSSNFGTQTYTCNVDHKPSNTKVDKTVERKCCVEC |
| | PPCPAPPVAGPSVFLFPPKPKDTLMISRTPEVTCVVVDVSHE |
| | DPEVQFNWYVDGVEVHNAKTKPREEQFNSTFRVVSVLTVV |
| | HQDWLNGKEYKCKVSNKGLPAPIEKTISKTKGQPREPQVYT |
| | LPPSREEMTKNQVSLTCLVKGFYPSDIAVEWESNGQPENNY |
| | KTTPPMLDSDGSFFLYSKLTVDKSRWQQGNVFSCSVMHEA |
| | LHNHYTQKSLSLSPG |
| antiCD40 | QVQLVESGGGVVQPGKSLRLSCAASGFTFSSNGIHWVRQAP |
| antibody Fab | GKGLEWVAVIWSDGSNKFYADSVKGRFTISRDNSKNTLYL |
| region heavy chain | QMNSLRAEDTAVYYCARASGSGSYYNFFDYWGQGTLVTVS |
| hIgG2 | SASTKGPSVFPLAPCSRSTSESTAALGCLVKDYFPEPVTVSW |
| (SEQ ID NO 63) | NSGALTSGVHTFPAVLQSSGLYSLSSVVTVPSSNFGTQTYTC |
| | NVDHKPSNTKVDKTVERKCCVE |
| antiCD40 | MGWSCIILFLVATATGVHSQVQLVESGGGVVQPGKSLRLSCA |
| antibody Fab | ASGFTFSSNGIHWVRQAPGKGLEWVAVIWSDGSNKFYADS |
| region heavy chain | VKGRFTISRDNSKNTLYLQMNSLRAEDTAVYYCARASGSGS |
| hIgG2 with signal | YYNFFDYWGQGTLVTVSSASTKGPSVFPLAPCSRSTSESTAA |
| peptide 1 | LGCLVKDYFPEPVTVSWNSGALTSGVHTFPAVLQSSGLYSL |
| (SEQ ID NO 64) | SSVVTVPSSNFGTQTYTCNVDHKPSNTKVDKTVERKCCVE |
| antiCD40 | QVQLVESGGGVVQPGKSLRLSCAASGFTFSSNGIHWVRQAP |
| antibody Fab | GKGLEWVAVIWSDGSNKFYADSVKGRFTISRDNSKNTLYL |
| region heavy chain | QMNSLRAEDTAVYYCARASGSGSYYNFFDYWGQGTLVTVS |
| hIgG2 | SASTKGPSVFPLAPCSRSTSESTAALGCLVKDYFPEPVTVSW |
| --TEV--6His tag | NSGALTSGVHTFPAVLQSSGLYSLSSVVTVPSSNFGTQTYTC |
| (SEQ ID NO 65) | NVDHKPSNTKVDKTVERKCCVEENLYFQSHHHHHH |
| antiCD40_hIgG2 | QVQLVESGGGVVQPGKSLRLSCAASGFTFSSNGIHWVRQAP |
| dK_HC--RL-- | GKGLEWVAVIWSDGSNKFYADSVKGRFTISRDNSKNTLYL |
| IFNβdM | QMNSLRAEDTAVYYCARASGSGSYYNFFDYWGQGTLVTVS |
| (SEQ ID NO 66) | SASTKGPSVFPLAPCSRSTSESTAALGCLVKDYFPEPVTVSW |
| | NSGALTSGVHTFPAVLQSSGLYSLSSVVTVPSSNFGTQTYTC |
| | NVDHKPSNTKVDKTVERKCCVECPPCPAPPVAGPSVFLFPP |
| | KPKDTLMISRTPEVTCVVVDVSHEDPEVQFNWYVDGVEVH |
| | NAKTKPREEQFNSTFRVVSVLTVVHQDWLNGKEYKCKVSN |
| | KGLPAPIEKTISKTKGQPREPQVYTLPPSREEMTKNQVSLTC |
| | LVKGFYPSDIAVEWESNGQPENNYKTTPPMLDSDGSFFLYS |
| | KLTVDKSRWQQGNVFSCSVMHEALHNHYTQKSLSLSPGPA |
| | PASYNLLGFLQRSSNFQCQKLLWQLNGRLEYCLKDRMNFDI |
| | PEEIKQLQQFQKEDAALTIYEMLQNIFAIFRQDSSSTGWNETI |
| | VENLLANVYHQINHLKTVLEEKLEKEDFTRGKLMSSLHLKR |
| | YYGRILHYLKAKEYSHCAWTIVRVEILRNFYFINRLTGYLRN |
| antiCD40_hIgG2 | QVQLVESGGGVVQPGKSLRLSCAASGFTFSSNGIHWVRQAP |
| dK_HC--RL-- | GKGLEWVAVIWSDGSNKFYADSVKGRFTISRDNSKNTLYL |
| IFNβdM_C17S | QMNSLRAEDTAVYYCARASGSGSYYNFFDYWGQGTLVTVS |
| (SEQ ID NO 67) | SASTKGPSVFPLAPCSRSTSESTAALGCLVKDYFPEPVTVSW |
| | NSGALTSGVHTFPAVLQSSGLYSLSSVVTVPSSNFGTQTYTC |
| | NVDHKPSNTKVDKTVERKCCVECPPCPAPPVAGPSVFLFPP |
| | KPKDTLMISRTPEVTCVVVDVSHEDPEVQFNWYVDGVEVH |
| | NAKTKPREEQFNSTFRVVSVLTVVHQDWLNGKEYKCKVSN |
| | KGLPAPIEKTISKTKGQPREPQVYTLPPSREEMTKNQVSLTC |
| | LVKGFYPSDIAVEWESNGQPENNYKTTPPMLDSDGSFFLYS |
| | KLTVDKSRWQQGNVFSCSVMHEALHNHYTQKSLSLSPGPA |
| | PASYNLLGFLQRSSNFQSQKLLWQLNGRLEYCLKDRMNFDI |
| | PEEIKQLQQFQKEDAALTIYEMLQNIFAIFRQDSSSTGWNETI |
| | VENLLANVYHQINHLKTVLEEKLEKEDFTRGKLMSSLHLKR |
| | YYGRILHYLKAKEYSHCAWTIVRVEILRNFYFINRLTGYLRN |
| antiCD40_hIgG2 | QVQLVESGGGVVQPGKSLRLSCAASGFTFSSNGIHWVRQAP |
| dK_HC--HL-- | GKGLEWVAVIWSDGSNKFYADSVKGRFTISRDNSKNTLYL |
| IFNβdM | QMNSLRAEDTAVYYCARASGSGSYYNFFDYWGQGTLVTVS |
| (SEQ ID NO 68) | SASTKGPSVFPLAPCSRSTSESTAALGCLVKDYFPEPVTVSW |
| | NSGALTSGVHTFPAVLQSSGLYSLSSVVTVPSSNFGTQTYTC |
| | NVDHKPSNTKVDKTVERKCCVECPPCPAPPVAGPSVFLFPP |
| | KPKDTLMISRTPEVTCVVVDVSHEDPEVQFNWYVDGVEVH |
| | NAKTKPREEQFNSTFRVVSVLTVVHQDWLNGKEYKCKVSN |
| | KGLPAPIEKTISKTKGQPREPQVYTLPPSREEMTKNQVSLTC |
| | LVKGFYPSDIAVEWESNGQPENNYKTTPPMLDSDGSFFLYS |
| | KLTVDKSRWQQGNVFSCSVMHEALHNHYTQKSLSLSPGAE |
| | AAAKEAAAKASYNLLGFLQRSSNFQCQKLLWQLNGRLEYC |
| | LKDRMNFDIPEEIKQLQQFQKEDAALTIYEMLQNIFAIFRQD |
| | SSSTGWNETIVENLLANVYHQINHLKTVLEEKLEKEDFTRG |
| | KLMSSLHLKRYYGRILHYLKAKEYSHCAWTIVRVEILRNFY |
| | FINRLTGYLRN |
| antiCD40_hIgG2 | QVQLVESGGGVVQPGKSLRLSCAASGFTFSSNGIHWVRQAP |
| dK_HC--HL-- | GKGLEWVAVIWSDGSNKFYADSVKGRFTISRDNSKNTLYL |
| IFNβdM_C17S | QMNSLRAEDTAVYYCARASGSGSYYNFFDYWGQGTLVTVS |
| (SEQ ID NO 69) | SASTKGPSVFPLAPCSRSTSESTAALGCLVKDYFPEPVTVSW |
| | NSGALTSGVHTFPAVLQSSGLYSLSSVVTVPSSNFGTQTYTC |
| | NVDHKPSNTKVDKTVERKCCVECPPCPAPPVAGPSVFLFPP |
| | KPKDTLMISRTPEVTCVVVDVSHEDPEVQFNWYVDGVEVH |
| | NAKTKPREEQFNSTFRVVSVLTVVHQDWLNGKEYKCKVSN |
| | KGLPAPIEKTISKTKGQPREPQVYTLPPSREEMTKNQVSLTC |
| | LVKGFYPSDIAVEWESNGQPENNYKTTPPMLDSDGSFFLYS |
| | KLTVDKSRWQQGNVFSCSVMHEALHNHYTQKSLSLSPGAE |
| | AAAKEAAAKASYNLLGFLQRSSNFQSQKLLWQLNGRLEYC |
| | LKDRMNFDIPEEIKQLQQFQKEDAALTIYEMLQNIFAIFRQD |
| | SSSTGWNETIVENLLANVYHQINHLKTVLEEKLEKEDFTRG |
| | KLMSSLHLKRYYGRILHYLKAKEYSHCAWTIVRVEILRNFY |
| | FINRLTGYLRN |
| antiCD40_LC-- | EIVMTQSPATLSVSPGERATLSCRASQSVRSNLAWYQQKPG |
| HL2--IFNβ_C17S | QAPRLLIYGASTRATGIPARFSGSGSGTEFTLTINSLQSEDFA |
| (SEQ ID NO 70) | VYYCQQHNKWITFGQGTRLEIKRTVAAPSVFIFPPSDEQLKS |
| | GTASVVCLLNNFYPREAKVQWKVDNALQSGNSQESVTEQD |
| | SKDSTYSLSSTLTLSKADYEKHKVYACEVTHQGLSSPVTKSF |
| | NRGECAEAAAKEAAAKAAEAAAKEAAAKAMSYNLLGFL |
| | QRSSNFQSQKLLWQLNGRLEYCLKDRMNFDIPEEIKQLQQF |
| | QKEDAALTIYEMLQNIFAIFRQDSSSTGWNETIVENLLANVY |
| | HQINHLKTVLEEKLEKEDFTRGKLMSSLHLKRYYGRILHYL |
| | KAKEYSHCAWTIVRVEILRNFYFINRLTGYLRN |
| antiCD40_LC-- | EIVMTQSPATLSVSPGERATLSCRASQSVRSNLAWYQQKPG |
| (G4S)3-- | QAPRLLIYGASTRATGIPARFSGSGSGTEFTLTINSLQSEDFA |
| IFNβ_C17S | VYYCQQHNKWITFGQGTRLEIKRTVAAPSVFIFPPSDEQLKS |
| (SEQ ID NO 71) | GTASVVCLLNNFYPREAKVQWKVDNALQSGNSQESVTEQD |
| | SKDSTYSLSSTLTLSKADYEKHKVYACEVTHQGLSSPVTKSF |
| | NRGECGGGGSGGGGSGGGGSMSYNLLGFLQRSSNFQSQK |
| | LLWQLNGRLEYCLKDRMNFDIPEEIKQLQQFQKEDAALTIY |
| | EMLQNIFAIFRQDSSSTGWNETIVENLLANVYHQINHLKTVL |
| | EEKLEKEDFTRGKLMSSLHLKRYYGRILHYLKAKEYSHCA |
| | WTIVRVEILRNFYFINRLTGYLRN |
| antiCD40_hIgG2 | QVQLVESGGGVVQPGKSLRLSCAASGFTFSSNGIHWVRQAP |
| dK_HC--(G4S)2-- | GKGLEWVAVIWSDGSNKFYADSVKGRFTISRDNSKNTLYL |
| IFNα2a | QMNSLRAEDTAVYYCARASGSGSYYNFFDYWGQGTLVTVS |
| (SEQ ID NO 72) | SASTKGPSVFPLAPCSRSTSESTAALGCLVKDYFPEPVTVSW |
| | NSGALTSGVHTFPAVLQSSGLYSLSSVVTVPSSNFGTQTYTC |
| | NVDHKPSNTKVDKTVERKCCVECPPCPAPPVAGPSVFLFPP |
| | KPKDTLMISRTPEVTCVVVDVSHEDPEVQFNWYVDGVEVH |
| | NAKTKPREEQFNSTFRVVSVLTVVHQDWLNGKEYKCKVSN |
| | KGLPAPIEKTISKTKGQPREPQVYTLPPSREEMTKNQVSLTC |
| | LVKGFYPSDIAVEWESNGQPENNYKTTPPMLDSDGSFFLYS |
| | KLTVDKSRWQQGNVFSCSVMHEALHNHYTQKSLSLSPGGG |
| | GGSGGGGSCDLPQTHSLGSRRTLMLLAQMRKISLFSCLKDR |
| | HDFGFPQEEFGNQFQKAETIPVLHEMIQQIFNLFSTKDSSAA |
| | WDETLLDKFYTELYQQLNDLEACVIQGVGVTETPLMKEDSI |
| | LAVRKYFQRITLYLKEKKYSPCAWEVVRAEIMRSFSLSTNL |
| | QESLRSKE |
| antiCD40_hIgG2 | QVQLVESGGGVVQPGKSLRLSCAASGFTFSSNGIHWVRQAP |
| dK_HC--(G4S)3-- | GKGLEWVAVIWSDGSNKFYADSVKGRFTISRDNSKNTLYL |
| IFNα2a | QMNSLRAEDTAVYYCARASGSGSYYNFFDYWGQGTLVTVS |
| (SEQ ID NO 73) | SASTKGPSVFPLAPCSRSTSESTAALGCLVKDYFPEPVTVSW |
| | NSGALTSGVHTFPAVLQSSGLYSLSSVVTVPSSNFGTQTYTC |
| | NVDHKPSNTKVDKTVERKCCVECPPCPAPPVAGPSVFLFPP |
| | KPKDTLMISRTPEVTCVVVDVSHEDPEVQFNWYVDGVEVH |
| | NAKTKPREEQFNSTFRVVSVLTVVHQDWLNGKEYKCKVSN |
| | KGLPAPIEKTISKTKGQPREPQVYTLPPSREEMTKNQVSLTC |
| | LVKGFYPSDIAVEWESNGQPENNYKTTPPMLDSDGSFFLYS |
| | KLTVDKSRWQQGNVFSCSVMHEALHNHYTQKSLSLSPGGG |
| | GGSGGGGSGGGGSCDLPQTHSLGSRRTLMLLAQMRKISLF |
| | SCLKDRHDFGFPQEEFGNQFQKAETIPVLHEMIQQIFNLFSTK |
| | DSSAAWDETLLDKFYTELYQQLNDLEACVIQGVGVTETPL |
| | MKEDSILAVRKYFQRITLYLKEKKYSPCAWEVVRAEIMRSF |
| | SLSTNLQESLRSKE |
| antiCD40_hlgG2 | QVQLVESGGGVVQPGKSLRLSCAASGFTFSSNGIHWVRQAP |
| dK_HC--(G4S)4-- | GKGLEWVAVIWSDGSNKFYADSVKGRFTISRDNSKNTLYL |
| IFNα2a | QMNSLRAEDTAVYYCARASGSGSYYNFFDYWGQGTLVTVS |
| (SEQ ID NO 74) | SASTKGPSVFPLAPCSRSTSESTAALGCLVKDYFPEPVTVSW |
| | NSGALTSGVHTFPAVLQSSGLYSLSSVVTVPSSNFGTQTYTC |
| | NVDHKPSNTKVDKTVERKCCVECPPCPAPPVAGPSVFLFPP |
| | KPKDTLMISRTPEVTCVVVDVSHEDPEVQFNWYVDGVEVH |
| | NAKTKPREEQFNSTFRVVSVLTVVHQDWLNGKEYKCKVSN |
| | KGLPAPIEKTISKTKGQPREPQVYTLPPSREEMTKNQVSLTC |
| | LVKGFYPSDIAVEWESNGQPENNYKTTPPMLDSDGSFFLYS |
| | KLTVDKSRWQQGNVFSCSVMHEALHNHYTQKSLSLSPGGG |
| | GGSGGGGSGGGGSGGGGSCDLPQTHSLGSRRTLMLLAQM |
| | RKISLFSCLKDRHDFGFPQEEFGNQFQKAETIPVLHEMIQQIF |
| | NLFSTKDSSAAWDETLLDKFYTELYQQLNDLEACVIQGVGV |
| | TETPLMKEDSILAVRKYFQRITLYLKEKKYSPCAWEVVRAEI |
| | MRSFSLSTNLQESLRSKE |
| antiCD40_hIgG2 | QVQLVESGGGVVQPGKSLRLSCAASGFTFSSNGIHWVRQAP |
| dK_HC--HL-- | GKGLEWVAVIWSDGSNKFYADSVKGRFTISRDNSKNTLYL |
| IFNα2a | QMNSLRAEDTAVYYCARASGSGSYYNFFDYWGQGTLVTVS |
| (SEQ ID NO 75) | SASTKGPSVFPLAPCSRSTSESTAALGCLVKDYFPEPVTVSW |
| | NSGALTSGVHTFPAVLQSSGLYSLSSVVTVPSSNFGTQTYTC |
| | NVDHKPSNTKVDKTVERKCCVECPPCPAPPVAGPSVFLFPP |
| | KPKDTLMISRTPEVTCVVVDVSHEDPEVQFNWYVDGVEVH |
| | NAKTKPREEQFNSTFRVVSVLTVVHQDWLNGKEYKCKVSN |
| | KGLPAPIEKTISKTKGQPREPQVYTLPPSREEMTKNQVSLTC |
| | LVKGFYPSDIAVEWESNGQPENNYKTTPPMLDSDGSFFLYS |
| | KLTVDKSRWQQGNVFSCSVMHEALHNHYTQKSLSLSPGAE |
| | AAAKEAAAKACDLPQTHSLGSRRTLMLLAQMRKISLFSCL |
| | KDRHDFGFPQEEFGNQFQKAETIPVLHEMIQQIFNLFSTKDSS |
| | AAWDETLLDKFYTELYQQLNDLEACVIQGVGVTETPLMKE |
| | DSILAVRKYFQRITLYLKEKKYSPCAWEVVRAEIMRSFSLST |
| | NLQESLRSKE |
| antiCD40_hIgG2 | QVQLVESGGGVVQPGKSLRLSCAASGFTFSSNGIHWVRQAP |
| dK_HC--(G4S)4-- | GKGLEWVAVIWSDGSNKFYADSVKGRFTISRDNSKNTLYL |
| IFNγ | QMNSLRAEDTAVYYCARASGSGSYYNFFDYWGQGTLVTVS |
| (SEQ ID NO 93) | SASTKGPSVFPLAPCSRSTSESTAALGCLVKDYFPEPVTVSW |
| | NSGALTSGVHTFPAVLQSSGLYSLSSVVTVPSSNFGTQTYTC |
| | NVDHKPSNTKVDKTVERKCCVECPPCPAPPVAGPSVFLFPP |
| | KPKDTLMISRTPEVTCVVVDVSHEDPEVQFNWYVDGVEVH |
| | NAKTKPREEQFNSTFRVVSVLTVVHQDWLNGKEYKCKVSN |
| | KGLPAPIEKTISKTKGQPREPQVYTLPPSREEMTKNQVSLTC |
| | LVKGFYPSDIAVEWESNGQPENNYKTTPPMLDSDGSFFLYS |
| | KLTVDKSRWQQGNVFSCSVMHEALHNHYTQKSLSLSPGGG |
| | GGSGGGGSGGGGSGGGGSQDPYVKEAENLKKYFNAGHS |
| | DVADNGTLFLGILKNWKEESDRKIMQSQIVSFYFKLFKNFK |
| | DDQSIQKSVETIKEDMNVKFFNSNKKKRDDFEKLTNYSVTD |
| | LNVQRKAIHELIQVMAELSPAAKTGKRKRSQMLFRGRRASQ |

In preferred embodiments, the interferon-associated antigen binding proteins described herein are interferon-fused antigen binding proteins comprising polypeptides derived from those specified in Table 9, in particular Table 9A or Table 9B, more particularly Table 9A below, and especially from the polypeptides of SEQ ID NO 38, SEQ ID NO 39, SEQ ID NO 40, SEQ ID NO 41, SEQ ID NO 42 or SEQ ID NO 43 above. In preferred embodiments, the interferon-associated antigen binding proteins described herein are interferon-fused antigen binding proteins consisting of polypeptides derived from those specified in Table 9, in particular Table 9A or Table 9B, more particularly Table 9A below, and especially from the polypeptides of SEQ ID NO 38, SEQ ID NO 39, SEQ ID NO 40, SEQ ID NO 41, SEQ ID NO 42 or SEQ ID NO 43 above. In more preferred embodiments, the interferon-fused antibody comprises the sequences as set forth in SEQ ID NO 38 and SEQ ID NO 3. In other more preferred embodiments, the interferon-fused antibody comprises the sequences as set forth in SEQ ID NO 39 and SEQ ID NO 3. In other more preferred embodiments, the interferon-fused antibody comprises the sequences as set forth in SEQ ID NO 40 and SEQ ID NO 3. In other more preferred embodiments, the interferon-fused antibody comprises the sequences as set forth in SEQ ID NO 41 and SEQ TD NO 9. In other more preferred embodiments, the interferon-fused antibody comprises the sequences as set forth in SEQ ID NO 42 and SEQ ID NO 9. In other more preferred embodiments, the interferon-fused antibody comprises the sequences as set forth in SEQ ID NO 43 and SEQ ID NO 9.

TABLE 9
Polypeptide combinations found in preferred interferon-fused antigen binding proteins of the
invention based on the antiCD40 antibody CP870, 893, their mean EC₅₀ values with regard to
the activation of CD40 and IFN-pathways and their productivity (i.e., yield per liter culture).
Each sequence combination as indicated is comprised twice in the respective IFA.
A

| Interferon - | | | | | |
|---|---|---|---|---|---|
| fused | | | | | |
| Antibody | Sequence | CD40 EC₅₀ | IFNβ EC₅₀ | IFNα EC₅₀ | Productivity |
| (IFA) | combination | (ng/mL) | (ng/mL) | (ng/mL) | (mg/L) |
| IFA1 | (SEQ ID NO 28) + | 74.1 | 1.64 | | 16.7 |
| | (SEQ ID NO 9) | | | | |
| IFA2 | (SEQ ID NO 29) + | 111 | 0.14 | | 17.8 |
| | (SEQ ID NO 9) | | | | |
| IFA8 | (SEQ ID NO 30) + | 39.7 | 2.9 | | 6.45 |
| | (SEQ ID NO 3) | | | | |
| IFA9 | (SEQ ID NO 31) + | 42.6 | 0.7 | | 3.4 |
| | (SEQ ID NO 3) | | | | |
| IFA10 | (SEQ ID NO 32) + | 26.5 | 4.5 | | 6.9 |
| | (SEQ ID NO 3) | | | | |
| IFA11 | (SEQ ID NO 33) + | 42.8 | 1.78 | | 5.1 |
| | (SEQ ID NO 3) | | | | |
| IFA12 | (SEQ ID NO 34) + | 105 | 3.64 | | 21.2 |
| | (SEQ ID NO 9) | | | | |
| IFA13 | (SEQ ID NO 35) + | 192 | 0.7 | | 11.5 |
| | (SEQ ID NO 9) | | | | |
| IFA19 | (SEQ ID NO 36) + | 110 | 1.3 | | 5.6 |
| | (SEQ ID NO 9) | | | | |
| IFA20 | (SEQ ID NO 37) + | 182 | 2.34 | | 4.2 |
| | (SEQ ID NO 9) | | | | |
| IFA25 | (SEQ ID NO 38) + | 13.3 | | 5.1 | 21 |
| | (SEQ ID NO 3) | | | | |
| IFA26 | (SEQ ID NO 39) + | 15.35 | | 4 | 8.6 |
| | (SEQ ID NO 3) | | | | |
| IFA27 | (SEQ ID NO 40) + | 17 | | 2.4 | 9.3 |
| | (SEQ ID NO 3) | | | | |
| IFA28 | (SEQ ID NO 41) + | 12.8 | | 4.5 | 75 |
| | (SEQ ID NO 9) | | | | |
| IFA29 | (SEQ ID NO 42) + | 11.1 | | 2 | 56.6 |
| | (SEQ ID NO 9) | | | | |
| IFA30 | (SEQ ID NO 43) + | 11.3 | | 1.6 | 46.6 |
| | (SEQ ID NO 9) | | | | |
| IFA34 | (SEQ ID NO 44) + | active (SN) | active (SN) | | no significant |
| | (SEQ ID NO 49) | | | | production |
| IFA35 | (SEQ ID NO 45) + | active (SN) | active (SN) | | no significant |
| | (SEQ ID NO 49) | | | | production |
| IFA36 | (SEQ ID NO 46) + | active (SN) | active (SN) | | no significant |
| | (SEQ ID NO 3) | | | | production |
| IFA37 | (SEQ ID NO 47) + | active (SN) | active (SN) | | no significant |
| | (SEQ ID NO 3) | | | | production |
| IFA126 | (SEQ ID NO 94) + | 364.05 | 2.075 | | not determined |
| | (SEQ ID NO 9) | | | | |

B

| Interferon - | | | | | | | | |
|---|---|---|---|---|---|---|---|---|
| fused | | CD40 | IFNα | IFNλ | IFNγ | IFNε | IFNω | |
| Antibody | Sequence | EC₅₀ | EC₅₀ | EC₅₀ | EC₅₀ | EC₅₀ | EC₅₀ | Productivity |
| (IFA) | combination | (ng/mL) | (ng/mL) | (ng/mL) | (ng/ml) | (ng/mL) | (ng/mL) | (mg/L) |
| IFA38 | (SEQ ID NO 81) + | 22.7 | 3.77 | | | | | 1.32 |
| | (SEQ ID NO 3) | | | | | | | |
| IFA39 | (SEQ ID NO 82) + | 17.5 | 2.95 | | | | | 1.25 |
| | (SEQ ID NO 9) | | | | | | | |
| IFA42 | (SEQ ID NO 83) + | 65.6 | | | 15.4 | | | 0.72 |
| | (SEQ ID NO 9) | | | | | | | |
| IFA43 | (SEQ ID NO 84) + | 50.8 | | | <0.001 | | | 0.55 |
| | (SEQ ID NO 3) | | | | | | | |
| IFA44 | (SEQ ID NO 85) + | 41.4 | | 0.153 | | | | 0.91 |
| | (SEQ ID NO 9) | | | | | | | |
| IFA45 | (SEQ ID NO 86) + | 25.8 | | <0.001 | | | | 1.09 |
| | (SEQ ID NO 3) | | | | | | | |
| IFA46 | (SEQ ID NO 87) + | 86.3 | | | | | 0.493 | 0.89 |
| | (SEQ ID NO 9) | | | | | | | |
| IFA49 | (SEQ ID NO 88) + | 65.8 | | | | 78.2 | | 0.61 |
| | (SEQ ID NO 3) | | | | | | | |
| IFA50 | (SEQ ID NO 41) + | 128 | 1.36 | | | | | 0.57 |
| | (SEQ ID NO 50) | | | | | | | |
| IFA51 | (SEQ ID NO 42) + | 123 | 1.43 | | | | | 0.48 |
| | (SEQ ID NO 50) | | | | | | | |

SN: supernatant.

In other preferred embodiments, the interferon-associated antigen binding proteins described herein are interferon-fused antigen binding proteins comprising polypeptides derived from those specified in Table 10 below. In preferred embodiments, the interferon-associated antigen binding proteins described herein are interferon-fused antigen binding proteins consisting of polypeptides derived from those specified in Table 10 below.

TABLE 10
Polypeptide combinations found in preferred interferon-fused antigen binding
proteins of the invention based on the antiCD40 antibody 3G5, their mean EC₅₀
values with regard to the activation of CD40 and IFN-pathways. Each sequence
combination as indicated is comprised twice in the respective IFA.
A

| Interferon - | | | | | |
|---|---|---|---|---|---|
| fused | | CD40 | IFNβ | IFNα | |
| Antibody | Sequence | EC₅₀ | EC₅₀ | EC₅₀ | Productivity |
| (IFA) | combination | (ng/ml) | (ng/mL) | (ng/mL) | mg/L |
| IFA106 | (seq ID NO 66) + | 190.5 | 10.30 | | 0.36 |
| | (seq ID NO 59) | | | | |
| IFA107 | (seq ID NO 67) + | 141.5 | 2.03 | | 0.28 |
| | (seq ID NO 59) | | | | |
| IFA108 | (seq ID NO 68) + | 37.3 | 1.27 | | 0.59 |
| | (seq ID NO 59) | | | | |
| IFA109 | (seq ID NO 69) + | 30 | 0.45 | | 0.4 |
| | (seq ID NO 59) | | | | |
| IFA114 | (seq ID NO 70) + | active (SN) | active (SN) | | no significant |
| | (seq ID NO 61) | | | | production |
| IFA115 | (seq ID NO 71) + | active (SN) | active (SN) | | no significant |
| | (seq ID NO 61) | | | | production |
| IFA121 | (seq ID NO 72) + | 14.2 | | 0.12 | 22.6 |
| | (seq ID NO 59) | | | | |
| IFA122 | (seq ID NO 73) + | 11.74 | | 0.07 | 16.8 |
| | (seq ID NO 59) | | | | |
| IFA123 | (seq ID NO 74) + | 12.85 | | 0.05 | 17.2 |
| | (seq ID NO 59) | | | | |
| IFA124 | (seq ID NO 75) + | 12.14 | | 0.04 | 21.6 |
| | (seq ID NO 59) | | | | |

B

| Interferon - | | | | | | | | |
|---|---|---|---|---|---|---|---|---|
| fused | | CD40 | IFNα | IFNλ | IFNγ | IFNε | IFNω | |
| Antibody | Sequence | EC₅₀ | EC₅₀ | EC₅₀ | EC₅₀ | EC₅₀ | EC₅₀ | Productivity |
| (IFA) | combination | (ng/mL) | (ng/mL) | (ng/mL) | (ng/mL) | (ng/mL) | (ng/mL) | (mg/L) |
| IFA125 | (seq ID NO 93) + | 8.8 | | | 0.142 | | | 1.79 |
| | (seq ID NO 59) | | | | | | | |

SN: supernatant.

### Nucleic Acids and Expression Vectors

In one aspect, a combination of polynucleotides encoding an interferon-associated antigen binding protein is provided. Methods of making an interferon-associated antigen binding protein comprising expressing these polynucleotides are also provided.

In some embodiments, a nucleic acid encoding an IFN or a functional fragment thereof being fused to an agonistic anti-CD40 antibody or an agonistic antigen binding fragment thereof, as disclosed herein is provided. In certain exemplary embodiments, the nucleic acid is encoding an IFN or a functional fragment thereof fused to an agonistic anti-CD40 antibody or an agonistic antigen binding fragment thereof according to any of the sequences set forth in SEQ ID NOs 93 to 94, or a nucleic acid sequence at least 80%, at least 85%, at least 90%, at least 95%, at least 98% or at least 99% identical to a nucleic acid encoding any of these sequences. In certain exemplary embodiments, said nucleic acid is at least 95%, at least 98% or at least 99% identical to a nucleic acid encoding any of SEQ ID NOs 93 to 94. In certain exemplary embodiments, the nucleic acid is encoding an IFN or a functional fragment thereof fused to an agonistic anti-CD40 antibody or an agonistic antigen binding fragment thereof according to any of the sequences set forth in SEQ ID NOs 81 to 88, or a nucleic acid sequence at least 80%, at least 85%, at least 90%, at least 95%, at least 98% or at least 99% identical to a nucleic acid encoding any of these sequences. In certain exemplary embodiments, said nucleic acid is at least 95%, at least 98% or at least 99% identical to a nucleic acid encoding any of SEQ ID NOs 81 to 88. In preferred embodiments, the nucleic acid is encoding an IFN or a functional fragment thereof fused to an agonistic anti-CD40 antibody or an agonistic antigen binding fragment thereof according to any of the sequences set forth in SEQ ID NOs 28 to 47, or a nucleic acid sequence at least 80%, at least 85%, at least 90%, at least 95%, at least 98% or at least 99% identical to a nucleic acid encoding any of these sequences. In even more specific embodiments, said nucleic acid is at least 95%, at least 98% or at least 99% identical to a nucleic acid encoding any of SEQ ID NOs 28 to 47. In other preferred embodiments, the nucleic acid is encoding an IFN or a functional fragment thereof fused to an agonistic anti-CD40 antibody or an agonistic antigen binding fragment thereof according to any of the sequences set forth in SEQ ID NOs 66 to 75, or a nucleic acid sequence at least 80%, at least 85%, at least 90%, at least 95%, at least 98% or at least 99% identical to a nucleic acid encoding any of these sequences. In even more specific embodiments, said nucleic acid is at least 95%, at least 98% or at least 99% identical to a nucleic acid encoding any of SEQ ID NOs 66 to 75.

In those embodiments wherein a nucleic acid encodes an IFN or a functional fragment thereof being fused to a light chain of the agonistic anti-CD40 antibody or the agonistic antigen binding fragment thereof, the nucleic acid may further encode a heavy chain of the agonistic anti-CD40 antibody or the agonistic antigen binding fragment thereof. In more specific embodiments, the heavy chain of the agonistic anti-CD40 antibody or the agonistic antigen binding fragment thereof comprises a sequence as set forth in SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 48, SEQ ID NO 49, or SEQ ID NO 50, or a nucleic acid sequence at least 80%, at least 85%, at least 90%, at least 95%, at least 98% or at least 99% identical to a nucleic acid encoding any of these sequences. In even more specific embodiments, said nucleic acid is at least 95%, at least 98% or at least 99% identical to a nucleic acid encoding SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 48, SEQ ID NO 49, or SEQ ID NO 50. In other more specific embodiments, the heavy chain of the agonistic anti-CD40 antibody or the agonistic antigen binding fragment thereof comprises a sequence as set forth in SEQ ID NO 61, SEQ ID NO 62, SEQ ID NO 63, SEQ ID NO 64 or SEQ ID NO 65, or a nucleic acid sequence at least at least 80%, at least 85%, at least 90%, at least 95%, at least 98% or at least 99% identical to a nucleic acid encoding any of these sequences. In such other even more specific embodiments, said nucleic acid is at least 95%, at least 98% or at least 99% identical to a nucleic acid encoding SEQ ID NO 61, SEQ ID NO 62, SEQ ID NO 63, SEQ ID NO 64 or SEQ ID NO 65.

In those embodiments where a nucleic acid encodes an IFN or a functional fragment thereof being fused to the heavy chain of the agonistic anti-CD40 antibody or the agonistic antigen binding fragment thereof, the nucleic acid may further encode a light chain of the agonistic anti-CD40 antibody or the agonistic antigen binding fragment thereof. In more specific embodiments, the light chain of the agonistic anti-CD40 antibody or the agonistic antigen binding fragment thereof comprises a sequence as set forth in SEQ ID NO 3, SEQ ID NO 4 or SEQ ID NO 5, or a nucleic acid sequence at least 80%, at least 85%, at least 90%, at least 95%, at least 98% or at least 99% identical to a nucleic acid encoding any of these sequences. In even more specific embodiments, said nucleic acid is at least 95%, at least 98% or at least 99% identical to a nucleic acid encoding SEQ ID NO 3, SEQ ID NO 4 or SEQ ID NO 5. In other more specific embodiments, the light chain of the agonistic anti-CD40 antibody or the agonistic antigen binding fragment thereof comprises a sequence as set forth in SEQ ID NO 59 or SEQ ID NO 60, or a nucleic acid sequence at least at least 80%, at least 85%, at least 90%, at least 95%, at least 98% or at least 99% identical to a nucleic acid encoding any of these sequences. In even more specific embodiments, said nucleic acid is at least 95%, at least 98% or at least 99% identical to a nucleic acid encoding SEQ ID NO 59 or SEQ ID NO 60.

In certain embodiments, the nucleic acids described herein may comprise a sequence encoding a sequence to increase the yield (e.g. a solubility tag) or facilitate purification of the expressed proteins (i.e., a purification tag). Purification tags are known to a person skilled in the art and may be selected from glutathione S-transferase (GST) tags, maltose binding protein (MBP) tags, calmodulin binding peptide (CBP) tags, intein-chitin binding domain (intein-CBD) tags, Streptavidin/Biotin-based tags (such as biotinylation signal peptide (BCCP) tags, Streptavidin-binding peptide (SBP) tags, His-patch ThioFusion tags, tandem affinity purification (TAP) tags, Small ubiquitin-like modifier (SUMO) tags, HaloTag® (Promega), Profinity eXact™ system (Bio-Rad). In some embodiments, the purification tag may be a polyhistidine tag (e.g., a His₆-, His₇-, His₈-, His₉- or His₁₀-tag). In other embodiments, the purification tag may be a Strep-tag (e.g., a Strep-tag® or a Strep-tag II®; IBA Life Sciences). In yet other embodiments, the purification tag may be a maltose binding protein (MBP) tag.

In some embodiments, the nucleic acid sequence may further comprise a sequence encoding a cleavage site for removal of the purification tag. Such cleavage sequences are known to a person skilled in the art and may be selected from a sequence recognized and cleaved by an endoprotease or an exoprotease. In some embodiments, an endoprotease for the removal of a purification tag may be selected from: Enteropeptidase, Thrombin, Factor Xa, TEV protease or Rhinovirus 3C protease. In some embodiments, an exoprotease for the removal of a purification tag may be selected from: Carboxypeptidase A, Carboxypeptidase B or DAPase. In preferred embodiments, the protease for the removal of a purification tag is TEV protease. In a more specific preferred embodiment, the nucleic acid comprises a sequence encoding a His₆-tag and a TEV cleavage site. In an even more specific preferred embodiment, said nucleic acid comprises a sequence encoding a sequence as set forth in SEQ ID NO 27.

The nucleic acid molecules of the invention may also comprise a sequence encoding a signal peptide. The skilled person is aware of the various signal peptides available to direct the expressed protein to the desired site of folding, assembly and/or maturation as well as to effect secretion of the final protein into the medium to facilitate downstream processing. Thus, in some embodiments, the signal peptide is a secretory signal peptide. The encoded signal peptide may comprise a sequence as set forth in SEQ ID NO: 1 or SEQ ID NO: 2. In some embodiments, the signal peptide comprises the sequence as set forth in SEQ ID NO: 1. In other embodiments, the signal peptide comprises the sequence as set forth in SEQ ID NO: 2.

Signal peptide 1 (SEQ ID NO 1) was used for synthesis of the polypeptide sequences as set forth in SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 44, SEQ ID NO 45, SEQ ID NO 46, SEQ ID NO 47, SEQ ID NO 50, SEQ ID NO 65, SEQ ID NO 66, SEQ ID NO 67, SEQ ID NO 68, SEQ ID NO 69, SEQ ID NO 70, SEQ ID NO 71, SEQ ID NO 72, SEQ ID NO 73, SEQ ID NO 74 and SEQ ID NO 75. Such signal peptide that is initially present at the N-terminus of the respective sequence of the polypeptide is cleaved during synthesis.

Signal peptide 2 (SEQ ID NO 2) was used for synthesis of the polypeptide sequences as set forth in SEQ ID NO 38, SEQ ID NO 39, SEQ ID NO 40, SEQ ID NO 41, SEQ ID NO 42 and SEQ ID NO 43. Such signal peptide that is initially present at the N-terminus of the respective sequence of the polypeptide is cleaved during synthesis.

For the synthesis of the polypeptide sequences as set forth in SEQ ID NO 81, SEQ ID NO 82, SEQ ID NO 83, SEQ ID NO 84, SEQ ID NO 85, SEQ ID NO 86, SEQ ID NO 87 and SEQ ID NO 88 the signal peptide MGWSCIILFLVATATGVHS (SEQ ID NO 1) was used. Such signal peptide that is initially present at the N-terminus of the respective sequence of the polypeptide is cleaved during synthesis.

For the synthesis of the polypeptide sequences as set forth in SEQ ID NO 93 and SEQ ID NO 94 the signal peptide 1 (SEQ ID NO 1) was used. Such signal peptide that is initially present at the N-terminus of the respective sequence of the polypeptide is cleaved during synthesis.

Polynucleotides encoding an IFN or a functional fragment thereof being fused to the agonistic anti-CD40 antibody or the agonistic antigen binding fragment thereof as disclosed herein are typically inserted in an expression vector for introduction into host cells that may be used to produce the desired quantity of the described or claimed interferon-associated antigen binding proteins. Accordingly, in certain aspects, the invention provides expression vectors comprising polynucleotides disclosed herein and host cells comprising these vectors and polynucleotides.

The term “vector” or “expression vector” is used herein for the purposes of the specification and claims, to mean vectors used in accordance with the present invention as a vehicle for introducing into and expressing a desired gene in a cell. As known to those skilled in the art, such vectors may easily be selected from the group consisting of plasmids, phages, viruses and retroviruses. In general, vectors compatible with the present invention will comprise a selection marker, appropriate restriction sites to facilitate cloning of the desired gene and the ability to enter and/or replicate in eukaryotic or prokaryotic cells.

Numerous expression vector systems may be employed for the purposes of this invention. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. In some embodiments the cloned variable region genes, one of them fused with a gene encoding an IFN or a functional fragment thereof, are inserted into an expression vector along with the heavy and light chain constant region genes (such as human genes) synthesized as discussed above.

In other embodiments, a vector system of the invention may comprise more than one vector. In some embodiments, a vector system may comprise a first vector for the expression of an IFN or a functional fragment thereof fused to a light chain of the agonistic anti-CD40 antibody or the agonistic antigen binding fragment thereof and a second vector for expression of a heavy chain of the agonistic anti-CD40 antibody or the agonistic antigen binding fragment thereof. Alternatively, such a vector system may comprise a first vector for the expression of an IFN or a functional fragment thereof fused to a heavy chain of the agonistic anti-CD40 antibody or the agonistic antigen binding fragment thereof and a second vector for expression of a light chain of the agonistic anti-CD40 antibody or the agonistic antigen binding fragment thereof.

In other embodiments, an interferon-associated antigen binding protein as described herein may be expressed using polycistronic constructs. In such expression systems, multiple gene products of interest such as those encoding an IFN or a functional fragment thereof being fused to a heavy chain of an agonistic anti-CD40 antibody or the agonistic antigen binding fragment thereof and encoding a light chain of said antibody, or those encoding an IFN or a functional fragment thereof being fused to a light chain of an agonistic anti-CD40 antibody or an agonistic antigen binding fragment thereof and encoding a heavy chain of said antibody or an agonistic antigen binding fragment thereof may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein. Those skilled in the art will appreciate that such expression systems may be used to effectively produce the full range of polypeptides disclosed in the instant application.

More generally, once a vector or a DNA sequence encoding an interferon-associated antigen binding protein of the present invention has been prepared, the expression vector may be introduced into an appropriate host cell. That is, the host cell may be transformed. Introduction of a plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, e.g., Ridgway, A. A. G. “Mammalian Expression Vectors” Chapter 24.2, pp. 470-472 Vectors, Rodriguez and Denhardt, Eds. (Butterworths, Boston, MA 1988). The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refer to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of the interferon-associated antigen binding protein unless it is clearly specified otherwise. In other words, recovery of polypeptide from the “cells” may mean either from spun down whole cells, or from the cell culture containing both the medium and the suspended cells.

In one embodiment, the host cell line used for expression of an interferon-associated antigen binding protein is of eukaryotic or prokaryotic origin. As used herein, the term “expression” may include the transcription and translation of more than one polypeptide chain (such as a heavy and a light chain of the antibody moiety of an interferon-associated antigen binding protein), which associate to form the final interferon-associated antigen binding protein. In one embodiment, the host cell line used for expression of an interferon-associated antigen binding protein is of bacterial origin. In one embodiment, the host cell line used for expression of an interferon-associated antigen binding protein is of mammalian origin; those skilled in the art can determine particular host cell lines which are best suited for the desired gene product to be expressed therein. Exemplary host cell lines include, but are not limited to, CHO K1 GS knockout from Horizon, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), HEK 293 (human kidney). In a preferred embodiment, HEK FS S11/254 cells may be used. In another preferred embodiment, CHO K1 GS from Horizon may be used. In one embodiment, the cell line provides for altered glycosylation, e.g., afucosylation, of the antibody expressed therefrom (e.g., PER.C6® (Crucell) or FUT8-knock-out CHO cell lines (POTELLIGENT™ cells) (Biowa, Princeton, NJ)). In one embodiment NS0 cells may be used. Host cell lines are typically available from commercial services, the American Tissue Culture Collection or from published literature.

In one embodiment, the host used for expression of an interferon-associated antigen binding protein is a non-human transgenic animal or transgenic plant.

Interferon-associated antigen binding proteins of the invention can also be produced transgenically through the generation of a non-human animal (e.g., mammal) or plant that is transgenic for the sequences of interest and production of the interferon-associated antigen binding protein in a recoverable form therefrom. In connection with the transgenic production in mammals, interferon-associated antigen binding proteins can be produced in, and recovered from, the milk of goats, cows, or other mammals. See, e.g., U.S. Pat. Nos. 5,827,690, 5,756,687, 5,750,172, and 5,741,957. Exemplary plant hosts are Nicotiana, Arabidopsis, duckweed, corn, wheat, potato, etc. Methods for expressing antibodies in plants, including a description of promoters and vectors, as well as transformation of plants is known in the art. See, e.g., U.S. Pat. No. 6,517,529, herein incorporated by reference. In some embodiments, non-human transgenic animals or plants are produced by introducing one or more nucleic acid molecules encoding an interferon-associated antigen binding protein of the invention into the animal or plant by standard transgenic techniques. See Hogan and U.S. Pat. No. 6,417,429. The transgenic cells used for making the transgenic animal can be embryonic stem cells or somatic cells. The transgenic non-human organisms can be chimeric, nonchimeric heterozygotes, and nonchimeric homozygotes. See, e.g., Hogan et al., Manipulating the Mouse Embryo: A Laboratory Manual 2nd ed., Cold Spring Harbor Press (1999); Jackson et al., Mouse Genetics and Transgenics: A Practical Approach, Oxford University Press (2000); and Pinkert, Transgenic Animal Technology: A Laboratory Handbook, Academic Press (1999). In some embodiments, the transgenic non-human animals have a targeted disruption and replacement by a targeting construct that encodes the sequence(s) of interest. The interferon-associated antigen binding proteins may be made in any transgenic animal. In a preferred embodiment, the non-human animals are mice, rats, sheep, pigs, goats, cattle or horses. The non-human transgenic animal expresses said interferon-associated antigen binding proteins in blood, milk, urine, saliva, tears, mucus and other bodily fluids.

In vitro production allows scale-up to give large amounts of the desired interferon-associated antigen binding proteins. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, a solution of an interferon-associated antigen binding protein, can be purified by the customary chromatography methods, for example gel filtration, ion-exchange chromatography, chromatography over DEAE-cellulose and/or (immuno-) affinity chromatography.

One or more genes encoding an interferon-associated antigen binding protein can also be expressed in non-mammalian cells such as bacteria or yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, interferon-associated antigen binding proteins according to the invention or components thereof (i.e., agonistic anti-CD40 antibodies or agonistic antigen binding fragments thereof, and IFNs or functional fragments of IFNs) can become part of inclusion bodies. The desired interferon-associated antigen binding proteins may then need to be isolated, optionally also refolded, and purified.

In addition to prokaryotes, eukaryotic microbes may also be used. Saccharomyces cerevisiae, or common baker's yeast, is the most commonly used among eukaryotic microorganisms although a number of other strains are commonly available. For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the TRP1 gene, which provides a selection marker for a mutant strain of yeast lacking the ability to grow in tryptophan, for example ATCC No. 44076 or PEP4-1 (Jones, Genetics, 85:12 (1977)). The presence of the trp1 lesion as a characteristic of the yeast host cell genome then provides an effective environment for detecting transformation by growth in the absence of tryptophan.

### Therapeutic Vectors

A nucleic acid sequence encoding an interferon-associated antigen binding protein can be inserted into a vector and used as a therapeutic vector, e.g., a vector that expresses an interferon-associated antigen binding protein of the invention. The construction of suitable, functional expression constructs and therapeutic expression vectors is known to one of ordinary skill in the art. Thus, in certain embodiments, the interferon-associated antigen binding protein may be administered to a subject by means of genetic delivery with RNA or DNA sequences, a vector or vector system encoding the interferon-associated antigen binding protein.

Therapeutic vectors can be delivered to a subject by, for example, intravenous injection, local administration (see U.S. Pat. No. 5,328,470) or by stereotactic injection (see, e.g., Chen et al., PNAS 91:3054-3057 (1994)). The pharmaceutical preparation of a therapeutic vector can include the vector in an acceptable diluent.

An interferon-associated antigen binding protein encoding nucleic acid, or nucleic acids, can be incorporated into a gene construct to be used as a part of a therapy protocol to deliver nucleic acids encoding an interferon-associated antigen binding protein. Expression vectors for in vivo transfection and expression of an interferon-associated antigen binding protein are provided.

Expression constructs of such components may be administered in any biologically effective carrier, e.g., any formulation or composition capable of effectively delivering the component nucleic acid sequence to cells in vivo, as are known to one of ordinary skill in the art. Approaches include, but are not limited to, insertion of the subject nucleic acid sequence(s) in viral vectors including, but not limited to, recombinant retroviruses, adenovirus, adeno-associated virus and herpes simplex virus-1, recombinant bacterial or eukaryotic plasmids and the like.

Retrovirus vectors and adeno-associated viral vectors can be used as a recombinant delivery system for the transfer of exogenous nucleic acid sequences in vivo, particularly into humans. Such vectors provide efficient delivery of genes into cells, and the transferred nucleic acids can be stably integrated into the chromosomal DNA of the host.

The development of specialized cell lines (termed “packaging cells”) which produce only replication-defective retroviruses has increased the utility of retroviruses for gene therapy, and defective retroviruses are characterized for use in gene transfer for gene therapy purposes (for a review see, e.g., Miller, Blood 76:271-78 (1990)). A replication-defective retrovirus can be packaged into virions, which can be used to infect a target cell through the use of a helper virus by standard techniques. Protocols for producing recombinant retroviruses and for infecting cells in vitro or in vivo with such viruses can be found in Current Protocols in Molecular Biology, Ausubel, et al., (eds.) Greene Publishing Associates, (1989), Sections 9.10-9.14, and other standard laboratory manuals. Non-limiting examples of suitable retroviruses include pLJ, pZIP, pWE and pEM, which are known to those of ordinary skill in the art. Examples of suitable packaging virus lines include *Crip, *Cre, *2 and *Am. (See, for example, Eglitis, et al., Science 230:1395-1398 (1985); Danos and Mulligan, Proc. Natl. Acad. Sci. USA 85:6460-6464 (1988); Wilson, et al., Proc. Natl. Acad. Sci. USA 85:3014-3018 (1988); Armentano, et al., Proc. Natl. Acad. Sci. USA 87:6141-6145 (1990); Huber, et al., Proc. Natl. Acad. Sci. USA 88:8039-8043 (1991); Ferry, et al., Proc. Natl. Acad. Sci. USA 88:8377-8381 (1991); Chowdhury, et al., Science 254:1802-1805 (1991); van Beusechem, et al., Proc. Natl. Acad. Sci. USA 89:7640-7644 (1992); Kay, et al., Human Gene Therapy 3:641-647 (1992); Dai, et al., Proc. Natl. Acad. Sci. USA 89:10892-10895 (1992); Hwu, et al., J. Immunol. 150:4104-4115 (1993); U.S. Pat. Nos. 4,868,116; 4,980,286; PCT Application WO 89/07136; PCT Application WO 89/02468; PCT Application WO 89/05345; and PCT Application WO 92/07573).

In another embodiment, adenovirus-derived delivery vectors are provided. The genome of an adenovirus can be manipulated such that it encodes and expresses a gene product of interest but is inactivated in terms of its ability to replicate in a normal lytic viral life cycle. See, for example, Berkner, et al., BioTechniques 6:616 (1988); Rosenfeld, et al., Science 252:431-434 (1991); and Rosenfeld, et al., Cell 68:143-155 (1992). Suitable adenoviral vectors derived from the adenovirus strain Ad type 5 d1324 or other strains of adenovirus (e.g., Ad2, Ad3, Ad7 etc.) are known to those of ordinary skill in the art. Recombinant adenoviruses can be advantageous in certain circumstances in that they are not capable of infecting non-dividing cells and can be used to infect a wide variety of cell types, including epithelial cells (Rosenfeld, et al. (1992), supra). Furthermore, the virus particle is relatively stable and amenable to purification and concentration and, as above, can be modified so as to affect the spectrum of infectivity. Additionally, introduced adenoviral DNA (and foreign DNA contained therein) is not integrated into the genome of a host cell, but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other delivery vectors (Berkner, et al. (1998), supra; Haj-Ahmand and Graham, J. Virol. 57:267 (1986)).

Yet another viral vector system useful for delivery of a nucleic acid sequence encoding an interferon-associated antigen binding protein, is the adeno-associated virus (AAV). AAV is a naturally occurring defective virus that requires another virus, such as an adenovirus or a herpes virus, as a helper virus for efficient replication and a productive life cycle. (For a review see Muzyczka, et al., Curr. Topics in Micro. and Immunol. 158:97-129 (1992)). It is also one of the few viruses that may integrate its DNA into non-dividing cells, and exhibits a high frequency of stable integration (see for example Flotte, et al., Am. J. Respir. Cell. Mol. Biol. 7:349-356 (1992); Samulski, et al., J. Virol. 63:3822-3828 (1989); and McLaughlin, et al., J. Virol. 62:1963-1973 (1989)). Vectors containing as little as 300 base pairs of AAV can be packaged and can integrate. Space for exogenous DNA is limited to about 4.5 kb. An AAV vector such as that described in Tratschin, et al., Mol. Cell. Biol. 5:3251-3260 (1985) can be used to introduce DNA into cells. A variety of nucleic acids have been introduced into different cell types using AAV vectors (see for example Hermonat, et al., Proc. Natl. Acad. Sci. USA 81:6466-6470 (1984); Tratschin, et al., Mol. Cell. Biol. 4:2072-2081 (1985); Wondisford, et al., Mol. Endocrinol. 2:32-39 (1988); Tratschin, et al., J. Virol. 51:611-619 (1984); and Flotte, et al., J. Biol. Chem. 268:3781-3790 (1993)).

In addition to viral transfer methods, non-viral methods can also be employed to cause expression of a nucleic acid sequence encoding an interferon-associated antigen binding protein in the tissue of a subject. Most non-viral methods of gene transfer rely on normal mechanisms used by mammalian cells for the uptake and intracellular transport of macromolecules. In some embodiments, non-viral delivery systems rely on endocytic pathways for the uptake of the subject gene by the targeted cell. Exemplary delivery systems of this type include liposomal derived systems, poly-lysine conjugates, and artificial viral envelopes. Other embodiments include plasmid injection systems such as are described in Meuli, et al., J. Invest. Dermatol. 116 (1):131-135 (2001); Cohen, et al., Gene Ther 7 (22):1896-905 (2000); or Tam, et al., Gene Ther. 7 (21):1867-74 (2000).

In clinical settings, the delivery systems can be introduced into a subject by any of a number of methods, each of which is familiar in the art. For instance, a pharmaceutical preparation of the delivery system can be introduced systemically, e.g., by intravenous injection. Specific transduction of the protein in the target cells occurs predominantly from specificity of transfection provided by the delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the delivery vehicle can be introduced by catheter (see, U.S. Pat. No. 5,328,470) or by stereotactic injection (e.g., Chen, et al., PNAS 91: 3054-3057 (1994)).

The pharmaceutical preparation of the therapeutic construct can consist essentially of the delivery system in an acceptable diluent, or can comprise a slow release matrix in which the delivery vehicle is imbedded. Alternatively, where the complete delivery system can be produced intact from recombinant cells, e.g., retroviral vectors, the pharmaceutical preparation can comprise one or more cells, which produce the delivery system.

### Methods of Treatment and Corresponding Uses

In one aspect, the invention provides methods of treating a patient in need thereof (e.g., a patient infected with Coronavirus, in particular SARS-CoV-2) comprising administering an effective amount of an interferon-associated antigen binding protein, or a nucleic acid sequence (e.g., mRNA) that encodes an interferon-associated antigen binding protein, as disclosed herein. The invention also provides for a use of an interferon-associated antigen binding protein, or a nucleic acid sequence (e.g., mRNA) that encodes an interferon-associated antigen binding protein, as disclosed herein, in the preparation of a medicament for the treatment of Coronavirus infection. In certain embodiments, the present invention provides kits and methods for the treatment of disorders and/or symptoms, e.g., Coronavirus-related disorders and/or Coronavirus-related symptoms, in a mammalian subject in need of such treatment. In certain exemplary embodiments, the subject is a human. The interferon-associated antigen binding proteins, or nucleic acid sequences that encode them, of the present invention are useful in a number of different applications. For example, in one embodiment, the subject interferon-associated antigen binding proteins, or nucleic acid sequences that encode them, are useful for rescuing Coronavirus-infected cells from cell death and/or from Coronavirus-induced cytopathic effect.

In another embodiment, the subject interferon-associated antigen binding proteins, or nucleic acid sequences that encode them, are useful for reducing one or more symptoms and/or complications associated with Coronavirus infection, as described herein (infra).

Accordingly, this invention also relates to a method of treating one or more disorders, symptoms and/or complications associated with Coronavirus infection in a human or other animal by administering to such human or animal an effective, non-toxic amount of an interferon-associated antigen binding protein, or a nucleic acid sequence that encodes it. One skilled in the art would be able, by routine experimentation, to determine what an effective, non-toxic amount of an interferon-associated antigen binding protein, or a nucleic acid sequence that encodes it, would be for the purpose of treating Coronavirus infection.

For example, a “therapeutically active amount” of an interferon-associated antigen binding protein of the present invention may vary according to factors such as the disease stage (e.g., acute vs. chronic), age, sex, medical complications (e.g., HIV co-infection, immunosuppressed conditions or diseases) and weight of the subject, and the ability of the interferon-associated antigen binding protein to elicit a desired response in the subject. The dosage regimen may be adjusted to provide the optimum therapeutic response. For example, several divided doses may be administered daily, or the dose may be proportionally reduced as indicated by the exigencies of the therapeutic situation.

In general, the compositions provided in the current invention may be used to prophylactically treat non-infected cells or therapeutically treat any Coronavirus-infected cells comprising an antigenic marker that allows for the targeting of the Coronavirus-infected cells by an interferon-associated antigen binding protein.

The treatment or prevention of a Coronavirus infection according to the methods and uses described and claimed herein may entail administering a TNFRSF agonist or a functional fragment thereof (e.g., a “in combination” with an IFN or a functional fragment thereof, and vice versa. If the TNFRSF agonist or the functional fragment thereof and the IFN or the functional fragment thereof are present in distinct pharmaceutical compositions, such administration in combination may be performed by simultaneous administration. Alternatively, the combined administration may be achieved in that case via sequential administration. For example, the TNFRSF agonist or the functional fragment thereof may be administered prior to the IFN or the functional fragment thereof. Alternatively, the IFN or the functional fragment thereof may be administered prior to the TNFRSF agonist or the functional fragment thereof. In the case of sequential administration, the administration will be performed in such a manner that the combined administration will lead to an enhancement of the beneficial effects of the treatment on, e.g., rescuing cells from Coronavirus-induced cell death and/or from Coronavirus-induced cytopathic effect preferably compared to the administration of the IFN or the functional fragment thereof alone. A skilled artisan will be readily able to determine suitable administration regimens, associated dosages, administration intervals, and, where sequential administration of distinct pharmaceutical compositions is chosen, intervals between the administration of said distinct pharmaceutical compositions, where such enhancement is achieved.

### Pharmaceutical Compositions and Administration Thereof

In certain embodiments, the interferon-associated antigen binding proteins of the invention or nucleic acid sequences (including vectors or vector systems) that encode them are comprised in a pharmaceutical composition. Methods of preparing and administering interferon-associated antigen binding proteins, or nucleic acid sequences that encode them, of the current invention to a subject are well known to or can be readily determined by those skilled in the art using this specification and the knowledge in the art as a guide. The route of administration of the interferon-associated antigen binding proteins, or nucleic acid sequences that encode them, of the current invention may be oral, parenteral, by inhalation or topical. The term “parenteral”, as used herein, includes intravenous, intraarterial, intraperitoneal, intramuscular, subcutaneous, rectal or vaginal administration. While all these forms of administration are clearly contemplated as being within the scope of the current invention, a form for administration would be a solution for injection, in particular for intravenous or intraarterial injection or drip. Usually, a suitable pharmaceutical composition for injection may comprise a buffering agent (e.g. acetate, phosphate or citrate buffer), a surfactant (e.g. polysorbate), optionally a stabilizing agent (e.g. human albumin), etc. In some embodiments, the buffering agent is acetate. In another embodiment, the buffering agent is formate. In yet another embodiment, the buffering agent is citrate. In related embodiments, the surfactant may be selected from the list comprising pluronics, PEG, sorbitan esters, polysorbates, triton, tromethamine, lecithin, cholesterol and tyloxapal. In preferred embodiments, the surfactant is polysorbate. In more preferred embodiments, the surfactant is polysorbate 20, polysorbate 40, polysorbate 60, polysorbate 80 or polysorbate 100, preferably polysorbate 20 or polysorbate 80.

In some embodiments, the interferon-associated antigen binding proteins, or nucleic acid sequences that encode them, can be delivered directly to the site of the adverse cellular population (e.g., the liver) thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the compositions and methods of the current invention, pharmaceutically acceptable carriers include, but are not limited to, 0.01-0.1 M, e.g., 0.05 M phosphate buffer, or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water-soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will typically be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, isotonic agents will be included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound such as an interferon-associated antigen binding protein, or a nucleic acid sequence encoding said interferon-associated antigen binding protein, of the present invention by itself or in combination with other active agents in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, exemplary methods of preparation include vacuum drying and freeze-drying, which yields a powder of an active ingredient plus any additional desired ingredient from a previously sterile-filtered solution thereof. The preparations for injections are processed, filled into containers such as ampules, bags, bottles, syringes or vials, and sealed under aseptic conditions according to methods known in the art. Further, the preparations may be packaged and sold in the form of a kit. Such articles of manufacture will typically have labels or package inserts indicating that the associated compositions are useful for treating a subject suffering from Coronavirus infection.

Effective doses of the compositions of the present invention, for the treatment of the above described Coronavirus infection-related conditions vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals, in particular non-human primates, can also be treated. Treatment dosages may be titrated using routine methods known to those of skill in the art to optimize safety and efficacy.

For treatment with an interferon-associated antigen binding protein, the dosage can range, e.g., from about 0.0001 to about 100 mg/kg, and more usually about 0.01 to about 5 mg/kg (e.g., about 0.02 mg/kg, about 0.25 mg/kg, about 0.5 mg/kg, about 0.75 mg/kg, about 1 mg/kg, about 2 mg/kg, etc.), of the host body weight. For example, dosages can be about 1 mg/kg body weight or about 10 mg/kg body weight or within the range of about 1 to about 10 mg/kg, e.g., at least about 1 mg/kg. Doses intermediate in the above ranges are also intended to be within the scope of the current invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimens entail administration about once per every two weeks or about once a month or about once every 3 to 6 months. Exemplary dosage schedules include about 1 to about 10 mg/kg or about 15 mg/kg on consecutive days, about 30 mg/kg on alternate days or about 60 mg/kg weekly.

Interferon-associated antigen binding proteins, or nucleic acid sequences expressing any of these, can be administered on multiple occasions. Intervals between single dosages can be weekly, monthly or yearly. Intervals can also be irregular as indicated by measuring blood levels of interferon-associated antigen binding proteins of components thereof in the patient. Alternatively, interferon-associated antigen binding proteins, or nucleic acid sequences expressing any of these can be administered as a sustained release formulation, in which case less frequent administration is required. Dosage and frequency vary depending on the half-life of the interferon-associated antigen binding proteins in the patient.

The term “half-life” or “t_{1/2}”, as referred to herein, relates to the stability and/or the rate of excretion of a compound, such as the interferon-associated antigen binding proteins of the invention. In practice, the half-life of a compound is usually measured in the serum and denotes the time after administration that the serum concentration is 50% of the serum concentration at the time of administration. The interferon-associated antigen binding proteins of the invention are characterized by a long serum half-life in mice. In some embodiments, the half-life of the interferon-associated antigen binding protein is at least 50 h, at least 60 h, at least 70 h, at least 80 h, at least 90 h or at least 100 h. In some embodiments, the half-life of the interferon-associated antigen binding protein is at least 100 h. In preferred embodiments, the half-life of the interferon-associated antigen binding protein in mice ranges from 116 to 158 h.

The half-life of a protein is related to its clearance. The term “clearance” or “clearance rate”, as used herein, refers to the volume of plasma cleared of the protein per unit time. Clearance of the interferon-associated antigen binding proteins of the invention is low. In some embodiments, clearance of the interferon-associated antigen binding protein is below 10 mL/h/kg, below 5 mL/h/kg, below 2.5 mL/h/kg, below 1 mL/h/kg, or below 0.5 mL/h/kg. In some embodiments, clearance of the interferon-associated antigen binding protein is below 5 mL/h/kg. In some embodiments, clearance of the interferon-associated antigen binding protein is below 1 mL/h/kg. In some embodiments, clearance of the interferon-associated antigen binding protein in mice ranges from 0.28 to 0.49 mL/h/kg.

The terms “volume of distribution”, “V_{D}”, “Vₛₛ” or “apparent volume of distribution” as used herein refer to the theoretical volume that would be necessary to contain the total amount of an administered compound such as the interferon-associated antigen binding protein of the invention at the same concentration that it is observed in the blood plasma and relates to the distribution of said compound between plasma and the rest of the body after oral or parenteral dosing. In certain embodiments, the volume of distribution Vss of the interferon-associated antigen binding protein is below 500 mL/kg, below 400 mL/kg, below 300 mL/kg, below 200 mL/kg, or below 100 mL/kg. In some embodiments, the volume of distribution Vss of the interferon-associated antigen binding protein is below 100 mL/kg. In some embodiments, the volume of distribution Vss of the interferon-associated antigen binding protein in mice ranges from 50 to 98 mL/kg.

Another related pharmacokinetic parameter is the systemic exposure. As used herein, the terms “systemic exposure”, “AUC” or “area under the curve” refer to the integral of the concentration-time curve. Systemic exposure might be represented by plasma (serum or blood) concentrations or the AUCs of parent compound and/or metabolite(s). The interferon-associated antigen binding proteins of the invention circulate in the blood with higher systemic exposure (AUC (0-inf)) than their parental antibody. In some embodiments, the parental antibody is CP870,893. In other embodiments, the parental antibody is 3G5. In some embodiments, the systemic exposure of the interferon-associated antigen binding protein is at least 600 μg*h/mL, at least 700 μg*h/mL, at least 800 μg*h/mL, at least 900 μg*h/mL or at least 1000 μg*h/mL, preferably at least 1000 μg*h/mL. In some embodiments, the systemic exposure of the interferon-associated antigen binding protein in mice ranges from 1033 μg*h/mL to 1793 μg*h/mL.

As previously discussed, an interferon-associated antigen binding protein of the present invention may be administered in a pharmaceutically effective amount for the in vivo treatment of mammalian disorders. In this regard, it will be appreciated that as disclosed an interferon-associated antigen binding protein, will be formulated to facilitate administration and promote stability of the active agent.

A pharmaceutical composition in accordance with the present invention can comprise a pharmaceutically acceptable, non-toxic, sterile carrier such as physiological saline, nontoxic buffers, preservatives and the like. A pharmaceutically effective amount of an interferon-associated antigen binding protein typically is an amount sufficient to mediate one or more of: a reduction of Coronavirus-induced cell death, in particular SARS-CoV-2-induced cell death; and a stimulation of the IFN signaling pathway in an infected cell. Of course, the pharmaceutical compositions of the present invention may be administered in single or multiple doses to provide for a pharmaceutically effective amount of the interferon-associated antigen binding protein.

In keeping with the scope of the present invention, interferon-associated antigen binding proteins, or nucleic acid sequences expressing any of them, may be administered to a human or other animal in accordance with the aforementioned methods of treatment in an amount sufficient to produce a therapeutic effect. The interferon-associated antigen binding proteins, or nucleic acid sequences expressing any of them, can be administered to such human or other animal in a conventional dosage form prepared by combining the interferon-associated antigen binding proteins, or nucleic acid sequences expressing any of them, with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of interferon-associated antigen binding proteins, or nucleic acid sequences expressing any of them, described in the current invention may prove to be effective.

It is to be understood that the methods described in this invention are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2nd edition).

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included,” is not limiting. The use of the term “comprising” shall include the term “consisting of” unless stated otherwise.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The section headings used herein are for organizational purposes only and are not to be construed as limiting the subject matter described. The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention using this disclosure as a guide. Having now described certain embodiments in detail, the same will be more clearly understood by reference to the following examples, which are included for purposes of illustration only and are not intended to be limiting.

### EXAMPLES

### Example I

### Generation of Interferon-Fused Antibodies (IFA) Based on Agonistic Anti-CD40 Antibody CP870,893 and Characterization on Reporter Cells

I.a—IFA Design

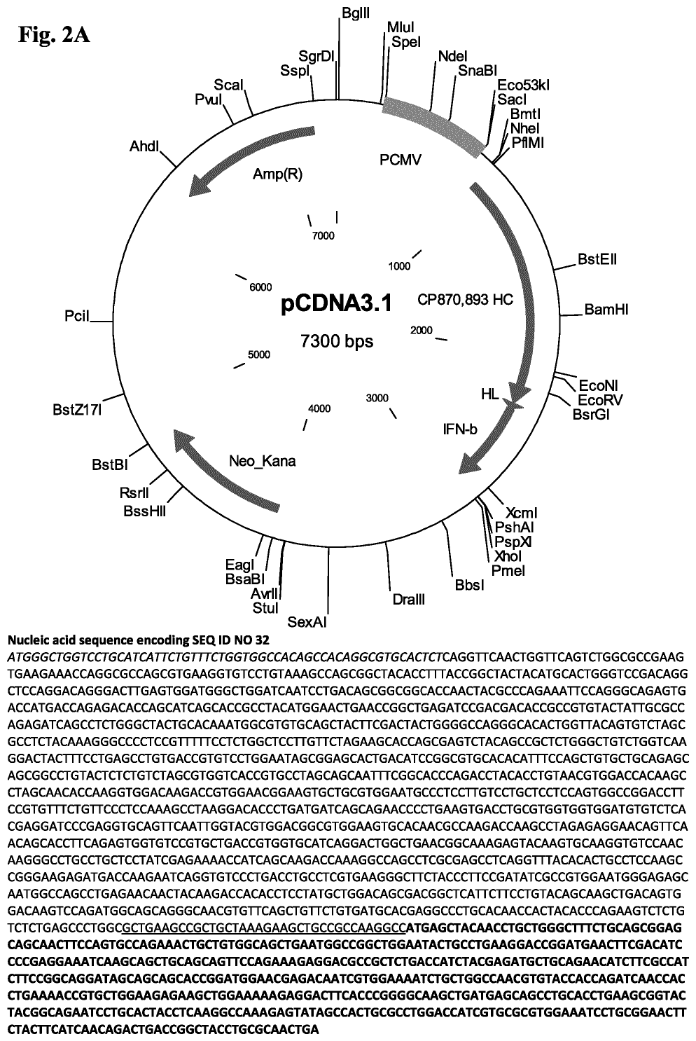

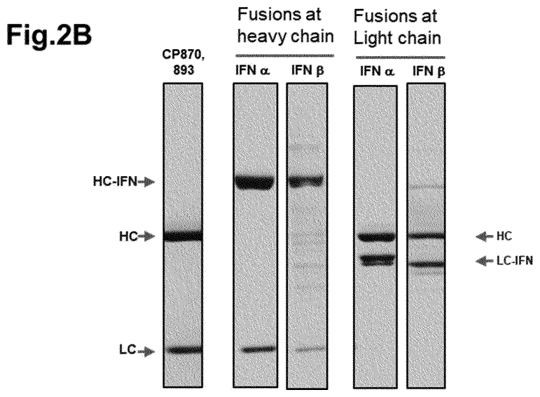

The sequence combinations of exemplary IFAs, designed with CP870,893 agonistic anti-CD40 antibody as backbone antibody, with the location of IFNs and the nature of the linkers are listed in Table 7 and Table 9. IFN was fused via a linker at the N- or the C-terminal part of the Light Chain (LC) or the Heavy Chain (HC), as indicated in Table 7. Nucleic acids encoding the HC, the LC or the fusions were synthesized with optimized mammalian expression codons and cloned into a eukaryotic expression vector such as pcDNA3.1 (Invitrogen). FIG. 2A depicts an exemplary map of a pcDNA3.1 plasmid encoding Seq ID NO 32 under the control of the pCMV promoter.

1.b—IFA Production

The Freestyle 293-F cells (Invitrogen) were transiently cotransfected with plasmids encoding both HC and LC at a HC/LC ratio of 4/6. Six days after transfection, the supernatant was collected, centrifuged and filtered through 0.22 μm filters. Purification process was performed in two purification steps, on AktaExpress chromatography system (GE Healthcare) using Protein A MabSelect Sure 5 mL 1.6/2.5 cm column (GE Healthcare) at a Flow rate of 5 mL/min. Sample binding was done in D-PBS1× pH 7.5 buffer, and elution with Glycine/HCl 0.1 M pH 3.0 buffer. Elution peak was stored in a loop then injected on HiTrap desalting 26/10 column (GE Healthcare) with a flow rate of 10 mL/min in D-PBS1×pH 7.5 buffer. Elution peak was collected on a 96-well microplate (2 mL fractions). Pool was performed according to the UV peak profile. After filtration on 0.22 μm filters (Sartorius MiniSart), quality control was performed including Bacterial Endotoxins using Endosafe® nexgen-PTS™ (Charles River), size exclusion Chromatography: using SEC 200 Increase 10/300 column (GE Healthcare) to determine purity and oligomers and SDS-PAGE under reducing and non-reducing conditions on NuPAGE gel System (Invitrogen) in MES SDS running buffer. The production yield is indicated in Table 9. For some IFAs, the production yield was very low. In that case, the agonistic CD40 activity and the IFN activity were assessed directly using the supernatant containing IFAs without any further purification.

Reduced SDS-PAGE analysis of purified IFAs indicated the presence of two major bands corresponding to the HC and the LC. When the IFN (whatever the IFN family member) was fused to the HC, a shift of its molecular weight was observed and the same phenomenon was observed for the LCs fused with any IFN (FIG. 2B).

I.c—IFA Characterization on Reporter Cells

HEK-Blue™ CD40L cells (InvivoGen Cat. #: hkb-cd40) or HEK-Blue™ IFN-α/β cells (InvivoGen, Cat. #: hkb-ifnαβ), were used to monitor, respectively, the activation of the NFκB pathway by CD40 agonists or of the IFN pathway induced by type I-IFN.

HEK-Blue™ CD40L cells were generated by stable transfection of HEK293 cells with the human CD40 gene and a NFκB-inducible Secreted Embryonic Alkaline Phosphatase (SEAP) construct (Invivogen) to measure the bioactivity of CD40 agonists. Stimulation of CD40 leads to NFκB induction and then production of SEAP, which is detected in the supernatant using QUANTI-Blue™ (Invivogen, Cat. #rep-qbs2).

HEK-Blue™ IFN-cells are designed to monitor the activation of the JAK/STAT/ISGF3 pathways induced by type I-IFNs. Activation of this pathway induces the production and release of SEAP. Levels of SEAP are readily assessable in the supernatant using QUANTI-Blue™

HEK-Blue™ IFN-α/β are used to monitor the activity of human IFNα or IFNβ.

Cells were seeded in 96-well plates (50,000 cells per well) and stimulated with the indicated concentration for each IFA or controls and incubated at 37° C. for 24 h. Supernatants were then collected and levels of SEAP were quantified after incubation of the supernatant for about 30 min with QuantiBlue™ and Optical Density (O.D.) assessment at 620 nm on an Ensight plate reader or PheraStar (Lab Biotech).

HEK-Blue™ Dual IFN-γ cells (InvivoGen, Cat. #: hkb-ifng) or HEK-Blue™ IFN-λ (InvivoGen, Cat. #: hkb-ifnl) may be used to respectively monitor the activity of type II- and type III-IFNs. HEK-Blue™ IFN-λ cells are designed to monitor the activity of IFN). HEK-Blue™ Dual IFN-γ cells allow the detection of bioactive human IFNγ.

I.d—Functional Activities of IFNα/β-Based IFAs on Reporter Cells

FIG. 3 shows examples of dose responses of IFAs, where IFNβ or a mutated version thereof as specified in Tables 7 was fused to the HC as indicated in Table 7, on HEK-Blue™ CD40L (FIGS. 3A-3B) and HEK-Blue™ IFN-α/β cells (FIGS. 3C-3D). Agonistic anti-CD40 activities of IFAs are summarized in Table 9 and examples are shown in FIG. 3A and FIG. 3B. Results indicate that all tested IFAs are functional to activate both the CD40 pathway and the IFN-α/β pathway in a dose dependent manner. For fusions to the C-terminus of the HC or LC, the EC₅₀ values for agonistic CD40 are ranging from 11.1 ng/mL to 192 ng/mL (Table 9). The mean EC₅₀ value for the parental antibody is 48 ng/mL and 57 ng/mL in the experiment shown in FIG. 3. IFAs with the IFN fused to the N-terminus of the HC or the LC were also able to activate the CD40 pathway, but the precise EC₅₀ values could not be determined for these IFAs since the activity was directly determined from the supernatant and not using purified proteins (FIG. 3B).

The IFN activity of various IFAs is summarized in Table 9 and examples are shown in FIGS. 3C to 3D. For fusions of IFNβ or mutated IFNβ (as specified in Table 7) to the C-terminus of the HC or LC, the IFN activity is variable depending on the linker sequence with EC₅₀ values ranging from 0.14 ng/mL to 4.5 ng/mL (FIG. 3C and Table 9). FIG. 3D shows that IFAs with IFNβ fused to the N-terminal part exhibit high IFN activity. The parental antibody used as negative control did not show any activity, whereas recombinant IFNβ did show a strong dose-dependent response. Altogether, these results demonstrate that fusion of IFNβ or a mutated version thereof as specified in Table 7 to an antibody, regardless the location, maintain both biological functions, although with differences in terms of potencies.

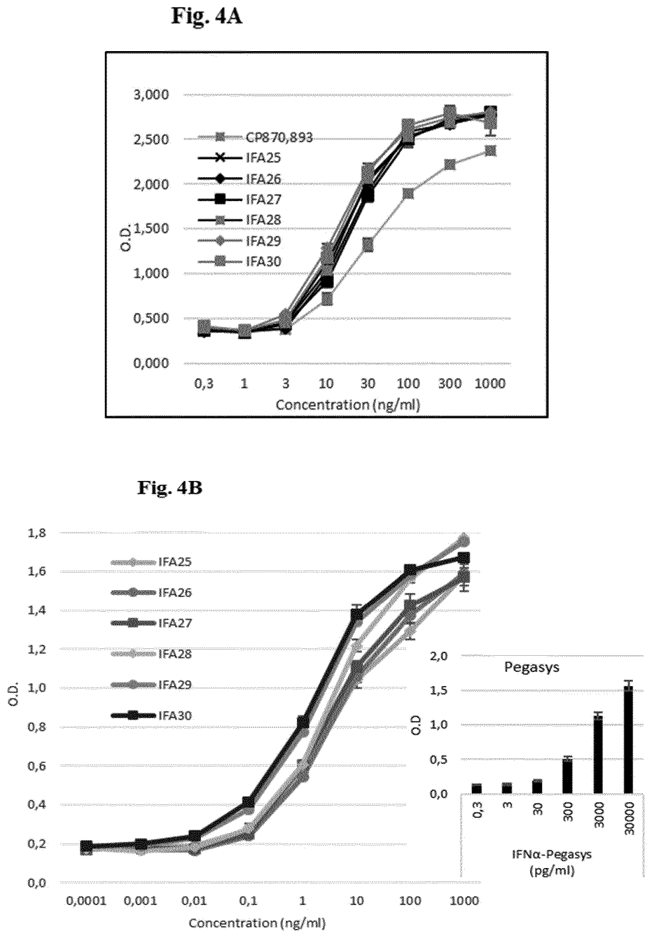

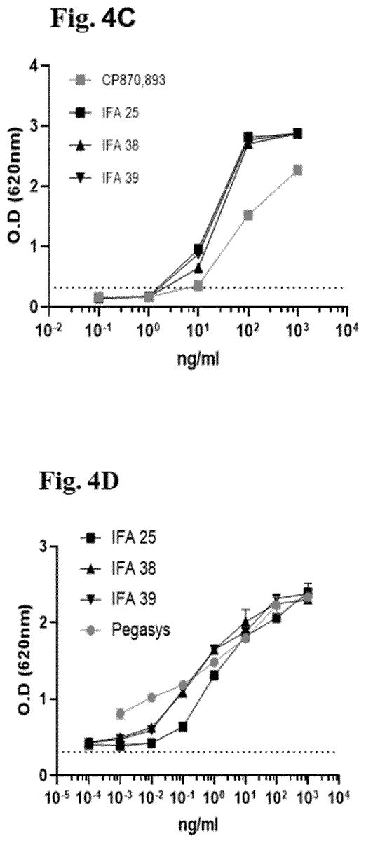

FIG. 4 shows examples of dose responses of IFAs, where IFNα was fused to the HC or the LC as indicated in Table 7, on HEK-Blue™ CD40L (FIG. 4A and FIG. 4 C) and HEK-Blue™ IFN-α/p cells (FIG. 4B and FIG. 4D). Results indicate that all tested IFAs are functional to activate both the CD40 pathway and the IFNα/0 pathway in a dose-dependent manner. Surprisingly, for all the IFNα-based IFAs, the potency on CD40 pathway was reproducibly higher than that of the parental antibody. The EC₅₀ values for IFNα-based IFAs ranged from 11.1 ng/mL to 22.7 ng/mL and the EC₅₀ for CP870,893 ranged from 30 ng/mL to 80 ng/mL (mean EC₅₀ value: 48 ng/mL).

The IFN activity of IFAs is variable depending on the linker sequence with EC₅₀ values ranging from 1.6 ng/mL to 5.1 ng/mL. In the same assay, PEGylated IFNα2a (Pegasys®) was also active in a dose-dependent manner with an EC₅₀ value of around 1 ng/mL.

I.e—Generation and Characterization of IFAs without the Fc Region

Suitable constructs according to the invention can also be interferon-associated antigen binding proteins without an Fc region. A construct encoding the heavy chain of the fab fragment of CP870,893 fused to a TEV-His tag was designed (SEQ ID NO 50) and cloned into the expression plasmid pcDNA3.1. This construct is cotransfected in HEK cells as described earlier, with LCs fused via different linkers to different IFNs such as SEQ ID NO 28, SEQ ID NO 29, SEQ ID NO 34, SEQ ID NO 35, SEQ ID NO 36, SEQ ID NO 37, SEQ ID NO 41, SEQ ID NO 42, or SEQ ID NO 43. Proteins and/or supernatants are evaluated in reporter cells. It will be understood by one of skill in the art that constructs for use in therapy will no longer contain the TEV-His tag. These constructs are likewise embodiments of the invention. Interferon-associated antigen binding proteins without the Fc part will be active against Coronavirus infection. Two IFAs were then produced and their functional characterization is described in Example V: IFA50: (SEQ ID NO 41)+(SEQ ID NO 50) and IFA51: (SEQ ID NO 42)+(SEQ ID NO 50).

### Example II

### Effect of IFAs on SARS-CoV-2 Infected Cells

II.a—Cell Culture

Vero E6 cells, which were extracted from an African green monkey kidney-derived cell line CCL81, were obtained from ElabScience (cat #EP-CL-0491). The adherent cell line was maintained in Dulbecco's Modified Eagle Medium (DMEM; Gibco cat #21885-025, Carlsbad, CA, USA) supplemented with 10% fetal bovine serum (FBS; Gibco, Carlsbad, CA, USA). Cells were cultured in a humidified atmosphere with 5% CO₂ at 37° C.

II.b—Virus Isolates

The following SARS-CoV-2 strains used in the experiments were obtained from BEI Resources:
Isolate USA-WA1/2020 (NR-52281)
Isolate Germany/BavPat1/2020 (NR-52370)
Isolate hCoV-19/South Africa/KRISP-EC-K005325/2020 (NR-54009)
Isolate USA/CA_CDC_5574/2020 (NR-54011)
Isolate hCoV-19/England/204820464/2020 (NR-54000)
Isolate hCoV-19/South Africa/KRISP-EC-K005321/2020 (NR-54008)
Isolate hCoV-19/Japan/TY7-503/2021 (NR-57982)
Isolate hCoV-19/USA/PHC658/2021 (NR-55611)
Isolate hCoV-19/USA/MD-HP20874/2021 (NR-56461)
   II.c—Sequences of Control IFAs

TABLE C
Sequences of control interferon-associated antigen binding protein based
on the isotypic control Evi5. Italic sequences correspond to signal
peptides. Bold non-italic sequences correspond to linkers.

| Name/ | |
|---|---|
| SEQ ID Number | Sequence |
| eVi-5 D1.3- | MGWSCIILFLVATATGVHS |
| h1v2_hIgG2 | EVKLQESGPGLVAPSQSLSITCTVSGFSLTGYGVNWVRQPP |
| dK_HC | GKGLEWLGMIWGDGNTDYNSALKSRLSISKDNSKSQVFLK |
| (SEQ ID NO 89) | MNSLHTDDTARYYCARERDYRLDYWGQGTLVTVSSASTK |
| | GPSVFPLAPCSRSTSESTAALGCLVKDYFPEPVTVSWNSGA |
| | LTSGVHTFPAVLQSSGLYSLSSVVTVPSSNFGTQTYTCNVD |
| | HKPSNTKVDKTVERKCCVECPPCPAPPVAGPSVFLFPPKPK |
| | DTLMISRTPEVTCVVVDVSHEDPEVQFNWYVDGVEVHNA |
| | KTKPREEQFNSTFRVVSVLTVVHQDWLNGKEYKCKVSNK |
| | GLPAPIEKTISKTKGQPREPQVYTLPPSREEMTKNQVSLTCL |
| | VKGFYPSDIAVEWESNGQPENNYKTTPPMLDSDGSFFLYSK |
| | LTVDKSRWQQGNVFSCSVMHEALHNHYTQKSLSLSPG |
| evi-5 D1.3-hk-LC | MGWSCIILFLVATATGVHS |
| (SEQ ID NO 90) | IELTQSPASLSASVGETVTITCRASGNIHNYLAWYQQKQGK |
| | SPQLLVYYTTTLADGVPSRFSGSGSGTQYSLKINSLQPEDFG |
| | SYYCQHFWSTPRTFGGGTKLELKRTVAAPSVFIFPPSDEQL |
| | KSGTASVVCLLNNFYPREAKVQWKVDNALQSGNSQESVT |
| | EQDSKDSTYSLSSTLTLSKADYEKHKVYACEVTHQGLSSPV |
| | TKSFNRGEC |
| eVi-5 D1.3- | MGWSCIILFLVATATGVHS |
| h1v2_hIgG2 | EVKLQESGPGLVAPSQSLSITCTVSGFSLTGYGVNWVRQPP |
| dK_HC--(G4S)2-- | GKGLEWLGMIWGDGNTDYNSALKSRLSISKDNSKSQVFLK |
| IFNα2A | MNSLHTDDTARYYCARERDYRLDYWGQGTLVTVSSASTK |
| (SEQ ID NO 91) | GPSVFPLAPCSRSTSESTAALGCLVKDYFPEPVTVSWNSGA |
| | LTSGVHTFPAVLQSSGLYSLSSVVTVPSSNFGTQTYTCNVD |
| | HKPSNTKVDKTVERKCCVECPPCPAPPVAGPSVFLFPPKPK |
| | DTLMISRTPEVTCVVVDVSHEDPEVQFNWYVDGVEVHNA |
| | KTKPREEQFNSTFRVVSVLTVVHQDWLNGKEYKCKVSNK |
| | GLPAPIEKTISKTKGQPREPQVYTLPPSREEMTKNQVSLTCL |
| | VKGFYPSDIAVEWESNGQPENNYKTTPPMLDSDGSFFLYSK |
| | LTVDKSRWQQGNVFSCSVMHEALHNHYTQKSLSLSPGGG |
| | GGSGGGGSCDLPQTHSLGSRRTLMLLAQMRKISLFSCLKD |
| | RHDFGFPQEEFGNQFQKAETIPVLHEMIQQIFNLFSTKDSSA |
| | AWDETLLDKFYTELYQQLNDLEACVIQGVGVTETPLMKED |
| | SILAVRKYFQRITLYLKEKKYSPCAWEVVRAEIMRSFSLST |
| | NLQESLRSKE |
| eVi-5 D1.3- | MGWSCIILFLVATATGVHS |
| h1v2_hIgG2 | EVKLQESGPGLVAPSQSLSITCTVSGFSLTGYGVNWVRQPP |
| dK_HC--(G4S)4-- | GKGLEWLGMIWGDGNTDYNSALKSRLSISKDNSKSQVFLK |
| IFNα2A | MNSLHTDDTARYYCARERDYRLDYWGQGTLVTVSSASTK |
| (SEQ ID NO 92) | GPSVFPLAPCSRSTSESTAALGCLVKDYFPEPVTVSWNSGA |
| | LTSGVHTFPAVLQSSGLYSLSSVVTVPSSNFGTQTYTCNVD |
| | HKPSNTKVDKTVERKCCVECPPCPAPPVAGPSVFLFPPKPK |
| | DTLMISRTPEVTCVVVDVSHEDPEVQFNWYVDGVEVHNA |
| | KTKPREEQFNSTFRVVSVLTVVHQDWLNGKEYKCKVSNK |
| | GLPAPIEKTISKTKGQPREPQVYTLPPSREEMTKNQVSLTCL |
| | VKGFYPSDIAVEWESNGQPENNYKTTPPMLDSDGSFFLYSK |
| | LTVDKSRWQQGNVFSCSVMHEALHNHYTQKSLSLSPGGG |
| | GGSGGGGSGGGGSGGGGSCDLPQTHSLGSRRTLMLLAQ |
| | MRKISLFSCLKDRHDFGFPQEEFGNQFQKAETIPVLHEMIQ |
| | QIFNLFSTKDSSAAWDETLLDKFYTELYQQLNDLEACVIQG |
| | VGVTETPLMKEDSILAVRKYFQRITLYLKEKKYSPCAWEV |
| | VRAEIMRSFSLSTNLQESLRSKE |

TABLE D
Polypeptide combinations found in control interferon-fused
antigen binding proteins based on the isotypic control
Evi5 (=(SEQ ID NO 89) + (SEQ ID NO 90); sequence
combination comprised twice). Each sequence combination
as indicated is comprised twice in the respective IFA.

| Interferon -fused Antibody (IFA) | Sequence combination |
|---|---|
| IFA201 | (seq ID NO 90) + (seq ID NO 92) |
| IFA202 | (seq ID NO 90) + (seq ID NO 91) |

II.d—Cytometry Analysis: CD40 Staining

Vero E6 cells (1.10⁶ cells/well) were resuspended in PEB buffer in the presence of FcR blocking antibodies (Miltenyi 130-059-901) for 15 mn at 4° C. After centrifugation at 1500 rpm for 2 mn at 4° C., cells were resuspended in 100 μl of buffer alone or in the presence of isotypic antibody (Miltenyi 130-113-438) or Anti-CD40-APCVio770 antibody (Miltenyi, 130-123-395) and incubated for 30 mn at 4° C. Then cells were centrifuged, washed, fixed for 10 mn at 4° C. with 4% PFA. After centrifugation and washing, wells were resuspended in PEB buffer and analyzed on MACSQuant16 cytometer.

II.e—Cell Viability Assay CellTiter-Glo

The CellTiter-Glo luminescent cell viability assay (CTG) was performed on each sample according to the manufacturer's instructions (Promega, catalog number: G7570). Briefly, cells were plated in triplicate in 96-well plates and incubated at 37° C., 5% CO₂. Twenty four hours post-seeding, cells were infected at a multiplicity of infection (MOI) of 0.05 (except for Gamma and Omicron variants, at MOI 0.5 and 0.1, respectively) for 1 h, washed once with PBS and then treated with Remdesivir (Sigma-Aldrich, St. Louis, MO, USA; Wang (2020) Cell Res. 30: 2-71) or with test items at the indicated concentrations. For the pre-treatment procedure (FIGS. 5F and 5G, FIG. 5F.bis and FIG. 5G.bis), the cells were treated with Remdesivir or with test items at the indicated concentrations for 1 h at 37° C., washed once with PBS and infected at a MOI of 0.05 for 72 h at 37° C., 5% CO₂. For the neutralization assay, the virus was mixed at a MOI of 0.05 with S309 (Pinto (2020) Nature July; 583(7815):290-295) at the indicated concentrations for 1 h at 37° C. to allow the antibody to bind to the Spike protein of the virus. The cells were then infected with the mixture virus/antibody and incubated for 72 h at 37° C., 5% CO₂. For all the procedures described above, at 72 h post-infection, the cells were washed once with PBS and incubated for 10 min with CellTiter-Glo reagent, and luminescence was measured using a 96-well plate reader (GloMax-96 microplate luminometer; Promega) in a microplate reader (Perkin Elmer Ensight) with an integration time of 0.1 s per well. Background luminescence was measured in medium without cells and subtracted from experimental values.

II.f—SARS-CoV-2 Infection Model Validation (Utilizing CTG Assay)

To validate the infection model, two experiments were carried out using neutralizing antibody and a polymerase inhibitor, Remdesivir. For the neutralization assay, the neutralizing antibody S309 and the virus (isolate USA-WA1/2020) were preincubated for 1 h before cell infection and cell viability was assessed 3 days after infection. FIG. 5A shows that S309 potently neutralized SARS-CoV-2 with an EC₅₀ of 858 ng/mL.

The effect of Remdesivir was also assessed by treating the cells with an increasing dose range of test product, one hour post infection for duration of 72 h. Results indicate that Remdesivir is functional to inhibit viral replication and virus-induced cytopathic effect in a dose-response manner with an EC₅₀ of 1.341 μM (FIG. 5B).

### II.g.1—Effect of IFA 27 on SARS-CoV-2 Infection (Utilizing CTG Assay)

We showed that Vero E6 cells express CD40 on their cell surface (FIG. 5C). Thus the effect of IFA27 on SARS-CoV-2 infection was assessed in comparison to Remdesivir in 2 experimental designs. Cells were treated with dose range of IFA27 or Remdesivir 1 h after infection with SARS-CoV-2 (FIGS. 5D and 5E). Cell viability was assessed 3 days later using the CTG assay. Results indicate that IFA27 potently rescued Vero E6 from SARS-CoV-2-induced cytopathic effect in a dose-dependent manner with an EC₅₀ of 8.10⁻⁵ μM (FIG. 5D). In the same experiment, the EC₅₀ for Remdesivir was >1 μM (FIG. 5E). In the second experimental setting, cells were preincubated for 1 h with IFA27 or with Remdesivir, washed with PBS and infected at a MOI of 0.05 for 72 h at 37° C. Cell viability was assessed by CTG approach. Results indicate that the short-term treatment with IFA27 (FIG. 5F) but not with Remdesivir (FIG. 5G) was able to rescue Vero E6 cells from cell death induced by SARS-CoV-2. Despite the short treatment duration, the EC₅₀ was 3.45·10⁻⁴ μM (FIG. 5F).

### II.g.2.—Effect of IFA 25 on SARS-CoV-2 Infection (Utilizing CTG Assay)

The effect of IFA25 on SARS-CoV-2 infection was assessed in comparison to Remdesivir in 2 experimental designs. Cells were treated with dose range of IFA25 or Remdesivir 1 h after infection with SARS-CoV-2 (FIGS. 5D.bis and 5E.bis). Cell viability was assessed 3 days later using the CTG assay. Results indicate that IFA25 potently rescued Vero E6 from SARS-CoV-2-induced cytopathic effect in a dose-dependent manner with an EC₅₀ of 2.231×10⁻⁵ μM (FIG. 5D.bis). In the same experiment, the EC₅₀ for Remdesivir was 1.756 μM (FIG. 5E.bis). In the second experimental setting, cells were pre-incubated for 1 h with IFA25 or with Remdesivir, washed with PBS and infected at a MOI of 0.05 for 72 h at 37° C. Cell viability was assessed by CTG approach. Results indicate that the short-term treatment with IFA25 (FIG. 5F.bis) but not with Remdesivir (FIG. 5G.bis) was able to rescue Vero E6 cells from cell death induced by SARS-CoV-2. Despite the short treatment duration, the IFA25 EC₅₀ was 4.813×10⁻⁵ M. Such an antiviral effect of IFA25 when applied either before or after the infection step demonstrates that it can protect both the infected cells and the surrounding uninfected cells present at the site of the infection in the clinical situation. Accordingly these results support the capability of the constructs of the invention not only to treat but also to prevent a Coronavirus infection. The protective effect on the surrounding uninfected cells furthermore makes the treatment particularly effective.

II.h—Effect of IFA25, IFA27 on SARS-CoV-2 Infection in Comparison to Control IFA, CP870, 893 and Pegasys (Utilizing CTG Assay)

We then evaluated the effect of IFA27 in comparison to the parental antibody (CP870,893) or to a control IFA (IFA201) with IFNA2A fused to a control antibody (without the CD40 agonistic activity) through the same (G4S)4 linker. Cells were treated 1 hour after infection and cell viability assessed 3 days later by CTG. Results indicate that CP870, 893 by itself did not have any effect on SARS-CoV-2-induced cell death. The control IFA demonstrated a dose effect activity due to the presence of IFNA2A but IFA27 was more than 100 times more potent than the control IFA on rescuing Vero E6 from SARS-CoV-2-induced cell death (FIG. 5H).

IFA25 exhibited very similar activity in comparison to IFA27 with a high potency compared to its control IFA (IFA202) or Pegasys® (FIG. 5I).

We then evaluated the effect of IFA25 in comparison to the parental antibody (CP870,893) or to a control IFA (IFA202) with IFNA2A fused to a control antibody (EVI5, without the CD40 agonistic activity) through the same (G4S)2 linker. Cells were treated 1 hour after infection and cell viability assessed 3 days later by CTG. Results indicate that CP870,893 by itself did not have a potent effect on protecting SARS-CoV-2-induced cell death. The control IFA202 demonstrated a dose effect activity due to the presence of IFNA2A but IFA25 was more potent than the control IFA on rescuing Vero E6 cells from SARS-CoV-2-induced cell death. Interestingly, IFA25, alone or in combination with the isotype EVI5 (to have the same antibody load as in the CP870,893 and IFA202 combination), was also more potent compared to CP870,893 and IFA202 combination thus demonstrating the benefit of having both CD40 and IFN functions within the same molecule (FIG. 5I.bis).

II.i—Effect of IFA25 on SARS-CoV-2 Isolate (Utilizing CTG Assay)

Vero E6 cells were infected with different isolates and treated with IFA25 for 3 days. Results indicate that, regardless the isolate, IFA25 is highly potent on preventing cells from dying upon SARS-CoV-2 infection, with EC₅₀ around 10⁻⁶ μM, demonstrating its potential broad use on any SARS-CoV-2 variant or even on any Coronavirus (FIGS. 5J-50).

In FIGS. 5P to 5R, additional results on further isolates indicate that, also with these isolates, IFA25 is highly potent on preventing cells from dying upon SARS-CoV-2 infection, with EC50 around 10⁻⁵ μM.

In all these experiments (FIGS. 5J-5R), the same protocol was applied and Remdesivir (RDV), a direct-acting antiviral used for the treatment of the COVID19, was included as a comparator. To compare the IFA25 and Remdesivir efficacies, the Log 10 absolute EC50 (LogAbsoluteEC50) were estimated and are reported in table 14. In all tested conditions, the potency of IFA25 to protect VeroE6 cell from death was superior to that of RDV. Moreover, IFA25 was found to be significantly different from RDV (Table 14), with a p value at p<0.00 1, using a paired t-Test on LogAbsoluteEC50 for treatments comparison with a pairing on SARS-CoV-2 lineages. This demonstrates IFA25 potential broad use on any SARS-CoV-2 variants or even on any Coronavirus.

II.j—Effect of IFA126 on SARS-CoV-2 Isolate (Utilizing CTG Assay)

The effect of IFA126 on SARS-CoV-2 infection was assessed in dose-range, in post-treatment design experiments, against 3 major variants: isolate USA-WA1/2020 (MOI 0.05), isolate hCoV-19/USA/PHC658/2021 (Delta variant, MOI 0.05) and isolate hCoV-19/USA/MD-HP20874/2021 (Omicron variant, MOI 0.1) (FIGS. 5S to 5U). Cell viability was assessed 3 days later using the CTG assay.

IFA126 potently rescued Vero E6 cells from SARS-CoV-2-induced cytopathic effect in a dose-dependent manner with an EC₅₀ of 9.5×10⁻⁶ μM against USA-WA1/2020 isolate (FIG. 5S), an EC₅₀ of 1.445×10⁻⁵ μM against Delta variant (FIG. 5T) and an EC₅₀ of 2.72×10⁻⁵ μM against Omicron variant (FIG. 5U).

II.k—Real-Time Cell Analysis System (RTCA xCELLigence; Nat Med. 2020 September; 26(9):1422-1427)

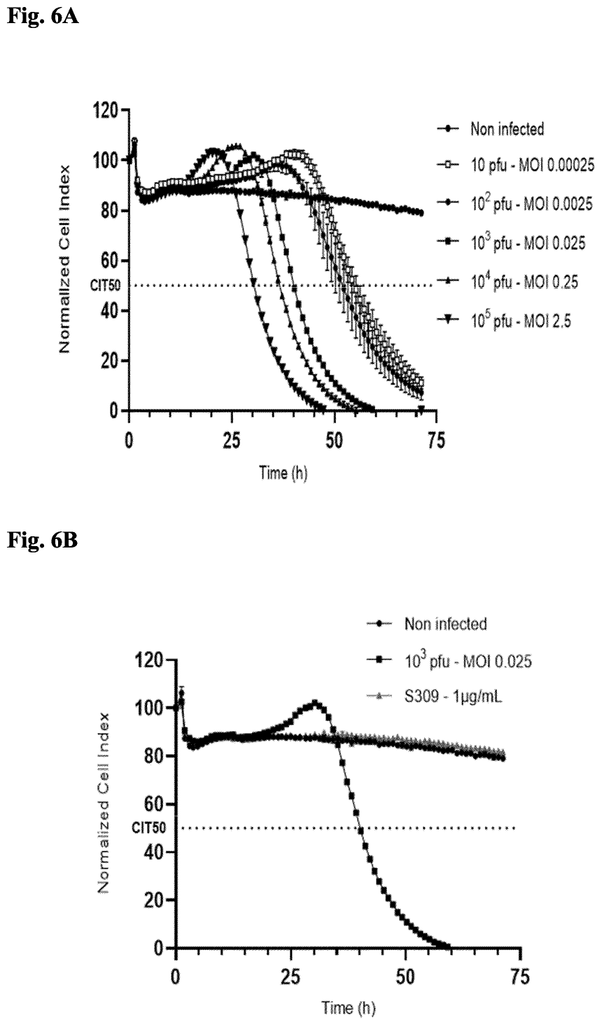

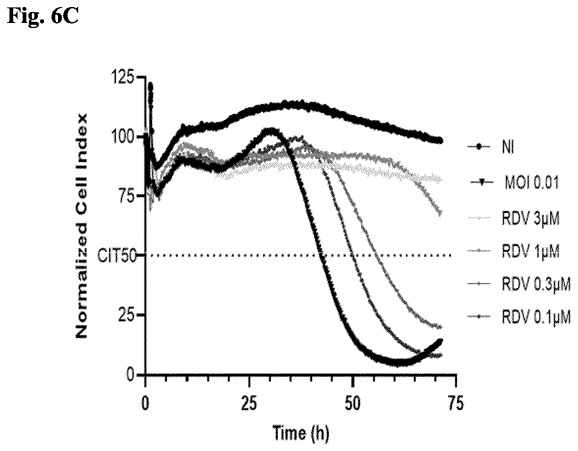

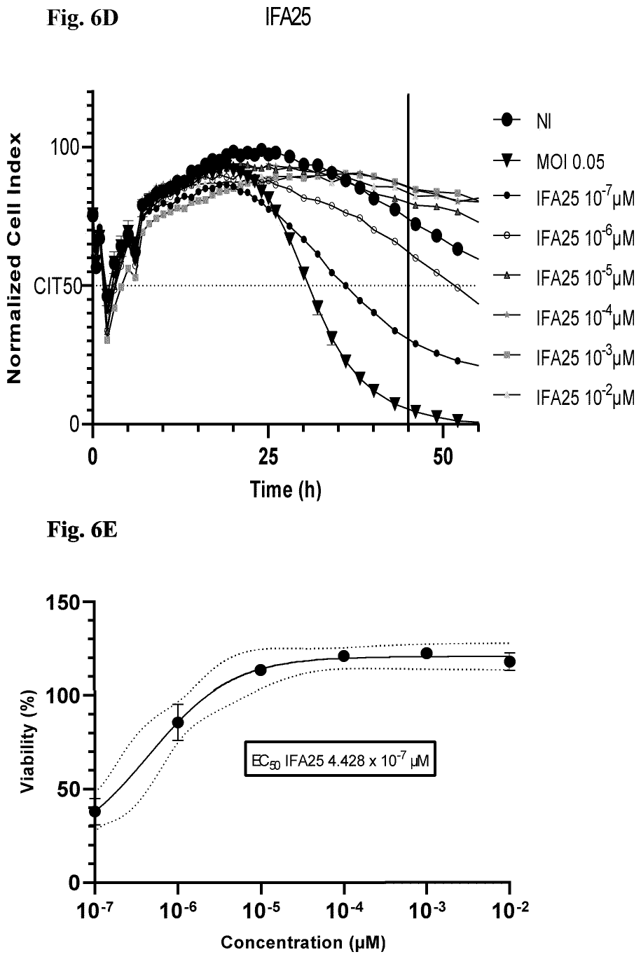

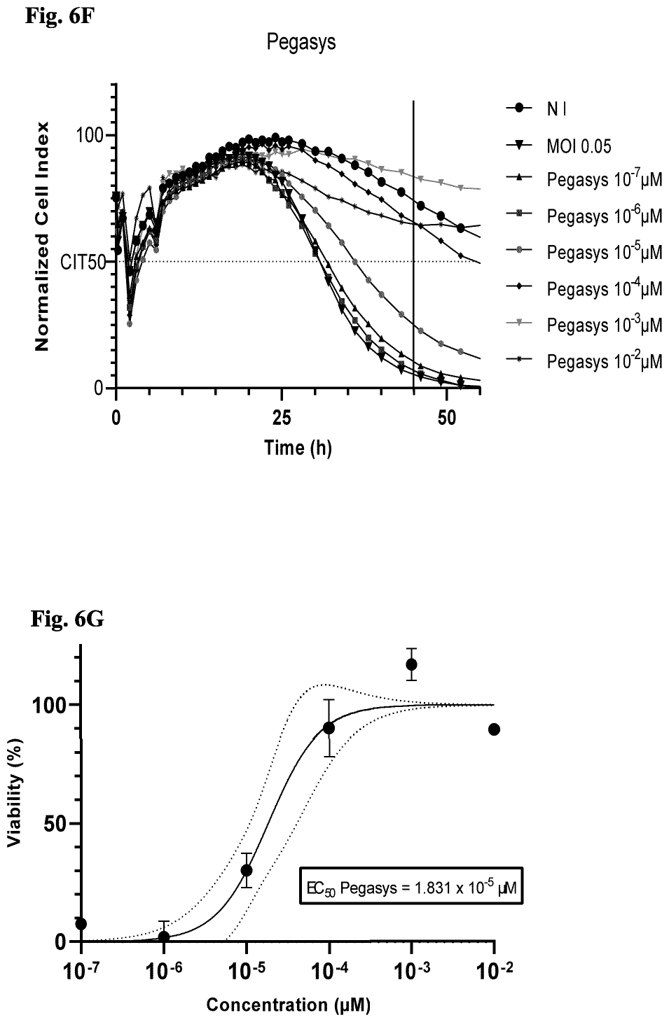

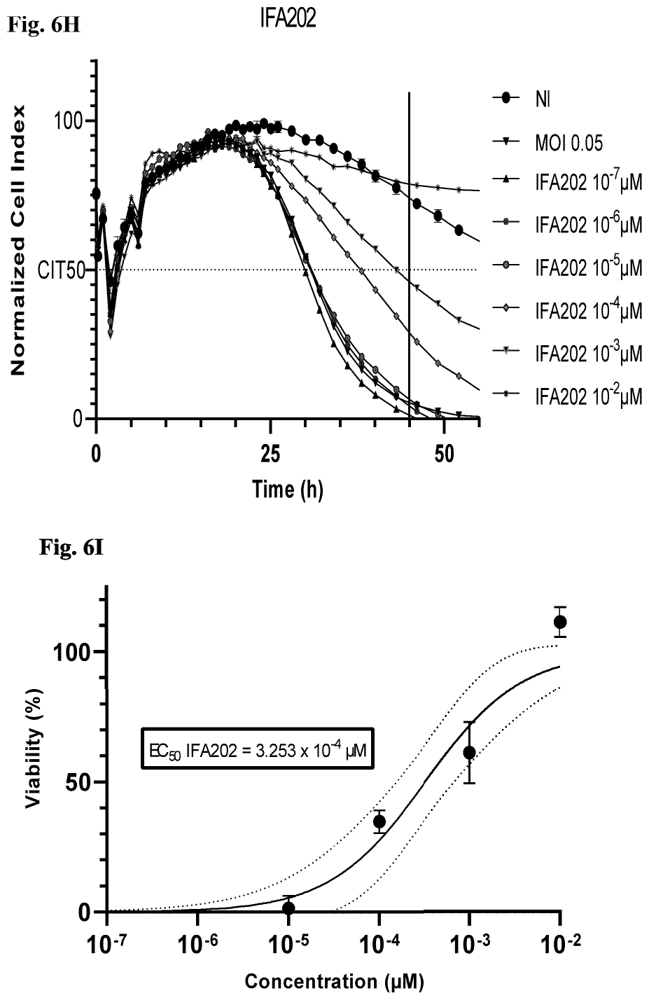

The RTCA instruments use microplates that contain gold biosensors integrated into the bottom of each well. The presence of adherent cells on the gold biosensors impedes current flow and the magnitude of the impedance depends on the number of cells, the size of the cells, the cell-substrate attachment quality, and cell-cell adhesion (barrier function). When infected with a virus, host cells often display microscopically visible changes that are collectively referred to as a cytopathic effect (CPE). CPE can include cell shrinkage or enlargement, deterioration/lysis, cell fusion, and/or the formation of inclusion bodies. Infected cells that progress through the continuum of the cytopathic effect (CPE) become increasingly less effective at impeding the flow of electric current. By measuring these changes continuously, RTCA tracks viral CPE in detail and yields quantitative kinetics. The device and the software used in the study were the xCELLigence RTCA SP (Agilent) and the RTCA Software 2.0 (Agilent) respectively, according to the manufacturer's instructions. The RTCA was performed on each sample as described below. Briefly, cells were plated in triplicate in 96-well E-plates (Agilent, cat #5232368001) and incubated at 37° C., 5% CO₂. Twenty four hours post-seeding, the plate was placed into the xCELLigence station located in an incubator at 37° C., 5% CO₂, and the recording of the electric current in each well was launched as follows: 61 consecutive reads at 1-minute interval, 100 consecutive reads at 15-minute interval, and 200 consecutive reads at 1-hour interval. The electric impedance (referred to as Cell Index (CI), FIG. 6) was monitored for 1 h to evaluate the background of the cell monolayer. The cells were then infected at a MOI of 0.05 for 1 h, washed once with PBS and then treated with Pegasys® or with test items at the indicated concentrations. The Cell Index was continuously monitored for 55 h. The data were analyzed using the software GraphPad Prism 8. For FIGS. 6D, 6F and 6H, the CI was normalized as follows: 100% corresponds to the highest CI recorded (at 25 h) for the non-infected condition (NI) and 0% corresponds to the CI obtained at 55 h when all cells have died. For the determination of the EC₅₀ in FIGS. 6E, 6G and 6I, the data for each concentration of the test items at the 45-hour time point were plotted using a 4PL non-linear regression analysis.

II.l—Validation of the Infection Model Using the Real-Time Cell Analysis System

The xCELLigence system was used to evaluate in real time the effect of IFAs on cell death upon infection of Vero E6 cells with SARS-CoV-2. We first appraised the effect of virus concentration on virus-induced cytopathic effect expressed here as cell index. Cells were kept non-infected or infected with different numbers of plaque forming units (PFU) of SARS-CoV-2 for 75 h. Results indicate a good correlation between cell index and the number of PFUs (FIG. 6A). We then confirmed that the neutralizing antibody S309 was able to prevent cell death induced by SARS-CoV-2 (FIG. 6B) and that Remdesivir was also able to prevent SARS-CoV-2-induced cytopathic effect in Vero E6 cells in a dose-dependent manner (FIG. 6C).

II.m—Effect of IFA25 on SARS-CoV-2 Infection Using the Real-Time Cell Analysis System

The real-time effect of IFA25 in comparison to Pegasys® and to the associated control IFA was monitored on Vero-E6 cells infected with SARS-CoV-2. Results indicate a potent effect of IFA25 in comparison to Pegasys® or to the control IFA (IFA202), thus confirming previous results using the CTG assay with an EC₅₀ 4.4·10⁻⁷ μM (FIG. 6D-E).

### Example III

### Cytokine Release

III.a—Cytokine Release Assessment (CRA) from Human Whole Blood Cells

Whole blood cells (WBC) ex vivo stimulation assay was used to investigate release of cytokines following IFA stimulation. WBC were collected from four healthy donors, diluted 1/3 in RPMI1640 (72400-021, Gibco) and distributed in sterile reaction tubes (300 μl). Cells were left unstimulated, stimulated with LPS (LipoPolySaccharide) K12 (tlrl-eklps, Invivogen) at 10 ng/mL as a positive control or with IFAs at 1 μg/mL and incubated for 24 h at 37 C. Supernatants were then collected and frozen at −20 C until the day of analysis.

Human pro-inflammatory cytokines were analyzed using multiplexing MSD assay (K15067L-4) which measures Tumor Necrosis Factor (TNF)-α, Interleukin (IL)-1β, IL-2, IL-6, IL-8, IL-10, IL-12/IL-23p40 and IFNγ. MSD plates were analyzed on the 1300 MESO QuickPlex SQ120 apparatus (MSD).

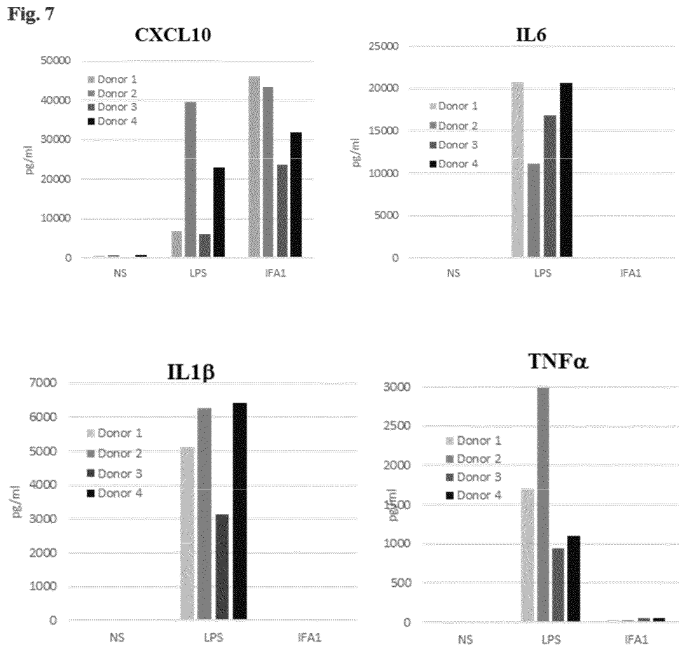

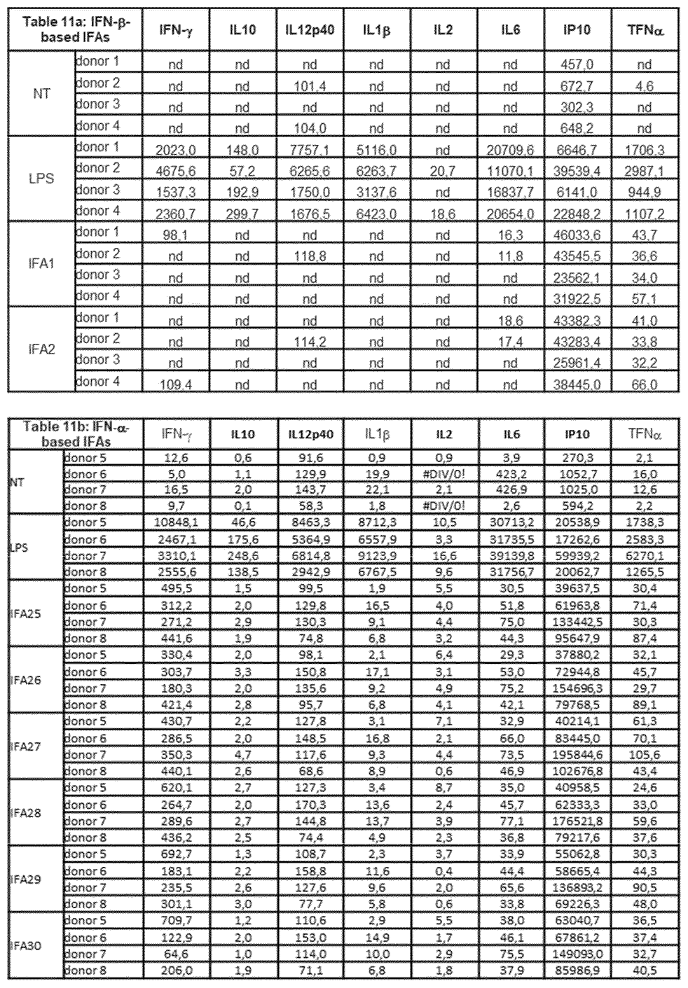

FIG. 7 depicts exemplary results from an in vitro Cytokine Release Assessment of Human WBC either non-stimulated, treated with LPS or with IFA1.

Further results from testing IFNβ-/mutated IFNβ- and IFNα-based IFAs are summarized in Tables 11a and 11b. Results show that for all donors, LPS induces very high level of the inflammatory cytokines (IL-1β, TNF-α, IL-6, IL-12p40 and IFNγ). It also induced IP10 (CXCL10) which is a biomarker of the IFN pathway and moderate level of IL-10. Two IFNβ- (Table 11a) and six IFNα- (Table 11b) based IFAs were tested. All of them induced the biomarker IP10. However, they did not induce IL-10, IL-1β and IL-2, and they induced only very low to moderate level of IFNγ, IL-6 and TNF-α, thus suggesting a favorable safety profile with regard to the induction of inflammatory cytokines.

### Example IV

### Pharmacokinetic Studies

IV.a—ELISA Assay Development for IFA Quantifications

For the ELISA quantification 96-wells plates (PLATES 96 wells Maxisorp, THERMO Scientifique; 442404) were coated overnight at 4 C with 100 μl of recombinant human CD40/TNFRSF5 Fc Chimera Protein, consisting of the extracellular domain of human CD40 fused to the Fc part of human IgG1 (CD40-Fc; R&D Systems; 1493-CDB-050) at 0.5 μg/mL in Sodium Carbonate (0.05 M, pH 9.6, C-3041, Sigma). After emptying by flipping, plates were then incubated for 1 hour at 37 C with PBS—0.05% Tween20—1% Milk (SIGMA; 70166-500g) followed by washing with PBS—0.05% Tween20. Samples and controls (100 μl of ½ serial dilutions) were then incubated for 90 minutes at 37 C followed by three washes (PBS—0.05% Tween20) and incubation with a secondary anti-IgG2-conjugate HRP (1/5000, ab99779, Abcam) antibody or anti-IFNα conjugate HRP (1/1000, eBIOSCIENCE/Invitrogen; BMS216MST) in PBS—0.05% Tween20—1% Milk. After three washes with PBS, 0.05% Tween2, TMB (Tetramethylbenzidin, Tebu Bio; TMBW-1000-01) was added and the plates incubated for 20 minutes in the dark. The reaction was stopped by adding 1M HCl. Plates were read at 450-650 nm with an Ensight plate reader (Perkin Elmer). Quantification of Pegasys was assessed using similar protocol steps but using human IFNα matched antibody pairs from eBioscience/Invitrogen. Capture was performed using 100 μL of human anti-IFNα antibody (eBioscience/Invitrogen; BMS216MST), at 1 g/mL in sodium carbonate (0.05 M, pH 9.6, C-3041, Sigma). For the detection, a secondary anti-IFNα conjugate HRP antibody (1/1000, Affymetrix eBioscience/BMS216MST; 15501707) in PBS—0.05% Tween20—1% Milk was applied.

IV.b—In Vivo Bioavailability in Mice

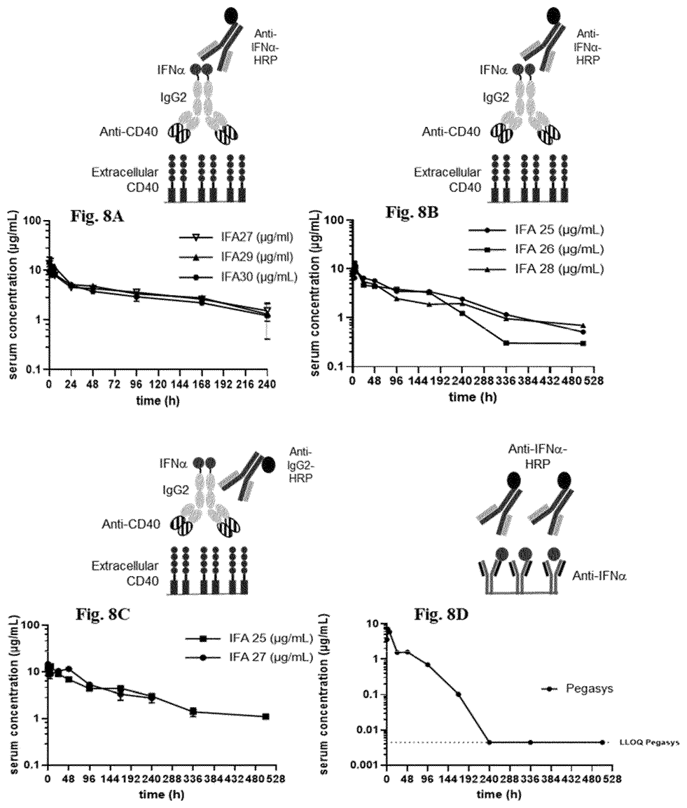

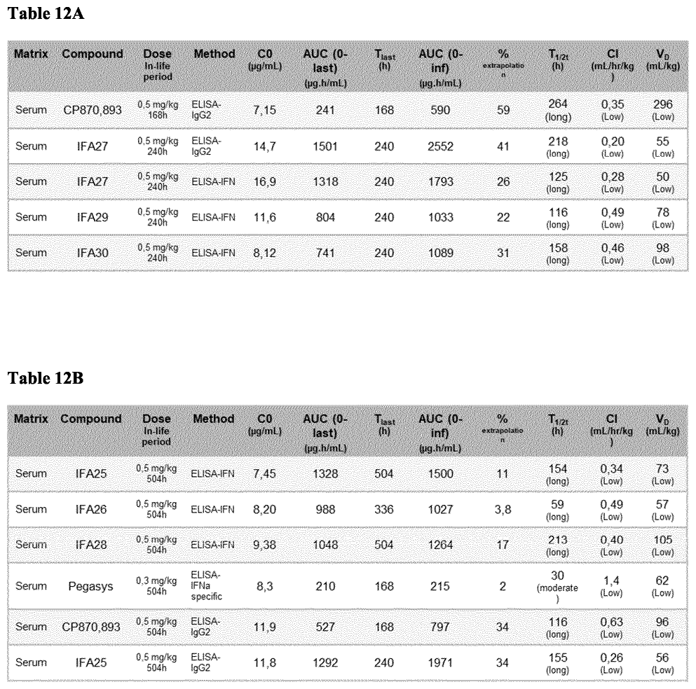

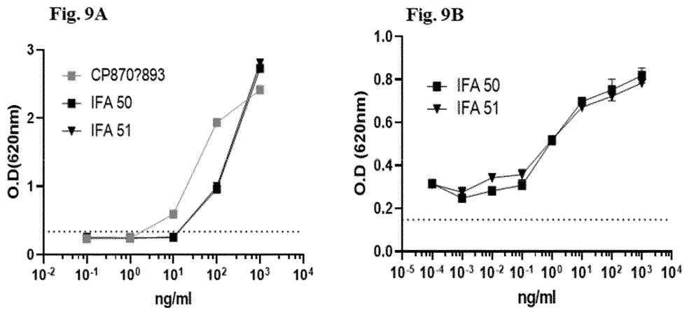

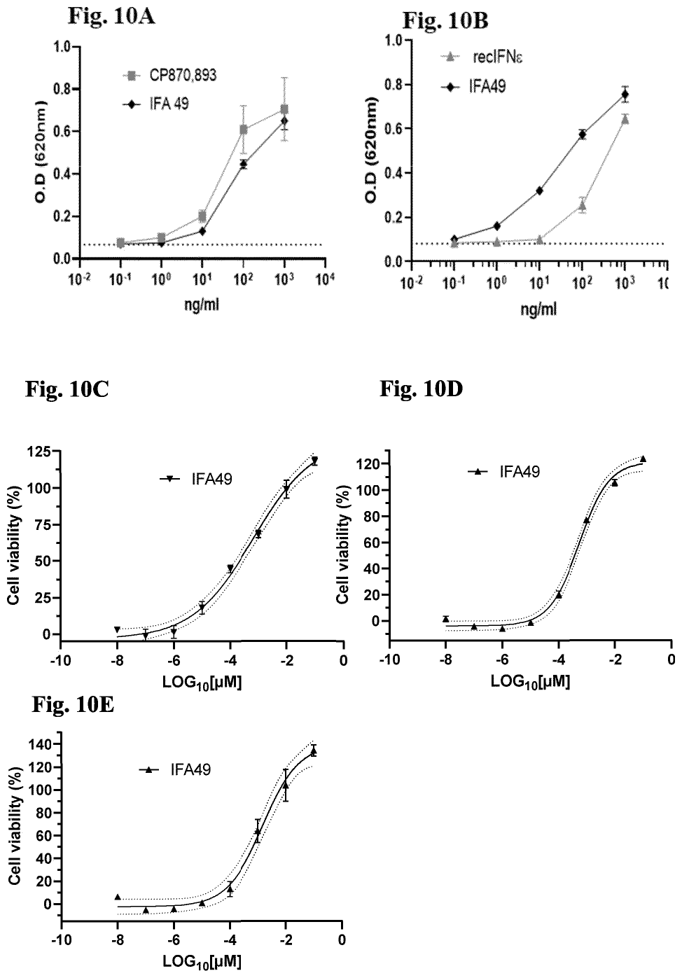

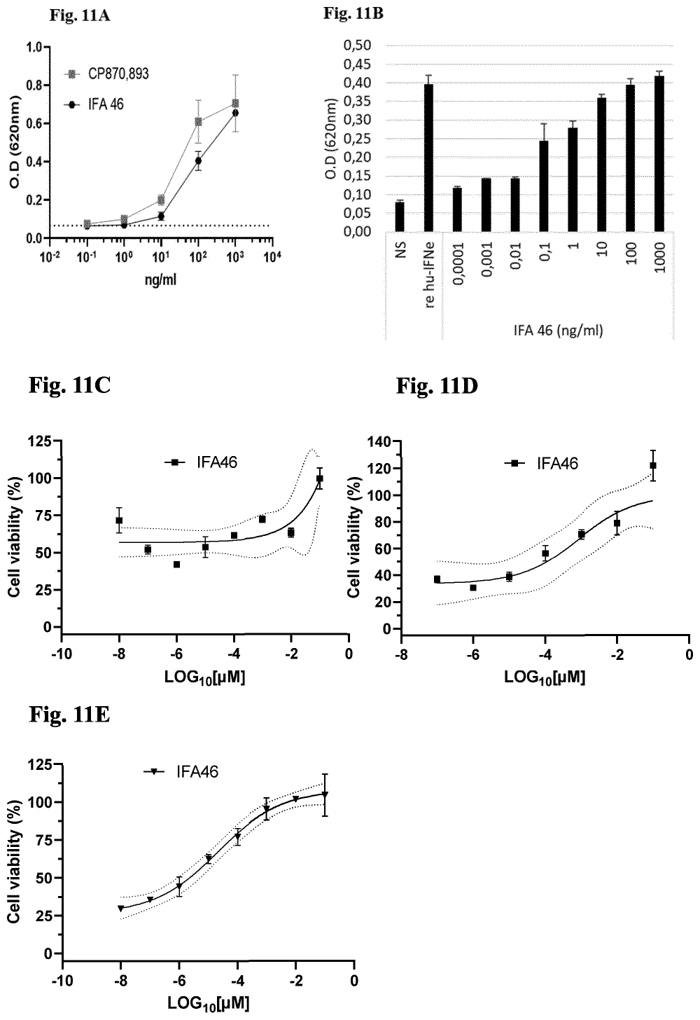

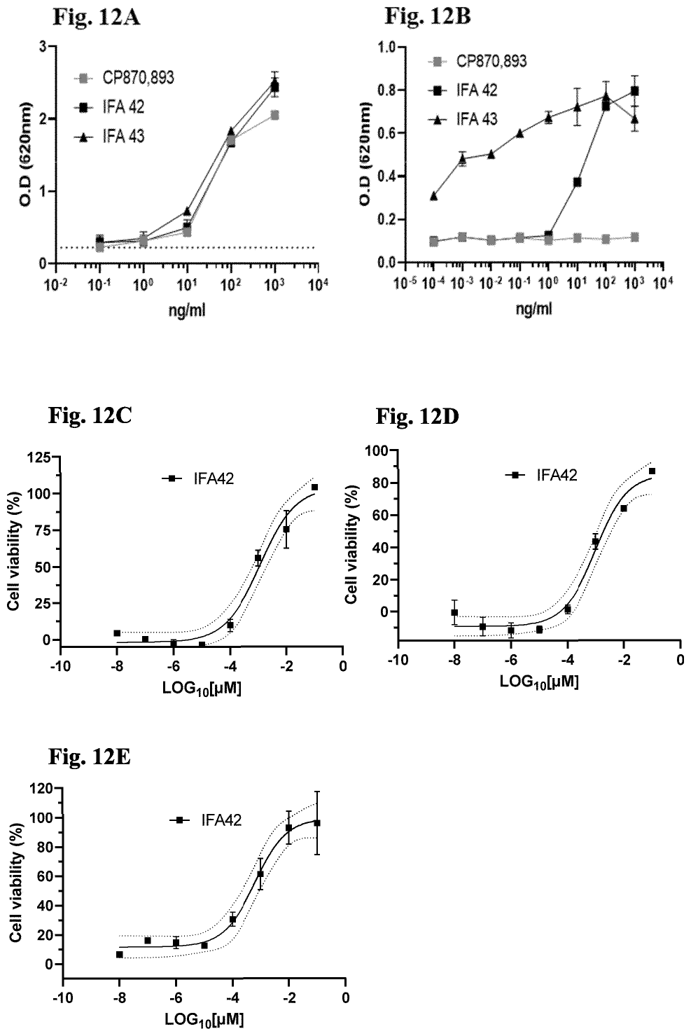

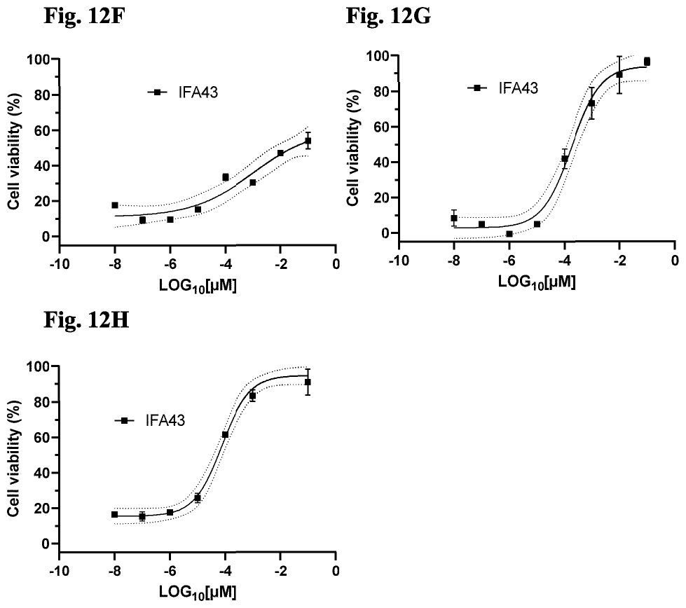

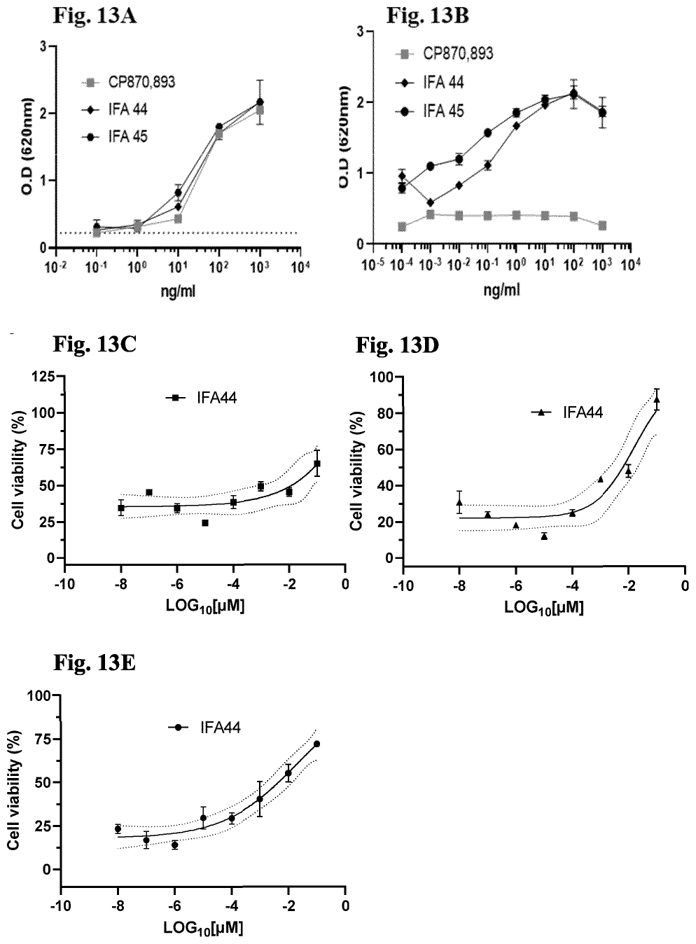

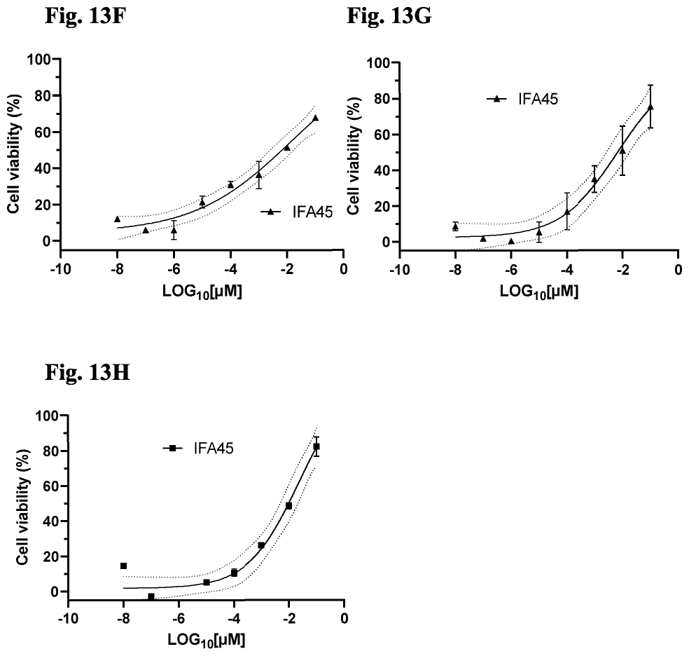

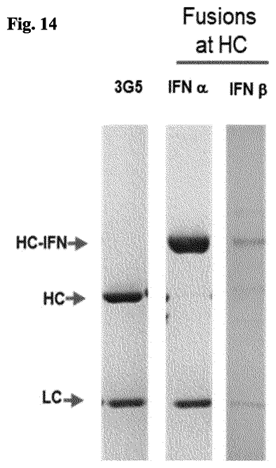

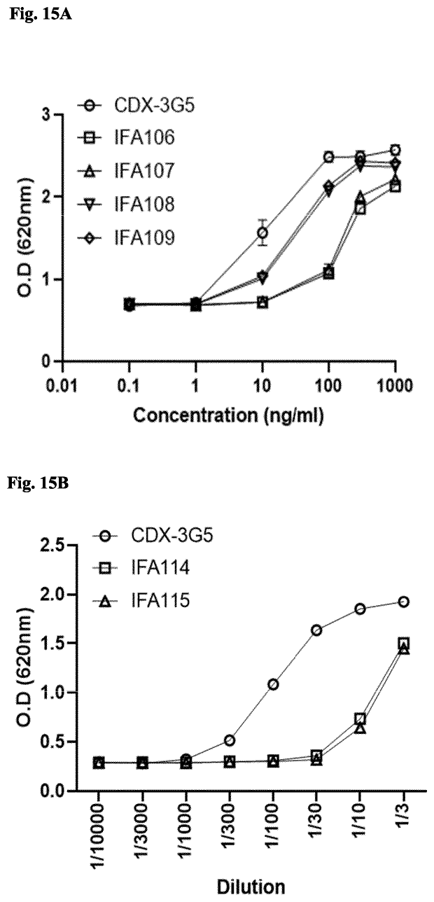

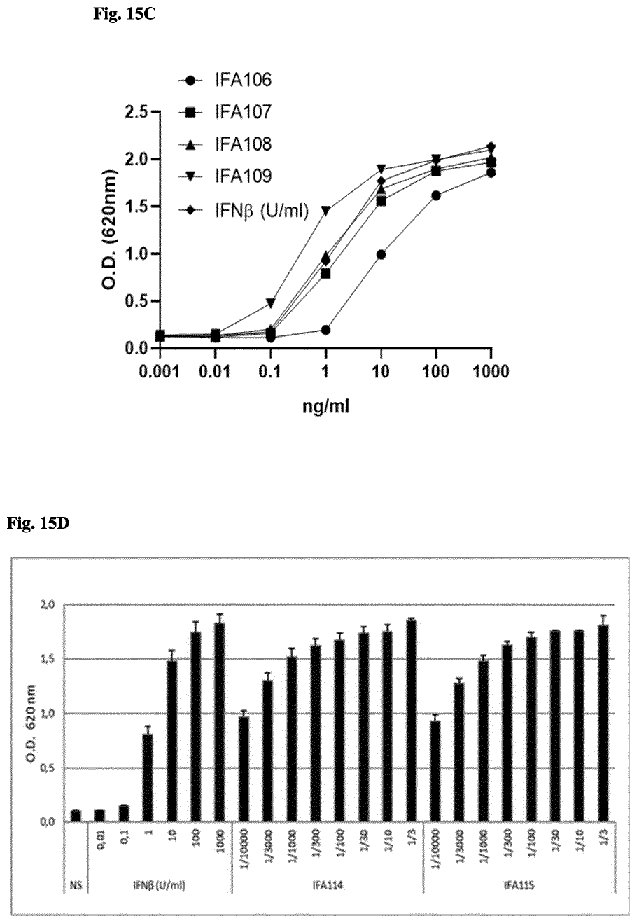

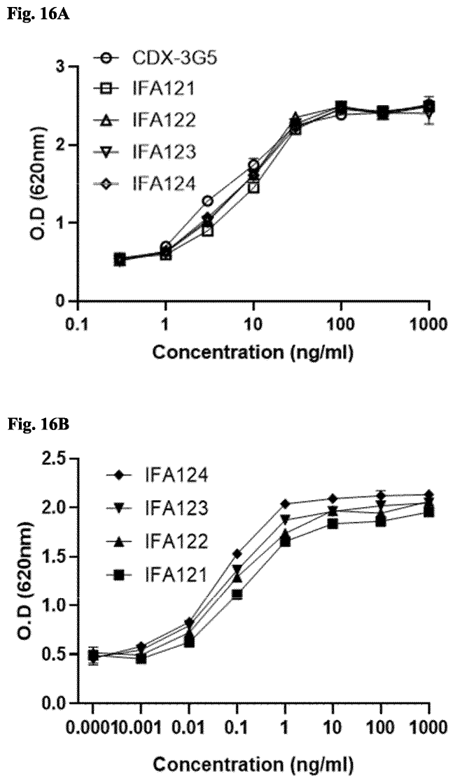

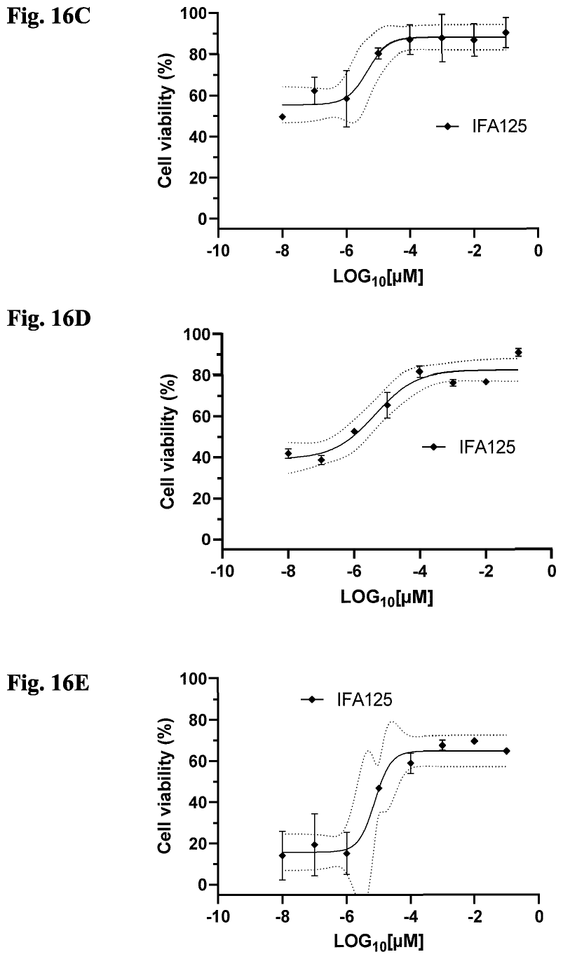

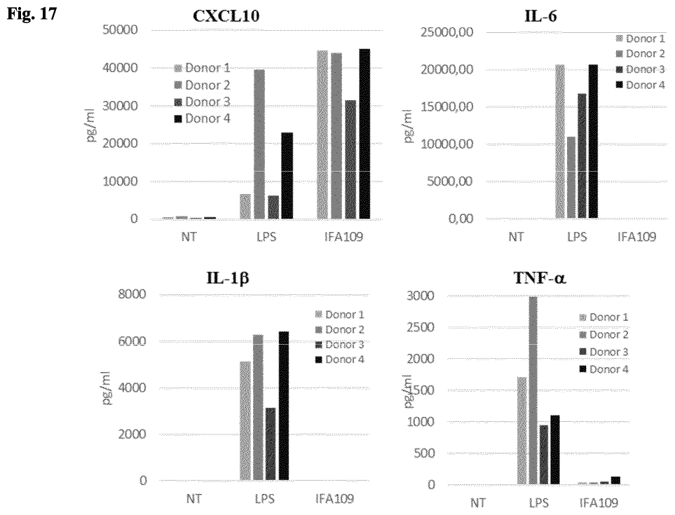

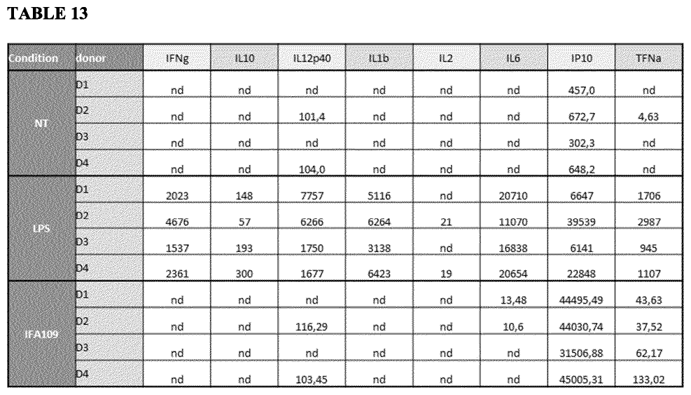

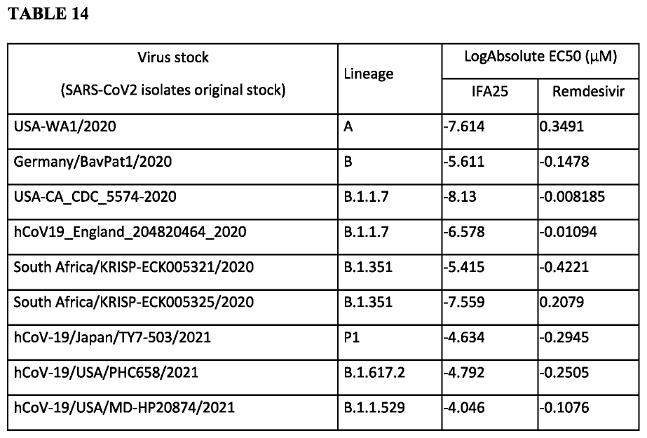

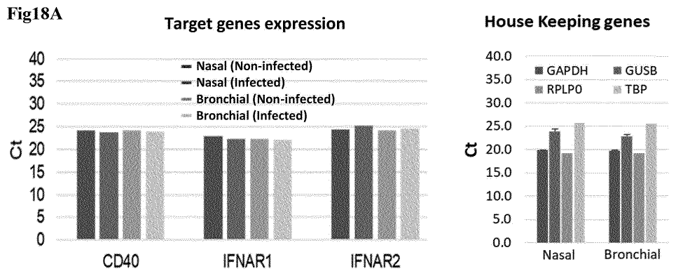

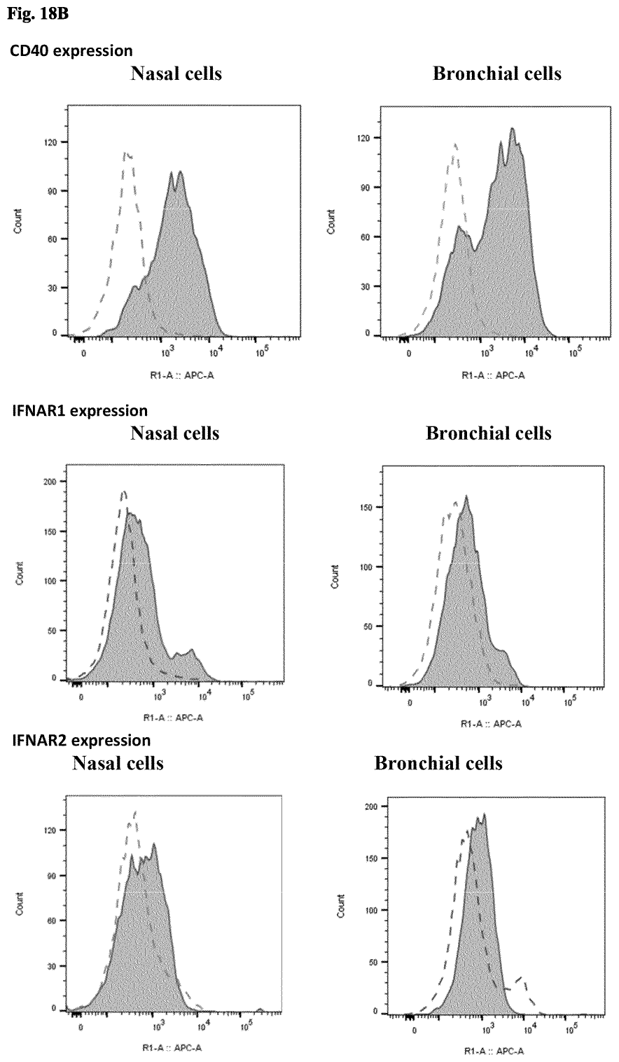

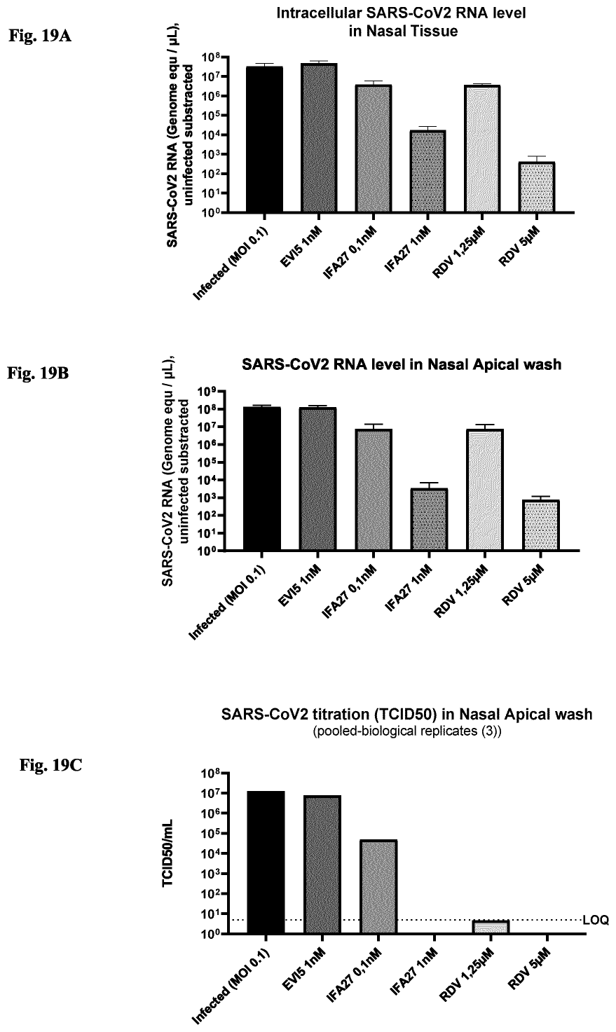

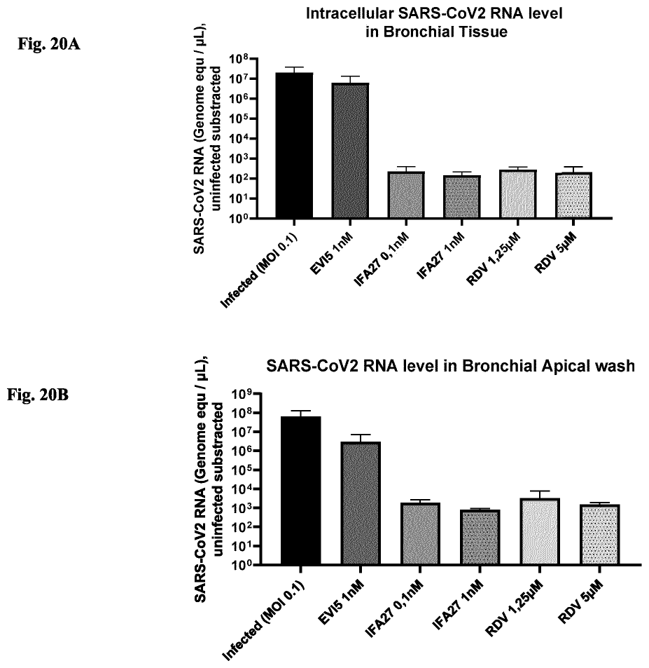

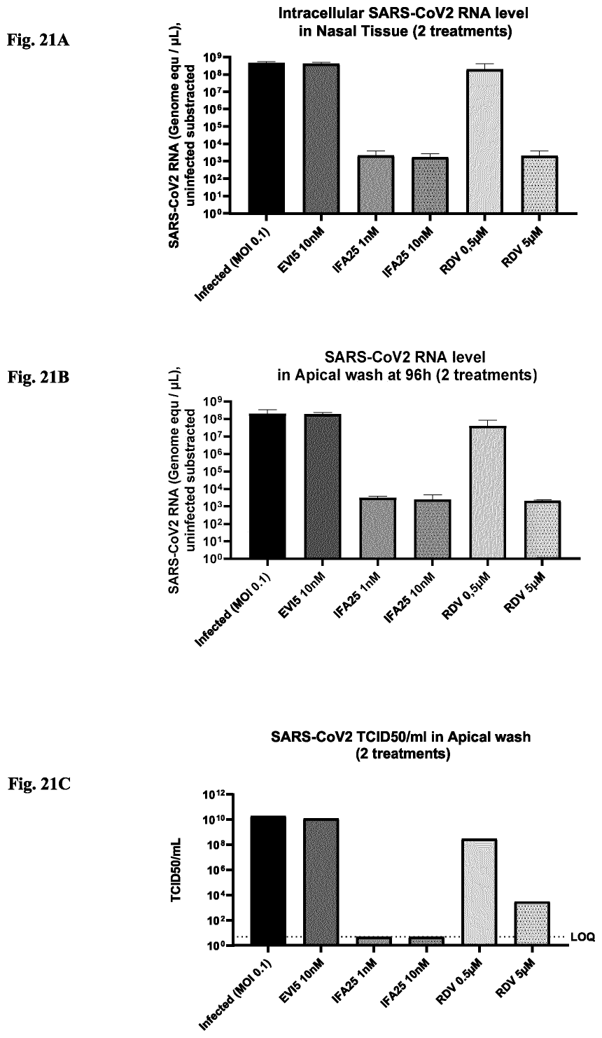

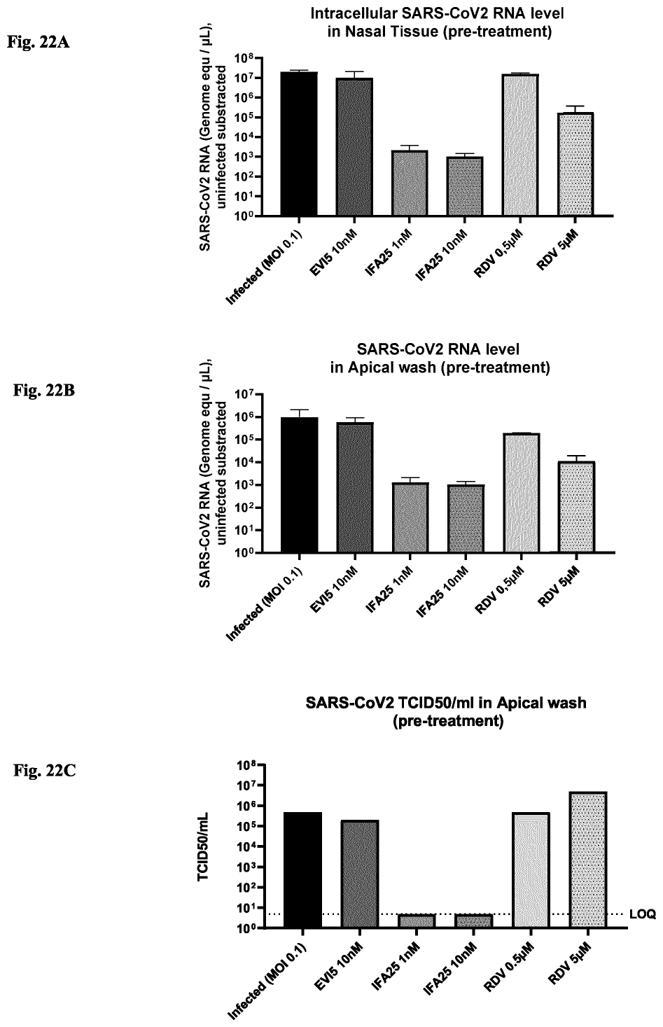

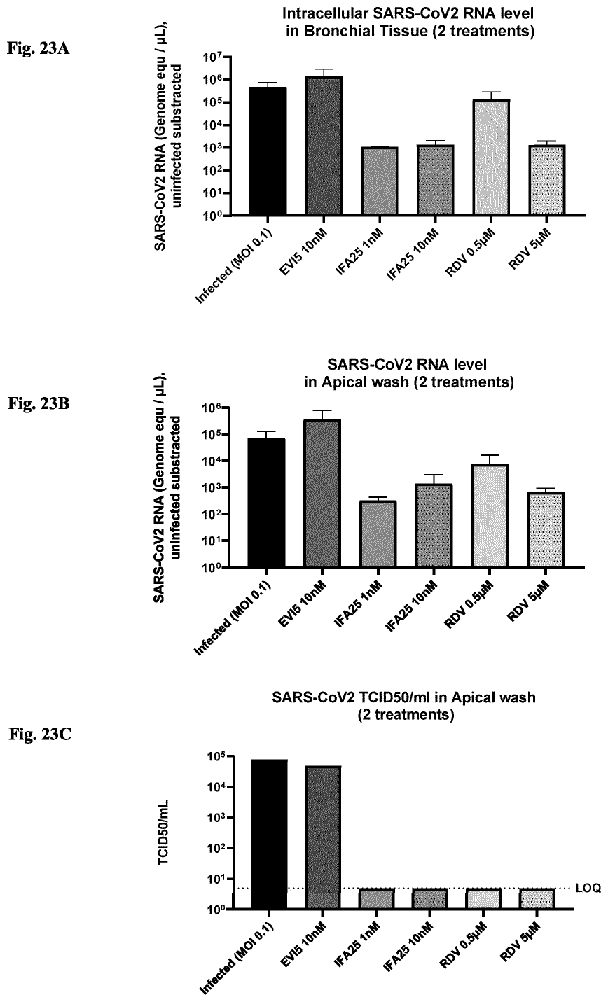

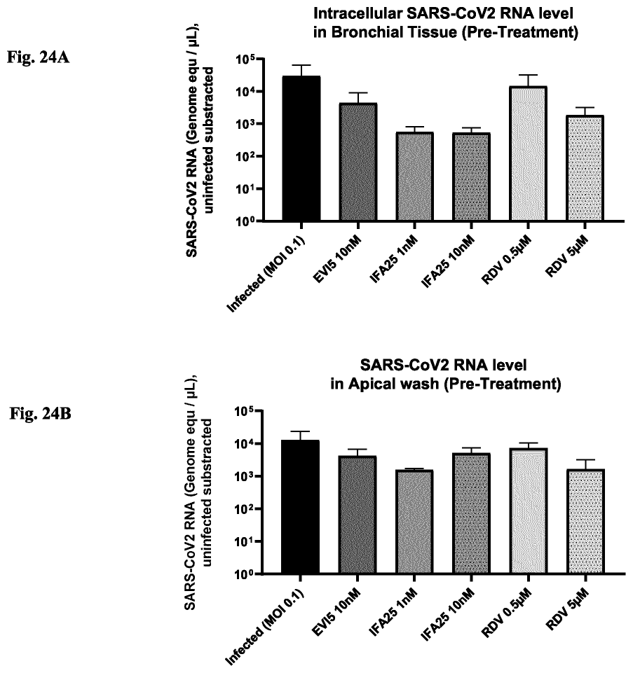

To determine the PK parameters, CP870,893, IFA25, IFA26, IFA27, IFA28, IFA29 and IFA30 were administrated at 0.5 mg/kg and Pegasys at 0.3 mg/kg i.v. bolus to male CD1-Swiss mice and blood samples were collected at different time points. Examples of quantification of circulating molecules using the ELISA approach described above and revealed with anti-IFNα-conjugated HRP are shown in FIGS. 8A and 8B, while examples of quantification revealed with anti-IgG2-conjugated HRP are shown in FIG. 8C; Pegasys quantification is shown in FIG. 8D. In one set of experiments summarized in Table 12A, PK parameters for CP870,893 were explored in a 7-day experiment and those for IFA27, IFA29 and IFA30 in 10-day experiments (quantification for IFA27 was performed using 2 different ELISA approaches). In another set of experiments summarized in Table 12B, the PK parameters for CP870,893 and IFA25, IFA26, IFA28 and Pegasys were explored in 21-day experiments (quantification for IFA25 was performed using 2 different ELISA approaches).

After a short distribution phase, the pharmacokinetic profiles of IFAs are characterized by a long serum half-life ranging from 116 to 218 h (Table 12A and Table 12B). Very similar PK profiles were obtained for the 6 tested IFAs with high circulating level even ten days after single dose administration. The pharmacokinetic parameters summarized in Table 12A/B indicate that these IFAs surprisingly circulate in the blood with higher systemic exposure (AUC (0-inf)) ranging from 1033 μg·h/mL to 2552 μg·h/mL for IFAs in comparison to 590 or 797 μg·h/mL, respectively, for the parental antibody CP870,893 (up to 3.2 fold), also reflecting lower clearance values for IFAs. The volume of distribution Vss was low and ranked from 50 to 105 mL/kg, slightly higher than the plasma vascular volume (50 mL/kg) in this species. For all IFAs, the clearance was ranked as low (0.28 to 0.49 mL/h/kg). Interestingly, the clearance of Pegasys (1.4 mL/hr/kg) is up to 7 fold higher than clearance of IFAs (e.g., 0.2 mL/hr/kg for IFA27) demonstrating a higher systemic exposure of IFAs.

### Example V

V.a—Functional Activities of IFAs without Fc Region on Reporter Cells

To determine whether the Fc part of IFAs is needed for activity, fusions of IFNα to the C-terminal part of the LC associated with a Fab fragment of the HC were designed and produced. IFNα was linked to the LC part with a (G4S)2 (IFA50) or (G4S)3 (IFA51) linker.

Evaluation on HEK-Blue™ CD40L cells demonstrated that such IFAs still exhibit agonistic CD40 activity (FIG. 9A) and activate the CD40 pathway with an EC₅₀ value of about 128 ng/ml (IFA 50) and 123 ng/mL (IFA51), respectively.

Evaluation of the IFN activity on HEK-Blue™ IFN-α/β cells showed that both tested IFAs exhibit IFN activity (FIG. 9B). EC₅₀ values are reported in Table 9B and are about 1.36 ng/ml for IFA50 and 1.43 ng/mL for IFA51.

V.b—Functional Activities of IFNε Based IFAs on Reporter Cells and on SARS-CoV-2 Infected Cells

Fusions of CP870,893 to a third type I interferon (IFN epsilon; IFNε) have also been designed and produced. Such IFAs were tested on HEK-Blue™ CD40L cells and it could be demonstrated that they maintain agonistic CD40 activity. Results for one such IFA (IFA49) are shown in FIG. 10A. Evaluation on HEK-Blue™ hIFN-α/β cells (which are in fact activated by any type I interferon) showed that IFA49 is also able to activate the IFN-I-pathways (FIG. 10B). EC₅₀ values are reported in Table 9B.

These results demonstrate that IFAs with IFNβ maintain both IFN and agonistic CD40 activity (i.e., are bifunctional).

The effect of IFA49 on SARS-CoV-2 infection was assessed in dose-range, in post-treatment design experiments, against 3 major variants: isolate USA-WA1/2020 (MOI 0.05), isolate hCoV-19/USA/PHC658/2021 (Delta variant, MOI 0.05) and isolate hCoV-19/USA/MD-HP20874/2021 (Omicron variant, MOI 0,1) (FIGS. 10C to 10E). Cell viability was assessed 3 days later using the CTG assay.

IFA49 potently rescued Vero E6 cells from SARS-CoV-2-induced cytopathic effect in a dose-dependent manner with an EC₅₀ of 6.565×10⁻⁴ μM against USA-WA1/2020 isolate (FIG. 10C), an EC₅₀ of 5.45×10⁻⁴ μM against Delta variant (FIG. 10D) and an EC₅₀ of 1.517×10⁻³ μM against Omicron variant (FIG. 10E).

V.c—Functional Activities of IFNω Based IFAs on Reporter Cells and on SARS-CoV-2 Infected Cells

Fusions of CP870,893 to a fourth type I interferon (IFN omega; IFNω) have also been designed and produced. Such IFAs were tested on HEK-Blue™ CD40L cells and results demonstrated that they maintain agonistic CD40 activity. Results for one such IFA (IFA46) are shown in FIG. 11A. Evaluation on HEK-Blue™ hIFN-α/β cells (which are in fact activated by any type I interferon) showed that IFA46 is also able to activate the IFN-I-pathways (FIG. 11B). EC₅₀ values are reported in Table 9B.

These results demonstrate that IFAs with IFNω maintain both IFN and agonistic CD40 activity (i.e., are bifunctional).

The effect of IFA46 on SARS-CoV-2 infection was assessed in dose-range, in post-treatment design experiments, against 3 major variants: isolate USA-WA1/2020 (MOI 0.05), isolate hCoV-19/USA/PHC658/2021 (Delta variant, MOI 0.05) and isolate hCoV-19/USA/MD-HP20874/2021 (Omicron variant, MOI 0.1) (FIGS. 11C to 11E). Cell viability was assessed 3 days later using the CTG assay.

IFA46 potently rescued Vero E6 cells from SARS-CoV-2-induced cytopathic effect in a dose-dependent manner with 100% rescue of USA-WA1/2020 isolate infected Vero E6 cells achieved at the highest concentration (FIG. 11C), with an EC₅₀ of 8.395×10⁻⁴ μM against Delta strain (FIG. 11D) and with an EC₅₀ of 2.111×10⁻⁵ μM against Omicron strain (FIG. 11E).

V.d—Functional Activities of IFNγ Based IFAs on Reporter Cells and on SARS-CoV-2 Infected Cells

Fusions of CP870,893 to type II Interferon (IFN gamma; IFNγ) have also been designed and produced. Evaluation of these IFAs on HEK-Blue™ CD40L cells demonstrate that they maintain agonistic CD40 activity, regardless of whether IFNγ is linked to the C-terminal part of the LC (IFA42) or of the HC (IFA43) (FIG. 12A). Evaluation of these IFAs on HEK-Blue™-IFNγ cells (FIG. 12B) showed that they are also able to activate the IFNγ-pathway. IFNγ activity differed somewhat between IFA42 (EC₅₀: 15 ng/ml) and IFA43 (EC₅₀: <0.01 ng/ml). EC₅₀ values are reported in Table 9B.

Taken together, these results demonstrate that IFAs with IFNγ maintain both IFN and agonistic CD40 activity (i.e., are bifunctional).

The effect of IFA42 and IFA43 on SARS-CoV-2 infection was assessed in dose-range, in post-treatment design experiments, against 3 major variants: isolate USA-WA1/2020 (MOI 0.05), isolate hCoV-19/USA/PHC658/2021 (Delta variant, MOI 0.05) and isolate hCoV-19/USA/MD-HP20874/2021 (Omicron variant, MOI 0.1) (FIGS. 12C to 12H). Cell viability was assessed 3 days later using the CTG assay.

Results indicate that IFA42 potently rescued Vero E6 cells from SARS-CoV-2-induced cytopathic effect in a dose-dependent manner with an EC₅₀ of 1.195×10⁻³ μM against USA-WA1/2020 isolate (FIG. 12C), an EC₅₀ of 0.9971×10⁻³ μM against Delta variant (FIG. 12D) and an EC50 of 0.6351×10⁻³ μM against Omicron variant (FIG. 12E). IFA43 potently rescued Vero E6 cells from SARS-CoV-2-induced cytopathic effect in a dose-dependent manner with an EC₅₀ of 1.063×10⁻³ μM against USA-WA1/2020 isolate (FIG. 12F), an EC₅₀ of 1.812×10⁻⁴ μM against Delta variant (FIG. 12G) and an EC₅₀ of 7.575×10⁻⁵ μM against Omicron variant (FIG. 12H).

V.e—Functional Activities of IFN Based IFAs on Reporter Cells and on SARS-CoV-2 Infected Cells

Fusions of CP870,893 to type III Interferon (IFN lambda; IFN)) have also been designed and produced. These IFAs were tested on HEK-Blue™ CD40L cells and results demonstrated that they also maintain agonistic CD40 activity, regardless of whether IFNλ is linked to the C-terminal part of the LC (IFA44) or of the HC (IFA45) (FIG. 13A). Evaluation of these IFAs on HEK-Blue™-IFNλ cells showed that they are also able to activate the IFNλ-pathway (FIG. 13B). EC₅₀ values are reported in Table 9B. These results also demonstrate that IFAs with IFNλ maintain both IFN and agonistic CD40 activity (i.e., are bifunctional).

The effect of IFA44 and IFA45 on SARS-CoV-2 infection was assessed in dose-range, in post-treatment design experiments, against 3 major variants: isolate USA-WA1/2020 (MOI 0.05), isolate hCoV-19/USA/PHC658/2021 (Delta variant, MOI 0.05) and isolate hCoV-19/USA/MD-HP20874/2021 (Omicron variant, MOI 0.1) (FIGS. 13C to 13H). Cell viability was assessed 3 days later using the CTG assay.

IFA44 potently rescued Vero E6 cells from SARS-CoV-2-induced cytopathic effect in a dose-dependent manner with more than 60% rescue of USA-WA1/2020 isolate-infected Vero E6 cells achieved at 10⁻¹ μM concentration (FIG. 13C), an EC₅₀ of 1.628×10⁻² μM against Delta variant (FIG. 13D) and an EC₅₀ of 2.132×10⁻² M against Omicron variant (FIG. 13E). IFA45 potently rescued Vero E6 cells from SARS-CoV-2-induced cytopathic effect in a dose-dependent manner with an EC₅₀ of 1.458×10⁻² μM against USA-WA1/2020 isolate (FIG. 13F), an EC₅₀ of 6.789×10⁻³ μM against Delta variant (FIG. 13G) and an EC₅₀ of 2.329×10⁻² μM against Omicron variant (FIG. 13H).

### Example VI

### Generation of Interferon-Fused Antibodies (IFA) Based on Anti-CD40 Antibody 3G5 and Characterization on Reporter Cells

VI.a—IFA Design

The sequence combinations of exemplary IFAs, designed with 3G5 anti-CD40 antibody (Celldex) as backbone antibody, with the location of IFNs and the nature of the linkers are listed in Table 8 and Table 10. IFN was fused via a linker at the C-terminal part of the Light Chain (LC) or the Heavy Chain (HC), as indicated in Table 8. Nucleic acids encoding the HC, the LC or the fusions were synthesized with optimized mammalian expression codons and cloned into a eukaryotic expression vector such as pcDNA3.1 (Invitrogen).

VI.b—IFA Production

IFA production was performed as described earlier and the production yield is indicated in Table 10. For some IFAs, the production yield was very low, mainly for the fusion of IFNβ to the C-terminal part of the LC. For these IFAs, the agonistic CD40 and the IFN activities were assessed directly using the supernatant containing IFAs without any further purification. Reduced SDS-PAGE analysis of purified IFAs indicated the presence of two major bands corresponding to the HC and LC. When IFN was fused to the HC, a shift of its molecular weight was observed. (FIG. 14).

VI.c—Functional Activities of IFNα/f-Based IFAs on Reporter Cells

Characterization of 3G5 IFAs on reporter cells was done on HEK-Blue™ CD40L (FIG. 15A-B, and FIG. 16A) and HEK-Blue™ IFN-α/β cells (FIG. 15C-D, and FIG. 16B) as previously described (see I.c).

### VI.c.1. IFNβ Based IFAs

FIG. 15—shows examples of dose responses of IFAs, where IFNβ was fused to the HC or the LC of 3G5, on HEK-Blue™ CD40L and HEK-Blue™ IFN-α/β cells (FIG. 15). Results summarized in Table 10 indicate that all tested IFNβ-based IFAs are functional and able to activate both the CD40 pathway and the IFNα/β pathway in a dose-dependent manner.

Examples of CD40 activity are shown in FIG. 15A and FIG. 15B. Fusion of IFNβ to the C-terminal part of the HC demonstrates high variable anti-CD40 activity and in all cases lower than the parental antibody with EC₅₀ values ranging from 30 ng/mL to 190.5 ng/mL (FIG. 15A and Table 10). The mean EC₅₀ value for the parental 3G5 antibody is 9.3 ng/mL.

For fusions on the C-terminal part the LC, the production yield was very low and the activity was assessed using supernatant-containing IFAs after overexpression in HEK-cells. Evaluation of these supernatants on HEK-Blue™ CD40L (FIG. 15B) demonstrates that these IFAs are active on CD40 pathway. For 3G5, the agonistic anti-CD40 activity is still detected when supernatant was diluted 300 times. Conversely, a 1/10 dilution was needed for the IFAs-containing supernatants to observe an activity (FIG. 15B).

The IFN activity of IFAs were tested on HEK-Blue™ IFN-α/β cells and results are summarized in Table 10. Examples are shown in FIG. 15C-D. For fusions of IFNβ at the C-terminal part of the HC, the IFN activity is variable depending on the linker sequence with EC₅₀ values ranging from 0.45 ng/mL to 10.3 ng/mL (FIG. 15C). For IFAs with IFNβ fusion at C-terminal part of the LC-containing supernatant, IFN activity is still detected even after a 10000-fold dilution of the supernatant (FIG. 15D).

### VI.c.2. IFNα Based IFAs

FIGS. 16A-B show examples of dose responses of IFAs, where IFNα was fused to the HC of 3G5, on HEK-Blue™ CD40L (FIG. 16A) and HEK-Blue™ IFNα/β cells (FIG. 16B).

Results indicate that all IFAs display a functional activation of both the CD40 pathway and the IFNα/β pathway in a dose-dependent manner (mean EC₅₀ values are reported in Table 10).

For all the IFNα-based IFAs, the potency on CD40 pathway was similar to the parental antibody with the mean EC₅₀ values ranging from 11.74 ng/mL to 14.2 ng/mL (FIG. 16A and Table 10). The mean EC₅₀ value for the parental 3G5 antibody is 9.3 ng/mL.

The IFN activities of IFNα-based IFAs were tested on HEK-Blue™ IFN-α/0 cells and demonstrate very high activity. The mean EC₅₀ values for the IFN activity of these IFAs ranged from 0.04 ng/mL to 0.12 ng/mL (FIG. 16B and Table 10).

### VI.c.3. IFNγ Based IFAs

Evaluation of IFA125 on HEK-Blue™ CD40L and on HEK-Blue™-IFNγ cells as previously described (see I.c) showed that IFA 125 is functional and able to activate both the CD40 pathway and the IFNγ pathway (Table 10B).

The effect of IFA125 on SARS-CoV-2 infection was assessed in dose-range, in post-treatment design experiments, against 3 major variants: isolate USA-WA1/2020 (MOI 0.05), isolate hCoV-19/USA/PHC658/2021 (Delta variant, MOI 0.05) and isolate hCoV-19/USA/MD-HP20874/2021 (Omicron variant, MOI 0.1) (FIGS. 16C to 16E). Cell viability was assessed 3 days later using the CTG assay.

IFA125 potently rescued Vero E6 cells from SARS-CoV-2-induced cytopathic effect in a dose-dependent manner with 100% rescue of USA-WA1/2020 isolate infected Vero E6 cells from 10⁻⁴ μM concentration (FIG. 16C), with an EC₅₀ of 4.283×10⁻⁶ μM against Delta variant (FIG. 16D) and an EC₅₀ of 7.482×10⁻⁶ M against Omicron variant (FIG. 16E).

VI.d—Generation and Characterization of IFAs without the Fc Region

Suitable constructs according to the invention can also be interferon-associated antigen binding proteins without an Fc region. A construct encoding the heavy chain of the Fab fragment of 3G5 fused to a TEV-His tag was designed (SEQ ID NO 65) and cloned into the expression plasmid pcDNA3.1. This construct is cotransfected in HEK cells as described earlier, with LCs fused via different linkers to IFNs such as SEQ ID NO 70, or SEQ ID NO 71. Proteins and/or supernatants are evaluated in reporter cells and/or their effect on Coronavirus-infected cells. It will be understood by one of skill in the art that constructs for use in therapy will no longer contain the TEV-His tag. These constructs are likewise embodiments of the invention. Interferon-associated antigen binding proteins without the Fc part will be active against Coronavirus infection.

### Example VII

Cytokine Release Assay (CRA) from Human Whole Blood Cells

A WBC ex vivo stimulation assay was used to investigate release of cytokines following IFA stimulation as described previously (see III.a). An example with IFA109 is shown in FIG. 17 and Table 13. The results indicate that all IFAs induce CXCL10 release. They did not induce IL-10, IL-10 and IL-2, and they induced only very low to moderate level of IFNγ, IL-6 and TNF-α, thus suggesting a favorable safety profile with regard to the induction of inflammatory cytokines.

### Example VIII

### Effect of IFAs on SARS-CoV-2 Infected Nasal and Bronchial Human Airway Epithelia

VIII.a—CD40 and IFNAR RNA Expression and Cytometry Analysis in Human Nasal and Bronchial Cells

CD40 and IFNAR expression were assessed by RT-qPCR analysis in primary human nasal and bronchial cells, cultured in the air-liquid interface system, in infected and non-infected condition. Total RNA was extracted from primary human nasal and bronchial cells (non-infected or infected) following manufacturer recommendation (SV96 Total RNA isolation system, cat #Z3505). Briefly, the SV 96 Total RNA Isolation System, combining the disruptive and protective properties of guanidine thiocyanate (GTC) and β-mercaptoethanol, was used to disrupt nucleoprotein complexes and inactivate the ribonucleases present in cell extracts.

The cell lysate was then applied to the Binding Plate for binding of total RNA to the columns. RNase-Free DNase I was applied directly to the silica membrane to digest contaminating genomic DNA. The bound total RNA was further purified from contaminating salts, proteins and cellular impurities by simple washing steps. Finally, total RNA was eluted from the membrane by the addition of Nuclease-Free Water. Once eluted, 500 ng of RNA were first used as template for cDNA synthesis following manufacturer instructions (cat #11754050). Then, TLDA receptor array card (Thermofisher Scientific) was performed to simultaneously quantify the mRNA level of specific receptors including CD40 (Taqman assay Hs00374176), IFNAR1 (Taqman assay Hs01066116) and IFNAR2 (Taqman assay Hs00174198). House-keeping genes mRNA expression including GAPDH (Taqman assay Hs99999905), GUSB (Taqman assay Hs99999908), TBP (Taqman assay Hs99999910) and RPLPO (Taqman assay Hs99999902) were also included in the assay to assess potential mRNA variation in all 4 conditions tested, with Ct value below 25 for each receptor (FIG. 18A).

The CD40 and IFNAR expression was also confirmed in non-infected nasal and bronchial cells cultured in the air-liquid interface system, at the protein level by cytometry analysis (FIG. 18B). Primary human nasal and bronchial cells (0.2×10⁶ cells/well) were resuspended in PBS 1×+2 mM EDTA+0.5% BSA+(PEB) buffer in the presence of a viability marker Aqua LIVE/DEAD (Invitrogen L34957) for 30 mn at 4° C. After centrifugation at 1300 rpm for 3 mn at 4° C., cells were resuspended in 100 μl of PEB in the presence of anti-CD40-APC antibody (Miltenyi, 130-110-947) or matching control isotype-APC (Miltenyi 130-113-434), in the presence of anti-IFNAR1-APC antibody (Bio-techne SAS, FAB245A) or matching control isotype-APC (Bio-techne SAS, IC002A) and in the presence of anti-IFNAR2-APC antibody (Miltenyi, 130-099-558) or matching control isotype-APC (Miltenyi, 130-113-434). Cells were incubated for 30 mn at 4° C. Then cells were centrifuged, washed, fixed for 10 mn at 4° C. with 4% PFA. After centrifugation and washing, cells were resuspended in PEB buffer, acquired on MACSQuant16 cytometer and analyzed using Flowjo software.

VIII.b—SARS-CoV-2 Infection Model in Primary Human Cells in the Air Liquid Interface System.

MucilAir™ (Epithelix) is an in vitro cell model of the human airway epithelium cultured at the air liquid interface. Ready-to-use epithelium were maintained in MucilAir™ culture medium (cat #EP05MM) in a dedicated incubator at 37° C., 95% humidity and 5% CO₂. In this MucilAir™ systems, the treatment can be applied before, during or after the infection step. For the IFA evaluation, two treatment designs, named respectively post-treatment and pre-treatment, were developed as follows.

Post-treatment design (2 or 3 treatments): At the day of infection, epithelia were washed twice at the apical face with Opti-MEM (cat #31985-062) then infected with SARS-CoV-2 isolate USA-WA1/2020 at 0.1 MOI for 1 h. Afterwards, cells were washed twice with Opti-MEM to remove residual viral inoculum and fresh MucilAir™ culture medium complemented with treatment of interest were added at the basal compartment. For the 96 h kinetic, a second treatment was applied at 48 h during the medium renewal. For the 168 h kinetic, second and third treatments were done at 48 hours and 96 hours during the medium renewal. Apical wash was collected, depending the kinetic, at 48 h, 96 h and 168 h in Opti-MEM for SARS-CoV-2 RT-qPCR (nucleospin 96 virus, cat #740691) and TCID50 assessment as describe below. At the end of the kinetic (96 h or 168 h) epithelium was lysed with RNA lysis kit (cat #Z3505) for intracellular SARS-CoV-2 RT-qPCR quantification as described below.

Pre-treatment design: The day of infection, fresh MucilAir™ culture medium complemented with treatment of interest were added at the basal compartment for 3 h prior infection. Later, Epithelia were washed twice at the apical face with Opti-MEM (cat #31985-062) then infected with SARS-CoV-2 isolate USA-WA1/2020 at 0.1 MOI for 1 h. Afterwards, cells were washed twice with Opti-MEM to remove residual viral inoculum and fresh MucilAir™ culture medium without treatment was added at the basal compartment for 48 h. At the end of the kinetic, apical wash was collected in Opti-MEM for SARS-CoV-2 RT-qPCR and TCID50 assessment as described below. Then, epithelium was lysed with RNA lysis kit (cat #Z3505) following manufacturer instructions for intracellular SARS-CoV-2 RT-qPCR quantification as describe below.

SARS-CoV-2 viral RNA quantification: For the viral RNA quantification in apical washes, briefly, 100 μl of wash per condition was extracted with a viral specific nucleic acid extraction kit (nucleospin 96 virus, cat #74061 from Macherey-Nagel) following manufacturer instructions. Then, Viral RNA was eluted in 100p of nuclease-free water prior quantification by RT-qPCR. For RT-qPCR quantification 5 μl of viral RNA was added to a reaction mixture of TaqMan Fast Virus 1-step Master mix (#4444434, Thermo Fisher), SARS-CoV-2 20× Taqman assay (#4332078, Forward primer 5′-GACCCCAAAATCAGCGAAAT-3′; reverse primer 5′-TCTGGTTACTGCCAGTTGAATCTG-3; probe FAM-ACCCCGCATTACGTTTGGTGGACC-MGB-NFQ) and nuclease-free water. After an initial reverse transcription step for 5 min at 50° C. to generate cDNA, enzyme inactivation step occurred for 20 sec at 95° C. The PCR product was amplified immediately in 40 PCR cycles (denaturation step for 3 sec at 95° C. followed by annealing and extension for 30 sec at 60° C.). SARS-CoV-2 copy numbers were calculated using a ten-fold serial dilution of an internal reference genomic SARS-CoV-2 RNA standard amplified in the same condition.

For the viral RNA quantification in MucilAir™ tissue, first the RNA were extracted using the RNA lysis kit (Cat #Z3505) as already described above. Then, total RNA were eluted in 100 μl of nuclease-free water for RT-qPCR amplification and quantification as described above.

SARS-CoV-2 virus titers determination: In brief, for each treatment condition, the same volume of apical washes from the biological replicates were pooled, thus representing one sample for each group. Then, serial ten-fold dilutions of these samples were prepare and applied to Vero E6 cells plated for 1 day. The CPE was evaluated after 3 days by CellTiter-Glo assay and used to identify the endpoint of infection. Five replicates were used to calculate the 50% cell tissue culture infectious doses per mL (TCID/mL) of apical wash.

VIII.c—Effect of IFA25 and IFA27 on SARS-CoV-2 Infection in Primary Human Cells System

We evaluated the effect of IFA25 and IFA27 in the primary human nasal and bronchial cells, cultured in air liquid interface system and infected with SARS-CoV-2 isolate USA-WA1/2020 at 0.1 MOI, according to example VIII.b. First, IFA27, tested at 0.1 nM and 1 nM, demonstrated a dose dependent effect to reduce the viral RNA in the nasal tissue (FIG. 19A) and nasal apical wash (FIG. 19B). Moreover, IFA27 treatment at the highest dose eliminated the de novo production of infectious virus as shown by the TCID₅₀ titer in apical washes (FIG. 19C). The antiviral effect was also observed in the bronchial cells as shown on the tissue viral RNA load (FIG. 20A) and apical wash viral RNA load (FIG. 20B). The direct-acting antiviral Remdesivir showed a dose dependent effect on viral RNA load and infectious virus titer at much higher concentrations (1.25 μM and 5 μM) than IFA27 when tested in the same experiments.

In addition, IFA25 exhibited an anti-viral activity as shown by the virus inhibition in nasal tissue (FIG. 21) and bronchial tissue (FIG. 23) at both the RNA and infectious virus levels. The antiviral effect of IFA25 was achieved at the lowest dose tested of 1 nM whereas the Remdesivir treatment exhibited a dose range effect and achieved an inhibition at concentrations much higher than IFA25. In addition, IFA25 and Remdesivir were tested in the pre-treatment design as described above. In the pre-treatment design, the effect of IFA25 remained highly potent against SARS-CoV-2 infection in the nasal (FIG. 22) and bronchial (FIG. 24) cells. A decrease of viral RNA load in the intracellular compartment (FIG. 22A and FIG. 24A) and in apical washes (FIG. 22B and FIG. 24B) was observed as well as a decrease in infectious virus titer in the apical wash of the nasal cells (FIG. 22C). An effect of Remdesivir in the pre-treatment design on the viral RNA load in the intracellular compartment (FIG. 22A and FIG. 24A) and in apical washes (FIG. 22B and FIG. 24B) was also observed but it did not reach the same level as compared to the post-treatment design. In addition, Remdesivir in the pre-treatment design did not produce any effect on the infectious virus titer in apical wash of nasal cells (FIG. 22C).

### EQUIVALENTS

The invention may be embodied in other specific forms without departing from the spirit or essential characteristics thereof. The foregoing embodiments are therefore to be considered in all respects illustrative rather than limiting of the invention. The scope of the claimed invention is thus indicated by the appended claims rather than by the foregoing description, and all changes that come within the meaning and range of equivalency of the claims are therefore intended to be embraced herein.

### Items

In view of the above, it will be appreciated that the present invention also relates to the following items:
1. An interferon-associated antigen binding protein comprising
   (I) an agonistic anti-CD40 antibody or an agonistic antigen binding fragment thereof, and
   (II) an Interferon (IFN) or a functional fragment thereof for use in the treatment or prevention of Coronavirus infection.
2. The interferon-associated antigen binding protein for the use of item 1, wherein the agonistic anti-CD40 antibody, or the agonistic antigen binding fragment thereof, comprises three light chain complementarity determining regions (CDRs) that are at least 90% identical to the CDRL1, CDRL2 and CDRL3 sequences within SEQ ID NO 3; and three heavy chain CDRs that are at least 90% identical to the CDRH1, CDRH2 and CDRH3 sequences within SEQ ID NO 6;
   preferably wherein the agonistic anti-CD40 antibody, or the agonistic antigen binding fragment thereof, comprises three light chain complementarity determining regions (CDRs) that are at least 95% identical to the CDRL1, CDRL2 and CDRL3 sequences within SEQ ID NO 3; and three heavy chain CDRs that are at least 95% identical to the CDRH1, CDRH2 and CDRH3 sequences within SEQ ID NO 6;
   more preferably wherein the agonistic anti-CD40 antibody, or the agonistic antigen binding fragment thereof, comprises three light chain complementarity determining regions (CDRs) that are at least 98% identical to the CDRL1, CDRL2 and CDRL3 sequences within SEQ ID NO 3; and three heavy chain CDRs that are at least 98% identical to the CDRH1, CDRH2 and CDRH3 sequences within SEQ ID NO 6; or still more preferably wherein the agonistic anti-CD40 antibody, or the agonistic antigen binding fragment thereof, comprises three light chain complementarity determining regions (CDRs) that are at least 99% identical to the CDRL1, CDRL2 and CDRL3 sequences within SEQ ID NO 3; and three heavy chain CDRs that are at least 99% identical to the CDRH1, CDRH2 and CDRH3 sequences within SEQ ID NO 6.
3. The interferon-associated antigen binding protein for the use of item 1 or 2, wherein the agonistic anti-CD40 antibody, or the agonistic antigen binding fragment thereof, comprises three light chain complementarity determining regions (CDRs) that are identical to the CDRL1, CDRL2 and CDRL3 sequences within SEQ ID NO 3; and three heavy chain CDRs that are identical to the CDRH1, CDRH2 and CDRH3 sequences within SEQ ID NO 6.
4. The interferon-associated antigen binding protein for the use of items 2 or 3, wherein each CDR is defined in accordance with the Kabat definition, the Chothia definition, the AbM definition, or the contact definition of CDR; preferably wherein each CDR is defined in accordance with the CDR definition of Kabat or the CDR definition of Chothia.
5. The interferon-associated antigen binding protein for the use of item 1, wherein the agonistic anti-CD40 antibody or the agonistic antigen binding fragment thereof comprises
   (I)
   (a) a heavy chain or a fragment thereof comprising a complementarity determining region (CDR) CDRH1 that is at least 90% identical to SEQ ID NO 56, a CDRH2 that is at least 90% identical to SEQ ID NO 57, and a CDRH3 that is at least 90% identical to SEQ ID NO 58; and
   (b) a light chain or a fragment thereof comprising a CDRL1 that is at least 90% identical to SEQ ID NO 52, a CDRL2 that is at least 90% identical to SEQ ID NO 53, and a CDRL3 that is at least 90% identical to SEQ ID NO 54;
   preferably (II)
   (a) a heavy chain or a fragment thereof comprising a complementarity determining region (CDR) CDRH1 that is at least 95% identical to SEQ ID NO 56, a CDRH2 that is at least 95% identical to SEQ ID NO 57, and a CDRH3 that is at least 95% identical to SEQ ID NO 58; and
   (b) a light chain or a fragment thereof comprising a CDRL1 that is at least 95% identical to SEQ ID NO 52, a CDRL2 that is at least 95% identical to SEQ ID NO 53, and a CDRL3 that is at least 95% identical to SEQ ID NO 54;
   more preferably (III)
   (a) a heavy chain or a fragment thereof comprising a complementarity determining region (CDR) CDRH1 that is at least 98% identical to SEQ ID NO 56, a CDRH2 that is at least 98% identical to SEQ ID NO 57, and a CDRH3 that is at least 98% identical to SEQ ID NO 58; and
   (b) a light chain or a fragment thereof comprising a CDRL1 that is at least 98% identical to SEQ ID NO 52, a CDRL2 that is at least 98% identical to SEQ ID NO 53, and a CDRL3 that is at least 98% identical to SEQ ID NO 54; or
   still more preferably (IV)
   (a) a heavy chain or a fragment thereof comprising a complementarity determining region (CDR) CDRH1 that is at least 99% identical to SEQ ID NO 56, a CDRH2 that is at least 99% identical to SEQ ID NO 57, and a CDRH3 that is at least 99% identical to SEQ ID NO 58; and
   (b) a light chain or a fragment thereof comprising a CDRL1 that is at least 99% identical to SEQ ID NO 52, a CDRL2 that is at least 99% identical to SEQ ID NO 53, and a CDRL3 that is at least 99% identical to SEQ ID NO 54.
6. The interferon-associated antigen binding protein for the use of item 1, wherein the agonistic anti-CD40 antibody or the agonistic antigen binding fragment thereof comprises
   (a) a heavy chain or a fragment thereof comprising a complementarity determining region (CDR) CDRH1 that is identical to SEQ ID NO 56, a CDRH2 that is identical to SEQ ID NO 57, and a CDRH3 that is identical to SEQ ID NO 58; and
   (b) a light chain or a fragment thereof comprising a CDRL1 that is identical to SEQ ID NO 52, a CDRL2 that is identical to SEQ ID NO 53, and a CDRL3 that is identical to SEQ ID NO 54.
7. The interferon-associated antigen binding protein for the use of any one of the preceding items, wherein the agonistic anti-CD40 antibody, or the agonistic antigen binding fragment thereof, comprises a light chain variable region V_{L} comprising the sequence as set forth in SEQ ID NO 51, or a sequence at least 90% identical thereto; and/or a heavy chain variable region V_{H} comprising the sequence as set forth in SEQ ID NO 55, or a sequence at least 90% identical thereto;
   preferably wherein the agonistic anti-CD40 antibody, or the agonistic antigen binding fragment thereof, comprises a light chain variable region V_{L} comprising a sequence that is at least 95% identical to the sequence as set forth in SEQ ID NO 51; and/or a heavy chain variable region V_{H} comprising a sequence that is at least 95% identical to the sequence as set forth in SEQ ID NO 55;
   more preferably wherein the agonistic anti-CD40 antibody, or the agonistic antigen binding fragment thereof, comprises a light chain variable region V_{L} comprising a sequence that is at least 98% identical to the sequence as set forth in SEQ ID NO 51; and/or a heavy chain variable region V_{H} comprising a sequence that is at least 98% identical to the sequence as set forth in SEQ ID NO 55;
   still more preferably the agonistic anti-CD40 antibody, or the agonistic antigen binding fragment thereof, comprises a light chain variable region V_{L} comprising a sequence that is at least 99% identical to the sequence as set forth in SEQ ID NO 51; and/or a heavy chain variable region V_{H} comprising a sequence that is at least 99% identical to the sequence as set forth in SEQ ID NO 55; or
   most preferably wherein the agonistic anti-CD40 antibody, or the agonistic antigen binding fragment thereof, comprises a light chain variable region V_{L} comprising the sequence as set forth in SEQ ID NO 51 and a heavy chain variable region V_{H} comprising the sequence as set forth in SEQ ID NO 55.
8. The interferon-associated antigen binding protein for the use of any one of the preceding items, wherein a heavy chain of the agonistic anti-CD40 antibody or the agonistic antigen binding fragment thereof, comprises a Fab region heavy chain comprising an amino acid sequence as set forth in SEQ ID NO 12, or a sequence at least 90%, preferably 95%, more preferably 98%, or still more preferably 99% identical thereto.
9. The interferon-associated antigen binding protein for the use of any one of the preceding items, wherein the agonistic anti-CD40 antibody or the agonistic antigen binding fragment thereof comprises a light chain (LC) that comprises a sequence as set forth in SEQ ID NO 3, or a sequence at least 90% identical thereto; and/or a heavy chain (HC) that comprises a sequence selected from the group consisting of SEQ ID NO 6, SEQ ID NO 9, SEQ ID NO 49 and SEQ ID NO 48, or a sequence at least 90% identical thereto;
   preferably wherein the agonistic anti-CD40 antibody or the agonistic antigen binding fragment thereof comprises a light chain (LC) that comprises a sequence that is at least 95% identical to the sequence as set forth in SEQ ID NO 3; and/or a heavy chain (HC) that comprises a sequence that is at least 95% identical to a sequence as set forth within the group of sequences consisting of SEQ ID NO 6, SEQ ID NO 9, SEQ ID NO 49 and SEQ ID NO 48;
   more preferably wherein the agonistic anti-CD40 antibody or the agonistic antigen binding fragment thereof comprises a light chain (LC) that comprises a sequence that is at least 98% identical to the sequence as set forth in SEQ ID NO 3; and/or a heavy chain (HC) that comprises a sequence that is at least 98% identical to a sequence as set forth within the group of sequences consisting of SEQ ID NO 6, SEQ ID NO 9, SEQ ID NO 49 and SEQ ID NO 48;
   still more preferably wherein the agonistic anti-CD40 antibody or the agonistic antigen binding fragment thereof comprises a light chain (LC) that comprises a sequence that is at least 99% identical to the sequence as set forth in SEQ ID NO 3; and/or a heavy chain (HC) that comprises a sequence that is at least 99% identical to a sequence as set forth within the group of sequences consisting of SEQ ID NO 6, SEQ ID NO 9, SEQ ID NO 49 and SEQ ID NO 48; or
   most preferably wherein the agonistic anti-CD40 antibody or the agonistic antigen binding fragment thereof comprises a light chain (LC) that comprises a sequence as set forth in SEQ ID NO 3; and a heavy chain (HC) that comprises a sequence selected from the group consisting of SEQ ID NO 6, SEQ ID NO 9, SEQ ID NO 49 and SEQ ID NO 48.
10. The interferon-associated antigen binding protein for the use of item 9, wherein the HC comprises the sequence as set forth in SEQ ID NO 6, or a sequence at least 90%, preferably 95%, more preferably 98%, or still more preferably 99% identical thereto.
11. The interferon-associated antigen binding protein for the use of item 9, wherein the HC comprises the sequence as set forth in SEQ ID NO 9, or a sequence at least 90%, preferably 95%, more preferably 98%, or still more preferably 99% identical thereto.
12. The interferon-associated antigen binding protein for the use of item 9, wherein the HC comprises the sequence as set forth in SEQ ID NO 49, or a sequence at least 90%, preferably 95%, more preferably 98%, or still more preferably 99% identical thereto.
13. The interferon-associated antigen binding protein for the use of item 9, wherein the HC comprises the sequence as set forth in SEQ ID NO 48, or a sequence at least 90%, preferably 95%, more preferably 98%, or still more preferably 99% identical thereto.
14. The interferon-associated antigen binding protein for the use of any one of the preceding items, wherein the IFN or the functional fragment thereof is a human interferon.
15. The interferon-associated antigen binding protein for the use of any one of the preceding items, wherein the IFN or the functional fragment thereof is selected from the group consisting of a Type I IFN, a Type II IFN and a Type III IFN, or a functional fragment thereof.
16. The interferon-associated antigen binding protein for the use of any one of the preceding items, wherein the IFN or the functional fragment thereof is a Type I IFN, or a functional fragment thereof.
17. The interferon-associated antigen binding protein for the use of item 16, wherein the type I IFN or the functional fragment thereof is IFNα, IFNβ, IFNω, or IFNε, or a functional fragment thereof.
18. The interferon-associated antigen binding protein for the use of item 16, wherein the type I IFN or the functional fragment thereof is IFNα or IFNβ, or a functional fragment thereof.
19. The interferon-associated antigen binding protein for the use of item 16, wherein the type I IFN or the functional fragment thereof is IFNω, or a functional fragment thereof.
20. The interferon-associated antigen binding protein for the use of item 16, wherein the type I IFN or the functional fragment thereof is IFNε, or a functional fragment thereof.
21. The interferon-associated antigen binding protein for the use of any of the items 1 to 14, wherein the IFN or the functional fragment thereof is IFNα, IFNβ, IFNγ, IFNλ, IFNω or IFNε, or a functional fragment thereof.
22. The interferon-associated antigen binding protein for the use of item 21, wherein the IFN or the functional fragment thereof is IFNα or IFNβ, or a functional fragment thereof.
23. The interferon-associated antigen binding protein for the use of item 22, wherein the IFN or the functional fragment thereof is IFNα, or a functional fragment thereof.
24. The interferon-associated antigen binding protein for the use of item 23, wherein the IFN or functional fragment thereof is IFNα2a, or a functional fragment thereof.
25. The interferon-associated antigen binding protein for the use of item 24, wherein the IFNα2a comprises the sequence as set forth in SEQ ID NO 17, or a sequence at least 90%, preferably 95%, more preferably 98%, or still more preferably 99% identical thereto.
26. The interferon-associated antigen binding protein for the use of item 22, wherein the IFN or the functional fragment thereof is IFNβ, or a functional fragment thereof.
27. The interferon-associated antigen binding protein for the use of item 26, wherein the IFNβ comprises the sequence as set forth in SEQ ID NO 14, or a sequence at least 90%, preferably 95%, more preferably 98%, or still more preferably 99% identical thereto.
28. The interferon-associated antigen binding protein for the use of item 26, wherein the IFNβ or the functional fragment thereof comprises one or two amino acid substitution(s) relative to SEQ ID NO 14, selected from C17S and N80Q.
29. The interferon-associated antigen binding protein for the use of item 28, wherein the IFNβ or the functional fragment thereof comprises the amino acid substitution C17S relative to SEQ ID NO 14.
30. The interferon-associated antigen binding protein for the use of item 29, wherein the IFNβ comprises the amino acid sequence as set forth in SEQ ID NO 15.
31. The interferon-associated antigen binding protein for the use of item 28, wherein the IFNβ or the functional fragment thereof comprises the amino acid substitutions C17S and N80Q relative to SEQ ID NO 14.
32. The interferon-associated antigen binding protein for the use of item 31, wherein the IFNβ comprises the amino acid sequence as set forth in SEQ ID NO 16.
33. The interferon-associated antigen binding protein for the use of item 21, wherein the IFN or a functional fragment thereof is IFNγ or IFNλ, or a functional fragment thereof.
34. The interferon-associated antigen binding protein for the use of item 33, wherein the IFN or a functional fragment thereof is IFNγ, or a functional fragment thereof.
35. The interferon-associated antigen binding protein for the use of item 34, wherein the IFNγ comprises the sequence as set forth in SEQ ID NO 19, or a sequence at least 90%, preferably 95%, more preferably 98%, or still more preferably 99% identical thereto.
36. The interferon-associated antigen binding protein for the use of item 33, wherein the IFN or a functional fragment thereof is IFNλ, or a functional fragment thereof.
37. The interferon-associated antigen binding protein for the use of item 36, wherein the IFNλ or the functional fragment thereof is IFNλ2, or a functional fragment thereof.
38. The interferon-associated antigen binding protein for the use of item 37, wherein the IFNλ2 comprises the sequence as set forth in SEQ ID NO 18, or a sequence at least 90%, preferably 95%, more preferably 98%, or still more preferably 99% identical thereto.
39. The interferon-associated antigen binding protein for the use of any one of the preceding items, wherein the IFN or the functional fragment thereof is non-covalently associated with the agonistic anti-CD40 antibody or the agonistic antigen binding fragment thereof.
40. The interferon-associated antigen binding protein for the use of item 39, wherein the IFN or the functional fragment thereof is non-covalently associated with the agonistic anti-CD40 antibody or the agonistic antigen binding fragment thereof via ionic, Van-der-Waals, and/or hydrogen bond interactions.
41. The interferon-associated antigen binding protein for the use of any one of items 1 to 38, wherein the IFN or the functional fragment thereof is covalently associated with the agonistic anti-CD40 antibody or the agonistic antigen binding fragment thereof.
42. The interferon-associated antigen binding protein for the use of item 41, wherein the IFN or the functional fragment thereof is fused to the agonistic anti-CD40 antibody or the agonistic antigen binding fragment thereof.
43. The interferon-associated antigen binding protein for the use of item 42, wherein the IFN or the functional fragment thereof is fused to a light chain of the agonistic anti-CD40 antibody or the agonistic antigen binding fragment thereof.
44. The interferon-associated antigen binding protein for the use of item 43, wherein the IFN or the functional fragment thereof is fused to the N-terminus of the light chain of the agonistic anti-CD40 antibody or the agonistic antigen binding fragment thereof.
45. The interferon-associated antigen binding protein for the use of item 43, wherein the IFN or the functional fragment thereof is fused to the C-terminus of the light chain of the agonistic anti-CD40 antibody or the agonistic antigen binding fragment thereof.
46. The interferon-associated antigen binding protein for the use of item 42, wherein the IFN or the functional fragment thereof is fused to a heavy chain of the agonistic anti-CD40 antibody or the agonistic antigen binding fragment thereof.
47. The interferon-associated antigen binding protein for the use of item 46, wherein the IFN or the functional fragment thereof is fused to the N-terminus of the heavy chain of the agonistic anti-CD40 antibody or the agonistic antigen binding fragment thereof.
48. The interferon-associated antigen binding protein for the use of item 46, wherein the IFN or the functional fragment thereof is fused to the C-terminus of the heavy chain of the agonistic anti-CD40 antibody or the agonistic antigen binding fragment thereof.
49. The interferon-associated antigen binding protein for the use of any one of items 42 to 48, wherein the agonistic anti-CD40 antibody or an agonistic antigen binding fragment thereof, and the IFN or the functional fragment thereof are fused to each other via a linker.
50. The interferon-associated antigen binding protein for the use of item 49, wherein the interferon-associated antigen binding protein comprises no amino acids other than those forming (I) said agonistic anti-CD40 antibody, or agonistic antigen binding fragment thereof, (II) said IFN or functional fragment thereof and (III) said linker.
51. The interferon-associated antigen binding protein for the use of any one of items 1 to 49, wherein the interferon-associated antigen binding protein comprises no amino acids other than those forming (I) said agonistic anti-CD40 antibody, or agonistic antigen binding fragment thereof and (II) said IFN or functional fragment thereof.
52. The interferon-associated antigen binding protein for the use of any one of items 49 to 50, wherein the linker is a peptide linker.
53. The interferon-associated antigen binding protein for the use of item 52, wherein the linker comprises at least 1, at least 2, at least 3, at least 4, or at least 5 amino acids.
54. The interferon-associated antigen binding protein for the use of item 53, wherein the linker comprises at least 4 amino acids.
55. The interferon-associated antigen binding protein for the use of item 53, wherein the linker comprises at least 11 amino acids.
56. The interferon-associated antigen binding protein for the use of item 53, wherein the linker comprises at least 12 amino acids.
57. The interferon-associated antigen binding protein for the use of item 53, wherein the linker comprises at least 13 amino acids.
58. The interferon-associated antigen binding protein for the use of item 53, wherein the linker comprises at least 15 amino acids.
59. The interferon-associated antigen binding protein for the use of item 53, wherein the linker comprises at least 20 amino acids.
60. The interferon-associated antigen binding protein for the use of item 53, wherein the linker comprises at least 21 amino acids.
61. The interferon-associated antigen binding protein for the use of item 53, wherein the linker comprises at least 24 amino acids.
62. The interferon-associated antigen binding protein for the use of item 52, wherein the linker comprises up to 10, up to 20, up to 30, up to 40, up to 50, up to 60, up to 70, up to 80, up to 90, or up to 100 amino acids.
63. The interferon-associated antigen binding protein for the use of item 62, wherein the linker comprises up to 80 amino acids.
64. The interferon-associated antigen binding protein for the use of item 62, wherein the linker comprises up to 40 amino acids.
65. The interferon-associated antigen binding protein for the use of item 62, wherein the linker comprises up to 24 amino acids.
66. The interferon-associated antigen binding protein for the use of item 62, wherein the linker comprises up to 21 amino acids.
67. The interferon-associated antigen binding protein for the use of item 62, wherein the linker comprises up to 20 amino acids.
68. The interferon-associated antigen binding protein for the use of item 62, wherein the linker comprises up to 15 amino acids.
69. The interferon-associated antigen binding protein for the use of item 62, wherein the linker comprises up to 13 amino acids.
70. The interferon-associated antigen binding protein for the use of item 62, wherein the linker comprises up to 12 amino acids.
71. The interferon-associated antigen binding protein for the use of item 62, wherein the linker comprises up to 11 amino acids.
72. The interferon-associated antigen binding protein for the use of item 62, wherein the linker comprises up to 4 amino acids.
73. The interferon-associated antigen binding protein for the use of any one of items 52 to 72, wherein the linker is selected from the group comprising acidic, basic and neutral linkers.
74. The interferon-associated antigen binding protein for the use of item 73, wherein the linker is an acidic linker.
75. The interferon-associated antigen binding protein for the use of item 73 or 74, wherein the linker comprises a sequence as set forth in SEQ ID NO 22 or SEQ ID NO 23.
76. The interferon-associated antigen binding protein for the use of item 73, wherein the linker is a basic linker.
77. The interferon-associated antigen binding protein for the use of item 73, wherein the linker is a neutral linker.
78. The interferon-associated antigen binding protein for the use of item 73 or 77, wherein the linker comprises a sequence as set forth in SEQ ID NO 20, SEQ ID NO 21, SEQ ID NO 24, SEQ ID NO 25 or SEQ ID NO 26.
79. The interferon-associated antigen binding protein for the use of any one of items 52 to 78, wherein the linker is selected from the group comprising rigid, flexible and helix-forming linkers.
80. The interferon-associated antigen binding protein for the use of item 79, wherein the linker is a rigid linker.
81. The interferon-associated antigen binding protein for the use of item 79 or 80, wherein the linker comprises a sequence as set forth in SEQ ID NO 20, SEQ ID NO 22 or SEQ ID NO 23.
82. The interferon-associated antigen binding protein for the use of item 79, wherein the linker is a flexible linker.
83. The interferon-associated antigen binding protein for the use of item 79 or 82, wherein the linker comprises a sequence as set forth in SEQ ID NO 21, SEQ ID NO 24, SEQ ID NO 25 or SEQ ID NO 26.
84. The interferon-associated antigen binding protein for the use of item 79, wherein the linker is a helix-forming linker.
85. The interferon-associated antigen binding protein for the use of item 79 or 84, wherein the linker comprises a sequence as set forth in SEQ ID NO 22 or SEQ ID NO 23.
86. The interferon-associated antigen binding protein for the use of any one of items 52 to 74, 76, 77, 79, 80, 82 or 84, wherein the linker comprises the amino acids glycine and serine.
87. The interferon-associated antigen binding protein for the use of item 86, wherein the linker comprises the sequence as set forth in SEQ ID NO 21, SEQ ID NO 24, SEQ ID NO 25, or SEQ ID NO 26.
88. The interferon-associated antigen binding protein for the use of item 86, wherein the linker further comprises the amino acid threonine.
89. The interferon-associated antigen binding protein for the use of item 88, wherein the linker comprises the sequence as set forth in SEQ ID NO 21.
90. The interferon-associated antigen binding protein for the use of item 52, wherein the linker comprises a sequence selected from the sequences as set forth in SEQ ID NOs 20 to 26.
91. The interferon-associated antigen binding protein for the use of item 90, wherein the linker comprises a sequence selected from the sequences as set forth in SEQ ID NO 24, SEQ ID NO 25 or SEQ ID NO 26.
92. The interferon-associated antigen binding protein for the use of item 91, wherein the linker comprises a sequence as set forth in SEQ ID NO 24.
93. The interferon-associated antigen binding protein for the use of item 91, wherein the linker comprises a sequence as set forth in SEQ ID NO 25.
94. The interferon-associated antigen binding protein for the use of item 91, wherein the linker comprises a sequence as set forth in SEQ ID NO 26.
95. The interferon-associated antigen binding protein for the use of any one of items 49, 50 or 52 to 94, wherein the IFN or a functional fragment thereof is fused to the C-terminus of a heavy chain of the agonistic anti-CD40 antibody, or the agonistic antigen binding fragment thereof, via the linker as set forth in Table 3, in particular Table 3A or Table 3B, more particularly Table 3A.
96. The interferon-associated antigen binding protein for the use of item 95, wherein the heavy chain of the agonistic anti-CD40 antibody, or the agonistic antigen binding fragment thereof, comprises a sequence as set forth in SEQ ID NO 6, SEQ ID NO 9, SEQ ID NO 12, SEQ ID NO 48 or SEQ ID NO 49.
97. The interferon-associated antigen binding protein for the use of items 95 or 96, wherein the IFNα2a comprises the sequence as set forth in SEQ ID NO 17.
98. The interferon-associated antigen binding protein for the use of items 95 or 96, wherein the IFNβ comprises the sequence as set forth in SEQ ID NO 14, SEQ ID NO 15 or SEQ ID NO 16.
99. The interferon-associated antigen binding protein for the use of item 98, wherein the IFNβ comprises the sequence as set forth in SEQ ID NO 14.
100. The interferon-associated antigen binding protein for the use of item 98, wherein the IFNβ comprises the sequence as set forth in SEQ ID NO 15.
101. The interferon-associated antigen binding protein for the use of item 98, wherein the IFNβ comprises the sequence as set forth in SEQ ID NO 16.
102. The interferon-associated antigen binding protein for the use of item 95 or 96, wherein the IFNγ comprises the sequence as set forth in SEQ ID NO 19.
103. The interferon-associated antigen binding protein for the use of item 95 or 96, wherein the IFNλ2 comprises the sequence as set forth in SEQ ID NO 18.
104. The interferon-associated antigen binding protein for the use of any one of items 95 to 103, wherein the interferon-associated antigen binding protein further comprises a light chain of an agonistic anti-CD40 antibody, or an agonistic antigen binding fragment thereof.
105. The interferon-associated antigen binding protein for the use of item 104, wherein the light chain comprises a sequence as set forth in SEQ ID NO 3.
106. The interferon-associated antigen binding protein for the use of any one of items 49, 50 or 52 to 94, wherein the IFN or a functional fragment thereof is fused to the N-terminus of a heavy chain of the agonistic anti-CD40 antibody, or the agonistic antigen binding fragment thereof, via the linker as set forth in Table 4, in particular Table 4A or Table 4B, more particularly Table 4A.
107. The interferon-associated antigen binding protein for the use of item 106, wherein the heavy chain of the agonistic anti-CD40 antibody, or the agonistic antigen binding fragment thereof, comprises a sequence as set forth in SEQ ID NO 6, SEQ ID NO 9, SEQ ID NO 49, SEQ ID NO 48, or SEQ ID NO 12.
108. The interferon-associated antigen binding protein for the use of items 106 or 107, wherein the IFNα2a comprises the sequence as set forth in SEQ ID NO 17.
109. The interferon-associated antigen binding protein for the use of items 106 or 107, wherein the IFNβ comprises the sequence as set forth in SEQ ID NO 14, SEQ ID NO 15 or SEQ ID NO 16.
110. The interferon-associated antigen binding protein for the use of item 109, wherein the IFNβ comprises the sequence as set forth in SEQ ID NO 14.
111. The interferon-associated antigen binding protein for the use of item 109, wherein the IFNβ comprises the sequence as set forth in SEQ ID NO 15.
112. The interferon-associated antigen binding protein for the use of item 109, wherein the IFNβ comprises the sequence as set forth in SEQ ID NO 16.
113. The interferon-associated antigen binding protein for the use of items 106 or 107, wherein the IFNγ comprises the sequence as set forth in SEQ ID NO 19.
114. The interferon-associated antigen binding protein for the use of items 106 or 107, wherein the IFNλ2 comprises the sequence as set forth in SEQ ID NO 18.
115. The interferon-associated antigen binding protein for the use of any one of items 106 to 114, wherein the interferon-associated antigen binding protein further comprises a light chain of an agonistic anti-CD40 antibody, or an agonistic antigen binding fragment thereof.
116. The interferon-associated antigen binding protein for the use of item 115, wherein the light chain comprises a sequence as set forth in SEQ ID NO 3.
117. The interferon-associated antigen binding protein for the use of any one of items 49, 50 or 52 to 94, wherein the IFN or a functional fragment thereof is fused to the C-terminus of a light chain of the agonistic anti-CD40 antibody, or the agonistic antigen binding fragment thereof, via the linker as set forth in Table 5, in particular Table 5A or Table 5B, more particularly Table 5A.
118. The interferon-associated antigen binding protein for the use of item 117, wherein the light chain of the agonistic anti-CD40 antibody, or the agonistic antigen binding fragment thereof, comprises a sequence as set forth in SEQ ID NO 3.
119. The interferon-associated antigen binding protein for the use of items 117 or 118, wherein the IFNα2a comprises the sequence as set forth in SEQ ID NO 17.
120. The interferon-associated antigen binding protein for the use of items 117 or 118, wherein the IFNβ comprises the sequence as set forth in SEQ ID NO 14, SEQ ID NO 15 or SEQ ID NO 16.
121. The interferon-associated antigen binding protein for the use of item 120, wherein the IFNβ comprises the sequence as set forth in SEQ ID NO 14.
122. The interferon-associated antigen binding protein for the use of item 120, wherein the IFNβ comprises the sequence as set forth in SEQ ID NO 15.
123. The interferon-associated antigen binding protein for the use of item 120, wherein the IFNβ comprises the sequence as set forth in SEQ ID NO 16.
124. The interferon-associated antigen binding protein for the use of items 117 or 118, wherein the IFNγ comprises the sequence as set forth in SEQ ID NO 19.
125. The interferon-associated antigen binding protein for the use of items 117 or 118, wherein the IFNλ2 comprises the sequence as set forth in SEQ ID NO 18.
126. The interferon-associated antigen binding protein for the use of any one of items 117 to 125, wherein the interferon-associated antigen binding protein further comprises a heavy chain of an agonistic anti-CD40 antibody, or an agonistic antigen binding fragment thereof.
127. The interferon-associated antigen binding protein for the use of item 126, wherein the heavy chain of the agonistic anti-CD40 antibody comprises a sequence as set forth in SEQ ID NO 6, SEQ ID NO 9, SEQ ID NO 49, SEQ ID NO 48, or SEQ ID NO 12.
128. The interferon-associated antigen binding protein for the use of any one of items 49, 50 or 52 to 94, wherein the IFN is fused to the N-terminus of a light chain of the agonistic anti-CD40 antibody, or the agonistic antigen binding fragment thereof, via the linker as set forth in Table 6, in particular Table 6A or Table 6B, more particularly Table 6A.
129. The interferon-associated antigen binding protein for the use of item 128, wherein the light chain of the agonistic anti-CD40 antibody, or the agonistic antigen binding fragment thereof, comprises a sequence as set forth in SEQ ID NO 3.
130. The interferon-associated antigen binding protein for the use of items 128 or 129, wherein the IFNα2a comprises the sequence as set forth in SEQ ID NO 17.
131. The interferon-associated antigen binding protein for the use of items 128 or 129, wherein the IFNβ comprises the sequence as set forth in SEQ ID NO 14, SEQ ID NO 15 or SEQ ID NO 16.
132. The interferon-associated antigen binding protein for the use of item 131, wherein the IFNβ comprises the sequence as set forth in SEQ ID NO 14.
133. The interferon-associated antigen binding protein for the use of item 131, wherein the IFNβ comprises the sequence as set forth in SEQ ID NO 15.
134. The interferon-associated antigen binding protein for the use of item 131, wherein the IFNβ comprises the sequence as set forth in SEQ ID NO 16.
135. The interferon-associated antigen binding protein for the use of items 128 or 129, wherein the IFNγ comprises the sequence as set forth in SEQ ID NO 19.
136. The interferon-associated antigen binding protein for the use of items 128 or 129, wherein the IFNλ2 comprises the sequence as set forth in SEQ ID NO 18.
137. The interferon-associated antigen binding protein for the use of any one of items 128 to 136, wherein the interferon-associated antigen binding protein further comprises a heavy chain of an anti-CD40 antibody, or an agonistic antigen binding fragment thereof.
138. The interferon-associated antigen binding protein for the use of item 137, wherein the heavy chain of the agonistic anti-CD40 antibody comprises a sequence as set forth in SEQ ID NO 6, SEQ ID NO 9, SEQ ID NO 49, SEQ ID NO 48, or SEQ ID NO 12.
139. The interferon-associated antigen binding protein for the use of any one of items 1 to 138, wherein the interferon-associated antigen binding protein comprises a sequence selected from SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 81, SEQ ID NO 82, SEQ ID NO 83, SEQ ID NO 84, SEQ ID NO 85, SEQ ID NO 86, SEQ ID NO 87, SEQ ID NO 88 and SEQ ID NO 94.
140. The interferon-associated antigen binding protein for the use of item 139, wherein the interferon-associated antigen binding protein comprises a sequence selected from SEQ ID NO 38, SEQ ID NO 39, SEQ ID NO 40, SEQ ID NO 41, SEQ ID NO 42 or SEQ ID NO 43.
141. The interferon-associated antigen binding protein for the use of items 139 or 140, wherein the interferon-associated antigen binding protein is an interferon-fused agonistic anti-CD40 antibody or an interferon-fused agonistic antigen binding fragment thereof comprising one of the sequence combinations disclosed in Table 9, in particular Table 9A or Table 9B, more particularly Table 9A.
142. The interferon-associated antigen binding protein for the use of item 141, wherein the interferon-associated antigen binding protein is an interferon-fused agonistic anti-CD40 antibody or an interferon-fused agonistic antigen binding fragment thereof comprising the sequences as set forth in SEQ ID NO 38 and SEQ ID NO 3.
143. The interferon-associated antigen binding protein for the use of item 141, wherein the interferon-associated antigen binding protein is an interferon-fused agonistic anti-CD40 antibody or an interferon-fused agonistic antigen binding fragment thereof comprising the sequences as set forth in SEQ ID NO 39 and SEQ ID NO 3.
144. The interferon-associated antigen binding protein for the use of item 141, wherein the interferon-associated antigen binding protein is an interferon-fused agonistic anti-CD40 antibody or an interferon-fused agonistic antigen binding fragment thereof comprising the sequences as set forth in SEQ ID NO 40 and SEQ ID NO 3.
145. The interferon-associated antigen binding protein for the use of item 141, wherein the interferon-associated antigen binding protein is an interferon-fused agonistic anti-CD40 antibody or an interferon-fused agonistic antigen binding fragment thereof comprising the sequences as set forth in SEQ ID NO 41 and SEQ ID NO 9.
146. The interferon-associated antigen binding protein for the use of item 141, wherein the interferon-associated antigen binding protein is an interferon-fused agonistic anti-CD40 antibody or an interferon-fused agonistic antigen binding fragment thereof comprising the sequences as set forth in SEQ ID NO 42 and SEQ ID NO 9.
147. The interferon-associated antigen binding protein for the use of item 141, wherein the interferon-associated antigen binding protein is an interferon-fused agonistic anti-CD40 antibody or an interferon-fused agonistic antigen binding fragment thereof comprising the sequences as set forth in SEQ ID NO 43 and SEQ ID NO 9.
148. The interferon-associated antigen binding protein for the use of any one of items 1 to 147, wherein the interferon-associated antigen binding protein activates both the CD40 and an IFN pathway.
149. The interferon-associated antigen binding protein for the use of item 148, wherein CD40 activity is determined using a whole blood surface molecule upregulation assay or an in vitro reporter cell assay.
150. The interferon-associated antigen binding protein for the use of item 149, wherein CD40 activity is determined using an in vitro reporter cell assay, optionally using HEK-Blue™ CD40L cells.
151. The interferon-associated antigen binding protein for the use of any one of items 148 to 150, wherein the interferon-associated antigen binding protein activates the CD40 pathway with an EC₅₀ of less than 400, 300, 200, 150, 100, 70, 60, 50, 40, 30, 25, 20, or 15 ng/mL.
152. The interferon-associated antigen binding protein for the use of item 151, wherein the interferon-associated antigen binding protein activates the CD40 pathway with an EC₅₀ ranging from 10 to 200 ng/mL.
153. The interferon-associated antigen binding protein for the use of item 152, wherein the interferon-associated antigen binding protein activates the CD40 pathway with an EC₅₀ ranging from 10 to 50 ng/mL, preferably 10 to 30 ng/mL.
154. The interferon-associated antigen binding protein for the use of any one of items 148 to 153, wherein the interferon-associated antigen binding protein activates the IFN pathway with an EC₅₀ of less than 100, 60, 50, 40, 30, 20, 10, or 1 ng/mL.
155. The interferon-associated antigen binding protein for the use of any one of items 148 to 154, wherein the interferon-associated antigen binding protein activates the IFN pathway with an EC₅₀ of less than 11 ng/mL, preferably less than 6 ng/mL.
156. The interferon-associated antigen binding protein for the use of any one of items 148 to 155, wherein the IFN pathway is the IFNα, IFNβ, IFNε, IFNγ, IFNω or IFNλ pathway.
157. The interferon-associated antigen binding protein for the use of item 156, wherein IFNβ activity is determined using an in vitro reporter cell assay, optionally using HEK-Blue™ IFN-α/β cells.
158. The interferon-associated antigen binding protein for the use of item 156, wherein IFNα activity is determined using an in vitro reporter cell assay, optionally using HEK-Blue™ IFN-α/β cells.
159. The interferon-associated antigen binding protein for the use of item 156, wherein IFNγ activity is determined using an in vitro reporter cell assay, optionally using HEK-Blue™ Dual IFN-γ cells.
160. The interferon-associated antigen binding protein for the use of item 156, wherein IFNλ activity is determined using an in vitro reporter cell assay, optionally using HEK-Blue™ IFN-λ cells.
161. The interferon-associated antigen binding protein for the use of any one of the preceding items, in particular items 148 to 160, wherein the expression level of one or more IFN pathway biomarkers is upregulated in a Coronavirus-infected cell upon treatment with the interferon-associated antigen binding protein, preferably at least 1.5-fold, more preferably at least 2-fold, most preferably at least 3-fold, as compared to the expression level of said biomarkers in said Coronavirus-infected cell that has not been treated with the interferon-associated antigen binding protein.
162. The interferon-associated antigen binding protein for the use of item 161, wherein the IFN pathway biomarker is a chemokine.
163. The interferon-associated antigen binding protein for the use of item 162, wherein the IFN pathway biomarker is the interferon stimulated gene ISG20.
164. The interferon-associated antigen binding protein for the use of item 162, wherein the IFN pathway biomarker is a C-X-C chemokine, selected from the group consisting of CXCL9, CXCL10 and CXCL11.
165. The interferon-associated antigen binding protein for the use of item 164, wherein the IFN pathway biomarker is CXCL10.
166. The interferon-associated antigen binding protein for the use of any one of the preceding items, in particular items 148 to 165, wherein the expression level of one or more of IL10, IL1β and IL2 is not significantly upregulated in a Coronavirus-infected cell upon treatment with the interferon-associated antigen binding protein, as compared to the expression level of said interleukins in said Coronavirus-infected cell that has not been treated with the interferon-associated antigen binding protein.
167. The interferon-associated antigen binding protein for the use of any one of the preceding items, wherein the systemic exposure of the interferon-associated antigen binding protein is increased compared to antibody CP870,893, preferably by at least 10%, more preferably by at least 15%, most preferably by at least 25%.
168. The interferon-associated antigen binding protein for the use of any one of the preceding items, wherein the systemic exposure of the interferon-associated antigen binding protein is at least 1000 μg*h/mL.
169. The interferon-associated antigen binding protein for the use of item 168, wherein the systemic exposure of the interferon-associated antigen binding protein ranges from 1033 μg*h/mL to 1793 μg*h/mL.
170. The interferon-associated antigen binding protein for the use of any one of the preceding items, wherein the half-life of the interferon-associated antigen binding protein is at least 100 h.
171. The interferon-associated antigen binding protein for the use of item 170, wherein the half-life of the interferon-associated antigen binding protein ranges from 116 to 158 h.
172. The interferon-associated antigen binding protein for the use of any one of the preceding items, wherein the clearance rate of the interferon-associated antigen binding protein is below 0.5 mL/h/kg.
173. The interferon-associated antigen binding protein for the use of item 172, wherein the clearance of the interferon-associated antigen binding protein ranges from 0.28 to 0.49 mL/h/kg.
174. The interferon-associated antigen binding protein for the use of any one of items 1 to 173, wherein the volume of distribution Vss of the interferon-associated antigen binding protein is below 100 mL/kg.
175. The interferon-associated antigen binding protein for the use of item 174, wherein the volume of distribution Vss of the interferon-associated antigen binding protein ranges from 50 to 98 mL/kg.
176. The interferon-associated antigen binding protein for the use of any one of the preceding items, wherein the use comprises administering the interferon-associated antigen binding protein to a subject in need thereof by means of genetic delivery with RNA or DNA sequences encoding the interferon-associated antigen binding protein, or a vector or vector system encoding the interferon-associated antigen binding protein.
177. The interferon-associated antigen binding protein for the use of any one of items 1 to 176, wherein the interferon-associated antigen binding protein is comprised in a pharmaceutical composition.
178. The interferon-associated antigen binding protein for the use of item 177, wherein the pharmaceutical composition is suitable for oral, parenteral, or topical administration or for administration by inhalation.
179. The interferon-associated antigen binding protein for the use of item 178, wherein the pharmaceutical composition is suitable for oral administration.
180. The interferon-associated antigen binding protein for the use of item 178, wherein the pharmaceutical composition is suitable for topical administration.
181. The interferon-associated antigen binding protein for the use of item 178, wherein the pharmaceutical composition is suitable for administration by inhalation.
182. The interferon-associated antigen binding protein for the use of item 178, wherein the pharmaceutical composition is suitable for parenteral administration.
183. The interferon-associated antigen binding protein for the use of item 182, wherein the pharmaceutical composition is suitable for intravenous, intraarterial, intraperitoneal, intramuscular, subcutaneous, rectal or vaginal administration.
184. The interferon-associated antigen binding protein for the use of item 183, wherein the pharmaceutical composition is suitable for injection, preferably for intravenous or intraarterial injection or drip.
185. The interferon-associated antigen binding protein for the use of any one of items 177 to 184, wherein the pharmaceutical composition comprises at least one buffering agent.
186. The interferon-associated antigen binding protein for the use of item 185, wherein the buffering agent is acetate, formate or citrate.
187. The interferon-associated antigen binding protein for the use of item 186, wherein the buffering agent is acetate.
188. The interferon-associated antigen binding protein for the use of item 186, wherein the buffering agent is formate.
189. The interferon-associated antigen binding protein for the use of item 186, wherein the buffering agent is citrate.
190. The interferon-associated antigen binding protein for the use of any one of items 177 to 189, wherein the pharmaceutical composition comprises a surfactant.
191. The interferon-associated antigen binding protein for the use of item 190, wherein the surfactant is selected from the list comprising pluronics, PEG, sorbitan esters, polysorbates, triton, tromethamine, lecithin, cholesterol and tyloxapal.
192. The interferon-associated antigen binding protein for the use of item 191, wherein the surfactant is polysorbate.
193. The interferon-associated antigen binding protein for the use of item 192, wherein the surfactant is polysorbate 20, polysorbate 40, polysorbate 60, polysorbate 80 or polysorbate 100.
194. The interferon-associated antigen binding protein for the use of item 193, wherein the surfactant is polysorbate 20.
195. The interferon-associated antigen binding protein for the use of item 193, wherein the surfactant is polysorbate 80.
196. The interferon-associated antigen binding protein for the use of any one of items 177 to 195, wherein the pharmaceutical composition comprises a stabilizing agent, optionally wherein the stabilizing agent is albumin.
197. The interferon-associated antigen binding protein for the use according to any one of items 1 to 196, wherein the agonistic anti-CD40 antibody or agonistic antigen binding fragment thereof comprises three light chain complementarity determining regions (CDRs) CDRL1, CDRL2 and CDRL3 that are at least 90% identical to the respective CDRL1, CDRL2 and CDRL3 sequences within SEQ ID NO 3; and three heavy chain CDRs, CDRH1, CDRH2 and CDRH3, that are at least 90% identical to the respective CDRH1, CDRH2 and CDRH3 sequences within SEQ ID NO 6.
198. The interferon-associated antigen binding protein for the use according to any one of items 1 to 197, wherein the agonistic anti-CD40 antibody or agonistic antigen binding fragment thereof comprises three light chain complementarity determining regions (CDRs) CDRL1, CDRL2 and CDRL3 that are identical to the respective CDRL1, CDRL2 and CDRL3 sequences within SEQ ID NO 3; and three heavy chain CDRs, CDRH1, CDRH2 and CDRH3, that are identical to the respective CDRH1, CDRH2 and CDRH3 sequences within SEQ ID NO 6.
199. The interferon-associated antigen binding protein for the use according to item 197 or item 198, wherein each CDR is defined in accordance with the Kabat definition, the Chothia definition, the AbM definition, or the contact definition of CDR; preferably wherein each CDR is defined in accordance with the CDR definition of Kabat or the CDR definition of Chothia.
200. The interferon-associated antigen binding protein for its use according to any one of items 2 to 199, wherein no amino acid substitutions, insertions or deletions are present within the complementarity determining regions of the heavy chain and light chain variable regions of the agonistic anti-CD40 antibody or agonistic antigen binding fragment thereof.
201. The interferon-associated antigen binding protein for its use according to any one of items 2 to 199, wherein no, one or two amino acid substitutions, insertions or deletions are independently present within each of the complementarity determining regions of the heavy chain and light chain variable regions of the agonistic anti-CD40 antibody or agonistic antigen binding fragment thereof.
202. A polynucleotide or polynucleotides encoding the interferon-associated antigen binding protein as defined in any one of the preceding items for use in the treatment or prevention of a Coronavirus infection in a subject in need thereof, wherein the treatment comprises expression in said subject of said interferon-associated antigen binding protein, from a polynucleotide or polynucleotides administered to said subject.
203. The interferon-associated antigen binding protein, or the polynucleotide or polynucleotides for their use according to any one of the preceding items, wherein the Coronavirus is selected from the group consisting of severe acute respiratory syndrome coronavirus 1 (SARS-CoV-1), severe acute respiratory syndrome coronavirus 2 (SARS-CoV-2) and Middle East respiratory syndrome coronavirus (MERS-CoV).
204. The interferon-associated antigen binding protein, or the polynucleotide or polynucleotides for their use according any one of the preceding items, wherein the Coronavirus is SARS-CoV-2 or a variant thereof.
205. The interferon-associated antigen binding protein, or the polynucleotide or polynucleotides for their use according to item 204, wherein the Coronavirus is a variant of SARS-CoV-2 as identified in the lineage reports available from https.//outbreak.info/situation-reports or https.//cov-lineages.org/global_report.html.
206. The interferon-associated antigen binding protein, or the polynucleotide or polynucleotides for their use according to item 204 or 205, wherein the SARS-CoV-2 variant is selected from the group consisting of A.23.1, B.1.1.7, B.1.351, B.1.427, B.1.429, B.1.525, B.1.526, B.1.526.1, B.1.526.2, B.1.617, B.1.617.1, B.1.617.2, B.1.617.3, P.1 or P.2.

### Matters

I view of the above, it will furthermore be appreciated that the present invention also relates to the following matters:
1. A tumor necrosis factor receptor superfamily (TNFRSF) agonist or a functional fragment thereof for use in the treatment or prevention of a Coronavirus infection, wherein the TNFRSF agonist or a functional fragment thereof is administered in combination with an interferon (IFN) or a functional fragment thereof.
2. An interferon (IFN) or a functional fragment thereof for use in the treatment or prevention of a Coronavirus infection, wherein the IFN or a functional fragment thereof is administered in combination with a tumor necrosis factor receptor superfamily (TNFRSF) agonist or a functional fragment thereof.
3. A combination of a tumor necrosis factor receptor superfamily (TNFRSF) agonist or a functional fragment thereof and an interferon (IFN) or a functional fragment thereof, for use in the treatment or prevention of a Coronavirus infection.
4. The TNFRSF agonist or functional fragment thereof, the IFN or functional fragment thereof, or the combination for their use according to any one of matters 1 to 3, wherein the TNFRSF agonist or functional fragment thereof is selected from the group consisting of a CD27 agonist, a CD30 agonist, a cluster of differentiation factor 40 (CD40) agonist, a HVEM agonist, an OX40 agonist, a TNFRSF12A agonist and a 4-1BB agonist, or functional fragments thereof.
5. The TNFRSF agonist or functional fragment thereof, the IFN or functional fragment thereof, or the combination for their use according to any one of the preceding matters, wherein the TNFRSF agonist or functional fragment thereof is a polypeptide or functional fragment thereof, or an antibody or functional fragment thereof.
6. The TNFRSF agonist or functional fragment thereof, the IFN or functional fragment thereof, or the combination for their use according to any one of matters 1 to 5, wherein the TNFRSF agonist or functional fragment thereof is selected from CD70, CD30L (TNFSF8), CD40L, LIGHT, OX40L, TWEAK and 4-1BBL, or functional fragments thereof, preferably wherein the TNFRSF agonist or functional fragment thereof is selected from CD40L, LIGHT and TWEAK, or functional fragments thereof.
7. The TNFRSF agonist or functional fragment thereof, the IFN or functional fragment thereof, or the combination for their use according to any one of matters 1 to 5, wherein the TNFRSF agonist or functional fragment thereof is an agonistic anti-CD40 antibody or an agonistic antigen binding fragment thereof.
8. The TNFRSF agonist or functional fragment thereof, the IFN or functional fragment thereof, or the combination for their use according to matter 7, wherein the agonistic anti-CD40 antibody or agonistic antigen binding fragment thereof comprises three light chain complementarity determining regions (CDRs) CDRL1, CDRL2 and CDRL3 that are at least 90% identical to the respective CDRL1, CDRL2 and CDRL3 sequences within SEQ ID NO 3; and three heavy chain CDRs, CDRH1, CDRH2 and CDRH3, that are at least 90% identical to the respective CDRH1, CDRH2 and CDRH3 sequences within SEQ ID NO 6;
   preferably wherein the agonistic anti-CD40 antibody or agonistic antigen binding fragment thereof comprises three light chain complementarity determining regions (CDRs) CDRL1, CDRL2 and CDRL3 that are at least 95% identical to the respective CDRL1, CDRL2 and CDRL3 sequences within SEQ ID NO 3; and three heavy chain CDRs, CDRH1, CDRH2 and CDRH3, that are at least 95% identical to the respective CDRH1, CDRH2 and CDRH3 sequences within SEQ ID NO 6;
   more preferably wherein the agonistic anti-CD40 antibody or agonistic antigen binding fragment thereof comprises three light chain complementarity determining regions (CDRs) CDRL1, CDRL2 and CDRL3 that are at least 98% identical to the respective CDRL1, CDRL2 and CDRL3 sequences within SEQ ID NO 3; and three heavy chain CDRs, CDRH1, CDRH2 and CDRH3, that are at least 98% identical to the respective CDRH1, CDRH2 and CDRH3 sequences within SEQ ID NO 6;
   or still more preferably wherein the agonistic anti-CD40 antibody or agonistic antigen binding fragment thereof comprises three light chain complementarity determining regions (CDRs) CDRL1, CDRL2 and CDRL3 that are at least 99% identical to the respective CDRL1, CDRL2 and CDRL3 sequences within SEQ ID NO 3; and three heavy chain CDRs, CDRH1, CDRH2 and CDRH3, that are at least 99% identical to the respective CDRH1, CDRH2 and CDRH3 sequences within SEQ ID NO 6.
9. The TNFRSF agonist or functional fragment thereof, the IFN or functional fragment thereof, or the combination for their use according to matter 8, wherein the agonistic anti-CD40 antibody or agonistic antigen binding fragment thereof comprises three light chain complementarity determining regions (CDRs) CDRL1, CDRL2 and CDRL3 that are identical to the respective CDRL1, CDRL2 and CDRL3 sequences within SEQ ID NO 3; and three heavy chain CDRs, CDRH1, CDRH2 and CDRH3, that are identical to the respective CDRH1, CDRH2 and CDRH3 sequences within SEQ ID NO 6.
10. The TNFRSF agonist or functional fragment thereof, the IFN or functional fragment thereof, or the combination for their use according to matter 8 or matter 9, wherein each CDR is defined in accordance with the Kabat definition, the Chothia definition, the AbM definition, or the contact definition of CDR; preferably wherein each CDR is defined in accordance with the CDR definition of Kabat or the CDR definition of Chothia.
11. The TNFRSF agonist or functional fragment thereof, the IFN or functional fragment thereof, or the combination for their use according to matter 7, wherein the agonistic anti-CD40 antibody or agonistic antigen binding fragment thereof comprises
   (I)
   (a) a heavy chain or a fragment thereof comprising a complementarity determining region (CDR) CDRH1 that is at least 90% identical to SEQ ID NO 56, a CDRH2 that is at least 90% identical to SEQ ID NO 57, and a CDRH3 that is at least 90% identical to SEQ ID NO 58; and
   (b) a light chain or a fragment thereof comprising a CDRL1 that is at least 90% identical to SEQ ID NO 52, a CDRL2 that is at least 90% identical to SEQ ID NO 53, and a CDRL3 that is at least 90% identical to SEQ ID NO 54;
   preferably (II)
   (a) a heavy chain or a fragment thereof comprising a complementarity determining region (CDR) CDRH1 that is at least 95% identical to SEQ ID NO 56, a CDRH2 that is at least 95% identical to SEQ ID NO 57, and a CDRH3 that is at least 95% identical to SEQ ID NO 58; and
   (b) a light chain or a fragment thereof comprising a CDRL1 that is at least 95% identical to SEQ ID NO 52, a CDRL2 that is at least 95% identical to SEQ ID NO 53, and a CDRL3 that is at least 95% identical to SEQ ID NO 54;
   more preferably (III)
   (a) a heavy chain or a fragment thereof comprising a complementarity determining region (CDR) CDRH1 that is at least 98% identical to SEQ ID NO 56, a CDRH2 that is at least 98% identical to SEQ ID NO 57, and a CDRH3 that is at least 98% identical to SEQ ID NO 58; and
   (b) a light chain or a fragment thereof comprising a CDRL1 that is at least 98% identical to SEQ ID NO 52, a CDRL2 that is at least 98% identical to SEQ ID NO 53, and a CDRL3 that is at least 98% identical to SEQ ID NO 54; or
   still more preferably (IV)
   (a) a heavy chain or a fragment thereof comprising a complementarity determining region (CDR) CDRH1 that is at least 99% identical to SEQ ID NO 56, a CDRH2 that is at least 99% identical to SEQ ID NO 57, and a CDRH3 that is at least 99% identical to SEQ ID NO 58; and
   (b) a light chain or a fragment thereof comprising a CDRL1 that is at least 99% identical to SEQ ID NO 52, a CDRL2 that is at least 99% identical to SEQ ID NO 53, and a CDRL3 that is at least 99% identical to SEQ ID NO 54.
12. The TNFRSF agonist or functional fragment thereof, the IFN or functional fragment thereof, or the combination for their use according to matter 11, wherein the agonistic anti-CD40 antibody or agonistic antigen binding fragment thereof comprises
   a. a heavy chain or a fragment thereof comprising a complementarity determining region (CDR) CDRH1 that is identical to SEQ ID NO 56, a CDRH2 that is identical to SEQ ID NO 57, and a CDRH3 that is identical to SEQ ID NO 58; and
   b. a light chain or a fragment thereof comprising a CDRL1 that is identical to SEQ ID NO 52, a CDRL2 that is identical to SEQ ID NO 53, and a CDRL3 that is identical to SEQ ID NO 54.
13. The TNFRSF agonist or functional fragment thereof, the IFN or functional fragment thereof, or the combination for their use according to any one of matters 7 to 12, wherein the agonistic anti-CD40 antibody or agonistic antigen binding fragment thereof comprises a light chain variable region VL comprising the sequence as set forth in SEQ ID NO 51, or a sequence at least 90% identical thereto; and/or a heavy chain variable region VH comprising the sequence as set forth in SEQ ID NO 55, or a sequence at least 90% identical thereto
   preferably wherein the agonistic anti-CD40 antibody or agonistic antigen binding fragment thereof comprises a light chain variable region VL comprising a sequence that is at least 95% identical to the sequence as set forth in SEQ ID NO 51; and/or a heavy chain variable region VH comprising a sequence that is at least 95% identical to the sequence as set forth in SEQ ID NO 55;
   more preferably wherein the agonistic anti-CD40 antibody or agonistic antigen binding fragment thereof comprises a light chain variable region VL comprising a sequence that is at least 98% identical to the sequence as set forth in SEQ ID NO 51; and/or a heavy chain variable region VH comprising a sequence that is at least 98% identical to the sequence as set forth in SEQ ID NO 55;
   still more preferably wherein the agonistic anti-CD40 antibody or agonistic antigen binding fragment thereof comprises a light chain variable region VL comprising a sequence that is at least 99% identical to the sequence as set forth in SEQ ID NO 51; and/or a heavy chain variable region VH comprising a sequence that is at least 99% identical to the sequence as set forth in SEQ ID NO 55; or
   most preferably wherein the agonistic anti-CD40 antibody or agonistic antigen binding fragment thereof comprises a light chain variable region VL comprising the sequence as set forth in SEQ ID NO 51 and a heavy chain variable region VH comprising the sequence as set forth in SEQ ID NO 55.
14. The TNFRSF agonist or functional fragment thereof, the IFN or functional fragment thereof, or the combination for their use according to any one of matters 7 to 13, wherein the heavy chain of the agonistic anti-CD40 antibody or agonistic antigen binding fragment thereof comprises a Fab region heavy chain comprising an amino acid sequence as set forth in SEQ ID NO 12, or a sequence at least 90%, preferably 95%, more preferably 98%, or still more preferably 99% identical thereto.
15. The TNFRSF agonist or functional fragment thereof, the IFN or functional fragment thereof, or the combination for their use according to any one of matters 7 to 14, wherein the agonistic anti-CD40 antibody or agonistic antigen binding fragment thereof comprises a light chain (LC) that comprises a sequence as set forth in SEQ ID NO 3, or a sequence at least 90% identical thereto; and/or a heavy chain (HC) that comprises a sequence selected from the group consisting of SEQ ID NO 6, SEQ ID NO 9, SEQ ID NO 49 and SEQ ID NO 48, or a sequence at least 90% identical thereto preferably wherein the agonistic anti-CD40 antibody or agonistic antigen binding fragment thereof comprises a light chain (LC) that comprises a sequence that is at least 95% identical to the sequence as set forth in SEQ ID NO 3; and/or a heavy chain (HC) that comprises a sequence that is at least 95% identical to a sequence as set forth within the group of sequences consisting of SEQ ID NO 6, SEQ ID NO 9, SEQ ID NO 49 and SEQ ID NO 48;
   more preferably wherein the agonistic anti-CD40 antibody or agonistic antigen binding fragment thereof comprises a light chain (LC) that comprises a sequence that is at least 98% identical to the sequence as set forth in SEQ ID NO 3; and/or a heavy chain (HC) that comprises a sequence that is at least 98% identical to a sequence as set forth within the group of sequences consisting of SEQ ID NO 6, SEQ ID NO 9, SEQ ID NO 49 and SEQ ID NO 48;
   still more preferably wherein the agonistic anti-CD40 antibody or agonistic antigen binding fragment thereof comprises a light chain (LC) that comprises a sequence that is at least 99% identical to the sequence as set forth in SEQ ID NO 3; and/or a heavy chain (HC) that comprises a sequence that is at least 99% identical to a sequence as set forth within the group of sequences consisting of SEQ ID NO 6, SEQ ID NO 9, SEQ ID NO 49 and SEQ ID NO 48; or
   most preferably wherein the agonistic anti-CD40 antibody or the agonistic antigen binding fragment thereof comprises a light chain (LC) that comprises a sequence as set forth in SEQ ID NO 3; and a heavy chain (HC) that comprises a sequence selected from the group consisting of SEQ ID NO 6, SEQ ID NO 9, SEQ ID NO 49 and SEQ ID NO 48.
16. The TNFRSF agonist or functional fragment thereof, the IFN or functional fragment thereof, or the combination for their use according to matter 15, wherein the HC comprises the sequence as set forth in SEQ ID NO 6, or a sequence at least 90%, preferably 95%, more preferably 98%, or still more preferably 99% identical thereto.
17. The TNFRSF agonist or functional fragment thereof, the IFN or functional fragment thereof, or the combination for their use according to matter 15, wherein the HC comprises the sequence as set forth in SEQ ID NO 9, or a sequence at least 90%, preferably 95%, more preferably 98%, or still more preferably 99% identical thereto.
18. The TNFRSF agonist or functional fragment thereof, the IFN or functional fragment thereof, or the combination for their use according to matter 15, wherein the HC comprises the sequence as set forth in SEQ ID NO 49, or a sequence at least 90%, preferably 95%, more preferably 98%, or still more preferably 99% identical thereto.
19. The TNFRSF agonist or functional fragment thereof, the IFN or functional fragment thereof, or the combination for their use according to matter 15, wherein the HC comprises the sequence as set forth in SEQ ID NO 48, or a sequence at least 90%, preferably 95%, more preferably 98%, or still more preferably 99% identical thereto.
20. The TNFRSF agonist or functional fragment thereof, the IFN or functional fragment thereof, or the combination for their use according to any one of matters 13 to 19, wherein no amino acid substitutions, insertions or deletions are present within the complementarity determining regions of the heavy chain and light chain variable regions of the agonistic anti-CD40 antibody or agonistic antigen binding fragment thereof.
21. The TNFRSF agonist or functional fragment thereof, the IFN or functional fragment thereof, or the combination for their use according to any one of matters 8 to 19, wherein no, one or two amino acid substitutions, insertions or deletions are independently present within each of the complementarity determining regions of the heavy chain and light chain variable regions of the agonistic anti-CD40 antibody or agonistic antigen binding fragment thereof.
22. The TNFRSF agonist or functional fragment thereof, the IFN or functional fragment thereof, or the combination for their use according to any one of the preceding matters, wherein said IFN or functional fragment thereof is a human interferon.
23. The TNFRSF agonist or functional fragment thereof, the IFN or functional fragment thereof, or the combination for their use according to any one of the preceding matters, wherein the IFN or functional fragment thereof is selected from the group consisting of a Type I IFN, a Type II IFN and a Type III IFN, or functional fragments thereof.
24. The TNFRSF agonist or functional fragment thereof, the IFN or functional fragment thereof, or the combination for their use according to any one of the preceding matters, wherein the IFN or the functional fragment thereof is a Type I IFN, or a functional fragment thereof.
25. The TNFRSF agonist or functional fragment thereof, the IFN or functional fragment thereof, or the combination for their use according to matter 24, wherein the type I IFN or the functional fragment thereof is IFNα, IFNβ, IFNω, or IFNε, or a functional fragment thereof.
26. The TNFRSF agonist or functional fragment thereof, the IFN or functional fragment thereof, or the combination for their use according to matter 24, wherein the type I IFN or the functional fragment thereof is IFNα or IFNβ, or a functional fragment thereof.
27. The TNFRSF agonist or functional fragment thereof, the IFN or functional fragment thereof, or the combination for their use according to matter 24, wherein the type I IFN or the functional fragment thereof is IFNω, or a functional fragment thereof.
28. The TNFRSF agonist or functional fragment thereof, the IFN or functional fragment thereof, or the combination for their use according to matter 24, wherein the type I IFN or the functional fragment thereof is IFNε, or a functional fragment thereof.
29. The TNFRSF agonist or functional fragment thereof, the IFN or functional fragment thereof, or the combination for their use according to any one of matters 1 to 23, wherein the IFN or functional fragment thereof is IFNα, IFNβ, IFNγ, IFNλ, IFNω or IFNε, or functional fragments thereof.
30. The TNFRSF agonist or functional fragment thereof, the IFN or functional fragment thereof, or the combination for their use according to matter 29, wherein the IFN or functional fragment thereof is IFNα or IFNβ, or a functional fragment thereof.
31. The TNFRSF agonist or functional fragment thereof, the IFN or functional fragment thereof, or the combination for their use according to matter 30, wherein the IFN or functional fragment thereof is IFNα, or a functional fragment thereof.
32. The TNFRSF agonist or functional fragment thereof, the IFN or functional fragment thereof, or the combination for their use according to matter 31, wherein the IFN or functional fragment thereof is IFNα2a, or a functional fragment thereof.
33. The TNFRSF agonist or functional fragment thereof, the IFN or functional fragment thereof, or the combination for their use according to matter 32, wherein the IFNα2a comprises the sequence as set forth in SEQ ID NO 17, or a sequence at least 90%, preferably 95%, more preferably 98%, or still more preferably 99% identical thereto.
34. The TNFRSF agonist or functional fragment thereof, the IFN or functional fragment thereof, or the combination for their use according to matter 30, wherein the IFN or functional fragment thereof is IFNβ, or a functional fragment thereof.
35. The TNFRSF agonist or functional fragment thereof, the IFN or functional fragment thereof, or the combination for their use according to matter 34, wherein the IFNβ comprises the sequence as set forth in SEQ ID NO 14, or a sequence at least 90%, preferably 95%, more preferably 98%, or still more preferably 99% identical thereto.
36. The TNFRSF agonist or functional fragment thereof, the IFN or functional fragment thereof, or the combination for their use according to matter 34, wherein the IFNβ or functional fragment thereof comprises one or two amino acid substitution(s) relative to SEQ ID NO 14, selected from C17S and N80Q.
37. The TNFRSF agonist or functional fragment thereof, the IFN or functional fragment thereof, or the combination for their use according to matter 36, wherein the IFNβ or functional fragment thereof comprises the amino acid substitution C17S relative to SEQ ID NO 14.
38. The TNFRSF agonist or functional fragment thereof, the IFN or functional fragment thereof, or the combination for their use according to matter 37, wherein the IFNβ comprises the amino acid sequence as set forth in SEQ ID NO 15.
39. The TNFRSF agonist or functional fragment thereof, the IFN or functional fragment thereof, or the combination for their use according to matter 36, wherein the IFNβ or functional fragment thereof comprises the amino acid substitutions C17S and N80Q relative to SEQ ID NO 14.
40. The TNFRSF agonist or functional fragment thereof, the IFN or functional fragment thereof, or the combination for their use according to matter 39, wherein the IFNβ comprises the amino acid sequence as set forth in SEQ ID NO 16.
41. The TNFRSF agonist or functional fragment thereof, the IFN or functional fragment thereof, or the combination for their use according to matter 29, wherein the IFN or functional fragment thereof is IFNγ or IFNλ, or a functional fragment thereof.
42. The TNFRSF agonist or functional fragment thereof, the IFN or functional fragment thereof, or the combination for their use according to matter 41, wherein the IFN or functional fragment thereof is IFNγ, or a functional fragment thereof.
43. The TNFRSF agonist or functional fragment thereof, the IFN or functional fragment thereof, or the combination for their use according to matter 42, wherein the IFNγ comprises the sequence as set forth in SEQ ID NO 19, or a sequence at least 90%, preferably 95%, more preferably 98%, or still more preferably 99% identical thereto.
44. The TNFRSF agonist or functional fragment thereof, the IFN or functional fragment thereof, or the combination for their use according to matter 41, wherein the IFN or functional fragment thereof is IFNλ, or a functional fragment thereof.
45. The TNFRSF agonist or functional fragment thereof, the IFN or functional fragment thereof, or the combination for their use according to matter 44, wherein the IFNλ or functional fragment thereof is IFNλ2, or a functional fragment thereof.
46. The TNFRSF agonist or functional fragment thereof, the IFN or functional fragment thereof, or the combination for their use according to matter 45, wherein the IFNλ2 comprises the sequence as set forth in SEQ ID NO 18, or a sequence at least 90%, preferably 95%, more preferably 98%, or still more preferably 99% identical thereto.
47. The TNFRSF agonist or functional fragment thereof, the IFN or functional fragment thereof, or the combination for their use according to any one of matters 1 to 46, wherein the IFN or functional fragment thereof is non-covalently associated with the TNFRSF agonist or functional fragment thereof.
48. The TNFRSF agonist or functional fragment thereof, the IFN or functional fragment thereof, or the combination for their use according to matter 47, wherein the IFN or functional fragment thereof is non-covalently associated with TNFRSF agonist or functional fragment thereof via ionic, Van-der-Waals, and/or hydrogen bond interactions.
49. The TNFRSF agonist or functional fragment thereof, the IFN or functional fragment thereof, or the combination for their use according to any one of matters 1 to 46, wherein the IFN or functional fragment thereof is covalently associated with the TNFRSF agonist or functional fragment thereof.
50. The TNFRSF agonist or functional fragment thereof, the IFN or functional fragment thereof, or the combination for their use according to any one of matters 1 to 46 or 49, wherein the IFN or functional fragment thereof is fused to the TNFRSF agonist or functional fragment thereof, preferably wherein the IFN or functional fragment thereof and the TNFRSF agonist or functional fragment thereof are fused to each other via a linker.
51. The TNFRSF agonist or functional fragment thereof, the IFN or functional fragment thereof, or the combination for their use according to matter 50, wherein the IFN or the functional fragment thereof is fused to an TNFRSF agonist or functional fragment thereof selected from CD70, CD30L (TNFSF8), CD40L, LIGHT, OX40L, TWEAK and 4-1BBL, or functional fragments thereof, preferably wherein the IFN or the functional fragment thereof is fused to an TNFRSF agonist or functional fragment thereof selected from CD40L, LIGHT and TWEAK, or functional fragments thereof.
52. The TNFRSF agonist or functional fragment thereof, the IFN or functional fragment thereof, or the combination for their use according to matter 50, wherein the TNFRSF agonist or functional fragment thereof and the IFN or functional fragment thereof are provided as an interferon-associated antigen binding protein, in which the TNFRSF agonist or functional fragment thereof is an antibody or antigen binding fragment thereof, preferably an agonistic anti-CD40 antibody or agonistic antigen binding fragment thereof, more preferably an agonistic anti-CD40 antibody or agonistic antigen binding fragment thereof comprising
   a. a heavy chain or a fragment thereof comprising a complementarity determining region (CDR) CDRH1 that is identical to SEQ ID NO 56, a CDRH2 that is identical to SEQ ID NO 57, and a CDRH3 that is identical to SEQ ID NO 58; and
   b. a light chain or a fragment thereof comprising a CDRL1 that is identical to SEQ ID NO 52, a CDRL2 that is identical to SEQ ID NO 53, and a CDRL3 that is identical to SEQ ID NO 54;
and wherein the IFN or a functional fragment thereof is fused to said antibody or antigen binding fragment thereof.
53. The TNFRSF agonist or functional fragment thereof, the IFN or functional fragment thereof, or the combination for their use according to matter 52, wherein the IFN or functional fragment thereof is fused to a light chain of the antibody or antigen binding fragment thereof.
54. The TNFRSF agonist or functional fragment thereof, the IFN or functional fragment thereof, or the combination for their use according to matter 53, wherein the IFN or the functional fragment thereof is fused to the N-terminus of the light chain of the antibody or antigen binding fragment thereof.
55. The TNFRSF agonist or functional fragment thereof, the IFN or functional fragment thereof, or the combination for their use according to matter 53, wherein the IFN or the functional fragment thereof is fused to the C-terminus of the light chain of the antibody or antigen binding fragment thereof.
56. The TNFRSF agonist or functional fragment thereof, the IFN or functional fragment thereof, or the combination for their use according to matter 52, wherein the IFN or the functional fragment thereof is fused to a heavy chain of the antibody or antigen binding fragment thereof.
57. The TNFRSF agonist or functional fragment thereof, the IFN or functional fragment thereof, or the combination for their use according to matter 56, wherein the IFN or the functional fragment thereof is fused to the N-terminus of the heavy chain of the antibody or antigen binding fragment thereof.
58. The TNFRSF agonist or functional fragment thereof, the IFN or functional fragment thereof, or the combination for their use according to matter 56, wherein the IFN or the functional fragment thereof is fused to the C-terminus of the heavy chain of the antibody or antigen binding fragment thereof.
59. The TNFRSF agonist or functional fragment thereof, the IFN or functional fragment thereof, or the combination for their use according to any one of matters 50 to 58, wherein the linker is a peptide linker.
60. The TNFRSF agonist or functional fragment thereof, the IFN or functional fragment thereof, or the combination for their use according to matter 59, wherein the linker comprises at least 1, at least 2, at least 3, at least 4, at least 5, at least 10, at least 11, at least 12, at least 13, at least 14, at least 15, at least 20, at least 21 or at least 24 amino acids.
61. The TNFRSF agonist or functional fragment thereof, the IFN or functional fragment thereof, or the combination for their use according to matter 59, wherein the linker comprises up to 4, up to 10, up to 11, up to 12, up to 13, up to 15, up to 20, up to 21, up to 24, up to 30, up to 40, up to 50, up to 60, up to 70, up to 80, up to 90, or up to 100 amino acids.
62. The TNFRSF agonist or functional fragment thereof, the IFN or functional fragment thereof, or the combination for their use according to any one of matters 59 to 61, wherein the linker is selected from the group comprising acidic, basic and neutral linkers.
63. The TNFRSF agonist or functional fragment thereof, the IFN or functional fragment thereof, or the combination for their use according to matter 62, wherein the linker is an acidic linker.
64. The TNFRSF agonist or functional fragment thereof, the IFN or functional fragment thereof, or the combination for their use according to matter 62 or matter 63, wherein the linker comprises a sequence as set forth in SEQ ID NO 22 or SEQ ID NO 23.
65. The TNFRSF agonist or functional fragment thereof, the IFN or functional fragment thereof, or the combination for their use according to matter 62, wherein the linker is a basic linker.
66. The TNFRSF agonist or functional fragment thereof, the IFN or functional fragment thereof, or the combination for their use according to matter 62, wherein the linker is a neutral linker.
67. The TNFRSF agonist or functional fragment thereof, the IFN or functional fragment thereof, or the combination for their use according to matter 62 or matter 66, wherein the linker comprises a sequence as set forth in SEQ ID NO 20, SEQ ID NO 21, SEQ ID NO 24, SEQ ID NO 25 or SEQ ID NO 26.
68. The TNFRSF agonist or functional fragment thereof, the IFN or functional fragment thereof, or the combination for their use according to any one of matters 59 to 67, wherein the linker is selected from the group comprising rigid, flexible and helix-forming linkers.
69. The TNFRSF agonist or functional fragment thereof, the IFN or functional fragment thereof, or the combination for their use according to matter 68, wherein the linker is a rigid linker.
70. The TNFRSF agonist or functional fragment thereof, the IFN or functional fragment thereof, or the combination for their use according to matter 68 or matter 69, wherein the linker comprises a sequence as set forth in SEQ ID NO 20. SEQ ID NO 22 or SEQ ID NO 23.
71. The TNFRSF agonist or functional fragment thereof, the IFN or functional fragment thereof, or the combination for their use according to matter 68, wherein the linker is a flexible linker.
72. The TNFRSF agonist or functional fragment thereof, the IFN or functional fragment thereof, or the combination for their use according to matter 68 or matter 71, wherein the linker comprises a sequence as set forth in SEQ ID NO 21, SEQ ID NO 24, SEQ ID NO 25 or SEQ ID NO 26.
73. The TNFRSF agonist or functional fragment thereof, the IFN or functional fragment thereof, or the combination for their use according to matter 68, wherein the linker is a helix-forming linker.
74. The TNFRSF agonist or functional fragment thereof, the IFN or functional fragment thereof, or the combination for their use according to matter 68 or matter 73, wherein the linker comprises a sequence as set forth in SEQ ID NO 22 or SEQ ID NO 23.
75. The TNFRSF agonist or functional fragment thereof, the IFN or functional fragment thereof, or the combination for their use according to any one of matters 59 to 63, 65, 66, 68, 69, 71 or 73, wherein the linker comprises the amino acids glycine and serine.
76. The TNFRSF agonist or functional fragment thereof, the IFN or functional fragment thereof, or the combination for their use according to matter 75, wherein the linker comprises the sequence as set forth in SEQ ID NO 21, SEQ ID NO 24, SEQ ID NO 25, or SEQ ID NO 26.
77. The TNFRSF agonist or functional fragment thereof, the IFN or functional fragment thereof, or the combination for their use according to matter 75, wherein the linker further comprises the amino acid threonine.
78. The TNFRSF agonist or functional fragment thereof, the IFN or functional fragment thereof, or the combination for their use according to matter 77, wherein the linker comprises the sequence as set forth in SEQ ID NO 21.
79. The TNFRSF agonist or functional fragment thereof, the IFN or functional fragment thereof, or the combination for their use according to matter 59, wherein the linker comprises a sequence selected from the sequences as set forth in SEQ ID NOs 20 to 26.
80. The TNFRSF agonist or functional fragment thereof, the IFN or functional fragment thereof, or the combination for their use according to matter 79, wherein the linker comprises a sequence selected from the sequences as set forth in SEQ ID NO 24, SEQ ID NO 25 or SEQ ID NO 26.
81. The TNFRSF agonist or functional fragment thereof, the IFN or functional fragment thereof, or the combination for their use according to matter 80, wherein the linker comprises a sequence as set forth in SEQ ID NO 24.
82. The TNFRSF agonist or functional fragment thereof, the IFN or functional fragment thereof, or the combination for their use according to matter 80, wherein the linker comprises a sequence as set forth in SEQ ID NO 25.
83. The TNFRSF agonist or functional fragment thereof, the IFN or functional fragment thereof, or the combination for their use according to matter 80, wherein the linker comprises a sequence as set forth in SEQ ID NO 26.
84. The TNFRSF agonist or functional fragment thereof, the IFN or functional fragment thereof, or the combination for their use according to any one of matters 59 to 83, wherein the IFN or functional fragment thereof is fused to the C-terminus of a heavy chain of the agonistic anti-CD40 antibody or the agonistic antigen binding fragment thereof, via the linker as set forth in Table 3, in particular Table 3A or Table 3B, more particularly Table 3A.
85. The TNFRSF agonist or functional fragment thereof, the IFN or functional fragment thereof, or the combination for their use according to matter 84, wherein the heavy chain of the agonistic anti-CD40 antibody, or the agonistic antigen binding fragment thereof, comprises a sequence as set forth in SEQ ID NO 6, SEQ ID NO 9, SEQ ID NO 12, SEQ ID NO 48 or SEQ ID NO 49.
86. The TNFRSF agonist or functional fragment thereof, the IFN or functional fragment thereof, or the combination for their use according to matter 84 or matter 85, wherein the IFNα2a comprises the sequence as set forth in SEQ ID NO 17.
87. The TNFRSF agonist or functional fragment thereof, the IFN or functional fragment thereof, or the combination for their use according to matter 84 or matter 85, wherein the IFNβ comprises the sequence as set forth in SEQ ID NO 14, SEQ ID NO 15 or SEQ ID NO 16.
88. The TNFRSF agonist or functional fragment thereof, the IFN or functional fragment thereof, or the combination for their use according to matter 87, wherein the IFNβ comprises the sequence as set forth in SEQ ID NO 14.
89. The TNFRSF agonist or functional fragment thereof, the IFN or functional fragment thereof, or the combination for their use according to matter 87, wherein the IFNβ comprises the sequence as set forth in SEQ ID NO 15.
90. The TNFRSF agonist or functional fragment thereof, the IFN or functional fragment thereof, or the combination for their use according to matter 87, wherein the IFNβ comprises the sequence as set forth in SEQ ID NO 16.
91. The TNFRSF agonist or functional fragment thereof, the IFN or functional fragment thereof, or the combination for their use according to matter 84 or matter 85, wherein the IFNγ comprises the sequence as set forth in SEQ ID NO 19.
92. The TNFRSF agonist or functional fragment thereof, the IFN or functional fragment thereof, or the combination for their use according to matter 84 or matter 85, wherein the IFNλ2 comprises the sequence as set forth in SEQ ID NO 18.
93. The TNFRSF agonist or functional fragment thereof, the IFN or functional fragment thereof, or the combination for their use according to any one of matters 84 to 92, wherein the agonistic anti-CD40 antibody or the agonistic antigen binding fragment thereof comprises a light chain, preferably wherein the light chain comprises a sequence as set forth in SEQ ID NO 3.
94. The TNFRSF agonist or functional fragment thereof, the IFN or functional fragment thereof, or the combination for their use according to any one of matters 59 to 83, wherein the IFN or functional fragment thereof is fused to the N-terminus of a heavy chain of the agonistic anti-CD40 antibody or the agonistic antigen binding fragment thereof, via the linker as set forth in Table 4, in particular Table 4A or Table 4B, more particularly Table 4A.
95. The TNFRSF agonist or functional fragment thereof, the IFN or functional fragment thereof, or the combination for their use according to matter 94, wherein the heavy chain of the agonistic anti-CD40 antibody or the agonistic antigen binding fragment thereof, comprises a sequence as set forth in SEQ ID NO 6, SEQ ID NO 9, SEQ ID NO 49, SEQ ID NO 48, or SEQ ID NO 12.
96. The TNFRSF agonist or functional fragment thereof, the IFN or functional fragment thereof, or the combination for their use according to matter 94 or matter 95, wherein the IFNα2a comprises the sequence as set forth in SEQ ID NO 17.
97. The TNFRSF agonist or functional fragment thereof, the IFN or functional fragment thereof, or the combination for their use according to matter 94 or matter 95, wherein the IFNβ comprises the sequence as set forth in SEQ ID NO 14, SEQ ID NO 15 or SEQ ID NO 16.
98. The TNFRSF agonist or functional fragment thereof, the IFN or functional fragment thereof, or the combination for their use according to matter 97, wherein the IFNβ comprises the sequence as set forth in SEQ ID NO 14.
99. The TNFRSF agonist or functional fragment thereof, the IFN or functional fragment thereof, or the combination for their use according to matter 97, wherein the IFNβ comprises the sequence as set forth in SEQ ID NO 15.
100. The TNFRSF agonist or functional fragment thereof, the IFN or functional fragment thereof, or the combination for their use according to matter 97, wherein the IFNβ comprises the sequence as set forth in SEQ ID NO 16.
101. The TNFRSF agonist or functional fragment thereof, the IFN or functional fragment thereof, or the combination for their use according to matter 94 or matter 95, wherein the IFNγ comprises the sequence as set forth in SEQ ID NO 19.
102. The TNFRSF agonist or functional fragment thereof, the IFN or functional fragment thereof, or the combination for their use according to matter 94 or matter 95, wherein the IFNλ2 comprises the sequence as set forth in SEQ ID NO 18.
103. The TNFRSF agonist or functional fragment thereof, the IFN or functional fragment thereof, or the combination for their use according to any one of matters 94 to 102, wherein the agonistic anti-CD40 antibody or agonistic antigen binding fragment thereof comprises a light chain, preferably wherein the light chain comprises a sequence as set forth in SEQ ID NO 3.
104. The TNFRSF agonist or functional fragment thereof, the IFN or functional fragment thereof, or the combination for their use according to any one of matters 59 to 83, wherein the IFN or functional fragment thereof is fused to the C-terminus of a light chain of the agonistic anti-CD40 antibody or the agonistic antigen binding fragment thereof, via the linker as set forth in Table 5, in particular Table 5A or Table 5B, more particularly Table 5A.
105. The TNFRSF agonist or functional fragment thereof, the IFN or functional fragment thereof, or the combination for their use according to matter 104, wherein the light chain of the agonistic anti-CD40 antibody or the agonistic antigen binding fragment thereof, comprises a sequence as set forth in SEQ ID NO 3.
106. The TNFRSF agonist or functional fragment thereof, the IFN or functional fragment thereof, or the combination for their use according to matter 104 or 105, wherein the IFNα2a comprises the sequence as set forth in SEQ ID NO 17.
107. The TNFRSF agonist or functional fragment thereof, the IFN or functional fragment thereof, or the combination for their use according to matter 104 or 105, wherein the IFNβ comprises the sequence as set forth in SEQ ID NO 14, SEQ ID NO 15 or SEQ ID NO 16.
108. The TNFRSF agonist or functional fragment thereof, the IFN or functional fragment thereof, or the combination for their use according to matter 107, wherein the IFNβ comprises the sequence as set forth in SEQ ID NO 14.
109. The TNFRSF agonist or functional fragment thereof, the IFN or functional fragment thereof, or the combination for their use according to matter 107, wherein the IFNβ comprises the sequence as set forth in SEQ ID NO 15.
110. The TNFRSF agonist or functional fragment thereof, the IFN or functional fragment thereof, or the combination for their use according to matter 107, wherein the IFNβ comprises the sequence as set forth in SEQ ID NO 16.
111. The TNFRSF agonist or functional fragment thereof, the IFN or functional fragment thereof, or the combination for their use according to matter 104 or 105, wherein the IFNγ comprises the sequence as set forth in SEQ ID NO 19.
112. The TNFRSF agonist or functional fragment thereof, the IFN or functional fragment thereof, or the combination for their use according to matter 104 or 105, wherein the IFNλ2 comprises the sequence as set forth in SEQ ID NO 18.
113. The TNFRSF agonist or functional fragment thereof, the IFN or functional fragment thereof, or the combination for their use according to any one of matters 104 to 112, wherein the agonistic anti-CD40 antibody or the agonistic antigen binding fragment thereof comprises a heavy chain, preferably wherein the heavy chain comprises a sequence as set forth in SEQ ID NO 6, SEQ ID NO 9, SEQ ID NO 49, SEQ ID NO 48, or SEQ ID NO 12.
114. The TNFRSF agonist or functional fragment thereof, the IFN or functional fragment thereof, or the combination for their use according to any one of matters 59 to 83, wherein the IFN or functional fragment thereof is fused to the N-terminus of a light chain of the agonistic anti-CD40 antibody or the agonistic antigen binding fragment thereof, via the linker as set forth in Table 6, in particular Table 6A or Table 6B, more particularly Table 6A.
115. The TNFRSF agonist or functional fragment thereof, the IFN or functional fragment thereof, or the combination for their use according to matter 114, wherein the light chain of the agonistic anti-CD40 antibody or the agonistic antigen binding fragment thereof, comprises a sequence as set forth in SEQ ID NO 3.
116. The TNFRSF agonist or functional fragment thereof, the IFN or functional fragment thereof, or the combination for their use according to matter 114 or matter 115, wherein the IFNα2a comprises the sequence as set forth in SEQ ID NO 17.
117. The TNFRSF agonist or functional fragment thereof, the IFN or functional fragment thereof, or the combination for their use according to matter 114 or matter 115, wherein the IFNβ comprises the sequence as set forth in SEQ ID NO 14, SEQ ID NO 15 or SEQ ID NO 16.
118. The TNFRSF agonist or functional fragment thereof, the IFN or functional fragment thereof, or the combination for their use according to matter 117, wherein the IFNβ comprises the sequence as set forth in SEQ ID NO 14.
119. The TNFRSF agonist or functional fragment thereof, the IFN or functional fragment thereof, or the combination for their use according to matter 117, wherein the IFNβ comprises the sequence as set forth in SEQ ID NO 15.
120. The TNFRSF agonist or functional fragment thereof, the IFN or functional fragment thereof, or the combination for their use according to matter 117, wherein the IFNβ comprises the sequence as set forth in SEQ ID NO 16.
121. The TNFRSF agonist or functional fragment thereof, the IFN or functional fragment thereof, or the combination for their use according to matter 114 or matter 115, wherein the IFNγ comprises the sequence as set forth in SEQ ID NO 19.
122. The TNFRSF agonist or functional fragment thereof, the IFN or functional fragment thereof, or the combination for their use according to matter 114 or matter 115, wherein the IFNλ2 comprises the sequence as set forth in SEQ ID NO 18.
123. The TNFRSF agonist or functional fragment thereof, the IFN or functional fragment thereof, or the combination for their use according to any one of matters 114 to 122, wherein the agonistic anti-CD40 antibody or the agonistic antigen binding fragment thereof comprises a heavy chain, preferably wherein the heavy chain comprises a sequence as set forth in SEQ ID NO 6, SEQ ID NO 9, SEQ ID NO 49, SEQ ID NO 48, or SEQ ID NO 12.
124. The TNFRSF agonist or functional fragment thereof, the IFN or functional fragment thereof, or the combination for their use according to any one of matters 52 to 123, wherein the interferon-associated antigen binding protein comprises a sequence selected from SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 81, SEQ ID NO 82, SEQ ID NO 83, SEQ ID NO 84, SEQ ID NO 85, SEQ ID NO 86, SEQ ID NO 87, SEQ ID NO 88 and SEQ ID NO 94.
125. The TNFRSF agonist or functional fragment thereof, or the IFN or functional fragment thereof, or the combination for their use according to matter 124, wherein the interferon-associated antigen binding protein comprises a sequence selected from SEQ ID NO 38, SEQ ID NO 39, SEQ ID NO 40, SEQ ID NO 41, SEQ ID NO 42 or SEQ ID NO 43.
126. The TNFRSF agonist or functional fragment thereof, or the IFN or functional fragment thereof, or the combination for their use according to matter 124 or matter 125, wherein the interferon-associated antigen binding protein is an interferon-fused agonistic anti-CD40 antibody or interferon-fused agonistic antigen binding fragment thereof comprising one of the sequence combinations disclosed in Table 9, in particular Table 9A or Table 9B, more particularly Table 9A.
127. The TNFRSF agonist or functional fragment thereof, or the IFN or functional fragment thereof, or the combination for their use according to matter 126, wherein the interferon-associated antigen binding protein is an interferon-fused agonistic anti-CD40 antibody or interferon-fused agonistic antigen binding fragment thereof comprising the sequences as set forth in SEQ ID NO 38 and SEQ ID NO 3.
128. The TNFRSF agonist or functional fragment thereof, or the IFN or functional fragment thereof, or the combination for their use according to matter 126, wherein the interferon-associated antigen binding protein is an interferon-fused agonistic anti-CD40 antibody or interferon-fused agonistic antigen binding fragment thereof comprising the sequences as set forth in SEQ ID NO 39 and SEQ ID NO 3.
129. The TNFRSF agonist or functional fragment thereof, or the IFN or functional fragment thereof, or the combination for their use according to matter 126, wherein the interferon-associated antigen binding protein is an interferon-fused agonistic anti-CD40 antibody or interferon-fused agonistic antigen binding fragment thereof comprising the sequences as set forth in SEQ ID NO 40 and SEQ ID NO 3.
130. The TNFRSF agonist or functional fragment thereof, or the IFN or functional fragment thereof, or the combination for their use according to matter 126, wherein the interferon-associated antigen binding protein is an interferon-fused agonistic anti-CD40 antibody or interferon-fused agonistic antigen binding fragment thereof comprising the sequences as set forth in SEQ ID NO 41 and SEQ ID NO 9.
131. The TNFRSF agonist or functional fragment thereof, or the IFN or functional fragment thereof, or the combination for their use according to matter 126, wherein the interferon-associated antigen binding protein is an interferon-fused agonistic anti-CD40 antibody or interferon-fused agonistic antigen binding fragment thereof comprising the sequences as set forth in SEQ ID NO 42 and SEQ ID NO 9.
132. The TNFRSF agonist or functional fragment thereof, or the IFN or functional fragment thereof, or the combination for their use according to matter 126, wherein the interferon-associated antigen binding protein is an interferon-fused agonistic anti-CD40 antibody or interferon-fused agonistic antigen binding fragment thereof comprising the sequences as set forth in SEQ ID NO 43 and SEQ ID NO 9.
133. An interferon-associated antigen binding protein comprising
   (I) an agonistic anti-CD40 antibody or an agonistic antigen binding fragment thereof, and
   (II) an Interferon (IFN) or a functional fragment thereof
for use in the treatment or prevention of a Coronavirus infection.
134. The interferon-associated antigen binding protein for the use of matter 133, wherein the agonistic anti-CD40 antibody or the agonistic antigen binding fragment thereof comprises
   (a) a heavy chain or a fragment thereof comprising a complementarity determining region (CDR) CDRH1 that is at least 90% identical to SEQ ID NO 56, a CDRH2 that is at least 90% identical to SEQ ID NO 57, and a CDRH3 that is at least 90% identical to SEQ ID NO 58; and
   (b) a light chain or a fragment thereof comprising a CDRL1 that is at least 90% identical to SEQ ID NO 52, a CDRL2 that is at least 90% identical to SEQ ID NO 53, and a CDRL3 that is at least 90% identical to SEQ ID NO 54.
135. The interferon-associated antigen binding protein for the use of matter 133, wherein the agonistic anti-CD40 antibody or the agonistic antigen binding fragment thereof comprises
   (a) a heavy chain or a fragment thereof comprising a complementarity determining region (CDR) CDRH1 that is identical to SEQ ID NO 56, a CDRH2 that is identical to SEQ ID NO 57, and a CDRH3 that is identical to SEQ ID NO 58; and
   (b) a light chain or a fragment thereof comprising a CDRL1 that is identical to SEQ ID NO 52, a CDRL2 that is identical to SEQ ID NO 53, and a CDRL3 that is identical to SEQ ID NO 54.
136. The interferon-associated antigen binding protein for the use of any one of matters 133 to 135, wherein the agonistic anti-CD40 antibody, or the agonistic antigen binding fragment thereof, comprises a light chain variable region VL comprising the sequence as set forth in SEQ ID NO 51, or a sequence at least 90% identical thereto; and/or a heavy chain variable region VH comprising the sequence as set forth in SEQ ID NO 55, or a sequence at least 90% identical thereto.
137. The interferon-associated antigen binding protein for the use of any one of matters 133 to 136, wherein the agonistic anti-CD40 antibody or the agonistic antigen binding fragment thereof comprises a light chain (LC) that comprises a sequence as set forth in SEQ ID NO 3, or a sequence at least 90% identical thereto; and/or a heavy chain (HC) that comprises a sequence selected from the group consisting of SEQ ID NO 6, SEQ ID NO 9, SEQ ID NO 12, SEQ ID NO 49 and SEQ ID NO 48, or a sequence at least 90% identical thereto.
138. The interferon-associated antigen binding protein for the use of any one of matters 133 to 137, wherein the IFN or the functional fragment thereof is selected from the group consisting of a Type I IFN, a Type II IFN and a Type III IFN, or a functional fragment thereof.
139. The interferon-associated antigen binding protein for the use of matter 138, wherein the type I IFN or the functional fragment thereof is IFNα or IFNβ, or a functional fragment thereof.
140. The interferon-associated antigen binding protein for the use of any one of matters 133 to 139, wherein the IFN or the functional fragment thereof is IFNα2a, or a functional fragment thereof, and wherein preferably the IFNα2a comprises the sequence as set forth in SEQ ID NO 17, or a sequence at least 90% identical thereto.
141. The interferon-associated antigen binding protein for the use of any one of matters 133 to 139, wherein the IFN or the functional fragment thereof is IFNβ or a functional fragment thereof, and wherein preferably the IFNβ comprises the sequence as set forth in SEQ ID NO 14, or a sequence at least 90% identical thereto.
142. The interferon-associated antigen binding protein for the use of any one of matters 133 to 141, wherein the IFN or the functional fragment thereof is fused to a light chain of the agonistic anti-CD40 antibody or the agonistic antigen binding fragment thereof, preferably to the C-terminus.
143. The interferon-associated antigen binding protein for the use of any one of matters 133 to 141, wherein the IFN or the functional fragment thereof is fused to a heavy chain of the agonistic anti-CD40 antibody or the agonistic antigen binding fragment thereof, preferably to the C-terminus.
144. The interferon-associated antigen binding protein for the use of any one of matters 133 to 143, wherein the agonistic anti-CD40 antibody or an agonistic antigen binding fragment thereof, and the IFN or the functional fragment thereof, are fused to each other via a linker, and wherein preferably the linker comprises a sequence as set forth in SEQ ID NO 20, SEQ ID NO 21, SEQ ID NO 24, SEQ ID NO 25 or SEQ ID NO 26.
145. The interferon-associated antigen binding protein for the use of any one of matters 133 to 144, wherein the interferon-associated antigen binding protein is an interferon-fused agonistic anti-CD40 antibody or an interferon-fused agonistic antigen binding fragment thereof comprising one of the sequence combinations disclosed in Table 9, in particular Table 9A or Table 9B, more particularly Table 9A.
146. The interferon-associated antigen binding protein for the use of any one of matters 133 to 145, wherein the use comprises administering the interferon-associated antigen binding protein to a subject in need thereof by means of genetic delivery with RNA or DNA sequences encoding the interferon-associated antigen binding protein, or a vector or vector system encoding the interferon-associated antigen binding protein.
147. The interferon-associated antigen binding protein for the use of
   (a) any one of matters 133 to 145, wherein the interferon-associated antigen binding protein is comprised in a pharmaceutical composition; or
   (b) matter 146, wherein the RNA or DNA sequences encoding said interferon-associated antigen binding protein, or the vector or vector system encoding said interferon-associated antigen binding protein are comprised in a pharmaceutical composition.
148. An interferon-associated antigen binding protein comprising an interferon-fused agonistic anti-CD40 antibody or interferon-fused agonistic antigen binding fragment thereof comprising the amino acid sequences as set forth in SEQ ID NO 38 and SEQ ID NO 3, for use in the treatment or prevention of a Coronavirus infection.
149. An interferon-associated antigen binding protein comprising an interferon-fused agonistic anti-CD40 antibody or interferon-fused agonistic antigen binding fragment thereof comprising the amino acid sequences as set forth in SEQ ID NO 39 and SEQ ID NO 3, for use in the treatment or prevention of a Coronavirus infection.
150. An interferon-associated antigen binding protein comprising an interferon-fused agonistic anti-CD40 antibody or interferon-fused agonistic antigen binding fragment thereof comprising the amino acid sequences as set forth in SEQ ID NO 40 and SEQ ID NO 3, for use in the treatment or prevention of a Coronavirus infection.
151. An interferon-associated antigen binding protein comprising an interferon-fused agonistic anti-CD40 antibody or interferon-fused agonistic antigen binding fragment thereof comprising the amino acid sequences as set forth in SEQ ID NO 41 and SEQ ID NO 9, for use in the treatment or prevention of a Coronavirus infection.
152. An interferon-associated antigen binding protein comprising an interferon-fused agonistic anti-CD40 antibody or interferon-fused agonistic antigen binding fragment thereof comprising the amino acid sequences as set forth in SEQ ID NO 42 and SEQ ID NO 9, for use in the treatment or prevention of a Coronavirus infection.
153. An interferon-associated antigen binding protein comprising an interferon-fused agonistic anti-CD40 antibody or interferon-fused agonistic antigen binding fragment thereof comprising the amino acid sequences as set forth in SEQ ID NO 43 and SEQ ID NO 9, for use in the treatment or prevention of a Coronavirus infection.
154. The TNFRSF agonist or functional fragment thereof, the IFN or functional fragment thereof, the combination for its use according to any one of matters 1 to 132, or the interferon-associated antigen binding proteins for their use according to any one of matters 133 to 153, wherein the interferon-associated antigen binding protein activates both the CD40 and an IFN pathway.
155. The TNFRSF agonist or functional fragment thereof, the IFN or functional fragment thereof, the combination, or the interferon-associated antigen binding protein for their use according to matter 154, wherein CD40 activity is determined using a whole blood surface molecule upregulation assay or an in vitro reporter cell assay.
156. The TNFRSF agonist or functional fragment thereof, the IFN or functional fragment thereof, the combination, or the interferon-associated antigen binding protein for their use according to matter 155, wherein CD40 activity is determined using an in vitro reporter cell assay, optionally using HEK-Blue™ CD40L cells.
157. The TNFRSF agonist or functional fragment thereof, the IFN or functional fragment thereof, the combination, or the interferon-associated antigen binding protein for their use according to any one of matters 154 to 156, wherein the interferon-associated antigen binding protein activates the CD40 pathway with an EC₅₀ of less than 400, 300, 200, 150, 100, 70, 60, 50, 40, 30, 25, 20, or 15 ng/mL.
158. The TNFRSF agonist or functional fragment thereof, the IFN or functional fragment thereof, the combination, or the interferon-associated antigen binding protein for their use according to matter 157, wherein the interferon-associated antigen binding protein activates the CD40 pathway with an EC₅₀ ranging from 10 to 200 ng/mL.
159. The TNFRSF agonist or functional fragment thereof, the IFN or functional fragment thereof, the combination, or the interferon-associated antigen binding protein for their use according to matter 158, wherein the interferon-associated antigen binding protein activates the CD40 pathway with an EC₅₀ ranging from 10 to 50 ng/mL, preferably 10 to 30 ng/mL.
160. The TNFRSF agonist or functional fragment thereof, the IFN or functional fragment thereof, the combination, or the interferon-associated antigen binding protein for their use according to any one of matters 154 to 159, wherein the interferon-associated antigen binding protein activates the IFN pathway with an EC₅₀ of less than 100, 60, 50, 40, 30, 20, 10, or 1 ng/mL.
161. The TNFRSF agonist or functional fragment thereof, the IFN or functional fragment thereof, the combination, or the interferon-associated antigen binding protein for their use according to any one of matters 154 to 160, wherein the interferon-associated antigen binding protein activates the IFN pathway with an EC₅₀ of less than 11 ng/mL, preferably less than 6 ng/mL.
162. The TNFRSF agonist or functional fragment thereof, the IFN or functional fragment thereof, the combination, or the interferon-associated antigen binding protein for their use according to any one of matters 154 to 161, wherein the IFN pathway is the IFNα, IFNβ, IFNε, IFNγ, IFNω or IFNλ pathway.
163. The TNFRSF agonist or functional fragment thereof, the IFN or functional fragment thereof, the combination, or the interferon-associated antigen binding protein for their use according to matter 162, wherein IFNβ activity is determined using an in vitro reporter cell assay, optionally using HEK-Blue™ IFN-α/β cells.
164. The TNFRSF agonist or functional fragment thereof, the IFN or functional fragment thereof, the combination, or the interferon-associated antigen binding protein for their use according to matter 162, wherein IFNα activity is determined using an in vitro reporter cell assay, optionally using HEK-Blue™ IFN-α/β cells.
165. The TNFRSF agonist or functional fragment thereof, the IFN or functional fragment thereof, the combination, or the interferon-associated antigen binding protein for their use according to matter 162, wherein IFNγ activity is determined using an in vitro reporter cell assay, optionally using HEK-Blue™ Dual IFN-γ cells.
166. The TNFRSF agonist or functional fragment thereof, the IFN or functional fragment thereof, the combination, or the interferon-associated antigen binding protein for their use according to matter 162, wherein IFNλ activity is determined using an in vitro reporter cell assay, optionally using HEK-Blue™ IFN-λ cells.
167. The TNFRSF agonist or functional fragment thereof, the IFN or functional fragment thereof, the combination, or the interferon-associated antigen binding protein for their use according to any one of matters 1 to 166, wherein the expression level of one or more IFN pathway biomarkers is upregulated in a Coronavirus-infected cell upon treatment with the interferon-associated antigen binding protein, preferably at least 1.5-fold, more preferably at least 2-fold, most preferably at least 3-fold, as compared to the expression level of said biomarkers in said Coronavirus-infected cell that has not been treated with the interferon-associated antigen binding protein.
168. The TNFRSF agonist or functional fragment thereof, the IFN or functional fragment thereof, the combination, or the interferon-associated antigen binding protein for their use according to matter 167, wherein the IFN pathway biomarker is a chemokine.
169. The TNFRSF agonist or functional fragment thereof, the IFN or functional fragment thereof, the combination, or the interferon-associated antigen binding protein for their use according to matter 168, wherein the IFN pathway biomarker is the interferon stimulated gene ISG20.
170. The TNFRSF agonist or functional fragment thereof, the IFN or functional fragment thereof, the combination, or the interferon-associated antigen binding protein for their use according to matter 168, wherein the IFN pathway biomarker is a C-X-C chemokine, selected from the group consisting of CXCL9, CXCL10 and CXCL11.
171. The TNFRSF agonist or functional fragment thereof, the IFN or functional fragment thereof, the combination, or the interferon-associated antigen binding protein for their use according to matter 170, wherein the IFN pathway biomarker is CXCL10.
172. The TNFRSF agonist or functional fragment thereof, the IFN or functional fragment thereof, the combination, or the interferon-associated antigen binding protein for their use according to any one of matters 1 to 171, wherein the expression level of one or more of IL10, IL1β and IL2 is not significantly upregulated in an Coronavirus-infected cell upon treatment with the interferon-associated antigen binding protein, as compared to the expression level of said interleukins in said Coronavirus-infected cell that has not been treated with the interferon-associated antigen binding protein.
173. The TNFRSF agonist or functional fragment thereof, the IFN or functional fragment thereof, the combination, or the interferon-associated antigen binding protein for their use according to any one of the matters 1 to 172, wherein the systemic exposure of the interferon-associated antigen binding protein is increased compared to antibody CP870,893, preferably by at least 10%, more preferably by at least 15%, most preferably by at least 25%.
174. The TNFRSF agonist or functional fragment thereof, the IFN or functional fragment thereof, the combination, or the interferon-associated antigen binding protein for their use according to any one of matters 52 to 173, wherein the systemic exposure of the interferon-associated antigen binding protein is at least 1000 μg*h/mL.
175. The TNFRSF agonist or functional fragment thereof, the IFN or functional fragment thereof, the combination, or the interferon-associated antigen binding protein for their use according to matter 174, wherein the systemic exposure of the interferon-associated antigen binding protein ranges from 1033 μg*h/mL to 1793 μg*h/mL.
176. The TNFRSF agonist or functional fragment thereof, the IFN or functional fragment thereof, the combination, or the interferon-associated antigen binding protein for their use according to any one of matters 52 to 175, wherein the half-life of the interferon-associated antigen binding protein is at least 100 h.
177. The TNFRSF agonist or functional fragment thereof, the IFN or functional fragment thereof, the combination, or the interferon-associated antigen binding protein for their use according to matter 176, wherein the half-life of the interferon-associated antigen binding protein ranges from 116 to 158 h.
178. The TNFRSF agonist or functional fragment thereof, the IFN or functional fragment thereof, the combination, or the interferon-associated antigen binding protein for their use according to any one of matters 52 to 177, wherein the clearance rate of the interferon-associated antigen binding protein is below 0.5 mL/h/kg.
179. The TNFRSF agonist or functional fragment thereof, the IFN or functional fragment thereof, the combination, or the interferon-associated antigen binding protein for their use according to matter 178, wherein the clearance of the interferon-associated antigen binding protein ranges from 0.28 to 0.49 mL/h/kg.
180. The TNFRSF agonist or functional fragment thereof, the IFN or functional fragment thereof, the combination, or the interferon-associated antigen binding protein for their use according to any one of matters 1 to 179, wherein the volume of distribution Vss of the interferon-associated antigen binding protein is below 100 mL/kg.
181. The TNFRSF agonist or functional fragment thereof, the IFN or functional fragment thereof, the combination, or the interferon-associated antigen binding protein for their use according to matter 180, wherein the volume of distribution Vss of the interferon-associated antigen binding protein ranges from 50 to 98 mL/kg.
182. The TNFRSF agonist or functional fragment thereof, the IFN or functional fragment thereof, the combination, or the interferon-associated antigen binding protein for their use according to any one of the preceding matters, wherein the use comprises administering said TNFRSF agonist or functional fragment thereof, said IFN or functional fragment thereof, said combination or said interferon-associated antigen binding protein to a subject in need thereof by means of
   a. genetic delivery with RNA or DNA sequences encoding said TNFRSF agonist or functional fragment thereof, said IFN or functional fragment thereof, said combination or said interferon-associated antigen binding protein; or
   b. a vector or vector system encoding said TNFRSF agonist or functional fragment thereof, said IFN or functional fragment thereof, said combination or said interferon-associated antigen binding protein.
183. The TNFRSF agonist or functional fragment thereof, the IFN or functional fragment thereof, the combination, or the interferon-associated antigen binding protein for their use according to matter 182, wherein said interferon-associated antigen binding protein is expressed in said subject from a polynucleotide or polynucleotides administered to said subject.
184. The TNFRSF agonist or functional fragment thereof, the IFN or functional fragment thereof, the combination, or the interferon-associated antigen binding protein for their use according to any one of matters 1 to 183, wherein the interferon-associated antigen binding protein is comprised in a pharmaceutical composition.
185. The TNFRSF agonist or functional fragment thereof, the IFN or functional fragment thereof, or the combination for their use according to any one of matters 1 to 184, wherein the TNFRSF agonist or the functional fragment thereof and the IFN or the functional fragment thereof are comprised in the same pharmaceutical composition or in distinct pharmaceutical compositions.
186. The TNFRSF agonist or functional fragment thereof, the IFN or functional fragment thereof, the combination, or the interferon-associated antigen binding protein for their use according to matter 184 or matter 185, wherein the pharmaceutical composition is suitable for oral, parenteral, or topical administration or for administration by inhalation.
187. The TNFRSF agonist or functional fragment thereof, the IFN or functional fragment thereof, the combination, or the interferon-associated antigen binding protein for their use according to matter 186, wherein the pharmaceutical composition is suitable for oral administration.
188. The TNFRSF agonist or functional fragment thereof, the IFN or functional fragment thereof, the combination, or the interferon-associated antigen binding protein for their use according to matter 186, wherein the pharmaceutical composition is suitable for topical administration.
189. The TNFRSF agonist or functional fragment thereof, the IFN or functional fragment thereof, the combination, or the interferon-associated antigen binding protein for their use according to matter 186, wherein the pharmaceutical composition is suitable for administration by inhalation.
190. The TNFRSF agonist or functional fragment thereof, the IFN or functional fragment thereof, the combination, or the interferon-associated antigen binding protein for their use according to matter 186, wherein the pharmaceutical composition is suitable for parenteral administration.
191. The TNFRSF agonist or functional fragment thereof, the IFN or functional fragment thereof, the combination, or the interferon-associated antigen binding protein for their use according to matter 184 or matter 185, wherein the pharmaceutical composition is suitable for intravenous, intraarterial, intraperitoneal, intramuscular, subcutaneous, rectal or vaginal administration.
192. The TNFRSF agonist or functional fragment thereof, the IFN or functional fragment thereof, the combination, or the interferon-associated antigen binding protein for their use according to matter 184 or matter 185, wherein the pharmaceutical composition is suitable for injection, preferably for intravenous or intraarterial injection or drip.
193. The TNFRSF agonist or functional fragment thereof, the IFN or functional fragment thereof, the combination, or the interferon-associated antigen binding protein for their use according to any one of matters 184 to 192, wherein the pharmaceutical composition comprises at least one buffering agent.
194. The TNFRSF agonist or functional fragment thereof, the IFN or functional fragment thereof, the combination, or the interferon-associated antigen binding protein for their use according to matter 193, wherein the buffering agent is acetate, formate or citrate.
195. The TNFRSF agonist or functional fragment thereof, the IFN or functional fragment thereof, the combination, or the interferon-associated antigen binding protein for their use according to matter 194, wherein the buffering agent is acetate.
196. The TNFRSF agonist or functional fragment thereof, the IFN or functional fragment thereof, the combination, or the interferon-associated antigen binding protein for their use according to matter 194, wherein the buffering agent is formate.
197. The TNFRSF agonist or functional fragment thereof, the IFN or functional fragment thereof, the combination, or the interferon-associated antigen binding protein for their use according to matter 194, wherein the buffering agent is citrate.
198. The TNFRSF agonist or functional fragment thereof, the IFN or functional fragment thereof, the combination, or the interferon-associated antigen binding protein for their use according to any one of matters 184 to 197, wherein the pharmaceutical composition comprises a surfactant.
199. The TNFRSF agonist or functional fragment thereof, the IFN or functional fragment thereof, the combination, or the interferon-associated antigen binding protein for their use according to matter 198, wherein the surfactant is selected from the list comprising pluronics, PEG, sorbitan esters, polysorbates, triton, tromethamine, lecithin, cholesterol and tyloxapal.
200. The TNFRSF agonist or functional fragment thereof, the IFN or functional fragment thereof, the combination, or the interferon-associated antigen binding protein for their use according to matter 199, wherein the surfactant is polysorbate.
201. The TNFRSF agonist or functional fragment thereof, the IFN or functional fragment thereof, the combination, or the interferon-associated antigen binding protein for their use according to matter 200, wherein the surfactant is polysorbate 20, polysorbate 40, polysorbate 60, polysorbate 80 or polysorbate 100.
202. The TNFRSF agonist or functional fragment thereof, the IFN or functional fragment thereof, the combination, or the interferon-associated antigen binding protein for their use according to matter 201, wherein the surfactant is polysorbate 20.
203. The TNFRSF agonist or functional fragment thereof, the IFN or functional fragment thereof, the combination, or the interferon-associated antigen binding protein for their use according to matter 201, wherein the surfactant is polysorbate 80.
204. The TNFRSF agonist or functional fragment thereof, the IFN or functional fragment thereof, the combination, or the interferon-associated antigen binding protein for their use according to any one of matters 184 to 203, wherein the pharmaceutical composition comprises a stabilizing agent, optionally wherein the stabilizing agent is albumin.
205. The interferon-associated antigen binding protein for its use according to any one of matters 133 to 204, wherein the agonistic anti-CD40 antibody or agonistic antigen binding fragment thereof comprises three light chain complementarity determining regions (CDRs) CDRL1, CDRL2 and CDRL3 that are at least 90% identical to the respective CDRL1, CDRL2 and CDRL3 sequences within SEQ ID NO 3; and three heavy chain CDRs, CDRH1, CDRH2 and CDRH3, that are at least 90% identical to the respective CDRH1, CDRH2 and CDRH3 sequences within SEQ ID NO 6.
206. The interferon-associated antigen binding protein for its use according to any one of matters 133 to 205, wherein the agonistic anti-CD40 antibody or agonistic antigen binding fragment thereof comprises three light chain complementarity determining regions (CDRs) CDRL1, CDRL2 and CDRL3 that are identical to the respective CDRL1, CDRL2 and CDRL3 sequences within SEQ ID NO 3; and three heavy chain CDRs, CDRH1, CDRH2 and CDRH3, that are identical to the respective CDRH1, CDRH2 and CDRH3 sequences within SEQ ID NO 6.
207. The interferon-associated antigen binding protein for its use according to matter 205 or matter 206, wherein each CDR is defined in accordance with the Kabat definition, the Chothia definition, the AbM definition, or the contact definition of CDR; preferably wherein each CDR is defined in accordance with the CDR definition of Kabat or the CDR definition of Chothia.
208. The interferon-associated antigen binding protein for its use according to any one of matters 133 to 207, wherein no amino acid substitutions, insertions or deletions are present within the complementarity determining regions of the heavy chain and light chain variable regions of the agonistic anti-CD40 antibody or agonistic antigen binding fragment thereof.
209. The interferon-associated antigen binding protein for its use according to any one of matters 133 to 207, wherein no, one or two amino acid substitutions, insertions or deletions are independently present within each of the complementarity determining regions of the heavy chain and light chain variable regions of the agonistic anti-CD40 antibody or agonistic antigen binding fragment thereof.
210. A polynucleotide or polynucleotides encoding the TNFRSF agonist or functional fragment thereof, the IFN or functional fragment thereof or the interferon-associated antigen binding protein as defined in any one of the preceding matters for use in the treatment or prevention of a Coronavirus infection in a subject in need thereof, wherein the treatment comprises expression in said subject of said TNFRSF agonist or functional fragment thereof and said IFN or functional fragment thereof, or expression in said subject of said interferon-associated antigen binding protein, from a polynucleotide or polynucleotides administered to said subject.
211. The TNFRSF agonist or functional fragment thereof, the IFN or functional fragment thereof, the combination, the interferon-associated antigen binding protein, or the polynucleotide or polynucleotides for their use according to any one of the preceding matters, wherein the Coronavirus is selected from the group consisting of severe acute respiratory syndrome coronavirus 1 (SARS-CoV-1), severe acute respiratory syndrome coronavirus 2 (SARS-CoV-2) and Middle East respiratory syndrome coronavirus (MERS-CoV).
212. The TNFRSF agonist or functional fragment thereof, the IFN or functional fragment thereof, the combination the interferon-associated antigen binding protein, or the polynucleotide or polynucleotides for their use according any one of the preceding matters, wherein the Coronavirus is SARS-CoV-2 or a variant thereof.
213. The TNFRSF agonist or functional fragment thereof, the IFN or functional fragment thereof, the combination the interferon-associated antigen binding protein, or the polynucleotide or polynucleotides for their use according to matter 212, wherein the Coronavirus is a variant of SARS-CoV-2 as identified in the lineage reports available from https://outbreak.info/situation-reports or https://cov-lineages.org/global_report.html.
214. The TNFRSF agonist or functional fragment thereof, the IFN or functional fragment thereof, the combination the interferon-associated antigen binding protein, or the polynucleotide or polynucleotides for their use according to matter 212 or 213, wherein the SARS-CoV-2 variant is selected from the group consisting of A.23.1, B.1.1.7, B.1.351, B.1.427, B.1.429, B.1.525, B.1.526, B.1.526.1, B.1.526.2, B.1.617, B.1.617.1, B.1.617.2, B.1.617.3, P.1 and P.2.

It will be understood by one of skill in the art that the present invention is also directed to a TNFRSF agonist or functional fragment thereof, an IFN or functional fragment thereof, a combination of the two, an interferon-associated antigen binding protein or a polynucleotide or polynucleotides for their use according to any one of the matters or items identified herein, wherein the TNFRSF agonist comprises SEQ ID NO 59, SEQ ID NO 61 or SEQ ID NO 63 as disclosed in Table 8 and/or wherein the interferon-associated binding protein is an interferon-fused agonistic anti-CD40 antibody comprising one of the sequence combinations disclosed in Table 10, or an interferon-fused agonistic antigen binding fragment thereof.

Furthermore, it will be understood by one of skill in the art that the present invention is furthermore also directed to methods for the treatment or prevention of a Coronavirus infection in a subject, in particular a Coronavirus infection as identified in matters 211 to 214, wherein a therapeutically effective amount of the TNFRSF agonist or functional fragment thereof, the IFN or functional fragment thereof, the combination of the two, the interferon-associated antigen binding protein or the polynucleotide or polynucleotides as referred to in any one of the aspects, embodiments, matters or items identified herein, or in the preceding paragraph, is administered to said subject.

It will be understood that in accordance with all aspects, embodiments, items and matters described and claimed herein, the subject to be treated is preferably a mammal, and most preferably a human subject.

Finally, it will be understood that all aspects, embodiments, items and matters described herein may be made the subject matter of further claims (in addition to the claims provided below).

1. A combination of a tumor necrosis factor receptor superfamily (TNFRSF) agonist or a functional fragment thereof and an interferon (IFN) or a functional fragment thereof, for use in the treatment or prevention of a Coronavirus infection.

2. An interferon-associated antigen binding protein comprising
(I) an agonistic anti-CD40 antibody or an agonistic antigen binding fragment thereof, and
(II) an Interferon (IFN) or a functional fragment thereof
for use in the treatment or prevention of a Coronavirus infection.

3. The interferon-associated antigen binding protein for the use of claim 2, wherein the agonistic anti-CD40 antibody or the agonistic antigen binding fragment thereof comprises
(a) a heavy chain or a fragment thereof comprising a complementarity determining region (CDR) CDRH1 that is at least 90% identical to SEQ ID NO 56, a CDRH2 that is at least 90% identical to SEQ ID NO 57, and a CDRH3 that is at least 90% identical to SEQ ID NO 58; and
(b) a light chain or a fragment thereof comprising a CDRL1 that is at least 90% identical to SEQ ID NO 52, a CDRL2 that is at least 90% identical to SEQ ID NO 53, and a CDRL3 that is at least 90% identical to SEQ ID NO 54.

4. The interferon-associated antigen binding protein for the use of claim 2, wherein the agonistic anti-CD40 antibody or the agonistic antigen binding fragment thereof comprises
(a) a heavy chain or a fragment thereof comprising a complementarity determining region (CDR) CDRH1 that is identical to SEQ ID NO 56, a CDRH2 that is identical to SEQ ID NO 57, and a CDRH3 that is identical to SEQ ID NO 58; and
(b) a light chain or a fragment thereof comprising a CDRL1 that is identical to SEQ ID NO 52, a CDRL2 that is identical to SEQ ID NO 53, and a CDRL3 that is identical to SEQ ID NO 54.

5. The interferon-associated antigen binding protein for the use of claim 2, wherein the agonistic anti-CD40 antibody, or the agonistic antigen binding fragment thereof, comprises a light chain variable region V_{L} comprising the sequence as set forth in SEQ ID NO 51, or a sequence at least 90% identical thereto; and/or a heavy chain variable region V_{H} comprising the sequence as set forth in SEQ ID NO 55, or a sequence at least 90% identical thereto.

6. The interferon-associated antigen binding protein for the use of claim 2, wherein the agonistic anti-CD40 antibody or the agonistic antigen binding fragment thereof comprises a light chain (LC) that comprises a sequence as set forth in SEQ ID NO 3, or a sequence at least 90% identical thereto; and/or a heavy chain (HC) that comprises a sequence selected from the group consisting of SEQ ID NO 6, SEQ ID NO 9, SEQ ID NO 49 and SEQ ID NO 48, or a sequence at least 90% identical thereto.

7. The interferon-associated antigen binding protein for the use of claim 2, wherein the IFN or the functional fragment thereof is selected from the group consisting of a Type I IFN, a Type II IFN and a Type III IFN, or a functional fragment thereof.

8. The interferon-associated antigen binding protein for the use of claim 7, wherein the type I IFN or the functional fragment thereof is IFNα or IFNβ, or a functional fragment thereof.

9. The interferon-associated antigen binding protein for the use of claim 2, wherein the IFN or the functional fragment thereof is IFNα2a, or a functional fragment thereof, and wherein preferably the IFNα2a comprises the sequence as set forth in SEQ ID NO 17, or a sequence at least 90% identical thereto.

10. The interferon-associated antigen binding protein for the use of claim 2, wherein the IFN or the functional fragment thereof is IFNβ, or a functional fragment thereof, and wherein preferably the IFNβ comprises the sequence as set forth in SEQ ID NO 14, or a sequence at least 90% identical thereto.

11. The interferon-associated antigen binding protein for the use of claim 2, wherein the IFN or the functional fragment thereof is fused to a light chain of the agonistic anti-CD40 antibody or the agonistic antigen binding fragment thereof, preferably to the C-terminus.

12. The interferon-associated antigen binding protein for the use of claim 2, wherein the IFN or the functional fragment thereof is fused to a heavy chain of the agonistic anti-CD40 antibody or the agonistic antigen binding fragment thereof, preferably to the C-terminus.

13. The interferon-associated antigen binding protein for the use of claim 2, wherein the agonistic anti-CD40 antibody or an agonistic antigen binding fragment thereof, and the IFN or the functional fragment thereof, are fused to each other via a linker, and wherein preferably the linker comprises a sequence as set forth in SEQ ID NO 20, SEQ ID NO 21, SEQ ID NO 24, SEQ ID NO 25 or SEQ ID NO 26.

14. The interferon-associated antigen binding protein for the use of claim 2, wherein the interferon-associated antigen binding protein is an interferon-fused agonistic anti-CD40 antibody or an interferon-fused agonistic antigen binding fragment thereof comprising one of the sequence combinations disclosed in Table 9, in particular Table 9A or Table 9B, more particularly Table 9A.

15. The interferon-associated antigen binding protein for the use of claim 2, wherein the use comprises administering the interferon-associated antigen binding protein to a subject in need thereof by means of genetic delivery with RNA or DNA sequences encoding the interferon-associated antigen binding protein, or a vector or vector system encoding the interferon-associated antigen binding protein.

16. The interferon-associated antigen binding protein for the use of claim 2, wherein the interferon-associated antigen binding protein is comprised in a pharmaceutical composition.
